SEQUENCE LISTING (88 sequences)

SEQ ID NO: 1 - protein, 379 aa, Homo sapiens [sequence omitted]

SEQ ID NO: 2 - protein, 379 aa, Homo sapiens
  REGION 1..379: note = hSTING[H]wt
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 3 - protein, 379 aa, Homo sapiens [sequence omitted]

SEQ ID NO: 4 - protein, 283 aa, Homo sapiens
  REGION 1..283: note = hSTING[H]truncated
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRNLQMTAASRCPRRFSGTCGRRKRKRLLWAA

SEQ ID NO: 5 - DNA, 1139 bp, Homo sapiens
  misc_feature 1..1139: note = hSTING[R]-S162A/G230I/Q266I (hSTINGmut)
  source 1..1139: mol_type = genomic DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatgggcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccattgaccgtgctggcatcaaggatcgggtttacagcaacagcatctatgagcttctggagaacgggcagcgggcgggcacctgtgtcctggagtacgccacccccttgatcactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctctta

SEQ ID NO: 6 - protein, 379 aa, Homo sapiens
  REGION 1..379: note = hSTING[R]-S162A/G230I/Q266I (hSTINGmut)
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWAYYIGYLRLILPELQARIRTYNQHYNNLLRGAVSQRLYILLPLDCGVPDNLSMADPNIRFLDKLPQQTIDRAGIKDRVYSNSIYELLENGQRAGTCVLEYATPLITLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 7 - DNA, 1137 bp, Mus musculus
  misc_feature 1..1137: note = mSTINGwt
  source 1..1137: mol_type = genomic DNA
atgccatactccaacctgcatccagccatcccacggcccagaggtcaccgctccaaatatgtagccctcatctttctggtggccagcctgatgatcctttgggtggcaaaggatccaccaaatcacactctgaagtacctagcacttcacctagcctcgcacgaacttggactactgttgaaaaacctctgctgtctggctgaagagctgtgccatgtccagtccaggtaccagggcagctactggaaggctgtgcgcgcctgcctgggatgccccatccactgtatggctatgattctactatcgtcttatttctatttcctccaaaacactgctgacatatacctcagttggatgtttggccttctggtcctctataagtccctaagcatgctcctgggccttcagagcttgactccagcggaagtctctgcagtctgtgaagaaaagaagttaaatgttgcccacgggctggcctggtcatactacattgggtacttgcggttgatcttaccagggctccaggcccggatccgaatgttcaatcagctacataacaacatgctcagtggtgcagggagccgaagactgtacatcctctttccattggactgtggggtgcctgacaacctgagtgtagttgaccccaacattcgattccgagatatgctgccccagcaaaacatcgaccgtgctggcatcaagaatcgggtttattccaacagcgtctacgagattctggagaacggacagccagcaggcgtctgtatcctggagtacgccacccccttgcagaccctgtttgccatgtcacaggatgccaaagctggcttcagtcgggaggatcggcttgagcaggctaaactcttctgccggacacttgaggaaatcctggaagatgtccccgagtctcgaaataactgccgcctcattgtctaccaagaacccacagacggaaacagtttctcactgtctcaggaggtgctccggcacattcgtcaggaagaaaaggaggaggttaccatgaatgcccccatgacctcagtggcacctcctccctccgtactgtcccaagagccaagactcctcatcagtggtatggatcagcctctcccactccgcactgacctcatctga

SEQ ID NO: 8 - protein, 378 aa, Mus musculus
  REGION 1..378: note = mSTINGwt
MPYSNLHPAIPRPRGHRSKYVALIFLVASLMILWVAKDPPNHTLKYLALHLASHELGLLLKNLCCLAEELCHVQSRYQGSYWKAVRACLGCPIHCMAMILLSSYFYFLQNTADIYLSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 9 - protein, 540 aa, Homo sapiens
  REGION 1..540: note = hIL-12sc
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 543 aa, Mus musculus
  REGION 1..543: note = mIL-12sc
MGPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 11 - DNA, 13 bp, synthetic construct
  misc_feature 1..13: note = ISRE
  source 1..13: mol_type = other DNA
agtttcgctttct

SEQ ID NO: 12 - DNA, 212 bp, synthetic construct
  misc_feature 1..212: note = ISREx4
  source 1..212: mol_type = other DNA
atctctgcgatgaacctcacagtttcgctttctatctctgcgatgaacctcacagtttcgctttctatctctgcgatgaacctcacagtttcgctttctatctctgcgatgaacctcacagtttcgctttctgtgcctcttatgatctggatctcgagccatggtcgaggcctgtaggcgtgtacggtgggaggcttatataagcagagctc

SEQ ID NO: 13 - DNA, 156 bp, synthetic construct
  misc_feature 1..156: note = ISREx4 short
  source 1..156: mol_type = other DNA
gctagcagtttcgctttctgctagcagtttcgctttctgctagcagtttcgctttctgctagcagtttcgctttctgtgcctcttatgatctggatctcgagccatggtcgaggcctgtaggcgtgtacggtgggaggcttatataagcagagctc

SEQ ID NO: 14 - DNA, 22 bp, synthetic construct
  misc_feature 1..22: note = miR122 target sequence
  source 1..22: mol_type = other DNA
caaacaccattgtcacactcca

SEQ ID NO: 15 - DNA, 130 bp, synthetic construct
  misc_feature 1..130: note = miR122 target sequence x5
  source 1..130: mol_type = other DNA
atccaaacaccattgtcacactccaagaccaaacaccattgtcacactccaagaccaaacaccattgtcacactccaagaccaaacaccattgtcacactccaagaccaaacaccattgtcacactccac

SEQ ID NO: 16 - protein, 376 aa, Human alphaherpesvirus 1
  REGION 1..376: note = thymidine kinase
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 17 - DNA, 103 bp, Adeno-associated dependoparvovirus A
  misc_feature 1..103: note = AAV ITR
  source 1..103: mol_type = genomic DNA
catcatcaataatataccttattttggattgaagccaatatgataatgagggggtggagtttgtgacgtggcgcggggcgtgggaacggggcgggtgacgtag

SEQ ID NO: 18 - DNA, 219 bp, synthetic construct
  misc_feature 1..219: note = short IFNb A-promoter
  source 1..219: mol_type = other DNA
caggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgcaggtagcagccgacaccagcctggcttccatc

SEQ ID NO: 19 - DNA, 219 bp, synthetic construct
  misc_feature 1..219: note = short IFNb B-promoter
  source 1..219: mol_type = other DNA
caggagcttgaataaaatgaatattagaagctgttagaataagagaaaatgacagaggaaaactgaaagggagaactgaaagcaaaaaattcctctgaggcagaaaggaccatcccttataaatagcacaggccatgaaggaagatcattctcactgcagcctttgacagcctttgcctcatcttgcaggtagcagccgacaccagcctggcttccatc

SEQ ID NO: 20 - DNA, 205 bp, synthetic construct
  misc_feature 1..205: note = short IFNb C-promoter
  source 1..205: mol_type = other DNA
caggagcttgaataaaatgaatattagaagctgttagaataagagaaaatgacagaggaaaactgaaagggagaactgaaagtgggcagaaaggaccatcccttataaatagcacaggccatgaaggaagatcattctcactgcagcctttgacagcctttgcctcatcttgcaggtagcagccgacaccagcctggcttccatc

SEQ ID NO: 21 - DNA, 185 bp, synthetic construct
  misc_feature 1..185: note = short IFNb D-promoter
  source 1..185: mol_type = other DNA
caggagcttgaataaaatgaatattagaagctgttagaataagagaaaatgacagaggaaaactgaaagggagaactgaaagtgggaaattcctctgaggcagaaaggaccatattctcactgcagcctttgacagcctttgcctcatcttgcaggtagcagccgacaccagcctggcttccatc

SEQ ID NO: 22 - DNA, 110 bp, synthetic construct
  misc_feature 1..110: note = short IFNb E-promoter
  source 1..110: mol_type = other DNA
ggagaactgaaagtgggaaattcctctgaggcagaaagattctcactgcagcctttgacagcctttgcctcatcttgcaggtagcagccgacaccagcctggcttccatc

SEQ ID NO: 23 - DNA, 556 bp, Mus musculus
  misc_feature 1..556: note = CXCL9-promoter
  source 1..556: mol_type = genomic DNA
gacacagacatctcgtgaaacatctggaaaaggtagcagggaacgggacagcagtagcttatgcaactaatctagcaattacaatgataggaaactgtagccacggtgagacgaaatggatttaccctcaacaaattaagaagctttgacttgtgaggaaaggcagtttgagtcgccatatagtgtcatgtcccacgtgggaagtacatctccaaccttcattgtacaacattaatatagtcattgtataatctattccacatccaggtagcaactttgcctgggggtggtttcacatcccttactataaactccccgtttatgtgaaatggaagtagaacatgcagaaattccctgggatctgagagtagggttttccccaggacgatcaatttgtggttagtttagttcttctaggtcagctgaggagaccagccaatcagagacgggaaggaaaagggatttcctaaataaatatgatccccaagaacatgctctctaaagacattctcggacttcactccaacacagtgactcaatagaactcagctctgcc

SEQ ID NO: 24 - DNA, 565 bp, Mus musculus
  misc_feature 1..565: note = CXCL10-promoter
  source 1..565: mol_type = genomic DNA
tctaactgttcactaagttgtatagactttggatctggctaaatttggcgtgtgatttttttttaaagtttattccaagtattttataataaagcatatgaagtaaaaagcaaaaataaacagtgtaaacacaacaaatgtaaaaaccatggttagaacctgacttagatatcagttctgtgttttatttatgagagaaggaagatgagaattaaagccatttccagacttattctgcaaggcactgcatctgatttctcaaacagctcacgctttggaaagtgaaacttacctcactcgttaaaaattaaaaggagcacaagaggggagagggaaattccaagttcatgggtcacaataaacacaagcaatgccctcggtttacaggggacttccctcgggttgcggagccttgctgagtcatctccaaagtcagccaatcaggactcagggagggaaactctttgcagataaatactcctcagcagccggcactcgagaagcgcttcatccaccgctgagagacatcccgagccaaccttccggaagcctccccatcagcacc

SEQ ID NO: 25 - DNA, 571 bp, Mus musculus
  misc_feature 1..571: note = IFNb1-promoter
  source 1..571: mol_type = genomic DNA
ccaggatgtggggttctctcccaaagggagctatggcccattgatgtgccatttctcttatagtacactattgtaagccccctgggagctcacagagaccctctcccaccatcccactatttcaaagggcagcagctgtgccatcctttcaggatgagcagctactctgcctggcttttcagtggacactttggctgtttgagagttcttttatcttcagggctgtctcctttctgttcttctctcctggatatttctcttcctttgctccagcaattggtgaaactgtacaagattttataaatccttagtttgtatatattttaacccagtacatagcatataaaatagccaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgcaggtagcagccgacaccagcctggcttccatc

SEQ ID NO: 26 - DNA, 202 bp, synthetic construct
  misc_feature 1..202: note = STATx4
  source 1..202: mol_type = other DNA
atctctgcgatgaacctcacttcgctagaaatctctgcgatgaacctcacttcgctagaaatatctctgcgatgaacctcacttcgctagaaatctctgcgatgaacctcacttcgctagaagtgcctcttatgatctggatctcgagccatggtcgaggcctgtaggcgtgtacggtgggaggcttatataagcagagctc

SEQ ID NO: 27 - DNA, 208 bp, synthetic construct
  misc_feature 1..208: note = IRSF3x4
  source 1..208: mol_type = other DNA
atctctgcgatgaacctcacgaaatagaaactatctctgcgatgaacctcacgaaatagaaactatctctgcgatgaacctcacgaaatagaaactatctctgcgatgaacctcacgaaatagaaactgtgcctcttatgatctggatctcgagccatggtcgaggcctgtaggcgtgtacggtgggaggcttatataagcagagctc

SEQ ID NO: 28 - DNA, 152 bp, synthetic construct
  misc_feature 1..152: note = short IRSF3x4
  source 1..152: mol_type = other DNA
gctagcgaaatagaaactgctagcgaaatagaaactgctagcgaaatagaaactgctagcgaaatagaaactgtgcctcttatgatctggatctcgagccatggtcgaggcctgtaggcgtgtacggtgggaggcttatataagcagagctc

SEQ ID NO: 29 - DNA, 200 bp, synthetic construct
  misc_feature 1..200: note = NF Bx4
  source 1..200: mol_type = other DNA
atctctgcgatgaacctcacgggactttccatctctgcgatgaacctcacgggactttccatctctgcgatgaacctcacgggactttccatctctgcgatgaacctcacgggactttccgtgcctcttatgatctggatctcgagccatggtcgaggcctgtaggcgtgtacggtgggaggcttatataagcagagctc

SEQ ID NO: 30 - DNA, 144 bp, synthetic construct
  misc_feature 1..144: note = short NF Bx4
  source 1..144: mol_type = other DNA
gctagcgggactttccgctagcgggactttccgctagcgggactttccgctagcgggactttccgtgcctcttatgatctggatctcgagccatggtcgaggcctgtaggcgtgtacggtgggaggcttatataagcagagctc

SEQ ID NO: 31 - DNA, 581 bp, synthetic construct
  misc_feature 1..581: note = IFNb-minimal-promoter
  source 1..581: mol_type = other DNA
ccccaggatgtggggttctctcccaaagggagctatggcccattgatgtgccatttctcttatagtacactattgtaagccccctgggagctcacagagaccctctcccaccatcccactatttcaaagggcagcagctgtgccatcctttcaggatgagcagctactctgcctggcttttcagtggacactttggctgtttgagagttcttttatcttcagggctgtctcctttctgttcttctctcctggatatttctcttcctttgctccagcaattggtgaaactgtacaagattttataaatccttagtttgtatatattttaacccagtacatagcatataaaatagcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgcaggtagcagccgacaccagcctggcttccatcggatccatg

SEQ ID NO: 32 - protein, 8 aa, synthetic construct
  VARIANT 2..4: note = 2A core consensus sequence motif, Xaa = Any Amino Acid
DXEXNPGP

SEQ ID NO: 33 - DNA, 16 bp, synthetic construct
  misc_feature 1..16: note = Primer mouse Actin fw
  source 1..16: mol_type = other DNA
cgcgtccacccgcgag

SEQ ID NO: 34 - DNA, 16 bp, synthetic construct
  misc_feature 1..16: note = Primer mouse Actin rev
  source 1..16: mol_type = other DNA
cctggtgcctagggcg

SEQ ID NO: 35 - DNA, 20 bp, synthetic construct
  misc_feature 1..20: note = Primer mouse IFNb fw
  source 1..20: mol_type = other DNA
ccctatggagatgacggaga

SEQ ID NO: 36 - DNA, 20 bp, synthetic construct
  misc_feature 1..20: note = Primer mouse IFNb rev
  source 1..20: mol_type = other DNA
ctgtctgctggtggagttca

SEQ ID NO: 37 - DNA, 20 bp, synthetic construct
  misc_feature 1..20: note = Primer mouse TNFa fw
  source 1..20: mol_type = other DNA
cttccagaactccaggcggt

SEQ ID NO: 38 - DNA, 18 bp, synthetic construct
  misc_feature 1..18: note = Primer mouse TNFa rev
  source 1..18: mol_type = other DNA
ggtttgctacgacgtggg

SEQ ID NO: 39 - DNA, 23 bp, synthetic construct
  misc_feature 1..23: note = Primer mouse STING fw
  source 1..23: mol_type = other DNA
catactacattgggtacttgcgg

SEQ ID NO: 40 - DNA, 24 bp, synthetic construct
  misc_feature 1..24: note = Primer mouse STING rev
  source 1..24: mol_type = other DNA
tggaataaacccgattcttgatgc

SEQ ID NO: 41 - DNA, 22 bp, synthetic construct
  misc_feature 1..22: note = Primer mouse scIL12 fw
  source 1..22: mol_type = other DNA
actggaccaaagggactatgag

SEQ ID NO: 42 - DNA, 20 bp, synthetic construct
  misc_feature 1..20: note = Primer mouse scIL12 rev
  source 1..20: mol_type = other DNA
tgatgaagaagctggtgctg

SEQ ID NO: 43 - DNA, 20 bp, synthetic construct
  misc_feature 1..20: note = Primer mouse IL6 fw
  source 1..20: mol_type = other DNA
acaaagccagagtccttcag

SEQ ID NO: 44 - DNA, 20 bp, synthetic construct
  misc_feature 1..20: note = Primer mouse IL6 rev
  source 1..20: mol_type = other DNA
tggatggtcttggtccttag

SEQ ID NO: 45 - DNA, 22 bp, synthetic construct
  misc_feature 1..22: note = Primer mouse CXCL9 fw
  source 1..22: mol_type = other DNA
tcgaggaaccctagtgataagg

SEQ ID NO: 46 - DNA, 21 bp, synthetic construct
  misc_feature 1..21: note = Primer mouse CXCL9 rev
  source 1..21: mol_type = other DNA
tgagggatttgtagtggatcg

SEQ ID NO: 47 - DNA, 20 bp, synthetic construct
  misc_feature 1..20: note = Primer mouse CXCL10 fw
  source 1..20: mol_type = other DNA
aagtgctgccgtcattttct

SEQ ID NO: 48 - DNA, 20 bp, synthetic construct
  misc_feature 1..20: note = Primer mouse CXCL10 rev
  source 1..20: mol_type = other DNA
gtggcaatgatctcaacacg

SEQ ID NO: 49 - DNA, 21 bp, synthetic construct
  misc_feature 1..21: note = Primer mouse IL15 fw
  source 1..21: mol_type = other DNA
cagaggccaactggatagatg

SEQ ID NO: 50 - DNA, 20 bp, synthetic construct
  misc_feature 1..20: note = Primer mouse IL15 rev
  source 1..20: mol_type = other DNA
gcaattccaggagaaagcag

SEQ ID NO: 51 - DNA, 22 bp, synthetic construct
  misc_feature 1..22: note = Primer mouse IL7 fw
  source 1..22: mol_type = other DNA
ctgagtgccacattaaagacaa

SEQ ID NO: 52 - DNA, 22 bp, synthetic construct
  misc_feature 1..22: note = Primer mouse IL7 rev
  source 1..22: mol_type = other DNA
gatttagaaaagcagcttcctt

SEQ ID NO: 53 - DNA, 20 bp, synthetic construct
  misc_feature 1..20: note = Primer human Actin fw
  source 1..20: mol_type = other DNA
attgccgacaggatgcagaa

SEQ ID NO: 54 - DNA, 20 bp, synthetic construct
  misc_feature 1..20: note = Primer human Actin rev
  source 1..20: mol_type = other DNA
cggactcgtcatactcctgc

SEQ ID NO: 55 - DNA, 20 bp, synthetic construct
  misc_feature 1..20: note = Primer human IFNb fw
  source 1..20: mol_type = other DNA
tgcagcagttccagaaggag

SEQ ID NO: 56 - DNA, 20 bp, synthetic construct
  misc_feature 1..20: note = Primer human IFNb rev
  source 1..20: mol_type = other DNA
agtctcattccagccagtgc

SEQ ID NO: 57 - DNA, 20 bp, synthetic construct
  misc_feature 1..20: note = Primer human TNFa fw
  source 1..20: mol_type = other DNA
gtgcttgttcctcagcctct

SEQ ID NO: 58 - DNA, 20 bp, synthetic construct
  misc_feature 1..20: note = Primer human TNFa rev
  source 1..20: mol_type = other DNA
atgggctacaggcttgtcac

SEQ ID NO: 59 - DNA, 20 bp, synthetic construct
  misc_feature 1..20: note = Primer human STING fw
  source 1..20: mol_type = other DNA
ggtgaccgtgctggcatcaa

SEQ ID NO: 60 - DNA, 20 bp, synthetic construct
  misc_feature 1..20: note = Primer human STING rev
  source 1..20: mol_type = other DNA
aaagtctgcaagggggtggc

SEQ ID NO: 61 - DNA, 22 bp, synthetic construct
  misc_feature 1..22: note = Primer human scIL12 fw
  source 1..22: mol_type = other DNA
ccaagtcaaagagtttggagat

SEQ ID NO: 62 - DNA, 22 bp, synthetic construct
  misc_feature 1..22: note = Primer human scIL12 rev
  source 1..22: mol_type = other DNA
aggtgaaacgtccagaataatt

SEQ ID NO: 63 - DNA, 20 bp, synthetic construct
  misc_feature 1..20: note = Primer human IL6 fw
  source 1..20: mol_type = other DNA
agtgaggaacaagccagagc

SEQ ID NO: 64 - DNA, 20 bp, synthetic construct
  misc_feature 1..20: note = Primer human IL6 rev
  source 1..20: mol_type = other DNA
ggtcaggggtggttattgca

SEQ ID NO: 65 - DNA, 21 bp, synthetic construct
  misc_feature 1..21: note = Primer human CXCL9 fw
  source 1..21: mol_type = other DNA
accaaccaagggactatccac

SEQ ID NO: 66 - DNA, 20 bp, synthetic construct
  misc_feature 1..20: note = Primer human CXCL9 rev
  source 1..20: mol_type = other DNA
tcaattttctcgcaggaagg

SEQ ID NO: 67 - DNA, 22 bp, synthetic construct
  misc_feature 1..22: note = Primer human CXCL10 fw
  source 1..22: mol_type = other DNA
tgctgccttatctttctgactc

SEQ ID NO: 68 - DNA, 21 bp, synthetic construct
  misc_feature 1..21: note = Primer human CXCL10 rev
  source 1..21: mol_type = other DNA
tgatgcaggtacagcgtacag

SEQ ID NO: 69 - DNA, 22 bp, synthetic construct
  misc_feature 1..22: note = Primer human IL15 fw
  source 1..22: mol_type = other DNA
caaagtaacagcaatgaagtgc

SEQ ID NO: 70 - DNA, 22 bp, synthetic construct
  misc_feature 1..22: note = Primer human IL15 rev
  source 1..22: mol_type = other DNA
caaactgttgtttgctaggatg

SEQ ID NO: 71 - DNA, 22 bp, synthetic construct
  misc_feature 1..22: note = Primer human IL7 fw
  source 1..22: mol_type = other DNA
ttcagaaggcacaacaatactg

SEQ ID NO: 72 - DNA, 22 bp, synthetic construct
  misc_feature 1..22: note = Primer human IL7 rev
  source 1..22: mol_type = other DNA
gttctttagtgcccatcaaaat

SEQ ID NO: 73 - DNA, 21 bp, synthetic construct
  misc_feature 1..21: note = Primer AAV2 ITR fw
  source 1..21: mol_type = other DNA
ggaacccctagtgatggagtt

SEQ ID NO: 74 - DNA, 16 bp, synthetic construct
  misc_feature 1..16: note = Primer AAV2 ITR rev
  source 1..16: mol_type = other DNA
cggcctcagtgagcga

SEQ ID NO: 75 - DNA, 20 bp, synthetic construct
  misc_feature 1..20: note = Primer luciferase fw
  source 1..20: mol_type = other DNA
aaccagcgccattctgatca

SEQ ID NO: 76 - DNA, 20 bp, synthetic construct
  misc_feature 1..20: note = Primer luciferase rev
  source 1..20: mol_type = other DNA
ccagtgtcttaccggtgtcc

SEQ ID NO: 77 - DNA, 22 bp, synthetic construct
  misc_feature 1..22: note = Primer eGFP fw
  source 1..22: mol_type = other DNA
atcaaggtgaacttcaagatcc

SEQ ID NO: 78 - DNA, 22 bp, synthetic construct
  misc_feature 1..22: note = Primer eGFP rev
  source 1..22: mol_type = other DNA
ttacttgtacagctcgtccatg

SEQ ID NO: 79 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 80 - protein, 475 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGAPDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 81 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 82 - protein, 21 aa, synthetic construct
GGSGNFDLLKLAGDVESNPGP

SEQ ID NO: 83 - DNA, 266 bp, synthetic construct
  source 1..266: mol_type = other DNA
acatgtgactttggtcagctagcaaagaattcaaaagatctaaactcgagaaaaggcctacggttaccccggcgggtttctgacatccggcgggtttctgacatccggcgggtttctgacatccggcgggtttctgacatccggcgggtttctgacatccggcgggtttctgacatccggcgggtttctgacatccggcgggtttctgacatccggcgggtttctgacatccggcgggtgactcacaaccccagaaacagacatcc

SEQ ID NO: 84 - DNA, 586 bp, synthetic construct
  source 1..586: mol_type = other DNA
cccctctccctcccccccccctaacgttactggcgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 85 - protein, 12 aa, synthetic construct
VQWRIRVAVIRK

SEQ ID NO: 86 - DNA, 418 bp, Homo sapiens
  source 1..418: mol_type = genomic DNA
ccatataaggagcaggaaggatcccccgccggaacaacccttatttgggcagcaccttatttggagtggcccgatatggcccggccgcttccggctctgggaggagggaagaaggcggagggaggggcaacgcgggaactccggagctgcgcgggtcccggaggccccggcggcggctagagctctaggcttccccgaagcctgggcgcctgggatgcgggcgcgggcgcgggccctagggtgcaggatggaggtgccgggcgctgtcggatggggggcttcacgtcactccgggtcctcccggccggtcctgccatattagggcttcctgcttcccatatatggccatgtacgtcacgacggaggcggacccgtgccgttccagacccttcaaatagaggcggatccggggagtcgc

SEQ ID NO: 87 - DNA, 10 bp, synthetic construct
  source 1..10: mol_type = other DNA
  misc_difference 3..8
ccwwwwwwgg

SEQ ID NO: 88 - DNA, 181 bp, Homo sapiens
  source 1..181: mol_type = genomic DNA
tgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatct

(19) United States
(12) Patent Application Publication
(10) Pub. No.: US 2025/0186623 A1
(43) Pub. Date: Jun. 12, 2025

(54) EXPRESSION SYSTEM FOR THE TREATMENT OF CANCER

(71) Applicant: FUNDACION PARA LA INVESTIGACION MEDICA APLICADA, Pamplona (Navarra) (ES)

(72) Inventors: Jesus María Prieto Valtueña, Pamplona (Navarra) (ES); Sonia MARCO MARTÍNEZ, Pamplona (Navarra) (ES); Beatriz HONORATO CÍA, Pamplona (Navarra) (ES); Shirley Mireya TENESACA CAYAMBE, Pamplona (Navarra) (ES); David James GOULD, London (GB)

(21) Appl. No.: 18/844,802

(22) PCT Filed: Mar. 7, 2023
(86) PCT No.: PCT/EP2023/055785

§ 371 Date: Sep. 6, 2024

(30) Foreign Application Priority Data
Mar. 7, 2022 (EP) 22382208.1

(51) Int. Cl. A61K 48/00; A61P 35/00; C07K 14/54; C07K 14/705; C12N 15/86

(52) U.S. Cl. CPC A61K 48/0066; A61K 48/0058; A61P 35/00; C07K 14/5434; C07K 14/705; C12N 15/86; C12N 2750/14122; C12N 2750/14143; C12N 2830/002; C12N 2830/42

(57) ABSTRACT

The invention relates to a recombinant expression system for the treatment of cancer based on the “Stimulator of interferon genes” (STING) and comprising two nucleic acid constructs. The invention further relates to an expression vector or a combination of two expression vectors encoding said nucleic acid construct(s) and to a viral particle or a combination of two viral particles comprising said expression vector(s). The invention also relates to a pharmaceutical composition that comprises said recombinant expression system, said expression vector(s) or said viral particle(s). The invention also relates to the use of said recombinant expression system, said expression vector(s), said viral particle(s) or said pharmaceutical composition for use in an immunotherapy, especially in the immunotherapy of a proliferative disorder like cancer. The invention finally relates to the administration of the expression vectors in combination with radiotherapy for the treatment of cancer.

### FIELD OF THE INVENTION

The invention relates to a recombinant expression system for the treatment of cancer based on the “Stimulator of interferon genes” (STING) and comprising two nucleic acid constructs. The invention further relates to an expression vector or a combination of two expression vectors encoding said nucleic acid construct(s) and to a viral particle (or a combination thereof) comprising said expression vector(s). The invention also relates to a pharmaceutical composition that comprises said recombinant expression system, said expression vector(s) or said viral particle(s). The invention finally relates to the use of said recombinant expression system, said expression vector(s), said viral particle(s) or said pharmaceutical composition for use in an immunotherapy, especially in the immunotherapy of a proliferative disorder like cancer.

### BACKGROUND OF THE INVENTION

Cancer is a group of diseases involving abnormal cell growth and proliferation with around 19 million new cases annually and it is the cause of around 10 million deaths annually (Global Cancer Observatory, 2020). While significant progress in the treatment of cancers has been made in the last decades, the annual mortality rate shows that still a huge number of people die because of cancer.

One severe obstacle for the treatment of cancer is that there are several different types of cancers each with individual challenges for its treatment. For example, some cancers are easily accessible and can therefore be treated locally (i.e. surgical removal, local radiotherapy) while others can only be treated by systemic approaches (i.e. chemotherapy, hormone therapy, immunotherapy).

A plethora of different treatments is available such as radiotherapy, chemotherapy, surgical removal, hormone therapy and immunotherapy. Some of these treatments target tumors by systemically interfering with cell proliferation, hence stopping tumor growth. However, these treatments cause severe side effects for the patient like fatigue, nausea and vomiting, anemia and infection, as also physiologically dividing cells like hematopoietic cells, cells in the mouth, digestive tract, and reproductive system are unwantedly targeted by these systemic treatments. Also, local therapies such as local radiotherapy cause severe side effects depending on the localization of the tumor. In contrast, the concept of immunotherapy is to stimulate the body's immune system to act against cancer cells, thereby aiming to reduce unwanted side effects. Immunotherapies can be categorized as active or passive. Active immunotherapy is characterized by immunization of patients with agents that increase the immune response towards a tumor antigen. Examples include therapeutic cancer vaccines, cell-based immunotherapies, and targeted antibody therapies. In contrast, passive immunotherapy does not directly target tumor cells, but enhances the ability of the immune system to attack cancer cells. Examples include checkpoint inhibitors and cytokines. However, immunotherapies so far show several disadvantages such as i) poor tumor targeting, ii) weak tumor cell killing ability, iii) high effort and thereby high cost, iv) tumor immune escape, v) over-stimulation of the immune system which can cause severe side effects (Tan et al. 2020, Biomed Pharmacother. 124: 109821).

STING (also known as Endoplasmic reticulum interferon stimulator (ERIS), Mediator of IRF3 activation (MITA) or Transmembrane protein 173 (TMEM173) is a four-transmembrane endoplasmic protein present in the endoplasmic reticulum (ER) which acts as a sensor of the presence of double-stranded DNA (dsDNA) in the cytosol. As such, STING is activated by DNA viruses, by DNA-damaging insults like radiotherapy and by micronuclei frequently present in the cytosol in malignant cells (Li et al. Journal of Hematology & Oncology 2019, 12:35). STING activates TANK-binding kinase 1 (TBK1) leading to phosphorylation and activation of interferon regulatory transcription factor 3 (IRF3) and upregulation of type I interferon (IFN) expression. STING also recruits IκB kinase (IKK) and activates the NF-κB pathway which promotes the production of pro-inflammatory molecules (including IL-6 and TNF-α) and cooperates with TBK1-IRF3 in the production of type I IFN (Li et al. Journal of Hematology & Oncology 2019, 12:35). Type I IFN stimulates immune-effector cells including dendritic cells (DC) and natural killer (NK) cells and promotes the production of chemokines like CXCL9 and CXCL10 which attract T lymphocytes to the tumor which are critical for anti-tumor immunity (Mowat, C. et al J. Exp. Med. 2021 Vol. 218 No. 9 e20210108). In fact, STING agonists, like cyclic dinucleotides (CDN), exert potent antitumor effects in different tumor models (Sivick K E et al Cell Reports 2018; 25, 3074-3085). The importance of STING in cancer pathology can also be derived from the fact that STING expression is usually suppressed or lost in the many cancers.

Unfortunately, significant STING polymorphism exists in the human population, which affects both the molecular responses induced by the protein's activation and the degree of sensitivity to stimulatory ligands. Consequently, this can greatly impact the efficacy and safety of molecular entities pursued for clinical purposes.

Based on these numerous findings, STING is now regarded as a master regulator in the cancer-immunity cycle.

A well described activator of STING is 5,6-Dimethylxanthenone-4-acetic acid (DMXAA) disclosed in WO 2004/039363 A1 but also other STING agonists are known in the art. Clinical trials with DMXAA revealed that it is well tolerated and only displayed minor side effects. However, while antitumor effects of STING were described in the animal model, the clinical trials revealed that DMXAA failed in the trials due to lack of efficacy. Further studies have demonstrated the reason for this therapeutic inefficacy, showing that DMXAA potently activates murine STING but is unable to activate human STING due to amino acid differences of the cyclic-dinucleotide (CDN)-binding site of STING.

Documents U.S. Pat. No. 5,126,132 A and US 2005/214274 A1 disclose cell-based immunotherapies where immune-competent cells from the patient like T-cells are isolated, cultivated and modified ex vivo so that a specific type of cancer is recognized by these cells, and then reinfused back into the patient. However, next to the elaborate process to generate these ex vivo-modified cells, they only target a specific type of cancer.

Immunotherapies using the cytokine interleukin-2 (IL-2) to stimulate the immune system in targeting cancer cells have been approved as early as 1992 for the use to treat melanoma and renal cancer. However, studies show that only around 7% to 16% of patients respond to this treatment at all and severe side effects are common (Marabondo et al. Expert Opin Drug Saf. 2017 December; 16(12): 1347-1357.).

The use of autologous cancer cells as vaccines to augment anti-tumor immunity has been explored for some time. However, due to the weak immunogenicity of many cancers, down regulation of MHC molecules, the lack of adequate costimulatory molecule expression and secretion of immuno-inhibitory cytokines by cancer cells, the response to such vaccines has not resulted in long term efficacy (see, e.g., Armstrong T D and Jaffee E M, Surg Oncol Clin N Am. 1 1(3): 681-96, 2002). Numerous cytokines have been shown to play a role in regulation of the immune response to tumors. For example, U.S. Pat. No. 5,098,702 describes using combinations of TNF, IL-2 and IFN-β in synergistically effective amounts to combat existing tumors. U.S. Pat. Nos. 5,078,996, 5,637,483 and 5,904,920 describe the use of GM-CSF for treatment of tumors. However, direct administration of cytokines for cancer therapy may not be practical, as they are often systemically toxic (Asher et al., J. Immunol. 146: 3227-3234, 1991).

Document US 2021/038684 A1 provides compositions and methods for the treatment of cancer using IL-2 immunotherapy. The methods of the invention comprise administering to a patient an interleukin-2/interleukin-2-receptor a fusion protein systemically, wherein administration results in a dose dependent increase in circulating NK cells and CD8+ cells in a patient in the absence of a dose dependent increase in circulating immunosuppressive T regulatory (Treg) cells. However, while aiming to overcome tumor immune escape this invention comprises the disadvantages known for cancer immunotherapy like weak tumor cell killing ability with only some patients responding to the treatment at all (Boni et al., Journal of Clinical Oncology 2021, 39:15_suppl, 2513-2513). Moreover, cytokine-based immunotherapies normally need to be performed repeatedly to ensure sufficient immune system stimulation by avoiding over-stimulation thereby causing additional medical costs and burden for the patient.

Hence, there is still a need for the efficient and safe treatment of proliferative diseases like cancer, where many different types of cancer are targeted efficiently, these treatments show no or minor unwanted side effects, and a wide variety of patients respond to the treatment.

The objective of the present invention thus is to provide an improvement or an alternative to the prior art.

This problem is solved by provision of a recombinant expression system according to claim 1 or 25, an expression vector according to claim 14 or 32 or a combination of two expression vectors according to claim 14, a viral particle according to claim 15 or 33 or a combination of two viral particles according to claim 15 and a pharmaceutical composition according to claim 16 or 34. Specific embodiments are subject matter of further dependent or independent claims.

### SUMMARY OF THE INVENTION

In a first aspect the present invention provides a recombinant expression system comprising
a. a first nucleic acid construct comprising a sequence encoding for a target STING, that is responsive to a STING agonist recognizing said target STING but not the human STING and natural variants thereof (human-non-reacting STING agonist), said sequence encoding the target STING is operatively linked to elements allowing expression and/or translation of said target STING; and/or
b. a second nucleic acid construct comprising a sequence encoding at least one immunomodulatory gene or protein, said sequence is operatively linked to a STING-responsive promoter or a radiation inducible promoter.

The expression system according to the invention combines many advantages of cancer immunotherapies while avoiding the disadvantages of said therapies.

It allows to express immunomodulatory peptides, especially cytokines such as interleukins or chemokines, in the tumor, thereby stimulating the immune system to target cancer cells of said tumor. Due to a preferred intratumor administration of said expression system, the cytokine expression is highly localized thereby circumventing the disadvantages and toxicity from systemic cytokine therapies known in the art.

The recombinant expression system includes two expression cassettes represented by the two nucleic acid constructs: a) one cassette represented by the first nucleic acid construct encoding a target STING that is responsive to a STING agonist that does not activate endogenous human STING (in the following also designated as “human-non-reacting STING agonist”) and b) another cassette represented by the second nucleic acid construct that encodes an immunomodulatory gene or protein under the control of a promoter that is activated upon STING activation. According to the invention the recombinant expression system comprises both cassettes or only one of them. The combined expression of both cassettes has the advantage that after intratumor injection of the vector(s) containing said expression cassettes, the systemic administration of the agonist of said target STING enables the selective activation of STING within the tumor without affecting the endogenous human STING in the rest of the body, thus avoiding toxicity. The intratumor activation of the target STING induces the production of IFN type I, chemokines and other cytokines within the tumor by induction of endogenous genes or by activation of the promoter of the second expression cassette that encodes an immunomodulatory gene or protein, like IL-12. In this manner the systemic administration of the agonist of said target STING triggers intratumor production of IFN type I, and other immunostimulatory peptides resulting from STING activation, together with the production of the immunomodulatory gene or protein expressed by the second expression cassette. The local intratumor production of this combination of immunomodulatory genes or proteins would promote antitumor immunity without toxicity. By injecting the tumor with long-term expression vectors (like adeno-associated virus vectors) containing the two expression cassettes, it is possible to induce the production of the mentioned combination of immunomodulatory genes or proteins within the tumor by repeating the systemic administration of the agonist of the target STING.

The inventors found that the first nucleic acid of the recombinant expression system according to the invention induces the upregulation of STING-associated genes or proteins upon specifically activating the target STING by administering a STING agonist that recognizes said target STING but not the human STING and natural variants thereof. It was further found that the upregulation of STING-associated genes or proteins are also induced by low dose radiotherapy. It could be shown that the enrichment in STING (e.g. by transducing the cells with a target STING) makes the cells more responsive to either STING agonists like DMXAA (canonical pathway) or to low dose radiotherapy (non-canonical pathway) separately and enables the full display of the synergism between the two stimuli (STING agonist and low dose radiotherapy) when they are applied together (see FIG. 9 A-C). Accordingly, in one aspect of the invention it is sufficient that the expression system includes only the cassette encoding a target STING that is responsive to a STING agonist that does not activate human STING.

Furthermore, the inventors found that the second nucleic acid of the recombinant expression system according to the invention induces the upregulation of the immunomodulatory transgene dependent on STING activity and that this activity can be specifically induced by a STING agonist that recognizes said target STING but not the human STING and natural variants thereof. Thus, the expression of the immunomodulatory transgene can be controlled by administration of said STING agonist.

Moreover, the inventors found that the recombinant expression system according to the invention is non-toxic and shows minor side effects in vivo. Of note, the inventors found that the recombinant expression system causes the reduction of tumors eventually leading to full remission when administered in vivo. Even more, by administering the recombinant expression system of the invention locally in the tumor, not only the targeted tumor is reduced in its size, but also non-targeted tumors show significant size reductions, also known as the abscopal effect.

### DETAILED DESCRIPTION OF THE INVENTION

The recombinant expression system generates a specific and basal immunological pathway in the transfected cancer cell that makes use of further endogenous pathway components.

Of note the term “recombinant expression system” includes any nucleotides, analogues thereof, and polymers thereof. Hence, any of the recombinant expression system disclosed herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA.

A more comprehensive definition is enclosed in the definition section of this application. The first component of this pathway is the so-called “target STING”, which is defined by its responsiveness to a STING agonist that is unable to activate the human STING allowing a selective activation of the target STING. The first component is given by the first nucleic acid construct of the invention and can be regarded as both “co-effector” and “activator” part of the pathway. Notably, the first component can be also given alone, making the cells expressing this first component responsive to STING agonists and low dose radiotherapy, whereby the full display of the synergism between these two stimuli is given (STING agonist and low dose radiotherapy) when they are applied together.

In the first nucleic acid construct according to the invention, the target STING is operatively linked to elements allowing expression and/or translation of said target STING. In the event that the recombinant expression system is a DNA-based expression system this element is typically a promoter that allows the transcription of an mRNA encoding the target STING. In the event that the recombinant expression system is an RNA-based expression system this element is typically an RNA element that allows the translation of the mRNA encoding the target STING such as e.g. a CAP, the 5′UTR, the 3′UTR or a poly A site.

The second component of this pathway is an artificial gene construct that includes an immunomodulatory gene or protein, said sequence is operatively linked to a STING-responsive promoter or a radiation inducible promoter. The immunomodulatory gene or protein activates both innate and adaptive immune responses in order to generate a specific cancer immune response. The second component is given by the second nucleic acid construct of the invention and can be regarded as “co-effector” part of the pathway.

Notably, the target STING differs from the native human STING due to its activation by human-non-reacting agonists such as DMXAA. However, upon activation, the target STING displays biological activity of human STING in that the target STING is still able to bind to and activate downstream effector proteins that are also activated by human STING such as iRhom2 and TANK-binding kinase 1 (TBK1). Upon activation, STING suffers a conformational change leading to TBK1 recruitment. TBK1, in turn, phosphorylates the transcription factors interferon regulatory factor 3 (IRF3) and nuclear factor-κB (NF-κB), that translocate to the nucleus and stimulate the production of pro-inflammatory molecules such as type I interferon, IL6, TNF-α or CXCL10. (Ahn, J. et al, Exp Mol Med 51, 1-10 (2019)).

It may be provided that the target STING is responsive to an agonist that does not react with human STING. Said human-non-reacting STING agonist can be selected from the group consisting of 5,6-dimethylxanthenone-4-acetic acid (DMXAA), 7-bromo-DMXAA, 7-iodo-DMXAA, 7-hydroxy-DMXAA, 7-formyl-DMXAA, 7-hydroxymethyl-DMXAA, 7-(2-hydroxyethyl)-DMXAA, flavone-8-acetic acid (FAA), 2,7-bis(2-dimethylamino ethoxy)fluoren-9-one (Tilorone) and 10-carboxymethyl-9-acridanone (CMA). The person skilled in the art recognizes that also analogues of the molecules disclosed herein and any of their stereoisomers may be used. In addition, one or more atoms of the aforementioned molecules, their analogues and/or their stereoisomers may be replaced by their respective isotopes.

The use of a human-non-reacting STING agonist in the context of this invention has some advantages when used in the treatment of humans. By definition, this human-non-reacting STING agonist is unable to bind human wild-type STING or natural variants thereof in a way that causes STING activation. Thereby it can be administered systemically without causing a systemic STING response, thus avoiding unwanted side effects. As the invention intends to express a target STING that is responsive to the human-non-reacting STING agonist it is thereby possible to specifically activate this transgenic STING, thus causing a local STING activation. Hence, in the context of the present invention, the human-non-reacting STING agonist has two roles: It is used to define the STING to be used as target STING in the gene therapy and furthermore it is used in the subsequent pharmacotherapy to activate this target STING, thus enabling a highly selective therapeutic approach.

Preferably, the target STING is responsive to DMXAA. A use of a DMXAA-responsive STING has the advantage that the STING agonist DMXAA is already proven in clinical studies as a molecule with beneficial safety and only minor side effects.

Furthermore, it could be shown that the activation of the target STING by DMXAA shows a dose dependent-induction of downstream STING targets such as IFN-β (see Example 1). This allows to control the immune response by simply adjusting the DMXAA dose during treatment.

Intratumor injections of DMXAA have been shown to effectively prime CD8+ T-cell responses and promote elimination of the established tumors in a STING-dependent fashion in mice (Corrales et al. Cell Reports, 2015; 11, 1018-1030). Clinical trials addressing the antitumor properties of DMXAA revealed that it shows only minor side effects. However, these trials revealed that DMXAA fails to activate human wild-type STING or natural variants thereof. Nonetheless, murine STING and rat STING are responsive towards DMXAA in that they show biological activity of STING.

It is further conceivable within the scope of the invention that the target STING encoded by the sequence of the first nucleic acid construct is a mutated form of a natural variant of human STING that further comprises at least one, at least two, or at least three amino acid substitutions that renders the human STING variant sensitive to the human-non-reacting STING agonist as determined in an in vitro-agonist determination assay.

The in vitro-agonist determination assay can be used to identify human-non-reacting STING agonists. There are several possible methods to perform such assays known by the person skilled in the art. In one non-limiting example a vector encoding a reporter (e.g. luciferase) under the control of a STING-responsive promoter can be constructed. Cells may be transformed, transfected or transduced with said vector and in addition with vectors that induce the expression of human STING or natural variants thereof, mutated human STING variants or non-human STING. These cells should then be brought in contact with a STING agonist (being preferably DMXAA) and the luciferase activity should be measured. If luciferase activity in cells transformed, transfected or transduced with vectors that induce the expression of mutated human STING variants or nun-human STING are detectable but the luciferase activity in cells transformed, transfected or transduced with vectors that induce the expression of human STING or natural variants thereof does not significantly differ to mock-treated control groups, the agonist is deemed “human-non-reacting”. In this context, not significantly different to mock-treated control groups means that the luciferase activity is lower than 30%, preferably lower than 10%, more preferably lower than 5% and particularly preferably lower than 1% of the luciferase activity obtained by using hSTING[R]-S162A/G230I/Q266I (SEQ ID NO: 6). Another example of an in vitro-agonist determination assay would be to directly measure the IFN-β levels when cells that are transformed, transfected or transduced with vectors that induce the expression of mutated human STING variants, nun-human STING, or human STING or natural variants thereof are contacted with a STING agonist. In this context, a human-non-reacting STING agonist would provide at least 25%, preferably at least 50%, more preferably at least 75% and even more preferably 90% of IFN-β levels as obtained by using hSTING[R]-S162A/G230I/Q266I (SEQ ID NO: 6).

It is further conceivable within the scope of the invention that the target STING encoded by the sequence of the first nucleic acid construct is a non-human STING, that is sensitive to the human-non-reacting STING agonist as determined in an in vitro-agonist determination assay, whereby the human-non-reacting STING agonist is preferably DMXAA. Non-limiting examples of non-human STINGs that are sensitive to human-non-reacting STING agonists as determined in an in vitro-agonist determination assay are murine STING or natural variants thereof, rat STING or natural variants thereof or a hybrid STING. In a preferred embodiment the non-human STING is a murine STING according to SEQ ID NO: 7, which encodes the protein of SEQ ID NO: 8.

Alternatively, a radiation inducible promoter may be provided. A radiation inducible promoter, as used herein, is a promoter that induces the expression of the therapeutic gene sequence when the cell is irradiated. These promoters may be natural, synthetic or artificially made selected from recombination products of natural promoters. For example, these promoters may contain at least one of the following sequences either individually or in repeated fashion:
a. eukaryotic radiation-inducible sequence CC(A/T)(6)GG (SEQ ID NO: 87), also known as CArG elements or [CC(A+T rich)6GG], such as that of early growth response-1 (Egr-1) promoter or pE9 promoter, preferably the sequence has 70%, 75%, 80%, 85%, 90%, 95%, or 99% sequence identity with respect to SEQ ID NO: 86 or is identical to SEQ ID NO: 86 (Egr1 promoter), or alternatively the sequence contains at least two sequences according to SEQ ID NO: 87, wherein the at least two sequences may be interspaced by additional nucleotides;
b. low frequency CCAAT box sequences such as that of the survivin promoter, preferably the sequence has 70%, 75%, 80%, 85%, 90%, 95%, or 99% sequence identity with respect to SEQ ID NO: 88 or is identical to SEQ ID NO: 88 (CCAAT rich domain of the survivin promoter);
c. Cyclin dependent kinase inhibitor 1 (also known as CDKN1A or p21) promoter;
d. late Herpes Simplex Virus 1 promoters, such as glycoprotein C promoter or Us 11 promoter;
e. combinations of the above or artificially mutated versions of the same.

In a preferred embodiment of the invention, the target STING is a DMXAA-sensitive human STING as determined in an in vitro-DMXAA determination assay. As described above the use of DMXAA for treatment in a human has the advantages that DMXAA has been shown to have beneficial safety, showing only minor side effects and due to its inability to activate human STING it can be administered systemically without causing systemic STING activation.

The in vitro-DMXAA determination assay can be used to identify STINGs that are responsive to DMXAA. Cells may be transduced with vectors that induce expression of any STING (e.g. human wild-type STING, murine STING). If upon addition of DMXAA, the cells respond by showing, in triplicated determinations, statistically significant elevations in mRNA levels for IFN-β and other cytokines determined by quantitative PCR (qPCR) compared to a mock-treated control, the expressed STING is deemed DMXAA-sensitive (see for Example FIG. 1). In this context, statistically significant elevations mean that the mRNA levels are at least 10%, preferably at least 20%, more preferably at least 30% and particularly at least 50% higher compared to mock-treated controls. An example of DMXAA-non-reacting STING is human wild-type STING or natural variants thereof. Another example of an in vitro-DMXAA determination assay would be to directly measure the IFN-β levels of cells that were transformed, transfected or transduced with a vector encoding STING (e.g. human wild-type STING, murine STING) after being contacted by DMXAA. In this context, a DMXAA-sensitive human STING would provide at least 25%, preferably at least 50%, more preferably at least 75% and even more preferably 90% of IFN-β levels as obtained by using hSTING[R]-S162A/G230I/Q266I (SEQ ID NO: 6) treated with DMXAA.

Advantageously, in the context of the invention, it may be provided that the target STING encoded by the sequence of the first nucleic acid construct is a DMXAA-sensitive human STING variant being selected from the group consisting of:
a. a human STING[R] variant further comprising at least 1, at least 2, or at least 3 amino acid substitutions selected from the group consisting of S162A, G230I, and Q266I;
b. a human STING[AQ] variant further comprising at least 1, at least 2, or at least 3 amino acid substitutions selected from the group consisting of S162A, A230I, Q266I, E260I and E260V;
c. a human STING[H] variant further comprising at least 1, at least 2, or at least 3 amino acid substitutions selected from the group consisting of S162A, G230I, and Q266I; and
d. a truncated human STING[H] variant further comprising at least 1, or at least 2 amino acid substitutions selected from the group consisting of S162A and G230I.

The skilled person notices that all of the aforementioned mutations lie within the cyclic-dinucleotide (CDN)-binding site (binding pocket) and/or the lid region of the binding pocket of STING. Thus, in another embodiment the endogenous human STING may contain a mutation of one of the amino acids lying within the binding pocket region and/or the lid region in order to render it a DMXAA-sensitive human STING.

Several natural variants of human STING have been identified, e.g. as a result of several single polymorphisms (SNPs) and SNPs haplotypes that produce several allelic polymorphic forms of the protein. The amino acid sequence of the most common natural variant or allele of human STING is SEQ ID NO: 1, which is referred as the wild-type human STING. The most common natural allelic variants of human STING include: hSTING[R] as depicted in SEQ ID NO: 1, commonly referred as hSTING R232, i.e. amino acid at position 232 is arginine, this STING is also considered the wild-type isoform; hSTING[H] as depicted in SEQ ID NO: 2, commonly referred as hSTING H232, i.e. amino acid at position 232 is histidine; hSTING[AQ] as depicted in SEQ ID NO: 3, whose sequence differs from hSTING[R] by incorporation of amino acid substitutions G230A, and R293Q. In addition, a truncated form of hSTING[H] as depicted in SEQ ID NO: 4 is characterized. A further natural variant is STING[HAQ] which possess the amino acid substitutions R71H, G230A and R293Q. Thus, the target STING is not identical to one of the known haplotypes of human STING, namely hSTING[R] as depicted in SEQ ID NO: 1, hSTING[H] as depicted in SEQ ID NO: 2, hSTING[AQ] as depicted in SEQ ID NO: 3, hSTING[H] as depicted in SEQ ID NO: 4, STING[Q] which possess the amino acid substitution R293Q or STING[HAQ] which possess the amino acid substitutions R71H, G230A and R293Q. Additionally, the target STING may be responsive to any of the human-non-reacting STING agonists selected from the group consisting of 5,6-dimethylxanthenone-4-acetic acid (DMXAA), 7-bromo-DMXAA, 7-iodo-DMXAA, 7-hydroxy-DMXAA, 7-formyl-DMXAA, 7-hydroxymethyl-DMXAA, 7-(2-hydroxyethyl)-DMXAA, flavone-8-acetic acid (FAA), 2,7-bis(2-dimethylamino ethoxy)fluoren-9-one (Tilorone) and 10-carboxymethyl-9-acridanone (CMA). Therefore, administration of the agonist causes the activation of the exogenous STING provided by the first nucleic acid of the recombinant expression system, wherein the endogenous STING which is encoded in the genome of the human subject is not activated.

In a further possibility it may be provided that the DMXAA-sensitive human STING variant encoded by the sequence of the first nucleic acid construct is selected from the group consisting of hSTING[R]-S162A/G230I/Q266I, hSTING[R]-S162A/Q266I, hSTING[R]-G230I, hSTING[R]-S162A, hSTING[R]-Q266I, hSTING[R]-S162A/G230I, hSTING[AQ]-S162A/A230I/Q266I, hSTING[AQ]-S162A, hSTING[AQ]-S162A/R169A, hSTING[AQ]-S162A/T267A, hSTING[AQ]-E260I, hSTING[AQ]-E260V, hSTING[H]-S162A/G230I/Q266I, hSTING[H]-S162A/Q266I, hSTING[H]-G230I, hSTING[H]-S162A, hSTING[H]-S162A/G230I and hSTING[H]-Q266I and preferably is hSTING[R]-S162A/G230I/Q266I.

Experiments that have been conducted show that the mutation S162A is crucial in allowing DMXAA to bind and activate human STING and that the mutations Q2661 and G230I or A230I increase the sensitivity towards DMXAA (Gao et al., 2014, Cell Reports 8, 1668-1676).

According to a further advantage it can be provided that the sequence of the first nucleic acid construct encoding for the DMXAA-sensitive human STING variant is selected from the group consisting of SEQ ID NO: 5 which is the sequence of hSTING[R]-S162A/G230I/Q266I or a polynucleotide sequence having at least 75%, at least 80%, at least 85%, at least 90% or at least 95% sequence identity with respect to SEQ ID NO: 5. The sequence identity of the polynucleotide sequence with respect to SEQ ID NO: 5 can be at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Notably, the polypeptide encoded by any of the aforementioned polynucleotides has the biological activity of being responsive to the agonist DMXAA as determined in an in vitro-DMXAA determination assay.

According to a further advantage it can be provided that the sequence of the first nucleic acid construct encoding for the DMXAA-sensitive human STING variant is selected from the group consisting of a polynucleotide sequence encoding a polypeptide comprising SEQ ID NO: 6 which is the sequence of hSTING[R]-S162A/G230I/Q266I or a polynucleotide sequence encoding a polypeptide having at least 75%, at least 80%, at least 85%, at least 90% or at least 95% sequence identity with respect to SEQ ID NO: 6. The sequence identity of the polypeptide encoded by the polynucleotide sequence with respect to SEQ ID NO: 6 can be at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Notably, the polypeptide encoded by any of the aforementioned polynucleotides has the biological activity of being responsive to a human-non-reacting STING agonist as determined in an in vitro-agonist determination assay.

Both, hSTING[R]-S162A/G230I/Q266I and murine STING, have extensively been researched and are known to be highly activated by DMXAA and CMA. In addition, murine STING may also be activated by FAA and Tilorone. Using a variant of human STING however may be advantageous over the use of non-human STING in the treatment of cancer as the former might show better interaction with signal-transducing molecules in human cells. In addition, the use of non-human STING might cause unwanted immunologic responses.

Notably, the polynucleotide sequences from the first nucleic acid construct as described hereinbefore may comprise at least one modification of the polypeptide that is an amino acid substitution, amino acid insertion, amino acid deletion, C-terminal truncation or N-terminal truncation. In addition, also polynucleotide sequences that hybridize under at stringent conditions to the complement of the aforementioned polynucleotide sequences are included according to the invention. Furthermore, fragments of at least 50 amino acid residues of the DMXAA-sensitive human STING variant are comprised according to the invention. Notably, the polypeptide encoded by any of the aforementioned polynucleotides has the biological activity of being responsive to a human-non-reacting STING agonist as determined in an in vitro-agonist determination assay.

Preferably, it may be provided that the promoter of the first nucleic acid construct is a constitutive promoter such as SV40, CMV0, UBC, EF1a, PGK, CAG, EFS, CBh, CBA, human β actin, Ac5, Polyhedrin, TEF1, GDS, CaMV35S, Ubi, H1 or U6. Preferably, the promoter is selected from the list consisting of SV40, CMV0, UBC, EF1a, PGK, CAG, EFS, CBh and CBA. Using a constitutive promoter in the context of the invention has the advantage that expression of the “activator” part of the pathway is ensured. In general, constitutive promoters show higher expression levels compared to inducible or tissue-specific promoters. However, the person skilled in the art recognizes that also cancer-specific promoters are feasible promoters of the first nucleic acid construct in the context of the invention.

In an alternative embodiment the promoter of the first nucleic acid construct is an inducible promoter, preferably selected from the list consisting of Telomerase-responsive promoter, IL-6 responsive promoter, TGF-β-inducible promoter, VEGFR-inducible promoter, Epidermal Growth factor receptor-inducible promoter, HER2/Neu-inducible promoter, MUC1 promoter, BRCA1 promoter, BRCA2 promoter, LALBA promoter, PSA promoter, ERBB2 promoter, CEA promoter, uPAR promoter and Thyroid transcription factor 1 element (TTF1).

Furthermore, it may be advantageous within the scope of the invention that the at least one immunomodulatory gene or protein encoded by the sequence of the second nucleic acid construct is selected from the group consisting of the family of interleukins, the family of chemokines, the family of colony-stimulating factors, the family of transforming growth factors, the family of tumor necrosis factors, any blocking peptide against immunosuppressive factors, any monoclonal antibody or any scFv or any nanobody reactive with immune checkpoint proteins, or a shRNA targeting immunosuppressive factors. A non-limiting list of immunomodulatory proteins is interleukin-1, interleukin-2, interleukin-3, interleukin-4, interleukin-5, interleukin-7, interleukin-8, interleukin-9, interleukin-11, single-chain interleukin-12, interleukin-12, interleukin-13, interleukin-14, interleukin-15, interleukin-15-sushi, interleukin-16, interleukin-17, interleukin-18, interleukin-19, interleukin-20, interleukin-21, interleukin-22, interleukin-23, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, granulocyte-macrophage colony-stimulating factor, type I interferons, IFN-7, TNF-α, FLT3-ligand, a blocking peptide targeting TGF-β, a blocking peptide targeting IL-10, a blocking peptide targeting FoxP3, a monoclonal antibody or single-chain variable fragment (scFv) or nanobody neutralizing PD1, PDL1, CTLA4, CD137, TIM3, LAG3, and a fragment or variant thereof. A non-limiting list of immunomodulatory genes consists of shRNA targeting TGF-β, a shRNA targeting IL-10, or a shRNA targeting FoxP3.

In a preferred embodiment of the invention, the at least one immunomodulatory gene or protein encoded by the sequence of the second nucleic acid construct is selected from the group consisting of interleukin-1 (IL-1), interleukin-2 (IL-2), interleukin-4 (IL-4), interleukin-5 (IL-5), interleukin-7 (IL-7), interleukin-12 (IL-12), single chain interleukin-12 (scIL-12), interleukin-15 (IL-15), interleukin-15-sushi (IL-15-sushi), interleukin-21 (IL-21), interleukin-23 (IL-23), granulocyte-macrophage colony-stimulating factor (GM-CSF), type I interferons (IFN-α and IFN-β), IFN-7, TNF-α, CXCL10, FLT3-ligand, a blocking peptide targeting TGF-β, a blocking peptide targeting IL-10, a blocking peptide targeting FoxP3, a monoclonal antibody or scFv or nanobody neutralizing PD1, PDL1, CTLA4, CD137, TIM3, LAG3, and a fragment or variant thereof.

The person skilled in the art recognizes that any immunomodulatory protein or a fragment or a variant thereof that stimulates the immune system into targeting cancer cells is a suitable immunomodulatory protein according to the invention.

There are several immunomodulatory proteins known in the art that are used in immunotherapies for cancer treatment. For example, IL-2 is a protein which has been used in immunotherapies for more than 20 years in the treatment of cancer. However, only very few patients respond at all to IL-2-related immunotherapies while these therapies are known to cause severe side effects. IL-12 re-programs myeloid-derived suppressor cells (MDSCs) to become efficient antigen-presenting cells (APC). In addition, IL-12 strongly stimulates T-cells and NK cells and increases IFN-γ production propitiating a Th1 type of response capable of controlling the growth of the treated tumor but also of distant metastasis (Lasek et al. Cancer Immunol. Immunother. 2014; 63:419 ff). Furthermore, IL-12 has anti-angiogenic effects, which may impair tumor vascularization and thereby limit tumor growth. IL-12 is a heterodimeric cytokine which is encoded by two separate genes. To facilitate its expression in recombinant expression systems it is feasible to use the recombinant single chain IL-12 transgene that encodes both subunits of IL-12. Thus, in a preferred embodiment the immunomodulatory protein according to the invention is IL-12.

In one embodiment, the at least one immunomodulatory protein is a complex or fusion protein of IL-15 with the Sushi domain of IL-15 receptor a chain to enhance the agonist activity of IL-15 via trans-representation such as provided by the compound P22339 (Hu, Q., Ye, X., Qu, X. et al., Sci Rep 2018, 7675).

It may optionally be possible that the at least one immunomodulatory protein is selected from the group consisting of the α-subunit of IL-12, the β-subunit of IL-12, single chain IL-12 comprising the α- and β-subunit of IL-12. In the context of the present invention, a single chain IL-12 (scIL-12) is defined as a fusion protein comprising the α- and β-subunit, wherein the peptide sequences either are directly connected or interspaced by a linker peptide, wherein the linker peptide may have a length ranging from 1 to 100 amino acids. Preferably the linker has a length ranging from 4 to 60 amino acids, more preferably from 8 to 40 amino acids and particularly preferably from 12 to 25 amino acids. The linker may have a length of 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2 or 1 amino acids. In addition, the at least one immunomodulatory protein may be a single chain IL-12 comprising or consisting of the amino acid sequence of SEQ ID NO: 9 or 10 (human or murine single chain IL-12), or a polypeptide having at least 70%, 75%, 80%, 85%, 90%, 95%, or 99% sequence identity with respect to SEQ ID NO: 9 or 10, wherein the polypeptide has the biological activity of interleukin-12. The sequence identity of the polypeptide sequence with respect to SEQ ID NO: 9 or 10 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of interleukin-12, it has to have at least 10%, preferably at least 20%, more preferably at least 30% and even more preferably at least 50% of the biological activity of an interleukin-12 assessed with one of the bioactivity tests described in the following compared to the bioactivity of a polypeptide according to SEQ ID NO: 9 or 10.

The biological activity of IL-12 can be estimated in vitro by the ability of the peptide to increase IFN-7 production by cells expressing both the IL-12 receptor and the corresponding signaling pathway, such as peripheral blood mononuclear cells or human macrophage cell lines. Alternatively reporter cell lines can be used as, for instance, the Promega bioluminescent IL-12 Bioassay (Cat. #JA2601, JA2605).

In vivo IL-12 bioactivity can be estimated by the ability to increase IFN-7 serum levels.

Moreover, the at least one immunomodulatory protein may be an IFN-β comprising or consisting of the amino acid sequence of SEQ ID NO: 79, or a polypeptide having at least 70%, 75%, 80%, 85%, 90%, 95%, or 99% sequence identity with respect to SEQ ID NO: 79, wherein the polypeptide has the biological activity of IFN-β. The sequence identity of the polypeptide sequence with respect to SEQ ID NO: 79 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of IFN-β, it has to have at least 10%, preferably at least 20%, more preferably at least 30% and even more preferably at least 50% of the biological activity of an IFN-β assessed with one of the bioactivity tests described in the following compared to the bioactivity of a polypeptide according to SEQ ID NO: 79.

The biological activity of IFN-β can be estimated by the ability to induce IFN type I responsive genes. To this aim, a human cell line (like HEK 293) may be incubated with the polypeptide and the expression of IFN-responsive genes may be determined by RT-qPCR. Alternatively, IFN-reporter cells encoding luciferase under the control of an ISRE-containing promoter can be used to estimate IFN-β bioactivity of the polypeptide by measuring luciferase expression in the luminometer 3 h after addition of the polypeptide to the cell culture.

In vivo IFN-β bioactivity can be estimated by the induction of IFN-responsive genes (RT-qPCR) in peripheral blood mononuclear cells.

Furthermore, the at least one immunomodulatory protein may be an IL-15-sushi, comprising or consisting of the amino acid sequence of SEQ ID NO: 80 or a polypeptide having at least 70%, 75%, 80%, 85%, 90%, 95%, or 99% sequence identity with respect to SEQ ID NO: 80, wherein the polypeptide has the biological activity of IL-15. The sequence identity of the polypeptide sequence with respect to SEQ ID NO: 80 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of interleukin-15, it has to have at least 10%, preferably at least 20%, more preferably at least 30% and even more preferably at least 50% of the biological activity of an interleukin-15 assessed with one of the bioactivity tests described in the following compared to the bioactivity of a polypeptide according to SEQ ID NO: 80.

The biological activity of IL-15 can be estimated in vitro by a CTLL-2 cell proliferation assay.

In vivo IL-15 bioactivity can be estimated by its ability to induce the proliferation of NK cells and T cells using FACS analysis.

Furthermore, the at least one immunomodulatory protein may be a CXCL10, comprising or consisting of the amino acid sequence of SEQ ID NO: 81 or a polypeptide having at least 70%, 75%, 80%, 85%, 90%, 95%, or 99% sequence identity with respect to SEQ ID NO: 81, wherein the polypeptide has the biological activity of CXCL10. The sequence identity of the polypeptide sequence with respect to SEQ ID NO: 81 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of CXCL10, it has to have at least 10%, preferably at least 20%, more preferably at least 30% and even more preferably at least 50% of the biological activity of a CXCL10 assessed with one of the bioactivity tests described in the following compared to the bioactivity of a polypeptide according to SEQ ID NO: 81. CXCL10 is also known as IP-10.

The bioactivity of CXCL10 may be estimated by its ability to attract T lymphocytes and natural killer cells either in vivo or in an in vitro migration assay.

In a preferred embodiment the second nucleic acid construct of the invention comprises two or more sequences each encoding a different immunomodulatory protein, wherein the first immunomodulatory protein is a single chain IL-12 comprising or consisting of the amino acid sequence of SEQ ID NO: 9 or 10, or a polypeptide having at least 70%, 75%, 80%, 85%, 90%, 95%, or 99% sequence identity with respect to SEQ ID NO: 9 or 10, wherein the polypeptide has the biological activity of interleukin-12. The second immunomodulatory protein may be either an IFN-β comprising or consisting of the amino acid sequence of SEQ ID NO: 79, or a polypeptide having at least 70%, 75%, 80%, 85%, 90%, 95%, or 99% sequence identity with respect to SEQ ID NO: 79, wherein the polypeptide has the biological activity of IFN-β; or the second immunomodulatory protein may be an IL-15-sushi, comprising or consisting of the amino acid sequence of SEQ ID NO: 80, or a polypeptide having at least 70%, 75%, 80%, 85%, 90%, 95%, or 99% sequence identity with respect to SEQ ID NO: 80, wherein the polypeptide has the biological activity of IL-15; or the second immunomodulatory protein may be a chemokine, preferably a CXCL10, comprising or consisting of the amino acid sequence of SEQ ID NO: 81, or a polypeptide having at least 70%, 75%, 80%, 85%, 90%, 95%, or 99% sequence identity with respect to SEQ ID NO: 81, wherein the polypeptide has the biological activity of CXCL10.

Also, other combinations of immunomodulatory proteins are feasible. The single-chain IL-12 may be the first immunomodulatory protein whereas the second immunomodulatory protein may be selected from the group consisting of peptides that block TGF-β, peptides that block FoxP3, or monoclonal antibodies against immune checkpoint inhibitors PD1 or PD-L1, or nanobodies against immune checkpoint inhibitors PD1 or PD-L1.

It should be noted that the terms first and second immunomodulatory protein in the context of the second nucleic acid comprising two immunomodulatory proteins do not describe the order of the coding sequences on the second nucleic acid but merely are used to illustrate the two different immunomodulatory proteins. Hence, the coding sequence of the second immunomodulatory protein may lie upstream of the coding sequence of the first immunomodulatory protein on the nucleic acid. Here, upstream means that the coding sequence of the second immunomodulatory protein lies in front of the coding sequence of the first immunomodulatory protein in the 5′ to 3′ direction on the nucleic acid.

A peptide blocking TGF-β is a peptide that interferes either with TGF-β itself or with a receptor that binds TGF-β so that at least some of the TGF-β signaling is reduced or even completely prevented. Examples of peptides blocking TGF-β are P17 and P144.

A peptide blocking FoxP3 is a peptide that interferes either with FoxP3 itself or with a receptor that binds FoxP3 so that at least some of the FoxP3 signaling is reduced or even completely prevented. An example of a peptide blocking FoxP3 is P60.

In another preferred embodiment the second nucleic acid construct is a bi- or polycistronic expression cassette, wherein the sequences encoding the immunomodulatory genes or proteins are separated by a sequence encoding an IRES; and/or the sequences encoding the immunomodulatory genes or proteins are separated by a sequence encoding a 2A peptide, preferably a sequence encoding a P2A peptide. The IRES sequence and the 2A peptide encoding sequence are translation regulatory elements (TRE).

In this context, proteins or RNA having anticancer properties are any of said molecules that are capable of retarding, stopping or reversing cancer progression.

A bi- or polycistronic expression cassette is a genetic construct that contains two or more separate coding regions or genes within a single transcript. Generally speaking, it contains two or more different sequences within a single transcript. To allow for separate translation of the two or more immunomodulatory proteins and/or transcription of the immunomodulatory genes, a kind of TRE is needed that allows for individual translation of the proteins.

One possible solution is to use an internal ribosomal entry site (IRES). An IRES is a specific sequence of RNA within a transcript that allows for translation initiation at a non-AUG codon, bypassing the normal cap-dependent initiation process. This mechanism allows for the regulation of gene expression and the co-expression of multiple proteins from a single mRNA molecule.

Another possible solution are 2A peptides, also known as 2A self-cleaving peptides. 2A peptides are a class of 18-22 aa-long peptides, which can induce ribosomal skipping during translation of a protein in a cell. These peptides share a core sequence motif of DxExNPGP (SEQ ID NO: 32) and are found in a wide range of viral families. They help generating polyproteins by causing the ribosome to fail at making a peptide bond. Because of their short length 2A peptides are ideal for use in viral vector systems that have a limited capacity such as AAV. Another advantage of these peptides is that they allow for equimolar expression of the coding sequences separated by these peptides. Members of the 2A peptide family are T2A, P2A, E2A and F2A. These peptides may be flanked by linker peptides of differing lengths which may result in improved functionality. For example, the F2A element preceded by a linker domain consisting of the amino acid sequence GGSG (SEQ ID NO: 82) showed significant expression of the proteins separated by this element.

TABLE 1
Examples of combinations of two immunomodulatory proteins separated
by translation regulatory elements (TRE) in a bicistronic vector

| 1^{st} immunomodulatory | | 2^{nd} immunomodulatory |
|---|---|---|
| protein | TRE | protein |
| single-chain IL-12^{A} | IRES | IFN-β^{B} |
| single-chain IL-12^{A} | IRES | IL-15-sushi^{C} |
| single-chain IL-12^{A} | IRES | CXCL10^{D} |
| single-chain IL-12^{A} | IRES | peptide blocking FoxP3 |
| single-chain IL-12^{A} | IRES | peptide blocking TGF-β |
| single-chain IL-12^{A} | IRES | monoclonal antibody against PD1 |
| single-chain IL-12^{A} | IRES | monoclonal antibody against PD-L1 |
| single-chain IL-12^{A} | IRES | nanobody against PD1 |
| single-chain IL-12^{A} | IRES | nanobody against PD-L1 |
| IFN-β^{B} | IRES | IL-15-sushi^{C} |
| single-chain IL-12^{A} | P2A | IFN-β^{B} |
| single-chain IL-12^{A} | P2A | IL-15-sushi^{C} |
| single-chain IL-12^{A} | P2A | CXCL10^{D} |
| single-chain IL-12^{A} | P2A | peptide blocking FoxP3 |
| single-chain IL-12^{A} | P2A | peptide blocking TGF-β |
| single-chain IL-12^{A} | P2A | monoclonal antibody against PD1 |
| single-chain IL-12^{A} | P2A | monoclonal antibody against PD-L1 |
| single-chain IL-12^{A} | P2A | nanobody against PD1 |
| single-chain IL-12^{A} | P2A | nanobody against PD-L1 |
| IFN-β^{B} | P2A | IL-15-sushi^{C} |
| single-chain IL-12^{A} | F2A | IFN-β^{B} |
| single-chain IL-12^{A} | F2A | IL-15-sushi^{C} |
| single-chain IL-12^{A} | F2A | CXCL10^{D} |
| single-chain IL-12^{A} | F2A | peptide blocking FoxP3 |
| single-chain IL-12^{A} | F2A | peptide blocking TGF-β |
| single-chain IL-12^{A} | F2A | monoclonal antibody against PD1 |
| single-chain IL-12^{A} | F2A | monoclonal antibody against PD-L1 |
| single-chain IL-12^{A} | F2A | nanobody against PD1 |
| single-chain IL-12^{A} | F2A | nanobody against PD-L1 |
| IFN-β^{B} | F2A | IL-15-sushi^{C} |

^{A}being preferably a human single chain IL-12 as depicted in SEQ ID NO: 9
^{B}being preferably a human IFN-β as depicted in SEQ ID NO: 79
^{C}being preferably a human IL-15-sushi as depicted in SEQ ID NO: 80
^{D}being preferably a human CXCL10 as depicted in SEQ ID NO: 81

Immunotherapies that involve systemic availability of the immunomodulatory protein often cause unwanted side effects. However, an advantage of this invention is that the recombinant expression system used herein induces the local expression of said immunomodulatory protein thereby inducing a highly local and targeted immune response. For example, the intratumor administration of the recombinant expression system according to the invention induces an immune response within or in close proximity to the tumor. Without being wished to be bound by theory, it is thought that the release of the immunomodulatory protein within or in the vicinity of the tumor causes T-cells to infiltrate the tumor vicinity and may cause the recognition of tumor cells that normally escape the recognition of the immune system, thereby enabling the immune system to target and kill these cells. Thus, tumor progression is at least retarded, preferably halted, and more preferably reversed.

In a preferred embodiment the immunomodulatory gene or protein of the invention is an immunostimulatory gene or protein. That is a gene or protein that stimulates the immune system such as by directly stimulating it (e.g. IL-12) or by inhibiting immunosuppressive molecules (e.g. blocking peptide or shRNA targeting TGF-β).

According to a further possibility it may be provided that the STING-responsive promoter of the second nucleic acid construct is selected from the group consisting of IFN-β-responsive promoter (preferably according to SEQ ID NO: 25), IFN-β-minimal promoter (preferably according to SEQ ID NO: 31), CXCL9-responsive promoter (preferably according to SEQ ID NO: 23), CXCL10-responsive promoter (preferably according to SEQ ID NO: 24), STAT-motif promoter (preferably according to SEQ ID NO: 26), IRSF3-motif promoter (preferably according to SEQ ID NO: 27 or SEQ ID NO: 28), ISRE-motif promoter (preferably according to SEQ ID NO: 12 or SEQ ID NO: 13), NFκB-motif promoter (preferably according to SEQ ID NO: 29 or 30) and STAT6-motif promoter. In a preferred embodiment, the STING responsive promoter comprises at least one interferon response element (ISRE).

By operatively linking the expression of the immunomodulatory gene or protein to the activity of STING using a STING-responsive promoter according to the invention several advantages are accomplished. Firstly, the expression of the immunomodulatory protein may be controlled by using a human-non-reacting STING agonist. Thus, it is possible to adapt the expression of the immunomodulatory protein according to the needs of the therapy rather than causing a continuous high-level expression of the protein. Secondly, a targeted and local expression of the immunomodulatory protein can be achieved thereby avoiding potential side effects by systemic expression of the protein.

It may optionally be possible that the STING-responsive promoter is selected from the list consisting of:
a. a nucleotide sequence comprising one to ten copies, preferably two to seven copies, more preferably three to five copies and particularly preferably 4 copies of a sequence having at least 75%, at least 80%, at least 85%, at least 90% or at least 95% sequence identity with respect to SEQ ID NO: 11 or is identical to SEQ ID NO: 11;
b. a nucleotide sequence according to a., wherein the copies are interspaced by up to forty nucleotides, preferably four to thirty-five nucleotides, more preferably ten to thirty nucleotides and particularly preferably fifteen to twenty-five nucleotides;
c. a nucleotide sequence according to SEQ ID NO: 12 which comprises four copies of the ISRE interspaced with 20 nucleotides;
d. a nucleotide sequence according to SEQ ID NO: 13 which comprises four copies of the ISRE interspaced with 6 nucleotides.

The use of STING-responsive promoters in the context of the invention ensures the controlled and targeted expression of the immunomodulatory protein. Moreover, the aforementioned STING-responsive promoters have been shown to have additional advantages. As elaborated in the examples of this application the STING-responsive promoters have been shown to be inducible by administering a human-non-reacting STING agonist in the presence of target STING. Moreover, the induction of these promoters has been shown to be specific in that in the absence of the selective STING agonist only limited expression occurs. In particular, promoters carrying four copies of the ISRE as depicted in SEQ ID NO: 12 are highly inducible while also displaying high specificity.

Preferably, the nucleotide sequence of the STING-responsive promoters as described in a. and b. do not contain an AP-1 site or an NFκB site. Even more preferably the nucleotide sequence of the STING-responsive promoters as described in a. and b. do not contain any further motif that contributes to inducibility, leakage or expression efficiency of the promoter. In other words, the nucleotide sequence of the promoter as described in a. or b. may contain several nucleotides prior to, in between and/or after the SEQ ID NO: 11, wherein these additional nucleotides are chosen in a way that (e.g. randomly) they do not form any transcription regulatory element. Particularly preferably, the nucleotide sequence of the STING-responsive promoters as described in a. and b. consist of one to ten copies, preferably two to seven copies, more preferably three to five copies and particularly preferably 4 copies of a sequence having at least 75%, at least 80%, at least 85%, at least 90% or at least 95% sequence identity with respect to SEQ ID NO: 11 or is identical to SEQ ID NO: 11.

In addition, the STING-responsive promoters as described in a. to d. may already represent a functional promoter, so that no other element is needed for these promoters to induce the expression of the operatively linked transgene.

According to a further possibility it may be provided that the second nucleic acid construct further comprises at least one sequence motif that inhibits transgenic expression of the immunomodulatory protein in tissues or cells not intended to express the protein, preferably being the liver, wherein the sequence motif is preferably a target sequence of a microRNA which is highly abundant in the liver.

It is known that several viral particles, especially AAV, show a tropism towards liver cells. This means that when viral particles gain access to the blood circulatory system, they preferentially transduce liver cells, eventually causing the expression of the encoded transgene in the liver. Also in the event of intratumor injections of said viral particles, some of these particles will access the blood circulatory system, as the tumor is highly vascularized. To prevent the unwanted expression of the transgene encoded by the viral particle in the liver it is therefore suitable to control its expression. Several methods are known by the person skilled in the art to achieve this goal.

In general, a suitable method to limit transgene expression to a specific tissue or a specific cell type is to use viral particles with a distinct tropism towards said tissues or cell types. A way to achieve this is by altering the serotype of an AAV or by altering the capsid of the AAV. Several AAV serotypes are known in the art each with distinct tropisms. However, as the invention is intended to target several different types of cancers, it would be necessary to optimize the serotype or capsid for each cancer or tumor type. Nonetheless, this may be a suitable way to achieve tissue- or cell type-specific expression of a transgene.

The skilled person may also use DNA- or RNA-binding repressors to avoid unwanted expression of the transgene encoded by the viral particle in tissues or cells not intended to express the transgene. Therefore, the second nucleic acid construct may comprise an operon that binds a repressor. The repressor may be operatively linked to a promoter that is active in hepatocytes, hence, inducing the expression of the repressor in these cells, thereby, inhibiting expression of the transgene.

Another possibility is to use RNA interference (RNAi). RNAi is characterized in that an RNA molecule reduces or inhibits expression of a nucleic acid sequence with which the RNA molecule shares substantial or total homology. Non-limiting examples of RNAi are small interfering RNA, small hairpin RNA or microRNA (miR). To reduce or inhibit transgene expression in a specific tissue using RNAi it may be conceivable that for example a shRNA targeting the nucleotide sequence of said transgene may be operatively linked to a tissue specific promoter. When said shRNA is administered it thereby reduces or inhibits transgene expression in the tissue. Although this method would be feasible in the context of the invention it may be disadvantageous in that an additional administration of said shRNA would be necessary to limit transgene expression in the target tissue, e.g. the liver.

An elegant way to avoid this drawback is to operatively link a miRNA target sequence to the nucleotide sequence of the transgene. Thereby, expression of the transgene will be reduced or inhibited in tissues where a miRNA that is associated to said miRNA target sequence is present. Accordingly, a preferred embodiment of the invention comprises that the sequence motif is a target sequence of a microRNA which is highly abundant in the liver. Thereby, a single administration of a nucleic acid construct is sufficient to avoid transgene expression in the liver while transcription in other tissues or organs is not impaired.

It is optionally conceivable that the microRNA abundant in the liver is selected from the group consisting of miR-122, miR-192, miR-199a, miR-101, miR-99a, let7a, let7b, let7c or let7f. Furthermore, it may be advantageous that the microRNA abundant in the liver is miR-122. The advantage of miR-122 is that it is highly expressed in the liver and, importantly, specifically expressed in the liver. Hence, by choosing a miR-122 target sequence a significant reduction of the transgene in the liver is ensured while expression in other tissues is not impaired. Moreover, it has been shown that miR-122 is downregulated in most liver tumors so that the recombinant expression system according to the invention is also suitable to treat liver cancer.

According to a preferred embodiment of the invention, the respective miR-122 target sequence is a miR-122 target sequence having at least 90% or at least 95% sequence identity with respect to SEQ ID NO: 14 or is identical to SEQ ID NO: 14. Preferably, the target sequence comprises 1 to 12, preferably 3 to 10, more preferably 4 to 8 and particularly preferably 5 copies of the miR-122 target sequence as depicted in the previous sentence.. Increasing the copy number of miR-122 target sequences increased the miR-mediated reduction, whereas no significant improvement was observed above the disclosed number of 5 copies. In a preferred embodiment the copies are interspaced by one to forty nucleotides, preferably one to thirty nucleotides, more preferably two to twenty nucleotides and particularly preferably two to eight nucleotides.

It is preferred that the nucleotide sequence comprises 5 copies of the miR-122 target sequence as depicted in SEQ ID NO: 15. As elaborated in the examples of this application, the nucleotide sequence as depicted in SEQ ID NO: 15 has been shown to substantially inhibit transgene expression in the liver thus causing the plasma levels of the transgene to be undetectable by the methods described in Example 4.

According to a further possibility, it may be provided that the second nucleic acid construct further comprises a transgene encoding a thymidine kinase, preferably a thymidine kinase according to SEQ ID NO: 16. The nucleotide sequence encoding the thymidine kinase is linked to the nucleotide sequence of the immunomodulatory gene or protein and may be interspaced by a 2A self-cleaving peptide (so called 2A peptides). 2A peptides constitute a class of 18-22 amino acid-long peptides, which can induce ribosomal skipping during translation of a protein in a cell. These peptides share a core sequence motif of “DXEXNPGP” and are found in a wide range of viral families. They help generating polyproteins by causing the ribosome to fail at making a peptide bond. The person skilled in the art recognizes that the use of a 2A peptide allows the co-expression of the immunomodulatory protein and the thymidine kinase. The co-expression of the thymidine kinase is advantageous in that it allows to assess tumor transduction by PET imaging and, in case of potential IL-12-mediated toxicity, to eliminate the cells expressing the cytokine by administering a thymidine kinase substrate such as for example acyclovir, penciclovir, famciclovir or ganciclovir.

The first and/or the second nucleic acid construct can further comprise a woodchuck hepatitis virus post-transcriptional regulatory element (WPRE). The WPRE is known to increase transgene expression, whereby it is most effective when placed downstream of the transgene (i.e., the target STING or the immunomodulatory gene or protein), proximal to the polyadenylation signal.

Expediently, the first and/or the second nucleic acid construct further comprise a polyadenylation signal located at the 3′position with respect to the target STING or the immunomodulatory gene or protein. The of length the of Poly(A) tail may be optimized for stabilization of mRNA and subsequent increased protein translation.

The skilled person is also aware of other means to increase protein translation such as
a. by sequence modification to optimize G/C content and/or excessively stabilizing secondary structure codon, or
b. alternating the 5′cap by adding synthetic or natural 5′cap analogues such as m7GpppG or an initiating trimer such as “CleanCap” or stabilization by means of capping enzymes, or
c. addition of regulatory elements
   c.1 in the 5′-untranslated region (UTR) such as optimization through appropriate cis regulatory sequences or modifying length and sequence to avoid excessively stable structure, or
   c.2 in the 3′-UTR through sequence modification to introduce stable elements or repeated motifs or synthetic or natural sequences that have been proven effective in other mRNAs.

In a special embodiment, the second nucleic acid construct of the recombinant expression system consists of a nucleic acid construct as given in the following table, wherein the different elements are shown in the 5′ to 3′direction as promotor, first open reading frame (ORF1), first translation regulatory element (TRE1), second open reading frame (ORF2), second translation regulatory element (TRE2) and target sequence for a microRNA (microRNA element).

TABLE 2
Embodiments of the second nucleic acid construct of the expression system

| Promoter | ORF1 | TRE1 | ORF2 | TRE2 | microRNA element |
|---|---|---|---|---|---|
| ISREx4 ^{A} | scIL12 ^{B} | | | | |
| ISREx4 ^{A} | scIL12 ^{C} | | | | |
| IFN-β minimal | scIL12 ^{B} | | | | |
| IFN-β minimal | scIL12 ^{C} | | | | |
| ISREx4 ^{A} | scIL12 ^{B} | | | | miR122 target site ^{E} |
| ISREx4 ^{A} | scIL12 ^{C} | | | | miR122 target site ^{E} |
| IFN-β minimal | scIL12 ^{B} | | | | miR122 target site ^{E} |
| IFN-β minimal | scIL12 ^{C} | | | | miR122 target site ^{E} |
| ISREx4 ^{A} | scIL12 ^{B} | | | WPRE | miR122 target site ^{E} |
| ISREx4 ^{A} | scIL12 ^{C} | | | WPRE | miR122 target site ^{E} |
| IFN-β minimal | scIL12 ^{B} | | | WPRE | miR122 target site ^{E} |
| IFN-β minimal | scIL12 ^{C} | | | WPRE | miR122 target site ^{E} |
| ISREx4 ^{A} | HSV-TK ^{D} | p2A | scIL12 ^{B} | WPRE | miR122 target site ^{E} |
| ISREx4 ^{A} | HSV-TK ^{D} | p2A | scIL12 ^{C} | WPRE | miR122 target site ^{E} |
| ISREx4 ^{A} | scIL12 ^{B} | p2A | HSV-TK ^{D} | WPRE | miR122 target site ^{E} |
| ISREx4 ^{A} | scIL12 ^{C} | p2A | HSV-TK ^{D} | WPRE | miR122 target site ^{E} |

^{A} being preferably 4 copies of ISRE, preferably given by SEQ ID NO: 12
^{B} being preferably a human single chain IL-12 as depicted in SEQ ID NO: 9
^{C} being preferably a murine single-chain IL-12 as depicted in SEQ ID NO: 10
^{D} being preferably a HSV thymidine kinase as depicted in SEQ ID NO: 16
^{E} being preferably 5 copies of miR-122 target sequence as depicted in SEQ ID NO: 15

Another aspect of the invention is an expression vector encoding the first and the second nucleic acid construct according to the invention. Using an expression vector comprising the first and second nucleic acid construct has certain advantages. Firstly, expression vectors are specifically designed to allow the introduction of the encoded nucleotide sequences into a host cell and commandeer their expression. Secondly, expression vectors are plasmids that are very stable and thereby protected from degradation. Thirdly, expression vectors are conceivable for gene delivery using different methods. A non-limiting example of an expression vector and a preferred embodiment of the invention is a viral vector, more preferably a viral vector selected from the list consisting of adeno-associated virus (AAV) vector, adenoviral vector, lentiviral vector, vaccine virus vector, or herpes simplex virus vector. Viral vectors in particular combine several advantages of expression vectors such as general safety, low toxicity and stability.

It is conceivable that the first and second nucleic acid construct of the recombinant expression system according to the invention are contained within a single expression vector. This has the advantage that one administration of said expression vector is sufficient to express the recombinant expression system according to the invention in the patient.

An alternative aspect of the invention is a combination of a first and a second expression vector, the first vector encodes the first nucleic acid construct, and the second vector encodes the second nucleic acid construct according to the invention. This has the advantage that the size of the respective nucleic acid construct is not as limited as it would be if both nucleic acid constructs would be combined in one vector. Size limitation is a frequently occurring problem with viral vectors. For example, adeno-associated viruses (AAV) are limited to a size of around 5 kb, whereas it has to be taken into consideration that the ITRs, the promoter and other elements are not included in the 5 kb.

Thus, in an alternative aspect, the recombinant expression system of the invention provides a combination of a first expression vector encoding the first nucleic acid construct and a second expression vector encoding the second nucleic acid construct.

It is also advantageous that the first and/or the second nucleic acid construct of the expression vector(s) further comprise a 5′ITR and a 3′ITR sequence, being preferably AAV ITR as depicted in SEQ ID NO: 17. It may be advantageous that the expression vector(s) is (are) an AAV vector and preferably has (have) the serotype AAV1, AAV3, AAV6, AAV8, AAV9, AAV2, AAV5, AAVrh.10, or any gain-of-function mutant of AAVrh.10, whereas AAV2 is most preferred. AAVs are commonly used in biotechnology and therefore well characterized. Advantages of using AAV vectors are that they are generally regarded as safe, display low toxicity and are stable. Moreover, AAVs are already approved by several governmental organizations around the world for use in human.

It is also advantageous that the first and/or the second nucleic acid construct of the expression vector(s) further comprise a 5′ITR, a W packaging signal and a 3′ITR sequence, being preferably the 5′ITR, the W packaging signal and the 3′ITR of an adenovirus (Ad). It may be advantageous that the expression vector(s) is (are) an adenoviral vector, preferably a high-capacity adenoviral vector. Adenoviruses are commonly used in biotechnology and therefore are well characterized. An advantage of adenoviral vectors is that they have a higher size limitation compared to AAV vectors. Moreover, adenoviruses are already approved by several governmental organizations around the world for use in human.

It is also advantageous that the expression vector(s) is (are) a lentiviral vector, a vaccine virus vector or a herpes simplex virus vector. These vectors are commonly used in biotechnology and therefore well characterized.

It is also conceivable that nanoparticles may be used to deliver the first and/or second nucleic acid of the invention. Nanoparticles as gene delivery systems are known in the art and have advantages like tunable size, shape and/or surface thereby allowing to regulate their biological behaviors. For effective delivery and uptake by cells the nucleic acid constructs would be packaged into nanoparticles, preferably liposomes, made with pharmaceutically acceptable carriers composed of varying proportions of physiologically compatible lipids, proteins, polysaccharides or synthetic polymers such as standard lipids compatible with biological membranes selected from the list consisting of phospholipids such as distearoylphosphatidylglycerol, distearoylphosphatidylcholine, (DLPC) 1,2-dilauroyl-sn-glycero-3-phosphocholine, (DMPC) 1,2-dimyristoyl-sn-glycero-3-phosphocholine, (DPPC) 1,2-dipalmitoyl-sn-glycero-3-phosphocholine, (DSPC) 1,2-distearoyl-sn-glycero-3-phosphocholine, (DOPC) 1,2-dioleoyl-sn-glycero-3-phosphocholine, (DMPE) 1,2-dimyristoyl-sn-glycero-3-phosphoethanolamine, (DPPE) 1,2-dipalmitoyl-sn-glycero-3-phosphoethanolamine, (DOPE) 1,2-dioleoyl-sn-glycero-3-phosphoethanolamine, (DOPS) 1,2-dioleoyl-sn-glycero-3-phospho-L-serine, (DPPS) 1,2-dipalmitoyl-sn-glycero-3-phospho-L-serine, (DMPS) 1,2-dimyristoyl-sn-glycero-3-phospho-L-serine, (DOPG) 2,3-Dimyristoyl-sn-glycero-1-phosphocholine, and their respective enantiomers, wherein distearoylphosphatidylglycerol and distearoylphosphatidylcholine are most preferred. Another suitable example is the use of cardiolipins preferably combined with cholesterol. Alternative nanoparticles are dendrimer nanoparticles, protamine liposomes, cationic polymers, cationic polymer liposomes, polysaccharide particles, cationic lipid particles, cationic lipid cholesterol nanoparticles, cationic lipid cholesterol PEG nanoparticles, ionizable lips preferably lipids positively charged under physiological conditions, including amino lipids such as MC3 (DLin-MC3-DMA), C12-200, cKK-E12, ZA3-Ep10, 200-01I0; (SM-102) heptadecan-9-yl 8-((2-hydroxyethyl) (6-oxo-6-(undecyloxy)hexyl)amino) octanoate, (ALC 0159) N,N-dimyristylamide of 2-hydroxyacetic acid, 0-pegylated to a PEG chain of approx. 2 kilodaltons, (ALC 0315) (4-hydroxybutyl)azanediyl]di(hexane-6,1-diyl) bis(2-hexyldecanoate), Lipofectamine, Trans-IT, InstantFECT, (DOTAP) 1,2-Dioleoyl-3-trimethylammonium. Other components may include peptides and polypeptides such as DP7-C the cholesterol-modified cationic peptide DP7 (VQWRIRVAVIRK, SEQ ID NO: 85), polymers such as polyethylene glycol preparations composed of polymer molecules of varying molecular sizes which may be noncovalently bound or covalently linked to lipids in the formulation.

Another aspect of the invention is a viral particle comprising the aforementioned viral vector which comprises the first and second nucleic acid construct of the invention. This has the advantage that one administration of said viral particle is sufficient to express the recombinant expression system according to the invention in the patient.

Yet another aspect of the invention is a combination of a first and a second viral particle, the first viral particle comprises a viral vector encoding the first nucleic acid construct and the second viral particle comprises a viral vector encoding the second nucleic acid construct according to the invention. This has the advantage that the size of the respective nucleotide sequence is not as limited as it would be if both nucleic acid constructs would be combined in one viral vector of a single viral particle. Size limitation is a frequently occurring problem with viral vectors. For example, adeno-associated viruses (AAV) are limited to a size of around 5 kb, whereas it has to be taken into consideration that the ITRs, the promoter and other elements are not included in the 5 kb.

The aforementioned viral particle(s) is (are) preferably (an) AAV or (an) adenoviral particle(s); more preferably (an) AAV viral particle(s). As described hereinbefore, Ad and AAV display several advantages over other viral particles such as that they are well characterized, frequently used and approved for treatment in humans. These viruses display further advantages that render them particularly useful in gene therapy such as that they are capable of transducing dividing as well as non-dividing cells and that they do not integrate into the host genome.

In a specific embodiment the combination of the first expression vector and the second expression vector, or the combination of the first viral particle and the second viral particle are provided as a kit-of-parts.

Another aspect of the invention is a pharmaceutical composition that comprises a pharmaceutically acceptable excipient together with the recombinant expression system, the expression vector(s) or the viral particle(s) according to the invention. Pharmaceutical compositions have the advantage that they are “ready-to-use” in that they may be administered directly to the patient for treatment. In addition, pharmaceutic compositions are tailored to optimize stability and availability of the ingredients according to the therapeutic need while avoiding unwanted side effects for the patient.

In another aspect of the invention the recombinant expression system, the expression vector(s), the viral particle(s), or the pharmaceutical composition, each according to the invention may be used in a therapy, preferably an immunotherapy or are provided for use in a therapy, preferably an immunotherapy.

In a preferred embodiment the immunotherapy is the immunotherapy of a proliferative disorder, more preferably the immunotherapy of cancer, even more preferably the immunotherapy of solid cancer. While it is conceivable that non-solid cancers may also be treated by the immunotherapy of the invention the use for the therapy in solid cancers has the advantage that these cancers are delimited locally and therefore are accessible by local administration. Hence, it is possible to administer the recombinant expression system, the expression vector(s), the viral particle(s) or the pharmaceutical composition according to the invention locally to the tumor by intratumor administration. This has the advantage that a local and targeted immune response may be induced, thus causing the retardation, the halt or reversal of tumor progression. In addition, although the invention takes measures to avoid systemic expression, for example by using miR target sequences, a local administration reduces the unwanted systemic expression even further.

As already described above cancer immunotherapies have several limitations such as i) poor tumor targeting, ii) weak tumor cell killing ability, iii) high effort and thereby high cost, iv) tumor immune escape, v) over-stimulation of the immune system which can cause severe side effects (Tan et al. 2020, Biomed Pharmacother. 124: 109821). The immunotherapy according to the invention is an extended immunotherapy as it also incorporates gene therapy. Hence, the immunotherapy according to the invention is not merely an immunotherapy by itself but a combination of immunotherapy and gene therapy, hereinafter termed “immunogene therapy” to effectively treat proliferative disorders like cancer.

It may further be possible that the cancer is selected from the list consisting of: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burkitt's lymphoma; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xeroderma pigmentosum, keratoacanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, urothelial cancer, hepatocellular carcinoma, hepatoblastoma, hepatoma, breast cancer, colon carcinoma, and head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, Sino nasal natural killer, neoplasms, plasma cell tumors, carcinoid tumors, neuroendocrine tumors, cholangiocarcinoma, and Merkel cell carcinoma.

In another preferred embodiment the recombinant expression system, expression vector(s), the viral particle(s) or pharmaceutical composition of the invention is (are) employed in a combination therapy with at least one other therapy selected from the group consisting of a therapy comprising at least one checkpoint inhibitor, a therapy comprising at least one adoptive cell therapy, a therapy comprising at least one cancer vaccine, a therapy comprising at least one exogenous cytokine and/or a therapy comprising a radiotherapy.

The inhibitor of the therapy comprising at least one checkpoint inhibitor may be selected of the group consisting of an inhibitor of PD-1, PD-L1, PD-L2, CTLA-4, LAG3, BTLA, B7H3, B7H4, TIM3, KIR, or A2aR, more preferably selected from the group consisting of an inhibitor of PD-1, PD-L1, PD-L2, or CTLA-4. Immune checkpoints are molecules expressed on T cells or cells interacting with T cells that help regulate the immune response and prevent excessive or prolonged immune activation. However, cancer cells can take advantage of these checkpoint proteins to evade detection by the immune system. By blocking these checkpoint proteins, immune checkpoint inhibitors can release the brakes on the immune response and allow the immune system to attack and destroy cancer cells. Using immune checkpoint inhibitors as an additional therapy is particularly advantageous in the context of this invention as the expression system of the invention causes infiltration of T cells into the tumor environment. As these T cells might be impaired in their function due to the immune checkpoints, means to overcome this hurdle may greatly benefit the treatment outcome.

A therapy comprising at least one adoptive cell therapy may be an adoptive NK cell therapy, adoptive dendritic cell therapy, or adoptive T-cell therapy, wherein the adoptive T-cell therapy preferably is a CAR-T-cell therapy or a therapy using tumor infiltrating leukocytes (TIL). In some cases, tumors silence the expression of MHC class I molecules and/or IFN-7 signaling molecules as a means to evade T cell immunity. Using adoptive cell therapy may circumvent these problems as these adopted cells are normally capable of recognizing and targeting tumor cells. Thus, synergistic effects may be expected in a combinatory approach.

Also, the combination of the expression system of the invention with a cancer vaccine or exogenous cytokines may cause synergistic effects as the recruited T cells are primed towards cancer cells by cancer vaccines or exogeneous cytokines.

In a preferred embodiment of the invention, the cancer is selected from the list consisting of bladder cancer, bone cancer, brain cancer, breast cancer, cancer of the thymus, cervical cancer, colon cancer, oesophageal cancer, gastric cancer, head & neck cancers, Hodgkin's lymphoma, liver cancer, lung cancer, melanoma, mesothelioma, multiple myeloma, Merkel cell carcinoma, non-Hodgkin's lymphoma, ovarian cancer, pancreatic cancer, prostate cancer, rectal cancer, urothelial cancer, renal cancer, sarcoma, skin cancer, testicular cancer, thyroid cancer, uterine cancer, plasma cell tumors, cholangiocarcinoma, and, neuroendocrine tumors or carcinoid tumors.

In yet another aspect, the invention relates to the recombinant expression system, the expression vector(s), the viral particle(s) or the pharmaceutical composition, of the invention for use in an immunotherapy, wherein the immunotherapy comprises the following steps:
a. administration of the recombinant expression system according to the invention; the expression vector(s) according to the invention, the viral particle according to the invention or the pharmaceutical composition according to the invention to a patient suffering from a proliferative disorder, being preferably cancer in order to yield a therapeutically effective transgenic expression of the target STING and the immunomodulatory protein, wherein the administration is preferably an intratumoral administration or a systemic administration;
b. optionally treating the patient before and/or concurrently to the administration according to step a) with a radiotherapy, being preferably a radiotherapy directed to the tissue or organ intended to express the transgene(s) from a); and/or;
c. treating the patient after the administration according step a) with a STING agonist which recognizes the transgenically expressed target STING but not the human STING and natural variants thereof; and/or with a radiotherapy, being preferably a radiotherapy directed to the tissue or organ intended to express the transgene(s) from a).

Of note, step c) provides three possible different treatment options. For example, only a STING agonist which recognizes the transgenically expressed target STING but not the human STING and natural variants thereof (human non-reacting STING agonist) may be applied. Another possibility is to only treat the patient with the radiotherapy. Finally, also both options may be combined.

In another embodiment the patient is treated before, concurrently and/or after the administration according to step a) with at least one other therapy selected from the group consisting of a therapy comprising at least one checkpoint inhibitor, a therapy comprising at least one adoptive cell therapy, a therapy comprising at least one cancer vaccine and/or a therapy comprising at least one exogenous cytokine. In a preferred embodiment the treatment with the at least one other therapy occurs after the administration according to step a).

In a similar aspect, the invention relates to a method for treating a proliferative disorder, being preferably cancer, in a patient, the method comprising:
a. administering the recombinant expression system according to the invention; the expression vector(s) according to the invention, the viral particle(s) according to the invention or the pharmaceutical composition according to the invention to the patient suffering from a proliferative disorder, being preferably cancer in order to yield a therapeutically effective transgenic expression of the target STING and the immunomodulatory protein, wherein the administration is preferably an intratumoral administration or a systemic administration;
b. optionally treating the patient before and/or concurrently to the administration according to step a) with a radiotherapy, being preferably a radiotherapy directed to the tissue or organ intended to express the transgene(s) from a); and/or;
c. treating the patient after the administration according step a) with a STING agonist which recognizes the transgenically expressed target STING but not the human STING and natural variants thereof, and/or with a radiotherapy, being preferably a radiotherapy directed to the tissue or organ intended to express the transgene(s) from a).

In another embodiment the patient is treated before, concurrently and/or after the administration according to step a) with at least one other therapy selected from the group consisting of a therapy comprising at least one checkpoint inhibitor, a therapy comprising at least one adoptive cell therapy, a therapy comprising at least one cancer vaccine and/or a therapy comprising at least one exogenous cytokine. In a preferred embodiment the treatment with the at least one other therapy occurs after the administration according to step a).

Notably, the embodiments described in the following apply to both, the recombinant expression system, the expression vector(s), the viral particle(s), or the pharmaceutical composition, of the invention for use in the immunotherapy and the method of treating a proliferative disorder.

It is conceivable that the use for immunotherapy or the method of treating a proliferative disorder according to the invention may be combined with other treatments that are anti-cancer therapies. Several anti-cancer therapies are known in the art such as chemotherapy, hormone therapy, radiotherapy, photodynamic therapy, stem cell transplants, surgery, targeted therapy, hyperthermia, or other immunotherapies. It may be advantageous to direct the radiotherapy to the tumor that has received intratumor administration according to step a).

It is also advantageous if in step c) the STING agonist is selected from the group consisting of 5,6-dimethylxanthenone-4-acetic acid (DMXAA), 7-bromo-DMXAA, 7-iodo-DMXAA, 7-hydroxy-DMXAA, 7-formyl-DMXAA, 7-hydroxymethyl-DMXAA, 7-(2-hydroxyethyl)-DMXAA, flavone-8-acetic acid (FAA), 2,7-bis(2-dimethylamino ethoxy)fluoren-9-one (Tilorone) and 10-carboxymethyl-9-acridanone (CMA). Preferably the STING agonist is DMXAA. DMXAA is already proven in clinical studies as a molecule with beneficial safety and lack of severe side effects. In addition, according to the invention DMXAA activates target STING from step a. without activating human wild-type STING or natural variants thereof, thereby rendering the treatment as generally safe and avoiding systemic STING activation.

It is also optionally conceivable that in step b) and/or step c) a radiotherapy with 100 Gy or less is used. Any ionizing radiation dose of less than 100 Gy, 96 Gy, 92 Gy, 88 Gy, 84 Gy, 80 Gy, 76 Gy, 72 Gy, 68 Gy, 64 Gy, 60 Gy, 56 Gy, 52 Gy, 48 Gy or 44 Gy may be used. It may also be conceivable that in step b) and/or step c) a low dose radiotherapy with 40 Gy or less, preferably 10 Gy or less and more preferably 8 Gy or less is used. Low-dose radiotherapy can be any radiotherapy with an ionizing radiation dose of less than 40 Gy, 38 Gy, 36 Gy, 34 Gy, 32 Gy, 30 Gy, 28 Gy, 26 Gy, 24 Gy, 22Gy, 20 Gy, 19 Gy, 18 Gy, 17 Gy, 16 Gy, 15 Gy, 14 Gy, 13 Gy, 12 Gy, 11 Gy, 10 Gy, 9 Gy, 8 Gy, 7 Gy, 6 Gy, 5 Gy, 4 Gy, 3 Gy, 2.5 Gy, 2.0 Gy, 1.5 Gy, 1 Gy, 0.5 Gy, 0.25 Gy, 0.2 Gy, 0.15 Gy, 0.1 Gy, 0.05 Gy, or 0.01 Gy. An advantage of using low-dose radiotherapy is that it is better tolerated by patients in that they experience less or no side effects that are otherwise common in radiotherapy. As used herein, the indicated doses refer to total doses, meaning that the total dose for a treatment in step b) or step c) does not exceed the claimed dose. Hence, a dose of 40 Gy as applied in step b) can be achieved by a single dose of 40 Gy or fractioned doses, for example four doses of 10 Gy, given over a period for up to several weeks or even months.

In another embodiment the radiation therapy in step c) comprises several separate radiation treatments, wherein the cumulated dose of the separate radiation treatments adds up to less than 100 Gy, preferably less than 40 Gy and more preferably to less than 25 Gy. The cumulated dose of the separate radiation treatments may be less than 100 Gy, 96 Gy, 92 Gy, 88 Gy, 84 Gy, 80 Gy, 76 Gy, 72 Gy, 68 Gy, 64 Gy, 60 Gy, 56 Gy, 52 Gy, 48 Gy, 44 Gy, 40 Gy, 38 Gy, 36 Gy, 34 Gy, 32 Gy, 30 Gy, 28 Gy, 26 Gy, 24 Gy, 22Gy, 20 Gy, 19 Gy, 18 Gy, 17 Gy, 16 Gy, 15 Gy, 14 Gy, 13 Gy, 12 Gy, 11 Gy, 10 Gy, 9 Gy, 8 Gy, 7 Gy, 6 Gy, 5 Gy, 4 Gy, 3 Gy, 2.5 Gy, 2.0 Gy, 1.5 Gy, 1 Gy, 0.5 Gy, 0.25 Gy, 0.2 Gy, 0.15 Gy, 0.1 Gy or 0.05 Gy.

Notably, radiotherapy may be either applied externally or internally. The person skilled in the art knows several means to provide external radiotherapy. For example, external radiotherapy may be provided by gamma ray, electron beam or proton accelerators adequate for therapeutic purposes. However, for some tumors it may be advantageous to use internal radiotherapy. Internal radiotherapy may be provided by administration of a pharmacologically acceptable forma and dose of a radioactive isotope for therapeutic purposes. Embodiments of internal radiotherapy may be brachytherapy or non-localized radiotherapy. Brachytherapy physically constraints radioactive deposits so that they are essentially not movable from the location they have been deposited. The radioactive deposits may be placed interstitially (interstitial radiotherapy) which is used for example for quite a significant percentage of prostate tumors and some head and neck tumors, or radioactive deposits may be placed inside a cavity (intracavity radiotherapy) which is used for example for some uterus, bronchus, or esophagus tumors. The non-localized radiotherapy may be either a partially disseminated local administration such as selective internal radiation therapy (SIRT) where a suspension of material is injected into particular arteries or veins with the objective that it settles close to the site of injection rather than experience wide distribution in the body, or it may be an entirely systemic approach such as use of soluble chemical substances synthesized with radioactive isotopes which find their spot of action by affinity or biologicals such as radiolabeled antibodies in order to better deliver the radiation to the target tumor. A classic example for the latter is iodine-131. Although different isotopes may be used for different treatment paradigms, the isotopes useful in this invention may be preferably selected from the list consisting of yttrium-90, iodine-131, lutetium-177, iridium-192, astatine-211, bismuth-213, actinium 225, samarium-153, radium-223, phosphorus-32, strontium-89, erbium-169, holmium-166, terbium-161, gallium-68, rhenium-188 or rhenium-186.

As the inventors found out, there is a synergism between canonical STING pathway activated by DIXAA and non-canonical STING pathway activated by radiotherapy in vitro (see Example 9) and also in vivo (see Example 10) supporting the beneficial combination of STING agonist treatment and radiotherapy in step c.

Furthermore, it is an advantage of the second nucleic acid construct that it can be applied systemically and the expression of the at least one immunomodulatory gene or protein can be induced by radiation. Thus, radiation has a two-fold effect, as it not only facilitates the transduction of the virus into the irradiated cells but, in addition, it also induces the expression of the at least one immunomodulatory gene or protein.

The above-mentioned first effect relates to an important concept of the invention, namely that radiotherapy marks the tumor for transgene expression from gene vectors, such as AAV vectors. This fact has very important clinical applications since the data of the present application indicate that tumor irradiation converts the tumor into a target for transgene (for instance IL-12) expression upon parenteral (e.g. intravenous) administration of an AAV vector. Using this approach, the inventors were able to cure subcutaneous tumors even upon intravenous administration of the vector. After intravenous administration of AAV.ISREx4.IL12miR122T to an animal with a subcutaneous tumor, the tumor is eliminated if it has been previously irradiated but not if it has not received any irradiation.

In these experiments tumor irradiation alone (with intravenous administration of a control virus encoding luciferase) is unable to induce tumor regression.

In another aspect, the invention comprises a recombinant expression system that consists of one nucleic acid construct comprising a sequence encoding at least two immunomodulatory proteins, said sequence is operatively linked to a STING-responsive promoter or a radiation inducible promoter and at least one sequence motif that inhibits transgenic expression of the immunomodulatory protein in tissues or cells not intended to express the protein.

As the inventors found out, an expression system that consists of a nucleic acid that comprises at least two immunomodulatory proteins under control of a STING-responsive promoter induces an upregulation of the immunomodulatory proteins upon radiation even in the absence of a target STING (example 15). Moreover, this expression system alone causes significant antitumor effects (example 17) without causing off-site expression (example 16).

This expression system combines many advantages of cancer immunotherapies while avoiding the disadvantages of said therapies.

It allows to express immunomodulatory peptides, especially cytokines such as interleukins or chemokines, in the tumor, thereby stimulating the immune system to target cancer cells of said tumor. Due to a preferred intratumor administration of said expression system, the cytokine expression is highly localized thereby circumventing the disadvantages and toxicity from systemic cytokine therapies known in the art.

Furthermore, the inventors found that the nucleic acid of this recombinant expression system induces the upregulation of the immunomodulatory transgenes even upon radiation alone Thus, the expression of the immunomodulatory transgenes can be controlled by administration of radiotherapy.

Moreover, the inventors found that this recombinant expression system is non-toxic and shows minor side effects in vivo. Of note, the inventors found that this recombinant expression system causes the reduction of tumors eventually leading to full remission when administered in vivo.

This recombinant expression system generates a specific and basal immunological pathway in the transfected cancer cell that makes use of further endogenous pathway components.

The nucleic acid construct of the expression system is an artificial gene construct that comprises a sequence encoding at least two immunomodulatory proteins, wherein said sequence is operatively linked to a STING-responsive promoter or a radiation inducible promoter. The immunomodulatory proteins activate both innate and adaptive immune responses in order to generate a specific cancer immune response.

Alternatively, a radiation inducible promoter may be provided. A radiation inducible promoter, as used herein, is a promoter that induces the expression of the therapeutic gene sequence when the cell is irradiated. These promoters may be natural, synthetic or artificially made selected from recombination products of natural promoters. For example, these promoters may contain at least one of the following sequences either individually or in repeated fashion:
a. eukaryotic radiation-inducible sequence CC(A/T)(6)GG (SEQ ID NO: 87), also known as CArG elements or [CC(A+T rich)6GG], such as that of early growth response-1 (Egr-1) promoter or pE9 promoter, preferably the sequence has 70%, 75%, 80%, 85%, 90%, 95%, or 99% sequence identity with respect to SEQ ID NO: 86 or is identical to SEQ ID NO: 86 (Egr1 promoter), or alternatively the sequence contains at least two sequences according to SEQ ID NO: 87, wherein the at least two sequences may be interspaced by additional nucleotides;
b. low frequency CCAAT box sequences such as that of the survivin promoter, preferably the sequence has 70%, 75%, 80%, 85%, 90%, 95%, or 99% sequence identity with respect to SEQ ID NO: 88 or is identical to SEQ ID NO: 88 (CCAAT rich domain of the survivin promoter);
c. Cyclin dependent kinase inhibitor 1 (also known as CDKN1A or p21) promoter;
d. late Herpes Simplex Virus 1 promoters, such as glycoprotein C promoter or Us 11 promoter;
e. combinations of the above or artificially mutated versions of the same.

By using a sequence motif that inhibits transgenic expression of the immunomodulatory proteins in tissues or cells not intended to express the proteins, it is possible to restrict expression of the proteins from distinct tissues, tissue regions or cell populations.

Furthermore, it may be advantageous within the scope of the invention that the at least two immunomodulatory proteins encoded by the sequence of the nucleic acid construct are selected from the group consisting of the family of interleukins, the family of chemokines, the family of colony-stimulating factors, the family of transforming growth factors, the family of tumor necrosis factors, any blocking peptide against immunosuppressive factors, any monoclonal antibody or any scFv or any nanobody reactive with immune checkpoint proteins, or a shRNA targeting immunosuppressive factors. A non-limiting list of immunomodulatory proteins is interleukin-1, interleukin-2, interleukin-3, interleukin-4, interleukin-5, interleukin-7, interleukin-8, interleukin-9, interleukin-11, single-chain interleukin-12, interleukin-12, interleukin-13, interleukin-14, interleukin-15, interleukin-15-sushi, interleukin-16, interleukin-17, interleukin-18, interleukin-19, interleukin-20, interleukin-21, interleukin-22, interleukin-23, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, granulocyte-macrophage colony-stimulating factor, type I interferons, IFN-γ, TNF-α, FLT3-ligand, a blocking peptide targeting TGF-β, a blocking peptide targeting IL-10, a blocking peptide targeting FoxP3, a monoclonal antibody or single-chain variable fragment (scFv) or nanobody neutralizing PD1, PDL1, CTLA4, CD137, TIM3, LAG3, and a fragment or variant thereof.

In a preferred embodiment of the invention, the at least two immunomodulatory proteins encoded by the sequence of the nucleic acid construct are selected from the group consisting of interleukin-1 (IL-1), interleukin-2 (IL-2), interleukin-4 (IL-4), interleukin-5 (IL-5), interleukin-7 (IL-7), interleukin-12 (IL-12), single chain interleukin-12 (scIL-12), interleukin-15 (IL-15), interleukin-15-sushi (IL-15-sushi), interleukin-21 (IL-21), interleukin-23 (IL-23), granulocyte-macrophage colony-stimulating factor (GM-CSF), type I interferons (IFN-α and IFN-β), IFN-γ, TNF-α, CXCL10, FLT3-ligand, a blocking peptide targeting TGF-β, a blocking peptide targeting IL-10, a blocking peptide targeting FoxP3, a monoclonal antibody or scFv or nanobody neutralizing PD1, PDL1, CTLA4, CD137, TIM3, LAG3, and a fragment or variant thereof.

The person skilled in the art recognizes that any immunomodulatory protein or a fragment or a variant thereof that stimulates the immune system into targeting cancer cells is a suitable immunomodulatory protein according to the invention.

There are several immunomodulatory proteins known in the art that are used in immunotherapies for cancer treatment. For example, IL-2 is a protein which has been used in immunotherapies for more than 20 years in the treatment of cancer. However, only very few patients respond at all to IL-2-related immunotherapies while these therapies are known to cause severe side effects. IL-12 re-programs myeloid-derived suppressor cells (MDSCs) to become efficient antigen-presenting cells (APC). In addition, IL-12 strongly stimulates T-cells and NK cells and increases IFN-γ production propitiating a Th1 type of response capable of controlling the growth of the treated tumor but also of distant metastasis (Lasek et al. Cancer Immunol. Immunother. 2014; 63:419 ff.). Furthermore, IL-12 has anti-angiogenic effects, which may impair tumor vascularization and thereby limit tumor growth. IL-12 is a heterodimeric cytokine which is encoded by two separate genes. To facilitate its expression in recombinant expression systems it is feasible to use the recombinant single chain IL-12 transgene that encodes both subunits of IL-12. Thus, in a preferred embodiment one of the immunomodulatory proteins according to this aspect of the invention is IL-12.

In one embodiment, one of the immunomodulatory proteins is a complex or fusion protein of IL-15 with the Sushi domain of IL-15 receptor a chain to enhance the agonist activity of IL-15 via trans-representation such as provided by the compound P22339 (Hu, Q., Ye, X., Qu, X. et al., Sci Rep 2018, 7675).

It may optionally be possible that one of the immunomodulatory proteins encoded by the sequence of the nucleic acid construct is selected from the group consisting of the α-subunit of IL-12, the β-subunit of IL-12, single chain IL-12 comprising the α- and β-subunit of IL-12. Of note, single chain IL-12 (scIL-12) is a fusion protein comprising the α- and β-subunit, wherein the peptide sequences either are directly connected or interspaced by a linker peptide, wherein the linker peptide may have a length ranging from 1 to 100 amino acids. Preferably the linker has a length ranging from 4 to 60 amino acids, more preferably from 8 to 40 amino acids and particularly preferably from 12 to 25 amino acids. The linker may have a length of 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2 or 1 amino acids. In addition one of the immunomodulatory proteins encoded by the sequence of the nucleic acid construct may be a single chain IL-12 comprising or consisting of the amino acid sequence of SEQ ID NO: 9 or 10 (human or murine single chain IL-12), or a polypeptide having at least 70%, 75%, 80%, 85%, 90%, 95%, or 99% sequence identity with respect to SEQ ID NO: 9 or 10, wherein the polypeptide has the biological activity of interleukin-12. The sequence identity of the polypeptide sequence with respect to SEQ ID NO: 9 or 10 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of interleukin-12, it has to have at least 10%, preferably at least 20%, more preferably at least 30% and even more preferably at least 50% of the biological activity of an interleukin-12 assessed with one of the bioactivity tests described in the following compared to the bioactivity of a polypeptide according to SEQ ID NO: 9 or 10.

The biological activity of IL-12 can be estimated in vitro by the ability of the peptide to increase IFN-γ production by cells expressing both the IL-12 receptor and the corresponding signaling pathway, such as peripheral blood mononuclear cells or human macrophage cell lines. Alternatively reporter cell lines can be used as, for instance, the Promega bioluminescent IL-12 Bioassay (Cat. #JA2601, JA2605).

In vivo IL-12 bioactivity can be estimated by the ability to increase IFN-γ serum levels.

Moreover, one immunomodulatory protein may be an IFN-β comprising or consisting of the amino acid sequence of SEQ ID NO: 79, or a polypeptide having at least 70%, 75%, 80%, 85%, 90%, 95%, or 99% sequence identity with respect to SEQ ID NO: 79, wherein the polypeptide has the biological activity of IFN-β. The sequence identity of the polypeptide sequence with respect to SEQ ID NO: 79 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of IFN-β, it has to have at least 10%, preferably at least 20%, more preferably at least 30% and even more preferably at least 50% of the biological activity of an IFN-β assessed with one of the bioactivity tests described in the following compared to the bioactivity of a polypeptide according to SEQ ID NO: 79.

The biological activity of IFN-β can be estimated by the ability to induce IFN type I responsive genes. To this aim, a human cell line (like HEK 293) may be incubated with the polypeptide and the expression of IFN-responsive genes may be determined by RT-qPCR. Alternatively, IFN-reporter cells encoding luciferase under the control of an ISRE-containing promoter can be used to estimate IFN-β bioactivity of the polypeptide by measuring luciferase expression in the luminometer 3 h after addition of the polypeptide to the cell culture.

In vivo IFN-β bioactivity can be estimated by the induction of IFN-responsive genes (RT-qPCR) in peripheral blood mononuclear cells.

Furthermore, one immunomodulatory protein may be an IL-15-sushi, comprising or consisting of the amino acid sequence of SEQ ID NO: 80, or a polypeptide having at least 70%, 75%, 80%, 85%, 90%, 95%, or 99% sequence identity with respect to SEQ ID NO: 80, wherein the polypeptide has the biological activity of IL-15. The sequence identity of the polypeptide sequence with respect to SEQ ID NO: 80 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of interleukin-15, it has to have at least 10%, preferably at least 20%, more preferably at least 30% and even more preferably at least 50% of the biological activity of an interleukin-15 assessed with one of the bioactivity tests described in the following compared to the bioactivity of a polypeptide according to SEQ ID NO: 80.

The biological activity of IL-15 can be estimated in vitro by a CTLL-2 cell proliferation assay.

In vivo IL-15 bioactivity can be estimated by its ability to induce the proliferation of NK cells and T cells using FACS analysis.

Furthermore, the at least one immunomodulatory protein may be a CXCL10, comprising or consisting of the amino acid sequence of SEQ ID NO: 81, or a polypeptide having at least 70%, 75%, 80%, 85%, 90%, 95%, or 99% sequence identity with respect to SEQ ID NO: 81, wherein the polypeptide has the biological activity of CXCL10. The sequence identity of the polypeptide sequence with respect to SEQ ID NO: 81 can be at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In the context of the invention, for a polypeptide to be viewed as having the biological activity of CXCL10, it has to have at least 10%, preferably at least 20%, more preferably at least 30% and even more preferably at least 50% of the biological activity of a CXCL10 assessed with one of the bioactivity tests described in the following compared to the bioactivity of a polypeptide according to SEQ ID NO: 81. CXCL10 is also known as IP-10.

The bioactivity of CXCL10 may be estimated by its ability to attract T lymphocytes and natural killer cells either in vivo or in an in vitro migration assay.

In a preferred embodiment the first immunomodulatory protein is a single chain IL-12 comprising or consisting of the amino acid sequence of SEQ ID NO: 9 or 10, or a polypeptide having at least 70%, 75%, 80%, 85%, 90%, 95%, or 99% sequence identity with respect to SEQ ID NO: 9 or 10, wherein the polypeptide has the biological activity of interleukin-12.

The second immunomodulatory protein may be either an IFN-β comprising or consisting of the amino acid sequence of SEQ ID NO: 79, or a polypeptide having at least 70%, 75%, 80%, 85%, 90%, 95%, or 99% sequence identity with respect to SEQ ID NO: 79, wherein the polypeptide has the biological activity of IFN-β; or the second immunomodulatory protein may be an IL-15-sushi, comprising or consisting of the amino acid sequence of SEQ ID NO: 80, or a polypeptide having at least 70%, 75%, 80%, 85%, 90%, 95%, or 99% sequence identity with respect to SEQ ID NO: 80, wherein the polypeptide has the biological activity of IL-15; or the second immunomodulatory protein may be a chemokine, preferably a CXCL10, comprising or consisting of the amino acid sequence of SEQ ID NO: 81, or a polypeptide having at least 70%, 75%, 80%, 85%, 90%, 95%, or 99% sequence identity with respect to SEQ ID NO: 81, wherein the polypeptide has the biological activity of CXCL10.

Also, other combinations of immunomodulatory proteins are feasible. The single-chain IL-12 may be the first immunomodulatory protein whereas the second immunomodulatory protein may be selected from the group consisting of peptides that block TGF-β, peptides that block FoxP3, or monoclonal antibodies against immune checkpoint inhibitors PD1 or PD-L1, or nanobodies against immune checkpoint inhibitors PD1 or PD-L1.

It should be noted that the terms first and second immunomodulatory protein in the context of the nucleic acid comprising two immunomodulatory proteins do not describe the order of the coding sequences on the nucleic acid but merely are used to illustrate the two different immunomodulatory proteins. Hence, the coding sequence of the second immunomodulatory protein may lie upstream of the coding sequence of the first immunomodulatory protein on the nucleic acid. Here, upstream means that the coding sequence of the second immunomodulatory protein lies in front of the coding sequence of the first immunomodulatory protein in the 5′ to 3′ direction on the nucleic acid.

A peptide blocking TGF-β is a peptide that interferes either with TGF-β itself or with a receptor that binds TGF-β so that at least some of the TGF-β signaling is reduced or even completely prevented. Examples of peptides blocking TGF-β are P17 and P144.

A peptide blocking FoxP3 is a peptide that interferes either with FoxP3 itself or with a receptor that binds FoxP3 so that at least some of the FoxP3 signaling is reduced or even completely prevented. An example of a peptide blocking FoxP3 is P60.

In another preferred embodiment the nucleic acid construct is a bi- or polycistronic expression cassette, wherein the sequences encoding the immunomodulatory proteins are separated by a sequence encoding an IRES; and/or the sequences encoding the immunomodulatory proteins are separated by a sequence encoding a 2A peptide, preferably a sequence encoding a P2A peptide. The IRES sequence and the 2A peptide encoding sequence are translation regulatory elements (TRE).

In this context, proteins or RNA having anticancer properties are any of said molecules that are capable of retarding, stopping or reversing cancer progression.

A bi- or polycistronic expression cassette is a genetic construct that contains two or more separate coding regions or genes within a single transcript. Generally speaking it contains two or more different sequences within a single transcript. To allow for separate translation of the two or more immunomodulatory proteins and/or transcription of the immunomodulatory genes, a kind of TRE is needed that allows for individual translation of the proteins.

One possible solution is to use an internal ribosomal entry site (IRES). An IRES is a specific sequence of RNA within a transcript that allows for translation initiation at a non-AUG codon, bypassing the normal cap-dependent initiation process. This mechanism allows for the regulation of gene expression and the co-expression of multiple proteins from a single mRNA molecule.

Another possible solution are 2A peptides, also known as 2A self-cleaving peptides. 2A peptides are a class of 18-22 aa-long peptides, which can induce ribosomal skipping during translation of a protein in a cell. These peptides share a core sequence motif of DxExNPGP (SEQ ID NO: 32) and are found in a wide range of viral families. They help generating polyproteins by causing the ribosome to fail at making a peptide bond. Because of their short length 2A peptides are ideal for use in viral vector systems that have a limited capacity such as AAV. Another advantage of these peptides is that they allow for equimolar expression of the coding sequences separated by these peptides. Members of the 2A peptide family are T2A, P2A, E2A and F2A. These peptides may be flanked by linker peptides of differing lengths which may result in improved functionality. For example, the F2A element preceded by a linker domain consisting of the amino acid sequence GGSG (SEQ ID NO: 82) showed significant expression of the proteins separated by this element.

TABLE 3
Examples of combinations of two immunomodulatory proteins separated
by translation regulatory elements (TRE) in a bicistronic vector

| 1^{st} immunomodulatory | | 2^{nd} immunomodulatory |
|---|---|---|
| protein | TRE | protein |
| single-chain IL-12^{A} | IRES | IFN-β^{B} |
| single-chain IL-12^{A} | IRES | IL-15-sushi^{C} |
| single-chain IL-12^{A} | IRES | CXCL10^{D} |
| single-chain IL-12^{A} | IRES | peptide blocking FoxP3 |
| single-chain IL-12^{A} | IRES | peptide blocking TGF-β |
| single-chain IL-12^{A} | IRES | monoclonal antibody against PD1 |
| single-chain IL-12^{A} | IRES | monoclonal antibody against PD-L1 |
| single-chain IL-12^{A} | IRES | nanobody against PD1 |
| single-chain IL-12^{A} | IRES | nanobody against PD-L1 |
| IFN-β^{B} | IRES | IL-15-sushi^{C} |
| single-chain IL-12^{A} | P2A | IFN-β^{B} |
| single-chain IL-12^{A} | P2A | IL-15-sushi^{C} |
| single-chain IL-12^{A} | P2A | CXCL10^{D} |
| single-chain IL-12^{A} | P2A | peptide blocking FoxP3 |
| single-chain IL-12^{A} | P2A | peptide blocking TGF-β |
| single-chain IL-12^{A} | P2A | monoclonal antibody against PD1 |
| single-chain IL-12^{A} | P2A | monoclonal antibody against PD-L1 |
| single-chain IL-12^{A} | P2A | nanobody against PD1 |
| single-chain IL-12^{A} | P2A | nanobody against PD-L1 |
| IFN-β^{B} | P2A | IL-15-sushi^{C} |
| single-chain IL-12^{A} | F2A | IFN-β^{B} |
| single-chain IL-12^{A} | F2A | IL-15-sushi^{C} |
| single-chain IL-12^{A} | F2A | CXCL10^{D} |
| single-chain IL-12^{A} | F2A | peptide blocking FoxP3 |
| single-chain IL-12^{A} | F2A | peptide blocking TGF-β |
| single-chain IL-12^{A} | F2A | monoclonal antibody against PD1 |
| single-chain IL-12^{A} | F2A | monoclonal antibody against PD-L1 |
| single-chain IL-12^{A} | F2A | nanobody against PD1 |
| single-chain IL-12^{A} | F2A | nanobody against PD-L1 |
| IFN-β^{B} | F2A | IL-15-sushi^{C} |

^{A}being preferably a human single chain IL-12 as depicted in SEQ ID NO: 9
^{B}being preferably a human IFN-β as depicted in SEQ ID NO: 79
^{C}being preferably a human IL-15-sushi as depicted in SEQ ID NO: 80
^{D}being preferably a human CXCL10 as depicted in SEQ ID NO: 81

Immunotherapies that involve systemic availability of the immunomodulatory protein often cause unwanted side effects. However, an advantage of this aspect of the invention is that the recombinant expression system used herein induces the local expression of said immunomodulatory proteins thereby inducing a highly local and targeted immune response. For example, the intratumor administration of the recombinant expression system according to this aspect of the invention induces an immune response within or in close proximity to the tumor. Without being wished to be bound by theory, it is thought that the release of the immunomodulatory proteins within or in the vicinity of the tumor causes T-cells to infiltrate the tumor vicinity and may cause the recognition of tumor cells that normally escape the recognition of the immune system, thereby enabling the immune system to target and kill these cells. Thus, tumor progression is at least retarded, preferably halted, and more preferably reversed.

In a preferred embodiment at least one of the immunomodulatory proteins of this aspect of the invention is an immunostimulatory protein. That is a protein that stimulates the immune system such as by directly stimulating it (e.g. IL-12) or by inhibiting immunosuppressive molecules (e.g. a blocking peptide targeting TGF-β).

According to a further possibility it may be provided that the STING-responsive promoter of the nucleic acid construct of this aspect of the invention is selected from the group consisting of IFN-β-responsive promoter (preferably according to SEQ ID NO: 25), IFN-β-minimal promoter (preferably according to SEQ ID NO: 31), CXCL9-responsive promoter (preferably according to SEQ ID NO: 23), CXCL10-responsive promoter (preferably according to SEQ ID NO: 24), STAT-motif promoter (preferably according to SEQ ID NO: 26), IRSF3-motif promoter (preferably according to SEQ ID NO: 27 or SEQ ID NO: 28), ISRE-motif promoter (preferably according to SEQ ID NO: 12 or SEQ ID NO: 13), NFκB-motif promoter (preferably according to SEQ ID NO: 29 or 30) and STAT6-motif promoter. In a preferred embodiment, the STING responsive promoter comprises at least one interferon response element (ISRE).

By operatively linking the expression of the immunomodulatory proteins to a STING-responsive promoter according to the invention a targeted and local expression of the immunomodulatory proteins can be achieved, thereby avoiding potential side effects by systemic expression of the proteins.

It may optionally be possible that the STING-responsive promoter of this aspect of the invention is selected from the list consisting of:
a. a nucleotide sequence comprising one to ten copies, preferably two to seven copies, more preferably three to five copies and particularly preferably 4 copies of a sequence having at least 75%, at least 80%, at least 85%, at least 90% or at least 95% sequence identity with respect to SEQ ID NO: 11 or is identical to SEQ ID NO: 11;
b. a nucleotide sequence according to a., wherein the copies are interspaced by up to forty nucleotides, preferably four to thirty-five nucleotides, more preferably ten to thirty nucleotides and particularly preferably fifteen to twenty-five nucleotides;
c. a nucleotide sequence according to SEQ ID NO: 12 which comprises four copies of the ISRE interspaced with 20 nucleotides;
d. a nucleotide sequence according to SEQ ID NO: 13 which comprises four copies of the ISRE interspaced with 6 nucleotides.

The use of STING-responsive promoters in the context of the invention ensures the controlled and targeted expression of the immunomodulatory proteins. Moreover, the aforementioned STING-responsive promoters have been shown to have additional advantages. As elaborated in the examples of this application the STING-responsive promoters have been shown to be inducible by administering a human-non-reacting STING agonist in the presence of target STING. Moreover, the induction of these promoters has been shown to be specific in that in the absence of the selective STING agonist only limited expression occurs. In particular, promoters carrying four copies of the ISRE as depicted in SEQ ID NO: 12 are highly inducible while also displaying high specificity. Noteworthy, expression can even be achieved in the absence of STING when applying radiotherapy.

Preferably, the nucleotide sequence of the STING-responsive promoters as described in a. and b. do not contain an AP-1 site or an NFκB site. Even more preferably the nucleotide sequence of the STING-responsive promoters as described in a. and b. do not contain any further motif that contributes to inducibility, leakage or expression efficiency of the promoter. In other words, the nucleotide sequences of the promoter as described in a. or b. may contain several nucleotides prior to, in between and/or after the ISRE of SEQ ID NO: 11, wherein these additional nucleotides are chosen in a way that (e.g. randomly) they do not form any transcription regulatory element. Particularly preferably, the nucleotide sequence of the STING-responsive promoters as described in a. and b. consist of one to ten copies, preferably two to seven copies, more preferably three to five copies and particularly preferably 4 copies of a sequence having at least 75%, at least 80%, at least 85%, at least 90% or at least 95% sequence identity with respect to SEQ ID NO: 11 or is identical to SEQ ID NO: 11.

In addition, the nucleotide sequence of the STING-responsive promoters as described in a. to d. may already represent a functional promoter, so that no other element is needed for these promoters to induce the expression of the operatively linked transgene.

According to a further possibility it may be provided that the nucleic acid construct of this aspect of the invention further comprises at least one sequence motif that inhibits transgenic expression of the immunomodulatory proteins in tissues or cells not intended to express the proteins, preferably being the liver, wherein the sequence motif is preferably a target sequence of a microRNA which is highly abundant in the liver.

It is known that several viral particles, especially AAV, show a tropism towards liver cells. This means that when viral particles gain access to the blood circulatory system, they preferentially transduce liver cells, eventually causing the expression of the encoded transgenes in the liver. Also, in the event of intratumor injections of said viral particles, some of these particles will access the blood circulatory system, as the tumor is highly vascularized. To prevent the unwanted expression of the transgenes encoded by the viral particle in the liver it is therefore suitable to control their expression. Several methods are known by the person skilled in the art to achieve this goal.

In general, a suitable method to limit transgene expression to a specific tissue or a specific cell type is to use viral particles with a distinct tropism towards said tissues or cell types. A way to achieve this is by altering the serotype of an AAV or by altering the capsid of the AAV. Several AAV serotypes are known in the art each with distinct tropisms. However, as the invention is intended to target several different types of cancers, it would be necessary to optimize the serotype or capsid for each cancer or tumor type. Nonetheless, this may be a suitable way to achieve tissue- or cell type-specific expression of a transgene.

The skilled person may also use DNA- or RNA-binding repressors to avoid unwanted expression of the transgenes encoded by the viral particle in tissues or cells not intended to express the transgenes. Therefore, the nucleic acid construct of this aspect of the invention may comprise an operon that binds a repressor. The repressor may be operatively linked to a promoter that is activated in tissues or cells not intended to express the transgenes, hence, inducing the expression of the repressor in these tissue or cells, thereby, inhibiting expression of the transgenes.

Another possibility is to use RNA interference (RNAi). RNAi is characterized in that an RNA molecule reduces or inhibits expression of a nucleic acid sequence with which the RNA molecule shares substantial or total homology. Non-limiting examples of RNAi are small interfering RNA, small hairpin RNA or microRNA (miR). To reduce or inhibit transgene expression in a specific tissue using RNAi it may be conceivable that for example a shRNA targeting the nucleotide sequence of said transgene may be operatively linked to a tissue specific promoter. When said shRNA is administered it thereby reduces or inhibits transgene expression in the tissue. Although this method would be feasible in the context of the invention it may be disadvantageous in that an additional administration of said shRNA would be necessary to limit transgene expression in the target tissue, e.g. the liver.

An elegant way to avoid this drawback is to operatively link a miRNA target sequence to the nucleotide sequence of the transgenes. Thereby, expression of the transgenes will be reduced or inhibited in tissues where a miRNA that is associated to said miRNA target sequence is present. Accordingly, a preferred embodiment of the invention comprises that the sequence motif is a target sequence of a microRNA which is highly abundant in the liver. Thereby, a single administration of a nucleic acid construct is sufficient to avoid transgene expression in the liver while transcription in other tissues or organs is not impaired.

It is optionally conceivable that the microRNA abundant in the liver is selected from the group consisting of miR-122, miR-192, miR-199a, miR-101, miR-99a, let7a, let7b, let7c or let7f. Furthermore, it may be advantageous that the microRNA abundant in the liver is miR-122. The advantage of miR-122 is that it is highly expressed in the liver and, importantly, specifically expressed in the liver. Hence, by choosing a miR-122 target sequence a significant reduction of the transgene in the liver is ensured while expression in other tissues is not impaired. Moreover, it has been shown that miR-122 is downregulated in most liver tumors so that the recombinant expression system according to this aspect of the invention is also suitable to treat liver cancer.

According to a preferred embodiment of this aspect of the invention, the respective miR-122 target sequence is a miR-122 target sequence having at least 90% or at least 95% sequence identity with respect to SEQ ID NO: 14 or is identical to SEQ ID NO: 14. Preferably, the target sequence comprises 1 to 12, preferably 3 to 10, more preferably 4 to 8 and particularly preferably 5 copies of the miR-122 target sequence as depicted in the previous sentence. Increasing the copy number of miR-122 target sequences increased the miR-mediated reduction, whereas no significant improvement was observed above the disclosed number of 5 copies. In a preferred embodiment the copies are interspaced by one to forty nucleotides, preferably one to thirty nucleotides, more preferably two to twenty nucleotides and particularly preferably two to eight nucleotides.

It is preferred that the nucleotide sequence comprises 5 copies of the miR-122 target sequence as depicted in SEQ ID NO: 15. As elaborated in the examples of this application, the nucleotide sequence as depicted in SEQ ID NO: 15 has been shown to substantially inhibit transgene expression in the liver thus causing the plasma levels of the transgenes to be undetectable by the methods described in Example 16.

According to a further possibility, it may be provided that the nucleic acid construct of this aspect of the invention further comprises a transgene encoding a thymidine kinase, preferably a thymidine kinase according to SEQ ID NO: 16. The nucleotide sequence encoding the thymidine kinase is linked to the nucleotide sequence of the immunomodulatory proteins and may be interspaced by a 2A self-cleaving peptide (so called 2A peptides). 2A peptides constitute a class of 18-22 amino acid-long peptides, which can induce ribosomal skipping during translation of a protein in a cell. These peptides share a core sequence motif of “DXEXNPGP” and are found in a wide range of viral families. They help generating polyproteins by causing the ribosome to fail at making a peptide bond. The person skilled in the art recognizes that the use of a 2A peptide allows the co-expression of the immunomodulatory protein and the thymidine kinase. The co-expression of the thymidine kinase is advantageous in that it allows to assess tumor transduction by PET imaging and, in case of potential IL-12-mediated toxicity, to eliminate the cells expressing the cytokine by administering a thymidine kinase substrate such as for example acyclovir, penciclovir, famciclovir or ganciclovir.

The nucleic acid construct of this aspect of the invention can further comprise a woodchuck hepatitis virus post-transcriptional regulatory element (WPRE). The WPRE is known to increase transgene expression, whereby it is most effective when placed downstream of the transgene (i.e., the immunomodulatory proteins), proximal to the polyadenylation signal.

Expediently, the nucleic acid construct of this aspect of the invention further comprises a polyadenylation signal located at the 3′position with respect to the immunomodulatory proteins. The of length the of Poly(A) tail may be optimized for stabilization of mRNA and subsequent increased protein translation.

The skilled person is also aware of other means to increase protein translation such as
a. by sequence modification to optimize G/C content and/or excessively stabilizing secondary structure codon, or
b. alternating the 5′cap by adding synthetic or natural 5′cap analogues such as m7GpppG or an initiating trimer such as “CleanCap” or stabilization by means of capping enzymes, or
c. addition of regulatory elements
   c.1 in the 5′-untranslated region (UTR) such as optimization through appropriate cis regulatory sequences or modifying length and sequence to avoid excessively stable structure, or
   c.2 in the 3′-UTR through sequence modification to introduce stable elements or repeated motifs or synthetic or natural sequences that have been proven effective in other mRNAs.

In a special embodiment, the nucleic acid construct of the recombinant expression system of this aspect of the invention consists of a nucleic acid construct as given in the following table, wherein the different elements are shown in the 5′ to 3′direction as promotor, first open reading frame (ORF1), first translation regulatory element (TRE1), second open reading frame (ORF2), second translation regulatory element (TRE2) and target sequence for a microRNA (microRNA element).

TABLE 4
Embodiments of the nucleic acid construct of the expression system

| | | | | | microRNA |
|---|---|---|---|---|---|
| Promoter | ORF1 | TRE1 | ORF2 | TRE2 | element |
| ISREx4 ^{A} | scIL12 ^{B} | P2A | IFN-β^{C} | WPRE | miR122 ^{E} |
| ISREx4 ^{A} | scIL12 ^{B} | P2A | IL-15-sushi^{D} | WPRE | miR122 ^{E} |
| ISREx4 ^{A} | scIL12 ^{B} | P2A | peptide blocking FoxP3 | WPRE | miR122 ^{E} |
| ISREx4 ^{A} | scIL12 ^{B} | P2A | peptide blocking TGF-β | WPRE | miR122 ^{E} |
| ISREx4 ^{A} | scIL12 ^{B} | P2A | monoclonal antibody against PD1 | WPRE | miR122 ^{E} |
| ISREx4 ^{A} | scIL12 ^{B} | P2A | monoclonal antibody against PD-L1 | WPRE | miR122 ^{E} |
| ISREx4 ^{A} | scIL12 ^{B} | P2A | nanobody against PD1 | WPRE | miR122 ^{E} |
| ISREx4 ^{A} | scIL12 ^{B} | P2A | nanobody against PD-L1 | WPRE | miR122 ^{E} |
| ISREx4 ^{A} | IFN-β^{C} | P2A | IL-15-sushi^{D} | WPRE | miR122 ^{E} |
| ISREx4 ^{A} | scIL12 ^{B} | IRES | IFN-β^{C} | WPRE | miR122 ^{E} |
| ISREx4 ^{A} | scIL12 ^{B} | IRES | IL-15-sushi^{D} | WPRE | miR122 ^{E} |
| ISREx4 ^{A} | scIL12 ^{B} | IRES | peptide blocking FoxP3 | WPRE | miR122 ^{E} |
| ISREx4 ^{A} | scIL12 ^{B} | IRES | peptide blocking TGF-β | WPRE | miR122 ^{E} |
| ISREx4 ^{A} | scIL12 ^{B} | IRES | monoclonal antibody against PD1 | WPRE | miR122 ^{E} |
| ISREx4 ^{A} | scIL12 ^{B} | IRES | monoclonal antibody against PD-L1 | WPRE | miR122 ^{E} |
| ISREx4 ^{A} | scIL12 ^{B} | IRES | nanobody against PD1 | WPRE | miR122 ^{E} |
| ISREx4 ^{A} | scIL12 ^{B} | IRES | nanobody against PD-L1 | WPRE | miR122 ^{E} |
| ISREx4 ^{A} | IFN-β^{C} | IRES | IL-15-sushi^{D} | WPRE | miR122 ^{E} |

^{A} being preferably 4 copies of ISRE, preferably given by SEQ ID NO: 12
^{B} being preferably a human single chain IL-12 as depicted in SEQ ID NO: 9
^{C}being preferably a human IFN-β as depicted in SEQ ID NO: 79
^{D}being preferably a human IL-15-sushi as depicted in SEQ ID NO: 80
^{E} being preferably 5 copies of miR-122 target sequence as depicted in SEQ ID NO: 15

Another aspect of the invention is an expression vector encoding the nucleic acid construct comprising a sequence encoding at least two immunomodulatory proteins. Using an expression vector comprising this nucleic acid construct has certain advantages. Firstly, expression vectors are specifically designed to allow the introduction of the encoded nucleotide sequences into a host cell and commandeer their expression. Secondly, expression vectors are plasmids that are very stable and thereby protected from degradation. Thirdly, expression vectors are conceivable for gene delivery using different methods. A non-limiting example of an expression vector and a preferred embodiment of this aspect of the invention is a viral vector, more preferably a viral vector selected from the list consisting of adeno-associated virus (AAV) vector, adenoviral vector, lentiviral vector, vaccine virus vector, or herpes simplex virus vector. Viral vectors in particular combine several advantages of expression vectors such as general safety, low toxicity and stability.

It is also advantageous that the nucleic acid construct comprising a sequence encoding at least two immunomodulatory proteins of the expression vector further comprises a 5′ITR and a 3′ITR sequence, being preferably AAV ITR as depicted in SEQ ID NO: 17. It may be advantageous that the expression vector is an AAV vector and preferably has the serotype AAV1, AAV3, AAV6, AAV8, AAV9, AAV2, AAV5, AAVrh.10, or any gain-of-function mutant of AAVrh.10, whereas AAV2 is most preferred. AAVs are commonly used in biotechnology and therefore well characterized. Advantages of using AAV vectors are that they are generally regarded as safe, display low toxicity and are stable. Moreover, AAVs are already approved by several governmental organizations around the world for use in human.

It is also advantageous that the nucleic acid construct comprising a sequence encoding at least two immunomodulatory proteins of the expression vector further comprises a 5′ITR, a W packaging signal and a 3′ITR sequence, being preferably the 5′ITR, the W packaging signal and the 3′ITR of an adenovirus (Ad). It may be advantageous that the expression vector is an adenoviral vector, preferably a high-capacity adenoviral vector. Adenoviruses are commonly used in biotechnology and are therefore well characterized. An advantage of adenoviral vectors is that they have a higher size limitation compared to AAV vectors. Moreover, adenoviruses are already approved by several governmental organizations around the world for use in human.

It is also advantageous that the expression vector is a lentiviral vector, a vaccine virus vector or a herpes simplex virus vector. These vectors are commonly used in biotechnology and therefore well characterized.

It is also conceivable that nanoparticles may be used to deliver the nucleic acid comprising a sequence encoding at least two immunomodulatory proteins. Nanoparticles as gene delivery systems are known in the art and have advantages like tunable size, shape and/or surface thereby allowing to regulate their biological behaviors. For effective delivery and uptake by cells the nucleic acid constructs would be packaged into nanoparticles, preferably liposomes, made with pharmaceutically acceptable carriers composed of varying proportions of physiologically compatible lipids, proteins, polysaccharides or synthetic polymers such as standard lipids compatible with biological membranes selected from the list consisting of phospholipids such as distearoylphosphatidylglycerol, distearoylphosphatidylcholine, (DLPC) 1,2-dilauroyl-sn-glycero-3-phosphocholine, (DMPC) 1,2-dimyristoyl-sn-glycero-3-phosphocholine, (DPPC) 1,2-dipalmitoyl-sn-glycero-3-phosphocholine, (DSPC) 1,2-distearoyl-sn-glycero-3-phosphocholine, (DOPC) 1,2-dioleoyl-sn-glycero-3-phosphocholine, (DMPE) 1,2-dimyristoyl-sn-glycero-3-phosphoethanolamine, (DPPE) 1,2-dipalmitoyl-sn-glycero-3-phosphoethanolamine, (DOPE) 1,2-dioleoyl-sn-glycero-3-phosphoethanolamine, (DOPS) 1,2-dioleoyl-sn-glycero-3-phospho-L-serine, (DPPS) 1,2-dipalmitoyl-sn-glycero-3-phospho-L-serine, (DMPS) 1,2-dimyristoyl-sn-glycero-3-phospho-L-serine, (DOPG) 2,3-Dimyristoyl-sn-glycero-1-phosphocholine, and their respective enantiomers, wherein distearoylphosphatidylglycerol and distearoylphosphatidylcholine are most preferred. Another suitable example is the use of cardiolipins preferably combined with cholesterol. Alternative nanoparticles are dendrimer nanoparticles, protamine liposomes, cationic polymers, cationic polymer liposomes, polysaccharide particles, cationic lipid particles, cationic lipid cholesterol nanoparticles, cationic lipid cholesterol PEG nanoparticles, ionizable lips preferably lipids positively charged under physiological conditions, including amino lipids such as MC3 (DLin-MC3-DMA), C12-200, cKK-E12, ZA3-Ep10, 200-0i10; (SM-102) heptadecan-9-yl 8-((2-hydroxyethyl) (6-oxo-6-(undecyloxy)hexyl)amino)octanoate, (ALC 0159) N,N-dimyristylamide of 2-hydroxyacetic acid, 0-pegylated to a PEG chain of approx. 2 kilodaltons, (ALC 0315) (4-hydroxybutyl)azanediyl]di(hexane-6,1-diyl) bis(2-hexyldecanoate), Lipofectamine, Trans-IT, InstantFECT, (DOTAP) 1,2-Dioleoyl-3-trimethylammonium. Other components may include peptides and polypeptides such as DP7-C the cholesterol-modified cationic peptide DP7 (VQWRIRVAVIRK, SEQ ID NO: 85), polymers such as polyethylene glycol preparations composed of polymer molecules of varying molecular sizes which may be noncovalently bound or covalently linked to lipids in the formulation.

Another aspect of the invention is a viral particle comprising the aforementioned viral vector which comprises the nucleic acid construct comprising a sequence encoding at least two immunomodulatory proteins. This has the advantage that one administration of said viral particle is sufficient to express the recombinant expression system in the patient.

The aforementioned viral particle is preferably an AAV or an adenoviral particle; more preferably an AAV viral particle. As described hereinbefore, Ad and AAV display several advantages over other viral particles such as that they are well characterized, frequently used and approved for treatment in humans. These viruses display further advantages that render them particularly useful in gene therapy such as that they are capable of transducing dividing as well as non-dividing cells and that they do not integrate into the host genome.

Another aspect of the invention is a pharmaceutical composition that comprises a pharmaceutically acceptable excipient together with the recombinant expression system comprising a sequence encoding at least two immunomodulatory proteins, the expression vector comprising a sequence encoding at least two immunomodulatory proteins or the viral particle comprising a sequence encoding at least two immunomodulatory proteins. Pharmaceutical compositions have the advantage that they are “ready-to-use” in that they may be administered directly to the patient for treatment. In addition, pharmaceutic compositions are tailored to optimize stability and availability of the ingredients according to the therapeutic need while avoiding unwanted side effects for the patient.

In another aspect of the invention the recombinant expression system, the expression vector, the viral particle, or the pharmaceutical composition, each comprising a sequence encoding at least two immunomodulatory proteins may be used in a therapy, preferably an immunotherapy or are provided for use in a therapy, preferably an immunotherapy.

In a preferred embodiment the immunotherapy is the immunotherapy of a proliferative disorder, more preferably the immunotherapy of cancer, even more preferably the immunotherapy of solid cancer. While it is conceivable that non-solid cancers may also be treated by the immunotherapy of the invention the use for the therapy in solid cancers has the advantage that these cancers are delimited locally and therefore are accessible by local administration. Hence, it is possible to administer the recombinant expression system, the expression vector, the viral particle or the pharmaceutical composition each comprising a sequence encoding at least two immunomodulatory proteins locally to the tumor by intratumor administration. This has the advantage that a local and targeted immune response may be induced, thus causing the retardation, the halt or reversal of tumor progression. In addition, a local administration reduces the unwanted systemic expression even further although the invention takes measures to avoid systemic expression.

As already described above cancer immunotherapies have several limitations such as i) poor tumor targeting, ii) weak tumor cell killing ability, iii) high effort and thereby high cost, iv) tumor immune escape, v) over-stimulation of the immune system which can cause severe side effects (Tan et al. 2020, Biomed Pharmacother. 124: 109821). The immunotherapy as described above is an extended immunotherapy as it also incorporates gene therapy. Hence, this immunotherapy is not merely an immunotherapy by itself but a combination of immunotherapy and gene therapy, hereinafter termed “immunogene therapy” to effectively treat proliferative disorders like cancer.

It may further be possible that the cancer is selected from the list consisting of. carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burkitt's lymphoma; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xeroderma pigmentosum, keratoacanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, urothelial cancer, hepatocellular carcinoma, hepatoblastoma, hepatoma, breast cancer, colon carcinoma, and head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, Sino nasal natural killer, neoplasms, plasma cell tumors, carcinoid tumors, neuroendocrine tumors, cholangiocarcinoma, and Merkel cell carcinoma.

In another preferred embodiment the recombinant expression system, expression vector, the viral particle or pharmaceutical composition, each comprising a sequence encoding at least two immunomodulatory proteins, is employed in a combination therapy with at least one other therapy selected from the group consisting of a therapy comprising at least one checkpoint inhibitor, a therapy comprising at least one adoptive cell therapy, a therapy comprising at least one cancer vaccine, a therapy comprising at least one exogenous cytokine and/or a therapy comprising a radiotherapy.

The inhibitor of the therapy comprising at least one checkpoint inhibitor may be selected of the group consisting of an inhibitor of PD-1, PD-L1, PD-L2, CTLA-4, LAG3, BTLA, B7H3, B7H4, TIM3, KIR, or A2aR, more preferably selected from the group consisting of an inhibitor of PD-1, PD-L1, PD-L2, or CTLA-4. Immune checkpoints are molecules expressed on T cells or cells interacting with T cells that help regulate the immune response and prevent excessive or prolonged immune activation. However, cancer cells can take advantage of these checkpoint proteins to evade detection by the immune system. By blocking these checkpoint proteins, immune checkpoint inhibitors can release the brakes on the immune response and allow the immune system to attack and destroy cancer cells. Using immune checkpoint inhibitors as an additional therapy is particularly advantageous in the context of this aspect of the invention as the expression system comprising a sequence encoding at least two immunomodulatory proteins causes infiltration of T cells into the tumor environment. As these T cells might be impaired in their function due to the immune checkpoints, means to overcome this hurdle may greatly benefit the treatment outcome.

A therapy comprising at least one adoptive cell therapy may be an adoptive NK cell therapy, adoptive dendritic cell therapy, or adoptive T-cell therapy, wherein the adoptive T-cell therapy preferably is a CAR-T-cell therapy or a therapy using tumor infiltrating leukocytes (TIL). In some cases, tumors silence the expression of MHC class I molecules and/or IFN-γ signaling molecules as a means to evade T cell immunity. Using adoptive cell therapy may circumvent these problems as these adopted cells are normally capable of recognizing and targeting tumor cells. Thus, synergistic effects may be expected in a combinatory approach.

Also the combination of the expression system of the invention with a cancer vaccine or exogenous cytokines may cause synergistic effects as the recruited T cells are primed towards cancer cells by cancer vaccines or exogeneous cytokines.

In a preferred embodiment of this aspect of the invention, the cancer is selected from the list consisting of bladder cancer, bone cancer, brain cancer, breast cancer, cancer of the thymus, cervical cancer, colon cancer, oesophageal cancer, gastric cancer, head & neck cancers, Hodgkin's lymphoma, liver cancer, lung cancer, melanoma, mesothelioma, multiple myeloma, Merkel cell carcinoma, non-Hodgkin's lymphoma, ovarian cancer, pancreatic cancer, prostate cancer, rectal cancer, renal cancer, urothelial cancer, sarcoma, skin cancer, testicular cancer, thyroid cancer, uterine cancer, plasma cell tumors, cholangiocarcinoma, and, neuroendocrine tumors or carcinoid tumors.

In yet another aspect, the invention relates to the recombinant expression system, the expression vector, the viral particle or the pharmaceutical composition, each comprising a sequence encoding at least two immunomodulatory proteins, for use in an immunotherapy, wherein the immunotherapy comprises the following steps:
a. administration of the recombinant expression system comprising a sequence encoding at least two immunomodulatory proteins; the expression vector comprising a sequence encoding at least two immunomodulatory proteins, the viral particle comprising a sequence encoding at least two immunomodulatory proteins or the pharmaceutical composition comprising a sequence encoding at least two immunomodulatory proteins to a patient suffering from a proliferative disorder, being preferably cancer in order to yield a therapeutically effective transgenic expression of the immunomodulatory proteins, wherein the administration is preferably an intratumoral administration or a systemic administration;
b. optionally treating the patient before and/or concurrently to the administration according to step a) with a radiotherapy, being preferably a radiotherapy directed to the tissue or organ intended to express the transgenes from a); and/or;
c. treating the patient after the administration according step a) with a radiotherapy, being preferably a radiotherapy directed to the tissue or organ intended to express the transgenes from a).

In a similar aspect, the invention relates to a method for treating a proliferative disorder, being preferably cancer, in a patient, the method comprising:
a. administering the recombinant expression system comprising a sequence encoding at least two immunomodulatory proteins; the expression vector comprising a sequence encoding at least two immunomodulatory proteins, the viral particle comprising a sequence encoding at least two immunomodulatory proteins or the pharmaceutical composition comprising a sequence encoding at least two immunomodulatory proteins to the patient suffering from a proliferative disorder, being preferably cancer in order to yield a therapeutically effective transgenic expression of the immunomodulatory proteins, wherein the administration is preferably an intratumoral administration or a systemic administration;
b. optionally treating the patient before and/or concurrently to the administration according to step a) with a radiotherapy, being preferably a radiotherapy directed to the tissue or organ intended to express the transgene from a); and/or;
c. treating the patient after the administration according step a) with a radiotherapy, being preferably a radiotherapy directed to the tissue or organ intended to express the transgenes from a).

In another embodiment the patient is treated before, concurrently and/or after the administration according to step a) with at least one other therapy selected from the group consisting of a therapy comprising at least one checkpoint inhibitor, a therapy comprising at least one adoptive cell therapy, a therapy comprising at least one cancer vaccine and/or a therapy comprising at least one exogenous cytokine. In a preferred embodiment the treatment with the at least one other therapy occurs after the administration according to step a).

Notably, the embodiments described in the following apply to both, the recombinant expression system, the expression vector, the viral particle, or the pharmaceutical composition, each comprising a sequence encoding at least two immunomodulatory proteins for use in the immunotherapy and the method of treating a proliferative disorder.

It is conceivable that the use for immunotherapy or the method of treating a proliferative disorder may be combined with other treatments that are anti-cancer therapies. Several anti-cancer therapies are known in the art such as chemotherapy, hormone therapy, radiotherapy, photodynamic therapy, stem cell transplants, surgery, targeted therapy, hyperthermia, or other immunotherapies. It may be advantageous to direct the radiotherapy to the tumor that has received intratumor administration according to step a).

It is also optionally conceivable that in step b) and/or step c) a radiotherapy with 100 Gy or less is used. Any ionizing radiation dose of less than 100 Gy, 96 Gy, 92 Gy, 88 Gy, 84 Gy, 80 Gy, 76 Gy, 72 Gy, 68 Gy, 64 Gy, 60 Gy, 56 Gy, 52 Gy, 48 Gy or 44 Gy may be used. It may also be conceivable that in step b) and/or step c) a low dose radiotherapy with 40 Gy or less, preferably 10 Gy or less and more preferably 8 Gy or less is used. Low-dose radiotherapy can be any radiotherapy with an ionizing radiation dose of less than 40 Gy, 38 Gy, 36 Gy, 34 Gy, 32 Gy, 30 Gy, 28 Gy, 26 Gy, 24 Gy, 22Gy, 20 Gy, 19 Gy, 18 Gy, 17 Gy, 16 Gy, 15 Gy, 14 Gy, 13 Gy, 12 Gy, 11 Gy, 10 Gy, 9 Gy, 8 Gy, 7 Gy, 6 Gy, 5 Gy, 4 Gy, 3 Gy, 2.5 Gy, 2.0 Gy, 1.5 Gy, 1 Gy, 0.5 Gy, 0.25 Gy, 0.2 Gy, 0.15 Gy, 0.1 Gy, 0.05 Gy, or 0.01 Gy. An advantage of using low-dose radiotherapy is that it is better tolerated by patients in that they experience less or no side effects that are otherwise common in radiotherapy. As used herein, the indicated doses refer to total doses, meaning that the total dose for a treatment in step b) or step c) does not exceed the claimed dose. Hence, a dose of 40 Gy as applied in step b) can be achieved by a single dose of 40 Gy or fractioned doses, for example four doses of 10 Gy, given over a period for up to several weeks or even months.

In another embodiment the radiation therapy in step c) comprises several separate radiation treatments, wherein the cumulated dose of the separate radiation treatments adds up to less than 100 Gy, preferably less than 40 Gy and more preferably to less than 25 Gy. The cumulated dose of the separate radiation treatments may be less than 100 Gy, 96 Gy, 92 Gy, 88 Gy, 84 Gy, 80 Gy, 76 Gy, 72 Gy, 68 Gy, 64 Gy, 60 Gy, 56 Gy, 52 Gy, 48 Gy, 44 Gy, 40 Gy, 38 Gy, 36 Gy, 34 Gy, 32 Gy, 30 Gy, 28 Gy, 26 Gy, 24 Gy, 22Gy, 20 Gy, 19 Gy, 18 Gy, 17 Gy, 16 Gy, 15 Gy, 14 Gy, 13 Gy, 12 Gy, 11 Gy, 10 Gy, 9 Gy, 8 Gy, 7 Gy, 6 Gy, 5 Gy, 4 Gy, 3 Gy, 2.5 Gy, 2.0 Gy, 1.5 Gy, 1 Gy, 0.5 Gy, 0.25 Gy, 0.2 Gy, 0.15 Gy, 0.1 Gy or 0.05 Gy.

Notably, radiotherapy may be either applied externally or internally. The person skilled in the art knows several means to provide external radiotherapy. For example, external radiotherapy may be provided by gamma ray, electron beam or proton accelerators adequate for therapeutic purposes. However, for some tumors it may be advantageous to use internal radiotherapy. Internal radiotherapy may be provided by administration of a pharmacologically acceptable forma and dose of a radioactive isotope for therapeutic purposes. Embodiments of internal radiotherapy may be brachytherapy or non-localized radiotherapy. Brachytherapy physically constraints radioactive deposits so that they are essentially not movable from the location they have been deposited. The radioactive deposits may be placed interstitially (interstitial radiotherapy) which is used for example for quite a significant percentage of prostate tumors and some head and neck tumors, or radioactive deposits may be placed inside a cavity (intracavity radiotherapy) which is used for example for some uterus, bronchus, or esophagus tumors. The non-localized radiotherapy may be either a partially disseminated local administration such as selective internal radiation therapy (SIRT) where a suspension of material is injected into particular arteries or veins with the objective that it settles close to the site of injection rather than experience wide distribution in the body, or it may be an entirely systemic approach such as use of soluble chemical substances synthesized with radioactive isotopes which find their spot of action by affinity or biologicals such as radiolabeled antibodies in order to better deliver the radiation to the target tumor. A classic example for the latter is iodine-131. Although different isotopes may be used for different treatment paradigms, the isotopes useful in this invention may be preferably selected from the list consisting of yttrium-90, iodine-131, lutetium-177, iridium-192, astatine-211, bismuth-213, actinium 225, samarium-153, radium-223, phosphorus-32, strontium-89, erbium-169, holmium-166, terbium-161, gallium-68, rhenium-188 or rhenium-186.

In a further aspect the invention relates to the Embodiments 1 to 18, as stated below:

Embodiment 1: A recombinant expression system that comprises
a. a first nucleic acid construct comprising a sequence encoding for a target STING, that is responsive to a STING agonist recognizing said target STING but not the human STING and natural variants thereof, said sequence encoding the target STING is operatively linked to elements allowing expression and/or translation of said target STING; and/or
b. a second nucleic acid construct comprising a sequence encoding at least one immunomodulatory gene or protein, said sequence is operatively linked to a STING-responsive promoter.

Embodiment 2: The recombinant expression system of embodiment 1, wherein the target STING is responsive to a human-non-reacting STING agonist selected from the group consisting of 5,6-dimethylxanthenone-4-acetic acid (DMXAA), 7-bromo-DMXAA, 7-iodo-DMXAA, 7-hydroxy-DMXAA, 7-formyl-DMXAA, 7-hydroxymethyl-DMXAA, 7-(2-hydroxyethyl)-DMXAA, flavone-8-acetic acid (FAA), 2,7-bis(2-dimethylamino ethoxy)fluoren-9-one (Tilorone) and 10-carboxymethyl-9-acridanone (CMA), and wherein preferably the target STING is responsive to DMXAA.

Embodiment 3: The recombinant expression system of embodiment 1 or 2, wherein the target STING encoded by the sequence of the first nucleic acid construct is
a. a mutated form of a natural variant of human STING that further comprises at least one, at least two, or at least three amino acid substitutions that renders the human STING variant sensitive to the human-non-reacting STING agonist as determined in an in vitro-agonist determination assay; or
b. a non-human STING that is sensitive to the human-non-reacting STING agonist as determined in an in vitro-agonist determination assay, preferably a murine STING or a hybrid STING, even more preferably a murine STING according to SEQ ID NO: 7; wherein the target STING is preferably a DMXAA-sensitive human STING as determined in an in vitro-DMXAA determination assay.

Embodiment 4: The recombinant expression system of embodiment 3, wherein the target STING encoded by the sequence of the first nucleic acid construct is a DMXAA-sensitive human STING variant that is selected from the group consisting of:
a. a human STING[R] variant further comprising at least 1, at least 2, or at least 3 amino acid substitutions selected from the group consisting of S162A, G230I, and Q266I;
b. a human STING[AQ] variant further comprising at least 1, at least 2, or at least 3 amino acid substitutions selected from the group consisting of S162A, A230I, Q266I, E260I and E260V;
c. a human STING[H] variant further comprising at least 1, at least 2, or at least 3 amino acid substitutions selected from the group consisting of S162A, G230I, and Q266I; and
d. a truncated human STING[H] variant further comprising at least 1, or at least 2 amino acid substitutions selected from the group consisting of S162A and G230I.

Embodiment 5: The recombinant expression system of embodiment 4, wherein the DMXAA-sensitive human STING variant encoded by the sequence of the first nucleic acid construct is selected from the group consisting of hSTING[R]-S162A/G230I/Q266I, hSTING[R]-S162A/Q266I, hSTING[R]-G230I, hSTING[R]-S162A, hSTING[R]-Q266I, hSTING[R]-S162A/G230I, hSTING[AQ]-S162A/A230I/Q266I, hSTING[AQ]-S162A, hSTING[AQ]-S162A/R169A, hSTING[AQ]-S162A/T267A, hSTING[AQ]-E2601, hSTING[AQ]-E260V, hSTING[H]-S162A/G230I/Q266I, hSTING[H]-S162A/Q266I, hSTING[H]-G230I, hSTING[H]-S162A, hSTING[H]-S162A/G230I and hSTING[H]-Q266I and preferably is hSTING[R]-S162A/G230I/Q266I.

Embodiment 6: The recombinant expression system of embodiment 4, wherein the sequence of the first nucleic acid construct encoding for the DMXAA-sensitive human STING variant is selected from the group consisting of:
a. SEQ ID NO: 5;
b. a polynucleotide sequence having at least 75%, at least 80%, at least 85%, at least 90% or at least 95% sequence identity with respect to SEQ ID NO: 5;
c. a polynucleotide sequence encoding a polypeptide comprising SEQ ID NO: 6;
d. a polynucleotide sequence encoding a polypeptide having at least 75%, at least 80%, at least 85%, at least 90% or at least 95% sequence identity with respect to SEQ ID NO: 6;
e. a polynucleotide sequence encoding a polypeptide to c) or d) with at least one modification of the polypeptide that is an amino acid substitution, amino acid insertion, amino acid deletion, C-terminal truncation or N-terminal truncation;
f. a polynucleotide sequence that hybridizes under at least moderately stringent conditions to the complement of the polynucleotide sequence of any of a) to e);
g. a region of the polynucleotide sequence of a) to f) encoding a fragment of at least 50 amino acid residues of the DMXAA-sensitive human STING variant;
wherein the polypeptide encoded by the polynucleotide of any of a) to g) has the biological activity of being responsive to the agonist DMXAA as determined in an in vitro-DMXAA determination assay.

Embodiment 7: The recombinant expression system of any of the above embodiments 1 to 6, wherein the promoter of the first nucleic acid construct is a constitutive promoter, preferably selected from the list consisting of SV40, CMV0, UBC, EF1a, PGK, CAG, EFS, CBh, CBA human β actin, Ac5, Polyhedrin, TEF1, GDS, CaMV35S, Ubi, H1 and U6; or the promoter of the first nucleic acid construct is an inducible promoter, preferably selected from the list consisting of Telomerase-responsive promoter, IL-6 responsive promoter, TGF-β-inducible promoter, VEGFR-inducible promoter, Epidermal Growth factor receptor-inducible promoter, HER2/Neu-inducible promoter, MUC1 promoter, BRCA1 promoter, BRCA2 promoter, LALBA promoter, PSA promoter, ERBB2 promoter, CEA promoter, uPAR promoter and Thyroid transcription factor1 (TTF1).

Embodiment 8: The recombinant expression system of any of the above embodiments 1 to 7, wherein the at least one immunomodulatory gene or protein encoded by the sequence of the second nucleic acid construct is selected from the group consisting of interleukin-1, interleukin-2, interleukin-3, interleukin-4, interleukin-5, interleukin-7, interleukin-8, interleukin-9, interleukin-11, interleukin-12, interleukin-13, interleukin-14, interleukin-15, interleukin-16, interleukin-17, interleukin-18, interleukin-19, interleukin-20, interleukin-21, interleukin-22, interleukin-23, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, granulocyte-macrophage colony-stimulating factor, type I interferons, IFN-γ, TNF-α, FLT3-ligand, a blocking peptide targeting TGF-β, a blocking peptide targeting IL-10, a blocking peptide targeting FoxP3, a monoclonal antibody or single-chain variable fragment (scFv) or nanobody neutralizing PD1, PDL1, CTLA4, CD137, TIM3, LAG3, and a fragment or variant thereof; or a shRNA targeting TGF-β, a shRNA targeting IL-10, or a shRNA targeting FoxP3.

Embodiment 9: The recombinant expression system of any of the above embodiments 1 to 8, wherein the at least one immunomodulatory protein is selected from the group consisting of:
a. the alpha subunit of IL-12;
b. the β-subunit of IL-12;
c. single chain IL-12 comprising the α- and β-subunit of IL-12;
d. a single chain IL-12 comprising or consisting of the amino acid sequence of SEQ ID NO: 9 or 10;
e. a polypeptide having at least 70%, 75%, 80%, 85%, 90%, 95%, or 99% sequence identity with respect to SEQ ID NO: 9 or 10, wherein the polypeptide has the biological activity of interlekin-12.

Embodiment 10: The recombinant expression system of any of the above embodiments 1 to 9, wherein the STING-responsive promoter of the second nucleic acid construct is selected from the group consisting of IFN-β-responsive promoter, IFN-β-minimal promoter, CXCL9-responsive promoter, CXCL10-responsive promoter, STAT-motif promoter, IRSF3-motif promoter, ISRE-motif promoter, NFκB-motif promoter, and STAT6-motif promoter, wherein the STING responsive promoter preferably comprises at least one interferon-stimulated response element (ISRE) and more preferably is selected from the list consisting of:
a. a nucleotide sequence comprising one to four copies of the ISRE as depicted in SEQ ID NO: 11, comprising preferably four copies of said ISRE;
b. a nucleotide sequence according to SEQ ID NO: 12;
c. a nucleotide sequence according to SEQ ID NO: 13.

Embodiment 11: The recombinant expression system of any of the above embodiments 1 to 10, wherein the second nucleic acid construct furthers comprises at least one sequence motif that inhibits transgenic expression of the immunomodulatory protein in the liver, wherein the sequence motif is preferably a target sequence of a microRNA that is highly abundant in the liver, more preferably is selected from the group consisting of miR1-22, miR-192, miR-199a, miR-101, miR-99a, let7a, let7b, let7c, let7f, and even more is a miR-122 target sequence is selected from the list consisting of:
a. a miR-122 target sequence as depicted in SEQ ID NO: 14;
b. 1 to 5 copies of the miR-122 target sequence of SEQ ID NO: 14, being preferably five copies of this miR-122 target sequence;
c. a nucleotide sequence comprising 5 copies of the miR-122 target sequence as depicted in SEQ ID NO: 15.

Embodiment 12: An expression vector encoding the first and/or the second nucleic acid construct according to any of the above embodiments 1 to 11 or a combination of a first and a second expression vector, the first vector encodes the first nucleic acid construct according to any of the above embodiments 1 to 11 and the second vector encodes the second nucleic acid construct according to any of the above embodiments 1 to 11, wherein the expression vector(s) is (are) preferably a viral vector, more preferably a viral vector selected from the list consisting of adeno-associated virus (AAV) vector, adenoviral vector, lentiviral vector, vaccine virus vector, or herpes simplex virus vector, even more preferably a adeno-associated vector having one or more of the following characteristics:
a. the first and/or the second nucleic acid construct sequence further comprise a 5′ITR and a 3′ITR sequence, being preferably AAV ITRs as depicted in SEQ ID NO; 17;
b. the AAV vector is preferably an AAV8, AAV1, AAV3, AAV6, AAV9, AAV2, AAV5, AAVrh.10, or any gain-of-function mutant of AAVrh.10.

Embodiment 13: A viral particle comprising the viral vector comprising the first and/or second nucleic acid construct of embodiment 12 or a combination of a first and a second viral particle, the first viral particle comprises a viral vector encoding the first nucleic acid construct and the second viral particle comprises a viral vector encoding the second nucleic acid construct of embodiment 12, wherein the viral particle(s) is (are) preferably (an) AAV or (an) Ad viral particle(s); more preferably (an) AAV viral particle(s).

Embodiment 14: A pharmaceutical composition that comprises a pharmaceutically acceptable excipient and:
a. the recombinant expression system of any of embodiments 1 to 11; or
b. the expression vector(s) of embodiment 12; or
c. the viral particle(s) of embodiment 13.

Embodiment 15: The recombinant expression system of any of embodiments 1 to 11; the expression vector(s) of embodiment 12; the viral particle(s) of embodiment 13 or the pharmaceutical composition according to embodiment 14 for use in an immunotherapy, preferably in the immunotherapy of a proliferative disorder, more preferably in the immunotherapy of cancer, even more preferably in the immunotherapy of solid cancer.

Embodiment 16: The recombinant expression system, expression vector(s), the viral particle(s) or pharmaceutical composition for use according to embodiment 15, wherein cancer is selected from the list consisting of: bladder cancer, bone cancer, brain cancer, breast cancer, cancer of the thymus, cervical cancer, colon cancer, oesophageal cancer, gastric cancer, head & neck cancers, Hodgkin's lymphoma, liver cancer, lung cancer, melanoma, mesothelioma, multiple myeloma, Merkel cell carcinoma, non-Hodgkin's lymphoma, ovarian cancer, pancreatic cancer, prostate cancer, rectal cancer, renal cancer, sarcoma, skin cancer, testicular cancer, thyroid cancer, uterine cancer, plasma cell tumors, cholangiocarcinoma, and, neuroendocrine tumors or carcinoid tumors.

Embodiment 17: The recombinant expression system, expression vector(s), the viral particle(s) or pharmaceutical composition for use according to embodiment 15 or 16, wherein the use in the immunotherapy is a combination of:
a. administration of the recombinant expression system of any of embodiments 1 to 11; the expression vector(s) of embodiment 12, the viral particle(s) of embodiment 13 or the pharmaceutical composition according to embodiment 14 to a patient suffering from a proliferative disorder, being preferably cancer in order to yield a therapeutically effective transgenic expression of the target STING and/or the immunomodulatory protein, wherein the administration is preferably an intratumoral administration;
b. optionally treating the patient before the administration according to step a) with a radiotherapy, being preferably a radiotherapy directed to the tissue or organ intended to express the transgene(s) from a); and
c. treating the patient after the administration according step a) with a STING agonist recognizing the transgenically expressed target STING but not the human STING and natural variants thereof and/or with a radiotherapy, being preferably a radiotherapy directed to the tissue or organ intended to express the transgene(s) from a).

Embodiment 18: The recombinant expression system, expression vector(s), the viral particle(s) or pharmaceutical composition for use according to embodiment 17, wherein in step c) a STING agonist according to embodiment 2 is used, being preferably DMXAA.

Embodiment 19: The recombinant expression system, expression vector(s), the viral particle(s) or pharmaceutical composition for use according to embodiment 17 or 18, wherein in step b) a moderate or low dose radiotherapy with 40 Gy or less, preferably 8 Gy or less is used.

It should also be expressly noted that, in the context of the present patent application, indefinite articles and numerical indications such as “a”, “an”, “one”, “two”, etc. are generally to be understood as “at least”-indications, i.e. as “at least one . . . ”, “at least two . . . ”, etc., unless it expressly follows from the respective context or it is obvious or technically imperative to a person skilled in the art that only “exactly one . . . ”, “exactly two . . . ”, etc. can be meant there.

Further advantages, features and details of the invention will be apparent from the following description, in which embodiments of the invention are described in detail with reference to the figures. In this connection, the features mentioned in the claims and in the description may each be essential to the invention individually or in any combination.

The foregoing explanation of the embodiments describes the present invention exclusively in the context of examples. Of course, individual features of the embodiments can be freely combined with each other, provided that this is technically reasonable, without leaving the scope of the present invention.

### Definitions

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Approximately: The term “approximately” as used herein may be applied to modify any quantitative comparison, value, measurement, or other representation that could permissibly vary without resulting in a change in the basic function to which it is related.

A/an: As used herein the specification, “a” or “an” may mean one or more. As used herein in the claim(s), when used in conjunction with the word “comprising,” the words “a” or “an” may mean one or more than one.

Or/and/or: The use of the term “or” in the claims is used to mean “and/or” unless explicitly indicated to refer to alternatives only or the alternatives are mutually exclusive, although the disclosure supports a definition that refers to only alternatives and “and/or.”

The term “about” means, in general, within a standard deviation of the stated value as determined using a standard analytical technique for measuring the stated value. The terms can also be used by referring to plus or minus 5% of the stated value.

Essentially: The term “essentially” is to be understood that methods or compositions include only the specified steps or materials and those that do not materially affect the basic and novel characteristics of those methods and compositions.

Stereoisomers: In addition, the compounds used in the present invention may contain one or more asymmetric carbon atoms and therefore exists in two or more stereoisomeric forms.

Where a compound contains an alkenyl or alkenylene group, cis (E) and trans (Z) isomerism may also occur. The present invention includes use of the individual stereoisomers of the compound and, where appropriate, the individual tautomeric forms thereof, together with mixtures thereof.

Analogues: As used herein the term “analogue” refers to a chemical compound that is structurally similar to another but differs slightly in composition, such as in the replacement of one atom by an atom of a different element or by a functional group.

Isotopes in compounds: The present invention also includes use of all suitable isotopic variations of the compounds described herein or a pharmaceutically acceptable salt thereof.

An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the compounds and pharmaceutically acceptable salts thereof include isotopes of hydrogen, carbon, nitrogen, oxygen, phosphorus, sulphur, fluorine and chlorine such as 2H, 3H, 13C, 14C, 15N, 17O, 18O, 31P, 32P, 35S, 18F and 36Cl, respectively. Certain isotopic variations of the compound and pharmaceutically acceptable salts thereof, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations of the compounds of the present invention and pharmaceutically acceptable salts thereof can generally be prepared by conventional procedures using appropriate isotopic variations of suitable reagents.

Cytokine: The term “cytokine” refers to the general class of biological molecules which effect/affect cells of the immune system. The definition is meant to include, but is not limited to, those biological molecules that act locally or may circulate in the blood, and which, when used in the compositions or methods of the present invention serve to regulate or modulate an individual's immune response to cancer. Cytokines may be a number of different substances such as interferons, interleukins, chemokines and growth factors. Exemplary cytokines for use in practicing the invention include but are not limited to interferons (e.g. IFN-α, IFN-β, and IFN-γ), interleukins (e.g., IL-1 to IL-29, in particular, IL-2, IL-7, IL-12, IL-15 and IL-18), tumor necrosis factors (e.g., TNF-α and TNF-β), chemokines (e.g. CXCL9, CXCL10), granulocyte-macrophage colony-stimulating factor (GM-CSF), TGF-β, FLT3-ligand, and fragments or variants thereof. Notably, exogenously provided cytokines are a type of immunotherapy.

Immunomodulatory: As used herein, the term “immunomodulatory” refers to the property of initiating or modifying (e.g., increasing or decreasing) an activity of a cell involved in an immune response. An immunomodulatory composition or method may increase an activity of a cell involved in an immune response, e.g., by increasing pro- or anti-inflammatory markers, and/or may decrease an activity of a cell involved in an immune response, e.g., by decreasing pro- or anti-inflammatory markers.

Agonist/human-non-reacting STING agonist: As used herein, the term “agonist” refers to a ligand that when bound to a protein stimulates its activity. The term “human-non-reacting STING agonist” refers to an agonist that substantially does not bind to human wild-type STING or natural variants thereof but binds to mutated human STING or STING from other species such as STING derived from mouse, rat or monkey. Optionally, the term “human-non-reacting STING agonist” refers to an agonist that binds human wild-type STING or natural variants thereof without inducing STING activity, whereas STING activity of mutated human STING or STING from other species such as STING derived from mouse, rat or monkey is induced upon binding of said agonist. Thus, the term “human-non-reacting” refers to an agonist not activating one of the endogenous human STING proteins encoded in the genome of the human subject (according to one of the known haplotypes of human STING), namely hSTING[R] as depicted in SEQ ID NO: 1, hSTING[H] as depicted in SEQ ID NO: 2, hSTING[AQ] as depicted in SEQ ID NO: 3, hSTING[H] as depicted in SEQ ID NO: 4, STING[Q] which possess the amino acid substitution R293Q or STING[HAQ] which possess the amino acid substitutions R71H, G230A and R293Q, wherein exogenously provided DMXAA-sensitive human STING reacts with the “human-non-reacting” agonist.

Protein/polypeptide: The terms “protein,” “peptide,” and “polypeptide,” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

Antibody/monoclonal antibody: As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, llama and mice, as well as non-mammalian species, such as shark immunoglobulins. As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Single-chain variable fragment/scFv: The terms “single-chain variable fragment” and “scFv” refer to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

The term “nanobody”, as used herein, refers to the smallest antigen binding fragment or single variable domain (VHH) derived from naturally occurring heavy chain antibody and is known to the person skilled in the art.

The term “blocking peptide” as used herein, refers to peptides that can interfere with the molecule that they are targeting in a way that said molecule is impaired in its interaction with its targets. For example, a blocking peptide targeting TGF-β impairs its immunosuppressive function.

Nucleic acid: The term “nucleic acid” includes any nucleotides, analogues thereof, and polymers thereof. The terms “polynucleotide”, “nucleic acid construct”, or “nucleotide sequence” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogues of either RNA or DNA made from nucleotide analogues and modified polynucleotides such as, though not limited to, methylated, protected and/or capped nucleotides or polynucleotides. The terms encompass poly- or oligo-ribonucleotides (RNA) and poly- or oligo deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges. The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. The skilled person is aware that he might substitute a variable number of nucleosides in a nucleic acid by modified nucleosides which may include derivatives of purine and pyrimidine bases such as pseudo uridine, N1-methyl pseudo uridine, 5-methyl uridine, 5-methyl Cytosine. 6-methyl Adenosine, 2-thiouridine in order to enhance translation efficiency, In some embodiments, the prefix poly- refers to a nucleic acid containing 2 to about 10,000, 2 to about 50,000, or 2 to about 100,000 nucleotide monomer units. In some embodiments, the “oligonucleotide” refers to a nucleic acid containing 2 to about 200 nucleotide monomer units.

Gene: As used herein, the term “gene” means a segment of DNA that contains all the information for the regulated biosynthesis of an RNA product, including promoters, exons, introns, and other untranslated regions that control expression. Also, shRNA are included by this definition.

Hybridize: The terms “hybridizing” or “hybridizes” as used herein is to be understood as two nucleic acid strands (e.g., an oligonucleotide and a target nucleic acid) forming hydrogen bonds between base pairs on opposite strands thereby forming a duplex.

Promoter: The term “promoter” is used herein in its ordinary sense to refer to a nucleotide region comprising a DNA regulatory sequence, wherein the regulatory sequence is derived from a gene and is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrase “operatively positioned”, “operatively linked”, “under control”, and “under transcriptional control” mean that a promoter is in a correct functional location and/or orientation in relation to a nucleic acid sequence to control transcriptional initiation and/or expression of that sequence.

Constitutive promoter: The term “constitutive promoter” refers to a nucleotide sequence which, when operably linked with a polynucleotide which encodes or specifies a gene product, causes the gene product to be produced in a cell under most or all physiological conditions of the cell.

Inducible promoter: The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

STING-responsive promoter: As used herein, the term “STING-responsive promoter” refers to a nucleotide sequence which, when operably linked with a polynucleotide which encodes or specifies a gene product, causes the gene product to be produced in a cell substantially only when a polypeptide that is regulated by STING activity is present in the cell. Non-limiting examples of such polypeptides are cytokines, especially interferons such as IFN-β, chemokines such as CXCL9 or CXCL10, transcription factors such as NFκB or STATs, interleukins such as IL-6, and TNF-α.

Mutation: The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

Identity: As used herein, the term “identity” refers to the overall relatedness between polymeric molecules, e.g., between polypeptide molecules and/or between nucleic acid molecules (e.g., DNA molecules and/or RNA molecules). In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Hybridization under stringent conditions: In the context of the present invention, a hybridization under stringent conditions refers to hybridization conditions, in which only long sequences with nearly perfect complementary matching will secure anneal. This term is well-known in the art of genetic engineering and has been used in the patent practice for numerous years as a quasi-structural feature for defining DNA claims. Notably, the respective claims define their subject-matter not only by the capability of hybridizing under stringent conditions, but also by a further functional feature, namely that the polypeptide encoded by the claimed DNA sequence has at least the functional property of STING.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Operably linked/operatively linked: As used herein, the terms “operably linked” or “operatively linked” refer to a juxtaposition wherein the components described are in a relationship permitting them to function in their intended manner. A control element such as a promoter “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to the functional element of interest.

RNA interference/microRNA/shRNA: As used herein, the term “RNA interference” refers generally to a process in which a RNA molecule reduces or inhibits expression of a nucleic acid sequence with which the RNA molecule shares substantial or total homology. Without wishing to be bound by any theory, it is believed that, in nature, the RNA interference pathway is initiated by a Type III endonuclease known as Dicer, which cleaves long double-stranded RNA (dsRNA) into double-stranded fragments typically of 21-23 base pairs with 2-base 3′ overhangs (although variations in length and overhangs are also contemplated), referred to as “short interfering RNAs” (“siRNAs”). Such siRNAs comprise two single-stranded RNAs (ssRNAs), with an “antisense strand” or “guide strand” that includes a region that is substantially complementary to a target sequence, and a “sense strand” or “passenger strand” that includes a region that is substantially complementary to a region of the antisense strand. Those of ordinary skill in the art will appreciate that a guide strand may be perfectly complementary to a target region of a target RNA or may have less than perfect complementarity to a target region of a target RNA. As used herein, the terms “microRNA” or “miRNA” or “miR” refer to a specific type of siRNA that are produced physiologically in the organism rather than produced synthetically. miRNA bind to respective miRNA target sequences and reduce or inhibit expression of a nucleic acid sequence with which the miRNA shares a substantial or total homology. As used herein, the term “shRNA” or “short hairpin RNAs” refers to individual transcripts that adopt stem-loop structures which are processed into siRNA by RNAi machinery. Typical shRNA molecules comprise two inverted repeats containing the sense and antisense target sequence separated by a loop sequence. The base-paired segment may be any suitable length that allows inactivation of a target gene in vivo, wherein one strand of the base-paired stem is complementary to the mRNA of said target gene. The loop of the shRNA stem-loop structure may be any suitable length that allows inactivation of the target gene in vivo. The base paired stem may be perfectly base paired or may have 1 or 2 mismatched base pairs.

Target gene/RNA: A “target gene”, as used herein, refers to a gene whose expression is to be modulated, e.g., upregulated or inhibited. As used herein, the term “target RNA” refers to an RNA to be degraded or translationally repressed or otherwise inhibited using one or more agents, e.g., one or more miRNAs or siRNAs. A target RNA may also be referred to as a target sequence or target transcript. The RNA may be a primary RNA transcript transcribed from the target gene (e.g., a pre-mRNA) or a processed transcript, e.g., mRNA encoding a polypeptide. As used herein, the term “target portion” or “target region” refers to a contiguous portion of the nucleotide sequence of a target RNA. In some embodiments, a target portion of an mRNA is at least long enough to serve as a substrate for RNA interference (RNAi)-mediated cleavage within that portion in the presence of a suitable miRNA or siRNA. A target portion may be from about 8-36 nucleotides in length, e.g., about 10-20 or about 15-30 nucleotides in length. A target portion length may have specific value or subrange within the afore-mentioned ranges. For example, in certain embodiments a target portion may be between about 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length.

Recombinant: As used herein, the term “recombinant” is intended to refer to nucleic acids or polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Natural variant: The term “natural variant” refers to variants of genes or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Said natural variants occur in nature and are not artificially generated. In contrast, when the term “variant” is used without the word “natural” being associated to it, the variants may comprise artificially generated variants such as mutations. In the case of human STING some known natural variants are STING[R], STING[AQ], STING[H], STING[HAQ] and a truncated form of STING[H].

Wild-type: As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e., two or more alleles exist) at a genetic locus in the individuals of a population. As used herein, “mutant” refers to a change in the sequence of a nucleic acid or its encoded protein, polypeptide, or peptide that is the result of recombinant DNA technology.

Non-human: As used herein the term “non-human” refers to a gene or protein that is not substantially derived from wild-type human genes or proteins. In the context of this invention targeted mutations of a human protein still render it human, whereas hybridizing parts of a human protein with parts of a homolog protein of another species render it non-human.

Hybrid STING: As used herein the term “hybrid STING” refers to STING where a part of the protein sequence is derived from human STING and another part is derived from a homolog STING of another species.

Target STING: As used herein, a target STING is a STING, which is responsive to a STING agonist which recognizes said target STING but not the human STING and natural variants thereof. Accordingly the target STING may be a mutated human STING but is not an endogenous human STING encoded in the genome of the human subject (according to one of the known haplotypes of human STING), namely hSTING[R] as depicted in SEQ ID NO: 1, hSTING[H] as depicted in SEQ ID NO: 2, hSTING[AQ] as depicted in SEQ ID NO: 3, hSTING[H] as depicted in SEQ ID NO: 4, STING[Q] which possess the amino acid substitution R293Q or STING[HAQ] which possess the amino acid substitutions R71H, G230A and R293Q. Hereby, the responsiveness to the STING agonist is determined in an “in vitro-DMXAA determination assay”. Non-limiting examples for a target STING is murine STING or the mutated human STING hSTING[R]-S162A/G230I/Q266I.

Cistron/monocistronic/bicistronic/polycistronic: The term “cistron”, as used herein, refers to a section of a DNA or RNA molecule that codes for a single protein or a single functional RNA molecule. The terms “monocistronic”, “bicistronic” or “polycistronic” refer to the number of cistrons on a DNA or RNA molecule. For example, a bicistronic vector comprising two cistrons under the control of one promoter leads to translation of two different proteins.

Exogenous/exogenic: The terms “exogenous” or “exogenic” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

Plasmid: A “plasmid,” a common type of a vector, is an extra-chromosomal DNA molecule separate from the chromosomal DNA that is capable of replicating independently of the chromosomal DNA. In certain cases, it is circular and double-stranded.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA or RNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” One of ordinary skill in the art understands that a “viral vector”, as described herein, includes viral components in addition to a transgene described herein, e.g., capsid proteins.

Expression system: By “expression system” or “expression construct” is meant one or more nucleic acid molecules that is/are capable of directing transcription. An expression system includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included. The term “expression system” and the term “recombinant expression system” therefore encompass DNA or RNA nucleic acid molecules that is/are capable of directing transcription. In the context if the present invention, the respective DNA or RNA nucleic acid molecules are also designated as “nucleic acid construct”.

AAV: The term “AAV” refers to adeno-associated virus and may be used to refer to the naturally occurring wild-type virus itself or derivatives thereof. The term covers all subtypes, serotypes and pseudotypes, and both naturally occurring and recombinant forms, except where required otherwise. The AAV genome is built of single stranded DNA and comprises inverted terminal repeats (ITRs) at both ends of the DNA strand, and two open reading frames: rep and cap, encoding replication and capsid proteins, respectively. A foreign polynucleotide can replace the native rep and cap genes. AAVs can be made with a variety of different serotype capsids which have varying transduction profiles or, as used herein, “tropism” for different tissue types. As used herein, the term “serotype” refers to an AAV which is identified by and distinguished from other AAVs based on capsid protein reactivity with defined antisera, e.g., AAV1, AAV2, AAV3, AAV4, AAV5, AAV6, AAV7, AAV8, AAV9, and AAVrh10. For example, serotype AAV2 is used to refer to an AAV which contains capsid proteins encoded from the cap gene of AAV2 and a genome containing 5′ and 3′ ITR sequences from the same AAV2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped rAAV are produced using standard techniques described in the art.

Adenovirus: The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

Lentivirus: As used herein, the term “lentivirus” refers to a group (or genus) of complex retroviruses. Illustrative lentiviruses include but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

Viral particle: As used herein, the term “viral particle” refers to all or part of a virion. For example, the viral particle comprises a recombinant genome and may further comprise a capsid. The viral particle may be a gene therapy vector. Herein, the terms “viral particle” and “vector” are used interchangeably. For the purpose of the present application, a “gene therapy” vector is a viral particle that can be used in gene therapy, i.e. a viral particle that comprises all the required functional elements to express a transgene, such as a CFI nucleotide sequence, in a host cell after administration. Suitable viral particles of the invention include a parvovirus, a retrovirus, a lentivirus or a herpes simplex virus. The parvovirus may be an adeno-associated virus (AAV). Optionally, the viral particle is an AAV or adenoviral particle.

Host cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, the cell is a human, monkey, ape, hamster, rat, or mouse cell. In some embodiments, the cell is eukaryotic and is selected from the following cells: CHO (e.g., CHO Kl, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, MC38 cell, myeloma cell, tumor cell, and a cell line derived from an aforementioned cell. In some embodiments, the cell comprises one or more viral genes.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically engineered animal, and/or a clone.

Treatment/treating: “Treatment” or “treating” includes (1) inhibiting a disease, disorder or condition in a subject or patient experiencing or displaying the pathology or symptomatology of the disease (e.g., arresting further development of the pathology and/or symptomatology), (2) ameliorating a disease, disorder or condition in a subject or patient that is experiencing or displaying the pathology or symptomatology of the disease (e.g., reversing the pathology and/or symptomatology), and/or (3) effecting any measurable decrease in a disease, disorder or condition in a subject or patient that is experiencing or displaying the pathology or symptomatology of the disease.

Prophylactically treating: “Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

Local administration/local delivery: As used herein, the term “local administration” or “local delivery”, in reference to delivery of a recombinant expression system, an expression vector, a viral particle or a pharmaceutical composition described herein, refers to delivery that does not rely upon transport of said products to its intended target tissue or site via the vascular system. Said products described herein may be delivered directly to its intended target tissue or site, or in the vicinity thereof, e.g., in close proximity to the intended target tissue or site. For example, said products may be delivered by injection or implantation of the composition or compound or by injection or implantation of a device containing the composition or compound. Following local administration in the vicinity of a target tissue or site, said products described herein, or one or more components thereof, may diffuse to the intended target tissue or site. It will be understood that once having been locally delivered a fraction of said products described herein (typically only a minor fraction of the administered dose) may enter the vascular system and be transported to another location, including back to its intended target tissue or site. As used herein, the term “local administration” or “local delivery”, in reference to delivery of a viral particle or a pharmaceutical composition described herein, refers to delivery that can rely upon transport of the viral vector or the pharmaceutical composition to its intended target tissue or site via the vascular system.

Concurrent administration: As used herein, the term “Concurrent administration” with respect to two or more compounds, e.g., therapeutic agents, is administration performed using doses and time intervals such that the administered compounds are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc.). Accordingly, the compounds may, but need not be, administered together as part of a single composition. In addition, the compounds may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). According to various embodiments of the disclosure, compounds administered within such time intervals may be considered to be administered at substantially the same time. In certain embodiments of the disclosure, concurrently administered compounds are present at effective concentrations within the body (e.g., in the blood and/or at a site of local complement activation) over the time interval. When administered concurrently, the effective concentration of each of the compounds needed to elicit a particular biological response may be less than the effective concentration of each compound when administered alone, thereby allowing a reduction in the dose of one or more of the compounds relative to the dose that would be needed if the compound was administered as a single compound. The effects of multiple compounds may, but need not be, additive or synergistic. The compounds may be administered multiple times. The non-negligible concentration of a compound may be, for example, less than approximately 5% of the concentration that would be required to elicit a particular biological response, e.g., a desired biological response.

Sequential administration: As used herein, the term “sequential administration” of two or more compounds refers to administration of two or more compounds to a subject such that the compounds are not present together in the subject's body, or at a relevant site of activity in the body, at greater than non-negligible concentrations. Administration of the compounds may, but need not, alternate. Each compound may be administered multiple times.

Systemic administration: As used herein, the term “systemic administration” and like terms are used herein consistently with their usage in the art to refer to administration of a compound (e.g., a therapeutic agent, expression vector, viral particle, pharmaceutical composition, and/or formulation) such that the compound becomes widely distributed in the body in significant amounts and has a biological effect, e.g., its desired effect, in the blood and/or reaches its desired site of action via the vascular system. Typical systemic routes of administration include administration by (i) introducing the compound directly into the vascular system or (ii) subcutaneous, oral, pulmonary, or intramuscular administration wherein the compound is absorbed, enters the vascular system, and is carried to one or more desired site(s) of action via the blood.

Intratumor administration: The term “intratumor administration” refers to the administration of a compound (e.g., a therapeutic agent, expression vector, viral particle, pharmaceutical composition, and/or formulation) such that the compound is present within the tumor after administration. Those of ordinary skill in the art will appreciate that the administration thereby can occur directly within the tumor or in close proximity to the tumor.

Subject: As used herein, the terms “subject”, “test subject” or “patient” refer to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Effective/therapeutically effective amount: As used herein, the term “effective” or “therapeutically effective amount” means at least the minimum amount of a compound (e.g., a therapeutic agent, expression vector, viral particle, pharmaceutical composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount. A therapeutically effective amount is also one in which any toxic or detrimental effects of the treatment are outweighed by the therapeutically beneficial effects. In the case of cancer or tumor, an effective amount of a compound may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder.

Combination of expression vectors/combination of viral particles: By the phrase “combination of expression vectors” or “combination of viral particles” is meant herein that the first expression vector or the first viral particle is administered to the patient before, during (including concurrently with preferably co-formulated with) and/or after treatment of an individual with the second expression vector or second viral particle, respectively. The combined expression vectors or viral particles may be used for simultaneous, sequential, or separate administration. In all cases, it is preferred that any of the herein-mentioned expression vectors and viral particles are administered in pharmaceutically effective amounts, i.e. an administration involving a total amount of each component of the combination that is sufficient to show a meaningful patient benefit. The formulations may conveniently be presented in unit dosage form by methods known to those skilled in the art.

Kit of parts: A “kit of parts” as used herein is a kit that provides a combination of the first and the second expression vector or a combination of the first and the second viral particle for administration in combination. It is preferred that the kit may for example contain the vectors/particles in dosage forms for administration. A dosage form contains a sufficient amount of one or more of the vectors/particles such that a desirable effect can be obtained when administered to a subject. Thus, it is preferred that the medical packaging comprises an amount of dosage units corresponding to the relevant dosage regimen. Preferably, the kit-of-parts contains instructions indicating the use of the dosage form to achieve a desirable affect and the amount of dosage form to be taken over a specified time period.

Immunotherapy: The term “immunotherapy”, as used herein, refers to a therapy, whereby the modulation or stimulation of the immune system is used to treat a disease, preferably a proliferative disease such as cancer, utilizing immunomodulatory genes or proteins. The immunotherapy may cause a stimulation of the immune system which is often used in immunotherapies targeting cancer. However, also other diseases may be treated with immunotherapies, such as autoimmune diseases, diabetes, cardiovascular diseases and inflammation. For example, IFN-β is used to treat multiple sclerosis. In addition, IL-2 has been shown to regulate the balance of regulatory and effector T cells thereby preserving β-cell function. Immunotherapies may be used to treat non-cancer diseases such as lupus, rheumatoid arthritis, diabetes.

Immune checkpoint inhibitor: The term “immune checkpoint inhibitor” or “checkpoint inhibitor”, as used herein, refers to a compound that inhibits a protein in the checkpoint signaling pathway. Proteins in the checkpoint signaling pathway include for example, CD27, CD28, CD40, CD 122, CD137, OX40, GITR, ICOS, A2AR, B7-H3, B7-H4, BTLA, CTLA-4, IDO, KIR, LAG3, PD-1, PD-L1, PD-L2, TIM-3, TIGIT, Lair1, CD244, HAVCR2, CD200, CD200R1, CD200R2, CD200R4, LILRB4, PILRA, ICOSL, 4-1BB or VISTA.

Immune checkpoint inhibitors are known in the art. For example, the immune checkpoint inhibitor can be a small molecule. A “small molecule” as used herein, is meant to refer to a composition that has a molecular weight in the range of less than about 5 kDa to 50 Da, for example less than about 4 kDa, less than about 3.5 kDa, less than about 3 kDa, less than about 2.5 kDa, less than about 2 kDa, less than about 1.5 kDa, less than about 1 kDa, less than 750 Da, less than 500 Da, less than about 450 Da, less than about 400 Da, less than about 350 Da, less than 300 Da, less than 250 Da, less than about 200 Da, less than about 150 Da, less than about 100 Da. Small molecules can be, e.g., nucleic acids, peptides, polypeptides, peptidomimetics, carbohydrates, lipids or other organic or inorganic molecules. Alternatively, the immune checkpoint inhibitor is an antibody or fragment thereof. For example, the antibody or fragment thereof is specific to a protein in the checkpoint signaling pathway, such as CD27, CD28, CD40, CD 122, CD137, OX40, GITR, ICOS, A2AR, B7-H3, B7-H4, BTLA, CTLA-4, IDO, KIR, LAG3, PD-1, PD-L1, PD-L2, TIM-3, TIGIT, Lair1, CD244, HAVCR2, CD200, CD200R1, CD200R2, CD200R4, LILRB4, PILRA, ICOSL, 4-1BB or VISTA. Exemplary, anti-immune checkpoint antibodies include for example ipilimumab (anti-CTLA-4), pembrolizumab (anti-PD-L1), nivolumab (anti-PD-L1), atezolizumab (anti-PD-L1), duralumab (anti-PD-L1) orrelatlimab (anti-LAG-3).

Adoptive cell therapy: The term “adoptive cell therapy”, as used herein, refers to therapies in which autologous or allogenic immune cells are infused into patients to treat certain diseases such as cancer. Various cell types can be used for this purpose, such as dendritic cells, natural killer (NK) cells or T-cells.

Adoptive T-cell therapy: The term “adoptive T-cell therapy”, as used herein, refers to therapies in which autologous or allogenic T-cells are infused into patients to treat certain diseases such as cancer. One type of adoptive T-cell therapy is to isolate tumor-infiltrating-leukocytes (TIL) from the tumor, optionally expand these cells outside of the patient and infuse them back into the patient. Another type of adoptive T-cell therapy is chimeric antigen receptor (CAR) T-cell therapy. CAR-T-cells may be designed in several ways that enhance tumor cytotoxicity and specificity, evade tumor immunosuppression, avoid host rejection, and prolong their therapeutic half-life.

Cancer vaccine: The term “cancer vaccine”, as used herein, refers to a vaccine that either treats existing cancer or prevents development of cancer.

Combination therapy: The term “combination therapy”, as used herein, refers to those situations in which two or more different compounds (e.g., a therapeutic agent, expression vector, viral particle, pharmaceutical composition, and/or formulation) are administered in overlapping regimens so that the subject is simultaneously exposed to both compounds. When used in combination therapy, two or more different compounds may be administered simultaneously or separately. This administration in combination can include simultaneous administration of the two or more compounds in the same dosage form, simultaneous administration in separate dosage forms, and separate administration. That is, two or more compounds can be formulated together in the same dosage form and administered simultaneously. Alternatively, two or more compounds can be simultaneously administered, wherein the compounds are present in separate formulations. In another alternative, a first compound can be administered followed by one or more additional compounds. In the separate administration protocol, two or more compounds may be administered a few minutes apart, or a few hours apart, a few days apart, or a few weeks apart. In some embodiments, two or more compounds may be administered 1-2 weeks apart.

Radiotherapy: The term “radiotherapy” refers hereinafter to the medical use of ionizing radiation, generally as part of cancer treatment to control or kill malignant cells. The ionizing radiation might be based on photon rays such as X-rays or gamma-rays or particle rays, wherein the particle of the particle ray is preferably selected from the group consisting of beta particle, alpha particle, neutron, muon, pion, proton or other heavier positive ions.. It may also be used as part of adjuvant therapy, to prevent tumor recurrence after surgery that removes a primary malignant tumor. Radiation therapy may be synergistic with other anti-cancer therapies, and may be used before, during, and after other anti-cancer therapies in susceptible cancers. The term “low-dose radiotherapy” refers hereinafter to radiotherapies with an ionizing radiation dose of less than 40 Gy, preferably 10 Gy or less and more preferably 8 Gy or less. Low-dose radiotherapy can be any radiotherapy with an ionizing radiation dose of less than 40 Gy, 38 Gy, 36 Gy, 34 Gy, 32 Gy, 30 Gy, 28 Gy, 26 Gy, 24 Gy, 22Gy, 20 Gy, 19 Gy, 18 Gy, 17 Gy, 16 Gy, 15 Gy, 14 Gy, 13 Gy, 12 Gy, 11 Gy, 10 Gy, 9 Gy, 8 Gy, 7 Gy, 6 Gy, 5 Gy, 4 Gy, 3 Gy, 2.5 Gy, 2.0 Gy, 1.5 Gy, 1 Gy, 0.5 Gy, 0.25 Gy, 0.2 Gy, 0.15 Gy, 0.1 Gy, 0.05 Gy, or 0.01 Gy.

Proliferative disease: As used herein, the term “proliferative disease” refers to conditions in which unregulated or abnormal growth, or both, of cells can lead to the development of an unwanted condition or disease, which may or may not be cancerous. Exemplary cell proliferative disorders of the disclosure encompass a variety of conditions wherein cell division is deregulated. Exemplary cell proliferative disorder include but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells.

Cancer/tumor: The terms “cancer” or “tumor” refer to the presence of cells possessing characteristics typical of cancer-causing cells, such as uncontrolled proliferation, immortality, invasive or metastatic potential, rapid growth, and certain characteristic morphological features. In some embodiments, such cells exhibit such characteristics in part or in full due to the expression and activity of immune checkpoint proteins, such as PD-1, PD-L1, PD-L2, and/or CTLA-4. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, a variety of cancers, carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, and Burkitt's lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xeroderma pigmentosum, keratoacanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, cholangiocarcinoma, breast cancer, colon carcinoma, and head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, Sino nasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, and some rare subtypes like bullous, erythrodermic and telangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, hepatocellular carcinoma, hepatoblastoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer, teratocarcinoma, chemotherapy refractory non-seminomatous germ-cell tumors, and Kaposi's sarcoma, and any metastasis thereof. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, brain tumor, lung carcinoma (including lung adenocarcinoma), small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, neuroendocrine tumors, carcinoid tumors, Merkel cell carcinoma, or skin cancer.

Anti-cancer: An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Pharmaceutical composition: The term “pharmaceutical composition” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

Synergism/synergistic effect: The terms “synergism” or “synergistic effect” refer to the combined effect of two or more compounds that is greater than the sum of the separate effects of the cancer compounds/therapies alone. in vitro-agonist determination assay/in vitro-DMXAA determination assay: The term “in vitro-agonist determination assay” refers to assays that allow the identification of human-non-reacting STING agonists in that said agonists activate variants of human STING or non-human STING but not human wild-type STING or natural variants thereof. The term “in vitro-DMXAA determination assay” refers to assay that allow to identify STINGs that are responsive to DMXAA.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### BRIEF DESCRIPTION OF THE DRAWINGS

FIG. 1, comprising Figures A-G, illustrate A) IFN-β expression of MC38 cells upon lentiviral transduction with either hSTINGwt or hSTINGmut and subsequent DMXAA treatment relative to vehicle treated cells; data shown are mean±SEM (n=3, ** P<0.01, two-way ANOVA followed by Sidak's multiple comparison test); B) IFN-β expression of Hep2G cells upon lentiviral transduction with either hSTINGwt or hSTINGmut and subsequent DMXAA treatment relative to vehicle treated cells; data shown are mean±SEM (n=3, ** P<0.01, two-way ANOVA followed by Sidak's multiple comparison test); C) IFN-β expression of Hep2G cells upon lentiviral transduction with mSTINGwt and subsequent DMXAA treatment relative to vehicle treated cells; data shown are mean±SEM (n=9, ** P<0.01, two-way ANOVA followed by Sidak's multiple comparison test); D) STING-derived cytokine expression of MC38 cells upon lentiviral transduction with either luciferase, hSTINGwt or hSTINGmut, or no transduction, and subsequent DMXAA treatment relative to vehicle treated cells; data shown are mean±SEM (n=3, * P<0.05, ** P<0.01, one sample at a time by one sample T test); E) STING-derived cytokine expression of Hep2G cells upon lentiviral transduction with either luciferase, hSTINGwt or hSTINGmut, or no transduction, and subsequent DMXAA treatment relative to vehicle treated cells; data shown are mean±SEM (n=3, * P<0.05, ** P<0.01, one sample at a time by one sample T test); F) time-course of STING-derived cytokine expression of MC38 cells upon lentiviral transduction with hSTINGmut and subsequent DMXAA treatment relative to vehicle treated cells; data shown are mean±SEM (n=3, * P<0.05, ** P<0.01, *** P<0,001, two-way ANOVA followed by Fisher LSD test); and G) time-course of STING-derived cytokine expression of MC38 cells upon lentiviral transduction with hSTINGmut and subsequent DMXAA treatment relative to vehicle treated cells; data shown are mean±SEM (n=3, * P<0.05, ** P<0.01, *** P<0,001, two-way ANOVA followed by Fisher LSD test).

FIG. 2 illustrates IFN-β serum levels of mice injected intravenously with an AAV8 vector encoding either mSTING or GFP and repeatedly treated with DMXAA at different doses (5, 10 or 20 mg/kg bodyweight) twice a week. Data shown are mean±SEM (n=4-12 mice per group) ** P<0.01, *** P<0.001, One-way ANOVA followed by Dunnett's multiple comparisons test.

FIG. 3, comprising Figures A-H, illustrate A) luciferase activity of MC38 cells that were lentivirally transduced with vectors encoding luciferase under the control of different modified IFN-β-promoters in the absence or presence of DMXAA; data are mean±SEM (n=2 wells per condition) ** p<0.01, *** P<0.001, **** P<0.0001, Two-way ANOVA followed by Sidak's multiple comparison test (Vehicle—DMXAA); B) luciferase activity of MC38 cells that were lentivirally transduced with vectors encoding luciferase under the control of different chemokine-promoters in the absence or presence of DMXAA; data are mean±SEM (n=2 wells per condition) **** P<0.0001, Two-way ANOVA followed by Tukey's multiple comparison test; C) IL-12 mRNA expression in MC38 cells that were lentivirally transduced with vectors encoding IL-12 under the control of either a IFN-β, a CXCL9- or a CXCL10-promoter at different timepoints after DMXAA treatment; data shown are mean±SEM (n=3, * P<0.05, two-way ANOVA followed by Kruskal-Wallis test; D) IL-12 protein levels in the supernatant of MC38 cells that were lentivirally transduced with vectors encoding IL-12 under the control of either a IFN-β, a CXCL9- or a CXCL10-promoter at different timepoints after DMXAA treatment; data are mean±SEM (n=2-8 wells/condition), **** P<0.0001, Two-way ANOVA followed by Sidak's multiple comparison test; E) and F) luciferase activity of MC38 cells that were transfected with plasmids encoding luciferase under the control of different synthetic promoters in the absence or presence of DMXAA; data are mean±SEM (n=2 wells per condition), * P<0.05, *** P<0.001, **** P<0.0001, Two-way ANOVA followed by Sidak's multiple comparisons test; and G) and H) luciferase activity of HEK293T cells that were transfected with plasmids encoding luciferase under the control of different synthetic promoters in the absence or presence of DMXAA; data are mean±SEM (n=2 wells per condition), **** P<0.0001, Two-way ANOVA followed by Sidak's multiple comparisons test.

FIG. 4, comprising Figures A-C, illustrate A) IL-12 protein levels in the serum of mice upon intravenous injection with an AAV8 encoding IL-12 under the control of either a IFN-Q-promoter or a ISRE-promoter in the absence of DMXAA; data are mean±SEM (n=2-3 animals per condition), * P<0.05 Two-way ANOVA followed by Tukey's multiple comparisons test; B) reduced IL-12 protein levels in the serum of mice upon intravenous injection with an AAV8 encoding single chain (sc)IL-12 operably linked to miR122 binding sites under the control of a ISRE-promoter compared to the said AAV8 vector lacking miR122 binding sites, in the absence of DMXAA; data are mean±SEM (n=4 animals per condition), **** P<0.0001, Student t test; and C) reduced IL-12 protein levels in the serum of mice upon intravenous injection with an AAV8 encoding hSTINGmut under a constitutive active promoter and an AAV8 encoding scIL-12 operably linked to miR122 binding sites under the control of a ISRE-promoter or said AAV8 without the miR122 binding sites in the presence or absence of DMXAA; data are mean±SEM (n=4-8 animals per condition), * P<0.05 Two-way ANOVA followed by Tukey's multiple comparisons test.

FIG. 5 illustrates the volume of subcutaneous MC38 tumors implanted in STING KO mice which were treated by intratumor co-injection of an AAV8 vector encoding hSTINGmut under the control of a constitutive promoter (AAV8-hSTINGmut) at the dose of 1×10¹¹ vg/mouse plus an AAV8 vector encoding scIL-12 operatively linked to miR122 binding sites and placed under the control of a ISRE promoter (AAV8-ISREx4IL12miR122T) at the dose of 0.5×10¹¹ vg/mouse. These animals received at the indicated time intervals either DMXAA (solid triangles) or vehicle (solid circles). In an additional control group, the tumor was injected with AAV8-hSTINGmut (1×10¹¹ vg/mouse) plus an AAV8 vector encoding GFP (0.5×10¹¹ vg/mouse) followed by vehicle (empty circles). Data are mean±SEM (n=4-6 animals per condition) *P<0.05, ** P<0.01, **** P<0.0001 Mixed-effects analysis followed by Tukey's multiple comparisons test.

FIG. 6, comprising FIGS. 6A and 6B, illustrate A) sections of subcutaneous MC38 tumor immunohistochemically stained with an anti-GFP antibody 3 days after AAV8-GFP intra-tumor injections. The transduction efficiency of AAV8-GFP injected tumors without radiation pre-treatment (left) is markedly reduced compared to tumors with 8 Gy radiation pre-treatment (right). B) The graph shows percentage of strongly GFP-labelled pixels related to the tumor area. Data are mean±SEM (n=3-6 animals per condition) *** P<0.001 Unpaired Student's t test.

FIG. 7 illustrates IFN-β protein levels in the supernatant of MC38 cells which either received radiation and/or transduction with a hSTINGmut encoding vector quantified at indicated time points after irradiation; data are mean±SEM (n=2-6 samples per condition), * P<0.05 Mixed-effects followed by Tukey's multiple comparisons test (simple effect). #P<0.05, ####P<0.0001 vs MC38 8Gy, Two way ANOVA followed by Tukey's multiple comparisons test (comparing values in a given time point).

FIG. 8, comprising Figures A-D, illustrate A) the time course of tumor volume progression in mm³ of MC38 subcutaneous tumors in C57BL/6 wild-type mice after either radiation or no radiation followed by intratumor injection of either AAV8-GFP, AAV8-hSTINGmut and/or AAV8-ISREx4-IL12miR122T as indicated. Data are mean±SEM (n=4-5 mice per group), A two-way analysis of variance (ANOVA) test with Tukey's post-test analysis were used to assess significance. Simple main effects analysis showed that treatment has a statistically significant effect on tumor growth (***p<0.001). B) The serum levels of IL-12 measured by ELISA on day 17 after treatment of the animals in A). Dots show individual values of each mouse, the middle horizontal line represents the median, and the error bars indicate the standard error of the mean (SEM). There were no statistically significant differences between groups as determined by one-way ANOVA (F (2, 7)=0.4375, p=0 0.6622). C) and D) refer to C57BL/6 wild-type mice with two subcutaneous MC38 tumors, one on the right flank and the other on the left flank; the former received treatment while the contralateral tumor was left untreated in order to analyse for abscopal effects. Right flank tumor was treated by local irradiation with 8 Gy followed by intratumor injection of either AAV8-GFP, AAV8-hSTINGmut and/or AAV8-ISREx4-IL12miR122T as indicated. C) shows the growth of the treated tumor and D) shows the growth of untreated tumor. Data are mean±SEM (n=6 mice per group). A two-way analysis of variance (ANOVA) test with Tukey's post-test analysis was used to assess significance. Simple main effects analysis showed that treatment has a statistically significant effect on the growth of the treated tumor (***p<0.001) accompanied by a statistically significant antitumor effect in the contralateral tumor at day 15 post-treatment (*p<0.01).

FIG. 9, comprising Figures A-F, illustrate A) IFN-β mRNA levels in MC38 cells upon stimulation with DMXAA or vehicle in non-transduced cells or cells transduced with AAV8-hSTINGmut after radiation with 8 Gy or no radiation (0 Gy); data shown are mean±SEM (n=3, * P<0.05, ** P<0.01, one-way ANOVA followed by Kruskal-Wallis test); B) IL-12 mRNA levels in MC38 cells upon stimulation with DMXAA or vehicle in cells transduced with AAV8-hSTINGmut plus AAV8-ISREx4-IL12miR122T after radiation with 8 Gy or no radiation (0 Gy); data shown are mean±SEM (n=3, *** P<0,001, one-way ANOVA followed by Kruskal-Wallis test); C) and D) IFN-β mRNA levels (in C) or IL-12 mRNA levels (in D) in HEK293 cells upon stimulation with DMXAA or vehicle in non-transduced cells, cells transduced with AAV8-hSTINGmut plus AAV8-GFP, cells transduced with AAV8-GFP plus AAV-ISREx4-IL12miR122T or cells transduced with AAV8-hSTINGmut plus AAV-ISREx4-IL12miR122T after radiation with 8 Gy or no radiation (0 Gy); data shown are mean±SEM (n=3, **P<0.01, ***P<0,001, **** P<0.0001, two-way ANOVA followed by Tukey multiple comparison test); E) IFN-β protein levels (pg/ml) obtained by ELISA in supernatants of cultured non-transduced MC38 cells treated with different doses of DMXAA (0, 5, 10, 20 and 50 μg/mL) 24 hours after radiation (0, 2 or 8 Gy) and collected 6, 24 or 48 h after DMXAA administration; data are mean±SEM (n=2-5 samples per condition), **** P<0.0001, Two-way ANOVA followed by Tukey's multiple comparisons test; and F) IFN-β protein levels (pg/ml) obtained by ELISA in supernatants of cultured non-transduced MC38 cells treated with DMXAA (50 pg/mL) or vehicle 24 hours after radiation (8 Gy) or no radiation (0 Gy) and collected 24 h after DMXAA administration; data are mean±SEM (n=3-4 samples per condition), *** P<0.001, **** P<0.0001, Two-way ANOVA followed by Tukey's multiple comparisons test.

FIG. 10, comprising Figure A-D, show results obtained in STING KO mice with subcutaneous MC38 tumors treated with 0 or 8 Gy irradiation followed by intratumor injection with AAV vectors encoding GFP (control) or hSTINGmut and/or HL12miR122T as indicated. Two days later they received intraperitoneal DMXAA or vehicle. The figure illustrates serum levels of A) IFN-β, B) IL-12, C) IFN-γ at the indicated time points. Figure D represents IL-12 levels in tumor extracts obtained 6 h after DMXAA or vehicle treatment Values were obtained by ELISA. Data are mean±SEM (n=4-5 mice per group), * P<0.05, **P<0.01, *** P<0.001, **** P<0.0001, Two-way ANOVA followed by Tukey's multiple comparisons test.

FIG. 11, comprising Figure A-B, show results of murine cells transduced with either AAV encoding GFP under the control of a constitutive promoter (CAG) (AAV8. CAG.GFP) or AAV8 encoding IL-12 coupled to miR122T under control of the ISREx4 promoter (AAV8-ISREx4-IL12miR122T) (MOI 10⁵, in both cases). Figure A shows transduction efficiencies of around 70% of AAV8-CAG.GFP treated MC38 cells independent of prior irradiation. Figure B illustrates that IL-12 production is significantly enhanced by MC38 cells that received irradiation prior to AAV8-ISREx4-IL12miR122T transduction. Data are mean±SEM (n=3 samples per condition).*** P<0.005 Unpaired T test.

FIG. 12, comprising Figure A-C, show results of B16 cells transduced with AAV8 encoding luciferase coupled to miR122T under control of the ISREx4 promoter (AAV8-ISREx4-LucmiR122T) (MOI 10⁵) treated with (8 Gy) or without radiation (0 Gy) in combination with either IFN-α, IFN-β, IFN-γ or vehicle. The figure illustrates induction of luciferase activity upon incubation with either IFN-α (A), IFN-β (B) or IFN-γ (C) even in the absence of irradiation, whereas additional irradiation causes synergistic increase in luciferase activity. Data are mean±SEM (n=3 samples per condition), *p<0.01, **P<0.01, **** P<0.0001, Two-way ANOVA followed by Tukey's multiple comparisons test.

FIG. 13, comprising Figure A-O, show the antitumor effects of a combinatory treatment of radiotherapy and intratumor injection of AAV8-ISREx4-IL12miR122T (0.5×10¹¹ vg) on a murine model of melanoma (A+B), a murine model of hepatocellular carcinoma (C-H) and a model of subcutaneous murine glioblastoma (I+J) together with intratumor and serum levels of IL-12 (K+L) and IFN-γ (M+N) and lastly the efficacy of the combinatory approach compared to treatments using only one or no component of the combinatory approach (O). A) Assessment of tumor volume at indicated time points after either treatment with AAV8-ISREx4-IL12miR122T combined with radiotherapy (triangles) or AAV8-ISREx4-LucmiR122T combined with radiotherapy (circles) in a murine model of melanoma (B16 cells). B) Hematoxylin & Eosin staining showing massive lymphocyte infiltration to the tumor tissue three months after AAV8-ISREx4-IL12miR122T combined with radiotherapy. C) and D) Assessment of tumor volume (C) or relative tumor volume (D) at indicated time points after either treatment with AAV8-ISREx4-IL12miR122T combined with radiotherapy (triangles) and AAV8-ISREx4-LucmiR122T combined with radiotherapy (circles) in a murine model of subcutaneous hepatocellular carcinoma generated by subcutaneous injection of PM299L hepatocarcinoma cells. E) Assessment of survival rate shows full survival of AAV8-ISREx4-IL12miR122T treated animals (dashed line) throughout the study period compared to early death of all AAV8-ISREx4-LucmiR122T treated animals (continuous line). F) and G) Assessment of tumor volume (F) or relative tumor volume (G) at indicated time points after either treatment with AAV8-ISREx4-IL12miR122T combined with radiotherapy (triangles, dashed line) and AAV8-ISREx4-LucmiR122T combined with radiotherapy (circles, continuous line) in a murine model of hepatocellular carcinoma generated by subcutaneous injection of NM53 G03 hepatocarcinoma cells. H) Assessment of survival rate shows full survival of AAV8-ISREx4-IL12miR122T treated animals (dashed line) throughout the study period compared to early death of all AAV8-ISREx4-LucmiR122T treated animals (continuous line). I) and J) Assessment of tumor volume at indicated time points after either treatment with AAV8-ISREx4-IL12miR122T combined with radiotherapy (I) and AAV8-ISREx4-LucmiR122T combined with radiotherapy (J) in a model of subcutaneous murine glioblastoma. The lines indicate the changes in tumor volume of individual animals during the study period. K)-N) Intratumor values of IL-12 (K), serum concentration of IL-12 (L), intratumor values of IFN-y (M) and serum concentration of IFN-γ (N) in mice with subcutaneous B16 (melanoma) tumors irradiated with 0 or 8 Gy followed by intratumor injection of AAV8-ISREx4-IL12miR122T (0.5×10¹¹ vg). O) Assessment of tumor volume at indicated time points after either no treatment (black circles), treatment with AAV8-ISREx4-LucmiR122T combined with radiotherapy (white circles), treatment with AAV8-ISREx4-IL12miR122T but no radiotherapy (squares) and treatment with AAV8-ISREx4-IL12miR122T combined with radiotherapy (downwards triangles) in a murine model of melanoma (B16 cells). The arrow indicates the start of the respective treatment. A), C), D), F), G) and 0) Data are mean±SEM (n=4-6 samples per condition) ** P<0.01, **** P<0.0001, Two-way ANOVA followed by Tukey's multiple comparisons test (A and O) or Sidak's multiple comparisons test (C, D, F,G). K-N) Data are mean±SEM (4-8 samples per condition). Two-way ANOVA followed by Tukey's multiple comparisons * P<0.05, ** P<0.01, *** P<0.001. FIG. 14, comprising figures A-B, show the antitumor effects of a combinatory treatment of radiotherapy and intratumor injection of AAV8-ISREx4-IL12miR122T (AAV-IL12T) on a murine model of melanoma compared to respective controls injected with AAV8-ISREx4-LucmiR122T (AAV.Luc), wherein some of the tumor cells lack MHC class I expression (B2Mko, A) or either the IFN-γ receptor (B16-IFNRGR-KO) or JAK1 (B16-JAK1-KO) (B). Data are mean±SEM (n=5-6 samples per condition). **P<0.01, **** P<0.0001, Two-way ANOVA followed by Tukey's multiple comparisons test.

FIG. 15, comprising figures A-P, shows the expression of either IL-12 or IFN-β in different tumor cell lines including melanoma (B16, figures A, B and K-P), glioblastoma (GL261, figures C and D), pancreatic cancer (KPC figures E and F), colorectal cancer (MC38 figures G and H) and breast cancer (TSA figures I and J). Cultured cells (85,000 per well) received 0 or 8 Gy and were then incubated in the presence or absence of a bicistronic vector comprising (A-J) scIL-12 and IFN-β separated by a P2A element under control of an ISREx4 promoter (AAV8-ISREx4-IL12-IFNbmiR122T), (K-L) scIL-12 and IFN-β separated by a F2A element preceded by the linker domain consisting of the amino acid sequence GGSG (SEQ ID NO: 82) under control of an ISREx4 promoter (AAV8-ISREx4-IL12-GGSGF2A-IFNbmiR122T), (M-N) scIL-12 and IFN-β separated by a short IRES element (IRES Gtx9, SEQ ID NO: 83) under control of an ISREx4 promoter (AAV8-ISREx4-IRESGtx9-IL12-IFNbmiR122T) or (O-P) scIL-12 and IFN-β separated by an IRES2 element (SEQ ID NO: 84) under control of an ISREx4 promoter (AAV8-ISREx4-IL12-IRES2-IFNbmiR122T). After 48 hours supernatant was collected and IL-12 and IFN-p were measured by ELISA. Both proteins were only detectable upon incubation with the vector, whereas expression of either protein was greatly increased upon radiation treatment. Data are mean±SEM (n=3 samples per condition). *P<0.05, **P<0.01, *** P<0.001, ****P<0.0001 One-way ANOVA followed by Sidak multiple comparison test.

FIG. 16, comprising Figure A-B, show blood serum levels of IL-12 (A) and IFN-β (B) of mice being intravenously injected with the bicistronic AAV8-ISREx4-IL12-IFNbmiR122T (black circles), with the monocistronic AAV8-ISREx4-IL12miR122T (grey circles) or with the monocistronic vector AAV8-ISREx4-IL12 lacking the miRNA122 target sequence (white circles). Data are mean±SEM (n=5-7 samples per condition). *p<0.01, **** P<0.0001, ns non-significant, Two-way ANOVA followed by Tukey's multiple comparisons test.

FIG. 17, comprising Figure A-B, show the antitumor effects of a combinatory treatment of radiotherapy and intratumor injection of either the bicistronic AAV8-ISREx4-IL12-IFNbmiR122T (rectangles in A, downwards triangles in B), the monocistronic AAV8-ISREx4-IL12miR122T (upwards triangles in A and B) and with the control vector AAV8-ISREx4-LucmiR122T (circles in A and B) on a murine model of melanoma wherein the tumor cells express MHC class I (A) or lack MHC class I expression (B). Data are mean±SEM (n=6 samples per condition). **** P<0.0001, Two-way ANOVA followed by Tukey's multiple comparisons test.

FIG. 18, comprising Figure A-D, show transgene expression to the irradiated subcutaneous tumor (NM53 hepatocarcinoma cells) when AAV8-ISREx4-LucmiR122T is administered by systemic route (intravenous administration) compared to the non-irradiated tumor on the opposite site. Figure B is a repetition experiment of the experiment in Figure A. Figure C shows luciferase activity in four mice with orthotopic NM53 liver cancer. Two mice were irradiated with 20 Gy and the other two were not irradiated, then all mice were injected intravenously with AAV8-ISREx4-LucmiR122T. Luciferase activity was measured 6 days after vector injection. It can be seen that luciferase activity is only present over the liver area in those cases where the tumors have been previously irradiated with 20 Gy. Furthermore, Figure D shows the time course of tumor volume progression in mm³ of MC38 subcutaneous tumors after either radiation (20Gy) or no radiation followed by intravenous injection of either AAV8-ISREx4-LucmiR122T (AAV.LucT) or AAV8-ISREx4-IL12miR122T (AAV.IL12T) as indicated. Data are mean±SEM (n=5-6 mice per group), **** P<0.0001, Two-way ANOVA followed by Tukey's post-hoc test.

FIG. 19, comprising Figure A-D, show the effect of different radiation doses on transgene expression from AAV8-ISREx4-LucmiR122T when the vector is administered by intra-tumor injection. Figure A shows luciferase activity when the vector is injected into the tumor after irradiation with 15 Gy and then subjected to 3 additional doses of 15 Gy (total dose=60 Gy). Figure B shows tumors that received 4 daily doses of 15 Gy and were then injected with the vector (total dose=60 Gy). In Figure C the tumor received 60 Gy followed by intratumor vector injection and two subsequent daily doses of 20 Gy (total dose=100 Gy). In all cases luciferase activity was assessed during the following days after vector injection (as indicated in the figure). Thus, Figures A-C show different irradiation and treatment paradigms whereas the data is collectively displayed in Figure D. * P<0.05. One-way ANOVA followed by Sidak's multiple comparison tests.

FIG. 20 shows the time course of tumor volume progression in mm³ of B16 subcutaneous tumors after radiation (8 Gy) and intratumoral injection of either AAV8-ISREx4-LucmiR122T (AAV.LucT) or AAV8-ISREx4-IL12miR122T (AAV.IL12T) followed by administering either the immune checkpoint inhibitor anti-PD1 (PD1) or the immune checkpoint inhibitor anti-CTLA-4 (αCTLA-4) or no further treatment, as indicated. Data are mean±SEM (n=5-6 mice per group), ns: non-significant, **** P<0.0001, Two-way ANOVA followed by Tukey's post-hoc test.

### EXAMPLE 1: STING RESPONSE TOWARDS DMXAA IN VITRO

In a first set of three experiments the response of different STINGs towards DMXAA was investigated.

### EXPERIMENT 1: DOSE RESPONSE OF DMXAA ON DIFFERENT STINGS

The objective of this experiment was to examine which STINGs are sensitive towards the STING agonist DMXAA and whether there is a dose-dependency towards DMXAA. As a readout, the expression of the downstream target IFN-β was measured by quantitative real-time PCR (qPCR).

### Material and Methods

To this end, either cells from the murine cell line MC38 or cells from the human cell line Hep2G were transduced in vitro by lentiviruses encoding for either wildtype murine STING (mSTINGwt), wildtype human STING (hSTINGwt) or human triple-mutant STING-S162A/G230I/Q266I (hSTINGmut). Transduced cells were sorted by flow cytometry based on expression of reporter proteins thus obtaining stable cell lines. Respective stable cells were seeded in 24-well plates at a density of 750 cells/mm² in growth medium. 24 h after seeding, the cells were incubated with different doses of DMXAA for another 3 h (MC38 cells) or 6 h (Hep2G cells). Cells were harvested and homogenized thereafter, and the mRNA extracted using the Maxwell® RSC simplyRNA Tissue Kit protocol (Promega, Madrid, Spain). RNA was reverse transcribed into cDNA using M-MLV reverse transcriptase (Promega, Madrid, Spain) according to the manufacturer's instructions. A quantitative real-time PCR was performed using CFX-Connect Real-Time PCR Detection System (BioRad, Hercules, Calif) to quantify expression levels. Every analysis was performed in 10 μL total volume containing 5 μL of 1χ IQ SYBR Green Supermix (Bio-Rad Life Science, Hercules, Calif.), 0.2 μL of each primer (15 μM, 300 nM final concentration), 2.6 μL of distilled water, and 2 μL of template cDNA. Primers annealing with each gene in the study are detailed in table 5. The expression of IFN-β was measured for either experimental group and expression levels relative to vehicle treated cells were quantified.

### Results and Discussion

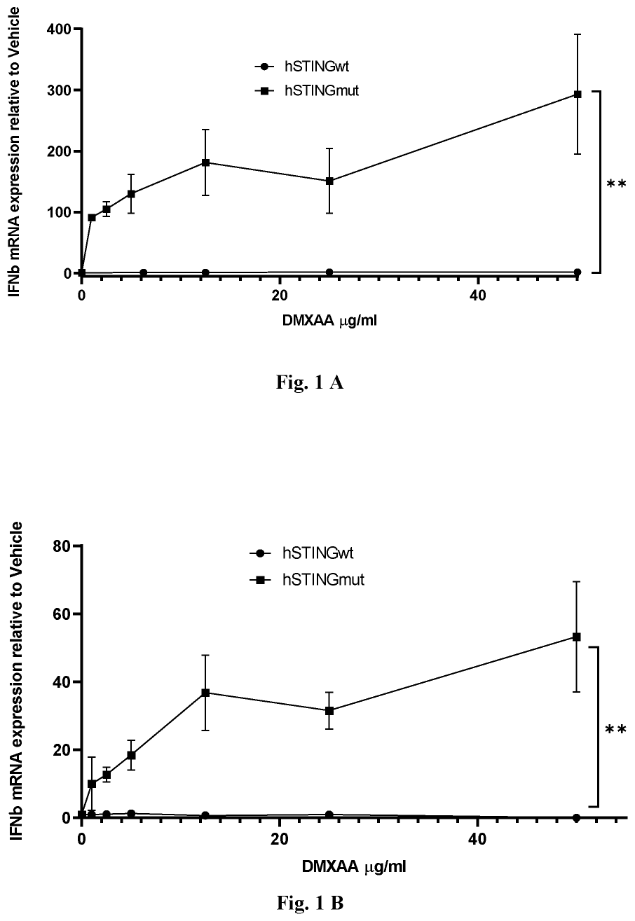

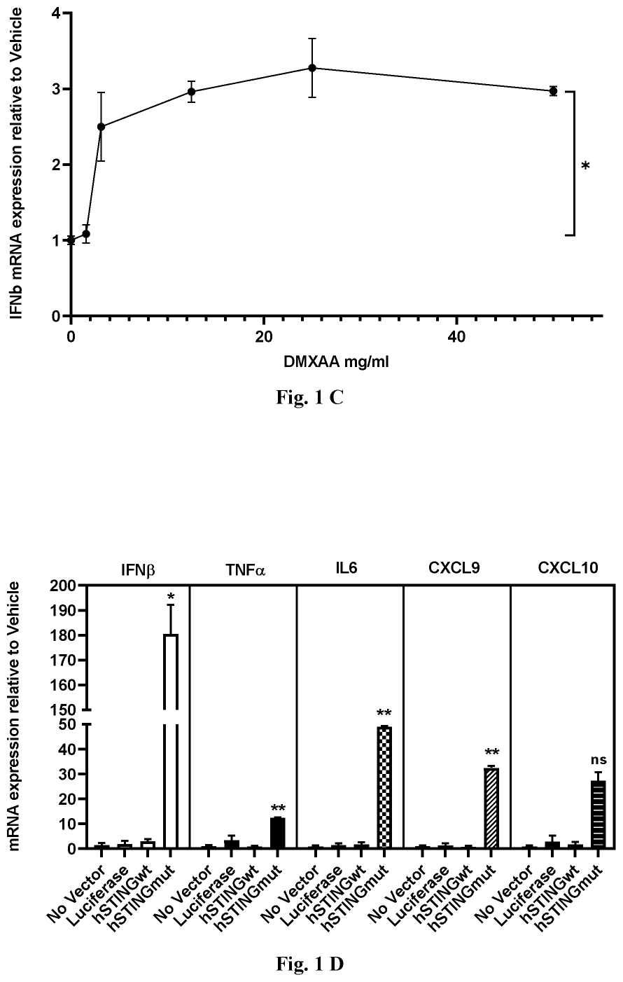

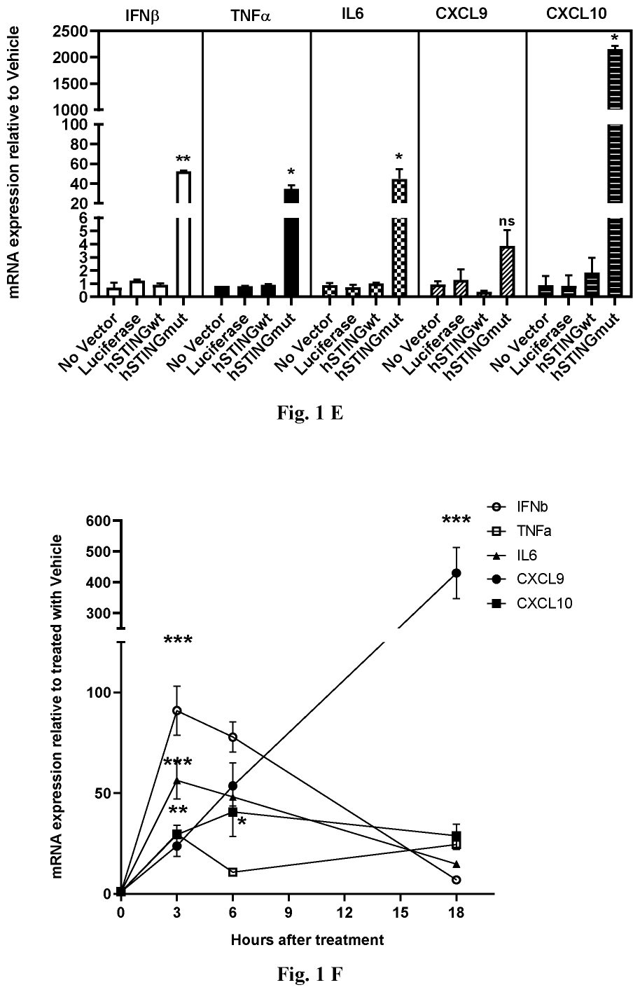

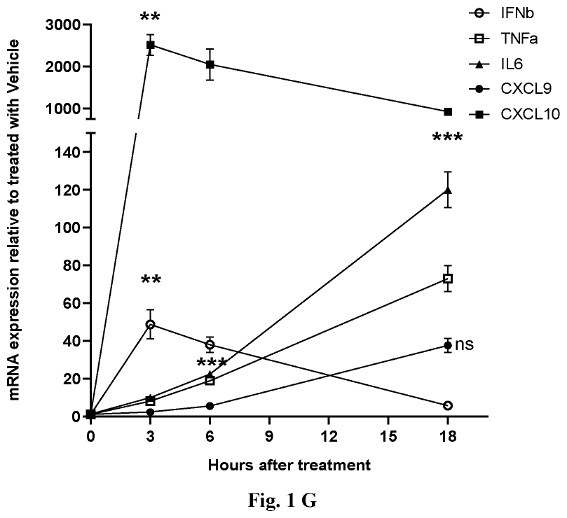

As shown in FIG. 1 A-C, IFN-β expression upon DMXAA treatment was elevated dose-dependently in either human or murine cells transduced with the DMXAA-sensitive STINGs hSTINGmut or mSTINGwt. In contrast no significant alteration in IFN-β expression was observed in the DMXAA-insensitive hSTINGwt. As IFN-β expression is upregulated upon STING activation this experiment shows that i) DMXAA is able to activate DMXAA-sensitive STINGs and these STINGs have biological activity of STING, and ii) the dose of DMXAA correlates with STING activity. Hence, DMXAA activates DMXAA-sensitive STINGs, but not hSTINGwt.

### EXPERIMENT 2: DMXAA MEDIATED EXPRESSION OF CYTOKINES

The objective of this experiment was to examine whether different STINGs show biological activity of STING upon DMXAA treatment. As a readout, the expression of STING-derived cytokines was measured by qPCR.

### Material and Methods

To this end, MC38 and Hep2G cells have been transduced with lentiviruses encoding luciferase, hSTINGwt, hSTINGmut to obtain stable cell lines as described above or were not transduced with any lentivirus (no vector). Respective cells were seeded in 24-well plates at a density of 750 cells/mm² in growth medium. 24 h after seeding, the cells were incubated with 50 μg/mL DMXAA for another 3 h (MC38 cells) or 6 h (Hep2G cells). Cells were harvested and prepared for qPCR measurements as described above. The expression of the cytokines IFN-β, TNF-α, IL-6, CXCL9 and CXCL10 were measured for either experimental group and expression levels relative to controls (no vector) were quantified.

### Results and Discussion

As shown in FIG. 1 D-E, expression of the aforementioned cytokines was elevated only in hSTINGmut-transduced cells whereas no significant alteration of expression levels was observed in any of the other groups. This furthermore corroborates the findings of the first experiment, that i) DMXAA is able to activate DMXAA-sensitive STINGs and these STINGs have biological activity of STING. In addition, this experiment shows that hSTINGmut displays the biological activity of STING in that it causes an upregulation of known STING downstream proteins (Liu et al., 2020 PNAS 117 (24)).

### EXPERIMENT 3: TIME-COURSE EXPRESSION OF STING-DERIVED CYTOKINES UPON DMXAA TREATMENT

The objective of this experiment was to examine the time-course of STING-derived cytokine expression upon DMXAA treatment. As a readout, the expression of STING-derived cytokines was measured by qPCR.

### Material and Methods

To this end, stable cell lines of MC38 and Hep2G cells encoding hSTINGmut have been obtained as described above. 24 h after seeding, the cells were incubated with 50 μg/mL DMXAA for the indicated time points. Cells were harvested and prepared for qPCR measurements as described above. The expression of the cytokines IFN-β, TNF-α, IL-6, CXCL9 and CXCL10 were measured for either experimental group and expression levels relative to controls (0 h after DMXAA treatment) were quantified.

### Results and Discussion

As shown in FIG. 1 F-G, while the temporal expression profiles for the different cytokines differed for each cytokine and between MC38 cells and Hep2G cells, overall, an increase in expression levels was observed upon DMXAA-treatment for any given timepoint. Thus, the expression of the investigated cytokines was upregulated DMXAA-dependently, and hence dependent on STING activity, whereas the different temporal expression patterns show that this expression is also dependent on the cell line used and the respective cytokine investigated.

### EXAMPLE 2: STING RESPONSE TOWARDS DMXAA IN VIVO

As the general sensitivity of hSTINGmut towards DMXAA was shown in vitro, the objective of this experiment was to examine the response of hSTINGmut towards DMXAA in vivo.

### Materials and Methods

C57 wild type mice, aged 6 to 10 weeks, were injected intravenously with an AAV8 vector encoding either hSTINGmut (AAV8-hSTINGmut, 1×10¹¹ viral genomes) or as a control GFP (AAV8-GFP, 1.5×10¹¹ viral genomes). Two weeks after transduction mice were repeatedly (twice per week) injected intraperitoneally with different DMXAA doses (5 mg DMXAA/kg bodyweight, 10 mg DMXAA/kg bodyweight and 20 mg DMXAA/kg bodyweight). Three hours after injection blood was collected from each mouse and IFN-β protein levels in the serum were determined with VeriKine-HS™ (High Sensitivity) Mouse IFN-β Serum ELISA (pbl Assay Science, Piscataway NJ USA).

### Results and Discussion

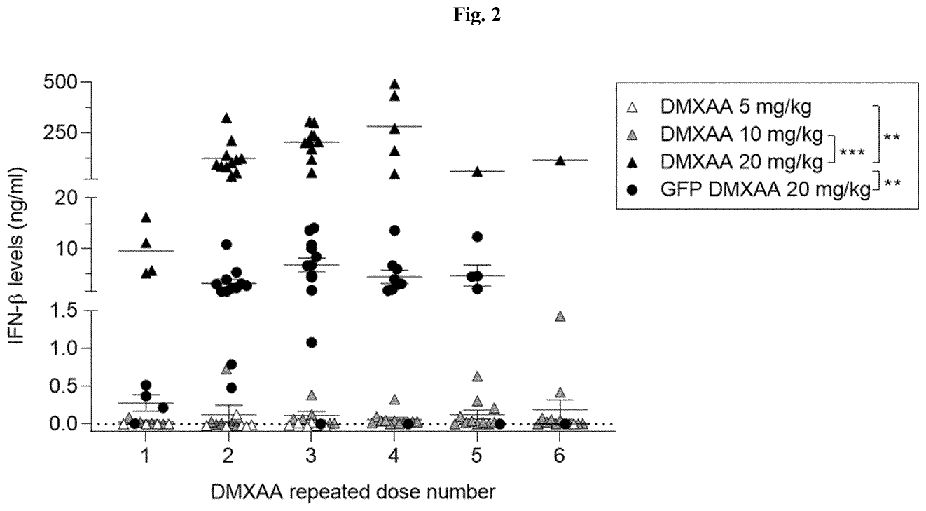

As shown in FIG. 2, administration of 20 mg DMXAA/kg bodyweight already caused an elevation of IFN-β levels in AAV8-GFP transduced control animals after a first injection while IFN-β levels increased even further after repeated injections. This was expected, as mice express murine STING (mSTINGwt) which is already sensitive towards DMXAA and shows IFN-β upregulation in vitro (s. example 1, experiment 1). Notably, IFN-β levels were more than 10-fold higher in mice transduced with AAV8-hSTINGmut at any given timepoint compared to AAV8-GFP controls. In both cases systemic STING stimulation caused high toxicity and mortality (as it can be seen by the drop in the number of remaining animals with successive DMXAA injections). However, doses of 5 or 10 mg DMXAA/kg bodyweight showed only very low levels of IFN-β in AAV8-hSTINGmut transduced mice and, moreover, did neither cause significant toxicity nor mortality. These data show that i) exogenic STING expression enhances the IFN response to STING agonists dose-dependently, ii) systemic STING activation is associated with unacceptable toxicity and iii) that the induction of IFN-β in the transduced tissue can be elicited on demand by injections of the STING agonist DMXAA.

### EXAMPLE 3: STING-ACTIVITY DEPENDENT PROMOTER

As DMXAA-induced STING-activity caused enhanced cytokine expression levels in cells upon transduction with either hSTINGmut or mSTINGwt, the objective of the following experiments was to develop a system where a target gene could be expressed dependent on STING-activity in the transduced cell. Therefore, the aim of the following 4 experiments was to construct vectors with a STING-activity responsive promoter.

### EXPERIMENT 1: IFN-β PROMOTER

One of the main cytokines upregulated upon DMXAA-mediated STING-activation is IFN-Q, making the IFN-β promoter a feasible target to examine its function as a STING-activity dependent promoter. Therefore, the objective of this experiment was to examine the specificity of different modified IFN-β promoters in their response to DMXAA-induced STING activity.

### Materials and Methods

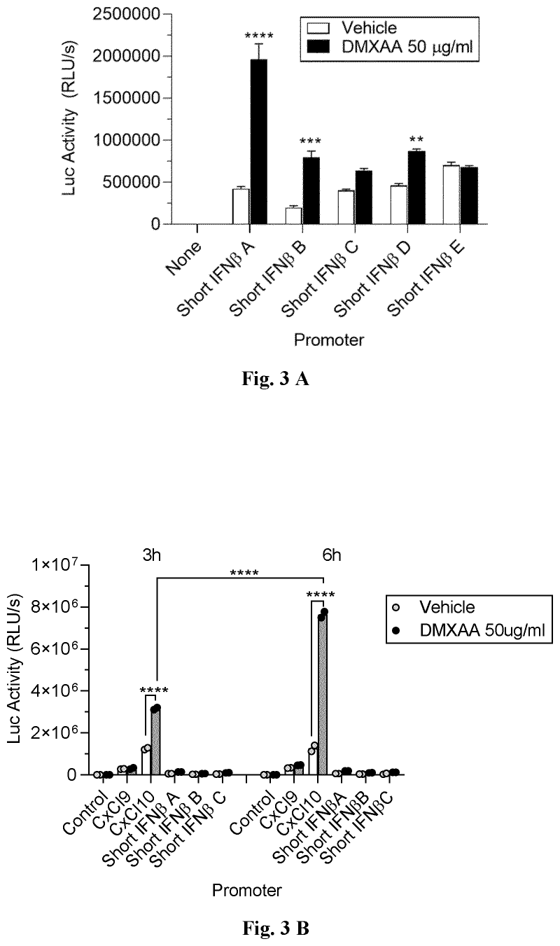

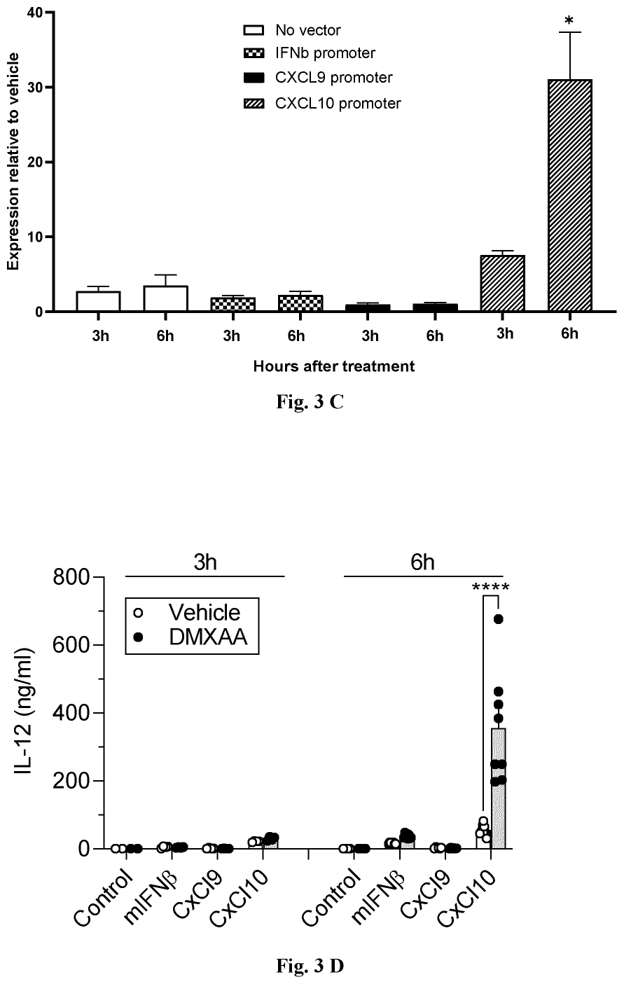

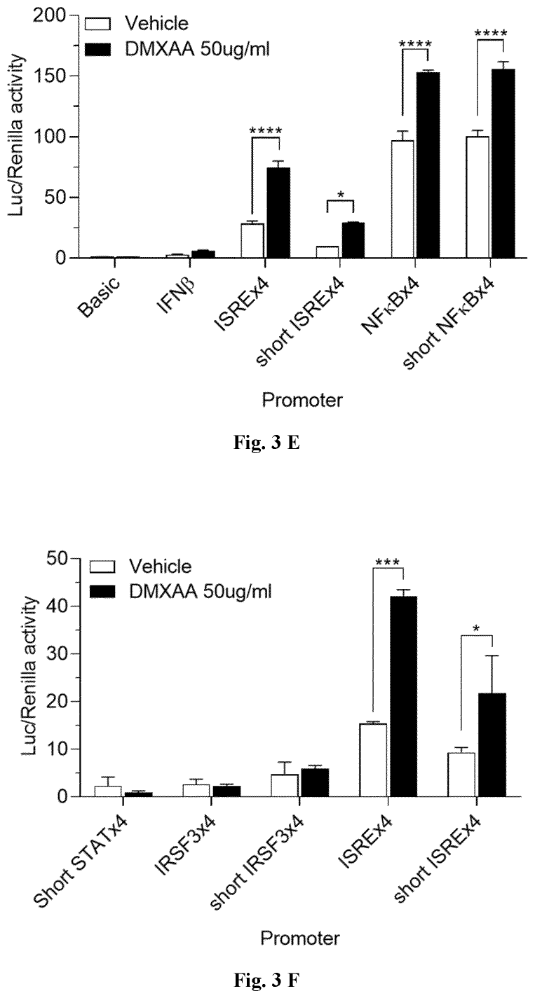

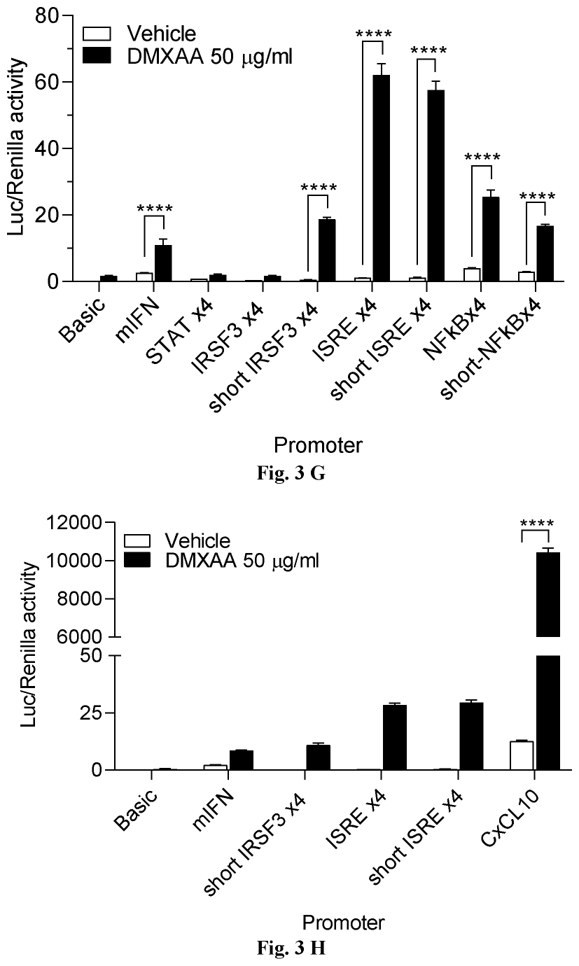

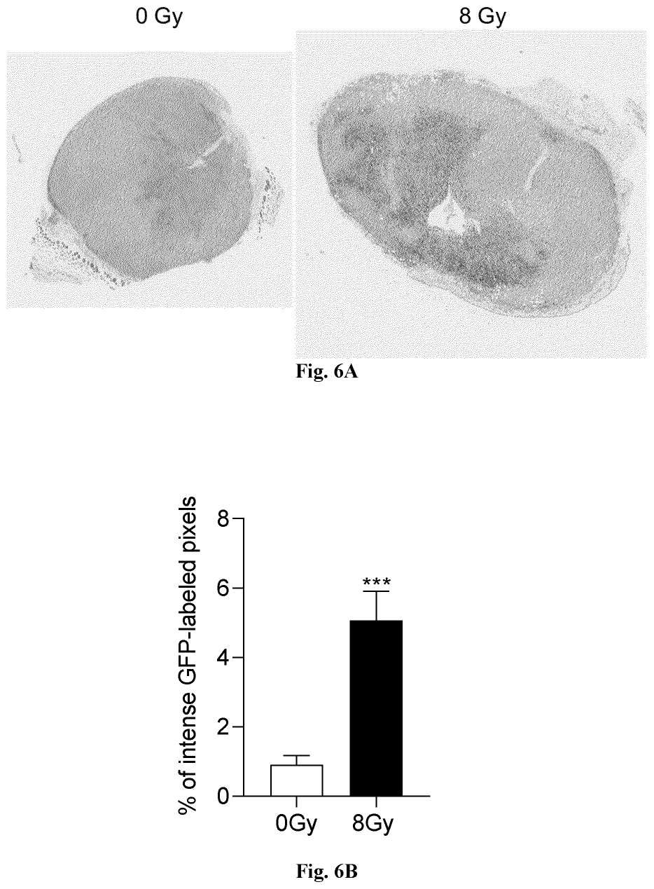

To this end, five different modifications of the IFN-β promoter (short IFN-β A according to SEQ ID NO: 18, short IFN-β B according to SEQ ID NO: 19, short IFN-β C according to SEQ ID NO: 20, short IFN-β D according to SEQ ID NO: 21, short IFN-β E according to SEQ ID NO: 22) were designed. Lentiviral vectors encoding luciferase under the control of these five different promoters were constructed. These vectors were used to lentivirally transduce MC38 cell lines stably expressing hSTINGmut as described above (example 1) while a control group did not receive further lentivirus transduction (none). 24 h after seeding, the cells were treated with DMXAA (50 μg/mL) or vehicle. After another 3 h (FIG. 3A) or 3 and 6 h (FIGS. 3B, 3C, 3D) or 6 h (FIGS. 3E, 3F, 3G and 3H), cells were homogenized in 50 μL of Passive Lysis Buffer (Promega) and frozen in dry ice. After 3 cycles of thaw-vortex-freeze to ensure lysis, cells were spun at 12000 g for 2 min at 4° C. 20 μL were added to a luminometer 96-well plate and luciferase activity was determined using the respective substrates D-Luciferin and Coelenterazine.

### Results and Discussion

As shown in FIG. 3 A, each of the aforementioned promoters showed luciferase activity upon DMXAA administration, whereas cells that were transduced with only hSTINGmut (that is, cells lacking vectors encoding luciferase, indicated as “none” in FIG. 3A) showed no luciferase activity irrespective of DMXAA administration. Notably, the short IFN-β. A promoter showed the highest luciferase activity upon DMXAA treatment compared to the other promoters. However, significant luciferase activity was observed even in the absence of DMXAA. This shows that although the short IFN-β A promoter correlates with the activation of hSTINGmut the specificity of this promoter is somewhat low as basal luciferase activity is observed even when hSTINGmut is not activated by DMXAA (leaky expression). Similarly, also the other promoters showed significant leaky expression. Moreover, the overall responsiveness of the other promoters towards DMXAA-activated STING was rather low.

### EXPERIMENT 2: CHEMOKINE PROMOTER

Next to IFN-β, also the chemokines CXCL9 and CXCL10 showed a distinct upregulation upon DMXAA-mediated STING activation, making the CXCL9- and CXCL10-promoters feasible targets to examine their function as STING-activity dependent promoters. Therefore, the objective of this experiment was to examine the specificity of these promoters in their response to DMXAA-induced STING activity.

### Materials and Methods

The CXCL9-promoter (CXCL9 according to SEQ ID NO: 23) and the CXCL10-promoter (CXCL10 according to SEQ ID NO: 24) were used for the following experiment. To this end, lentiviral vectors encoding luciferase under the control of these two different promoters were constructed. These vectors were used to lentivirally transduce MC38 cell lines stably expressing hSTINGmut as described above (example 1) while a control group did not receive further lentivirus transduction (control). In addition, the lentiviral vectors encoding the short IFN-β A promoter, the short IFN-β B promoter, the short IFN-β C promoter described in experiment 1 were used as a comparison. 24 h after seeding, the cells were treated with DMXAA (50 μg/mL) or vehicle. After another 3 or 6 h, cells were harvested and luciferase activity measured as described in experiment 1.

### Results and Discussion

As shown in FIG. 3 B, each of the aforementioned promoters showed luciferase activity upon DMXAA administration, whereas cells that were transduced with hSTINGmut alone (Control) showed no luciferase activity irrespective of DMXAA administration. Notably, the CXCL9-promoter and the CXCL10-promoter showed significantly higher luciferase activities compared to the IFN-β promoters. However, significant luciferase activity was observed for both chemokine promoters even in the absence of DMXAA. This shows that although these promoters cause a high luciferase expression upon DMXAA administration the specificity of these promoters is somewhat low as basal luciferase activity is observed even when hSTINGmut is not activated by DMXAA (leaky expression).

### EXPERIMENT 3: FUNCTIONALITY OF STING-ACTIVITY DEPENDENT PROMOTERS

Experiments 1 and 2 prove that the IFN-β, the CXCL9- and the CXCL10-promoter, when operably linked to a luciferase transgene, cause its upregulation upon DMXAA-induced STING activation. The objective of the following experiment was to further prove the functionality of these promoters by operably linking them to the expression of the immunomodulatory single chain interleukin-12 (scIL-12).

### Materials and Methods

The CXCL9-promoter and the CXCL10-promoter from experiment 2, and a full length IFN-β-promoter (IFN-β according to SEQ ID NO: 25) were used for the following experiment. To this end, lentiviral vectors encoding murine scIL-12 under the control of these three different promoters were constructed. These vectors were used to lentivirally transduce MC38 cell lines stably expressing hSTINGmut as described above (example 1) while a control group did not receive further lentivirus transduction (no vector). 24 h after seeding, the cells were treated with DMXAA (50 μg/mL) or vehicle. After the indicated time points, cells were harvested. The cell lysate was used to measure scIL-12 mRNA expression by qPCR as described before (example 1, experiment 1), whereas the supernatant was used to measure secreted scIL-12 protein in NUNC MaxiSorp flat-bottom 96-well plates using an ELISA assay kit (OptEIA, Mouse IL-12 (p70), BD Biosciences, San Diego, CA USA), following manufacturer's instructions.

### Results and Discussion

As shown in FIG. 3 C-D, cells that were transduced with hSTINGmut alone showed low expression of scIL-12 mRNA in the cell and did not secrete scIL-12 in detectable levels, irrespective of DMXAA administration. The functionality of the IFN-β-promoter and CXCL9-promoter in terms of scIL-12 mRNA or protein expression was poor even upon DMXAA treatment. In contrast, scIL-12 expression was highly upregulated 6 h after DMXAA treatment and control of the CXCL10-promoter, and significant amounts of scIL-12 protein were secreted into the supernatant. However, in accordance with the previous experiments, the CXCL10-promoter showed significant leakage in that scIL-12 was also produced in the absence of DMXAA. While this shows that the general concept of STING-activity dependent promoters is feasible, the considerable leakage of the promoters used in these experiments deems it necessary to examine other promoters with high inducibility upon DMXAA administration together with high specificity towards STING activity.

### EXPERIMENT 4: SYNTHETIC PROMOTERS

As described above, the specificity of the promoters used in the previous experiments was somewhat poor. Therefore, the aim of the following experiment was to find other promoters that combine high inducibility upon DMXAA administration together with high specificity towards STING activity. In order to achieve this, synthetic promoters have been generated.

### Materials and Methods

Seven different promoters were generated, namely short STATx4 (4 STAT motifs separated by 6 nucleotides according to SEQ ID NO: 26), IRSF3×4 (4 IRSF3 motifs separated by 20 nucleotides according to SEQ ID NO: 27), short IRSF3×4 (4 IRSF3 motifs separated by 6 nucleotides according to SEQ ID NO: 28), ISREx4 (4 ISRE motifs separated by 20 nucleotides according to SEQ ID NO: 12), short ISREx4 (4 ISRE motifs separated by 6 nucleotides according to SEQ ID NO: 13), NFκBx4 (4 NFκB motifs separated by 20 nucleotides according to SEQ ID NO: 29) and short NFκBx4 (4 NFκB motifs separated by 6 nucleotides according to SEQ ID NO: 30). All the above-mentioned promoter sequences given by the SEQ ID: Nos: 12, 13 and 26 to 30, do not contain additional transcription regulatory elements that affect transcription efficiency of the transgenes. These promoters by themselves are functional promoters and do not require any additional elements to provide transcription of the operatively linked transgenes. pGL3-basic plasmids containing the luciferase sequence under the control of the respective promoters were designed. To make it clear, the pGL3-basic plasmids do not contain any promotor-like sequences but the promotors are exclusively incorporated into the plasmid by the above listed sequences SEQ ID: Nos: 12, 13 and 26 to 30. In addition, a plasmid containing the luciferase sequence under the control of the murine IFN-β-promoter (mIFN-β) and a plasmid containing the luciferase sequence under the control of CXCL10-promoter were used as controls. Either MC38 cells or human HEK293T cells, both which stably expressed hSTINGmut as described above (example 1), were transfected with the indicated plasmids using Lipofectamine3000 according to the manufacturers protocol, whereas a negative control was mock-transfected (Basic). 24 h after transfection, the cells were treated with DMXAA (50 μg/mL) or vehicle, and luciferase activity was measured after another 48 h as described in experiment 1.

### Results and Discussion

As shown in FIG. 3 E-F, both ISRE-promoters as well as both NFκB-promoters caused increased luciferase activity in MC38 cells upon DMXAA-mediated STING activation. However, the NFκB-promoters also displayed considerable luciferase activity in the absence of DMXAA treatment. In contrast, the ISRE-promoters showed high inducibility upon DMXAA stimulation with low leakage. The short STAT-promoter and both IRSF-promoters showed poor inducibility. The validity of these assays are corroborated by the fact that no luciferase activity was observed in mock-transfected controls (Basic) irrespective of DMXAA treatment.

As shown in FIG. 3 G-H, both ISRE-promoters as well as both NFκB-promoters caused increased luciferase activity in HEK293T cells upon DMXAA-mediated STING activation. Moreover, compared to murine MC38 cells the leakage of either promoter was considerably reduced in HEK293T cells. However, while no detectable leakage could be observed for the ISRE-promoters, the NFκB-promoters still displayed some leakage in the absence of DMXAA treatment. The highest inducibility was observed for the CXCL10-promoter which, however, displayed considerable leakage. The other promoters showed a rather low inducibility but also limited leakage. Therefore, the ISREx4-promoter showed the highest inducibility while simultaneously displaying undetectable leakage.

Taken together, it should be noted that the ISREx4-promoter exhibits leakage in MC38 cells but not in 293T cells. The former is a tumor cell line while the latter is a non-malignant cell line of embryonic origin. Tumor cell lines frequently have chromosomal abnormalities and micronuclei in the cytoplasm which may sustain smoldering STING activation leading to low-level IFN-β production and low-grade activation of STING-responsive promoters.

### EXAMPLE 4: STING ACTIVITY-DEPENDENT IL-12 EXPRESSION IN VIVO

The ISREx4-promoter showed the highest inducibility while maintaining very low leakage, thereby making it the most promising promoter for in vivo applications. Hence, the aim of the following experiments was to analyze the translatability of STING-activity mediated IL-12 expression in vivo.

### EXPERIMENT 1: IL-12 PRODUCTION IN THE ABSENCE OF DMXAA-MEDIATED STING-INDUCTION IN VIVO

The objective of the first experiment was to examine whether IL-12 is produced in transduced cells in vivo even in the absence of DMXAA-mediated STING activation.

### Materials and Methods

An adeno-associated virus (AAV) of the serotype 8 (AAV8) was constructed that encodes scIL-12 under the control of the ISREx4-promoter (AAV8-ISREx4-IL12) as well as an AAV8 control vector encoding scIL-12 under the control of the murine IFN-β-promoter (AAV8-mIFN-β-IL12). To this end, three different doses, namely 1×10¹⁰ viral genomes, 5×10¹⁰ viral genomes and 1×10¹¹ viral genomes, of AAV8-ISREx4-IL12 or AAV8-pIFN-β-IL12 were injected intravenously in different groups of C57 wild type mice, aged 6 to 10 weeks (n=3 per group), and IL-12 serum levels were measured 7 days thereafter as described in example 2.

### Results and Discussion

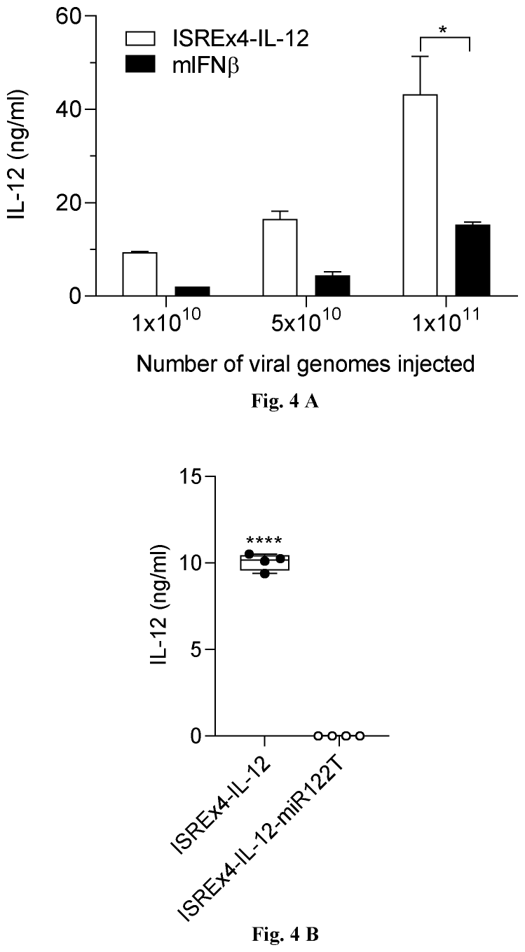

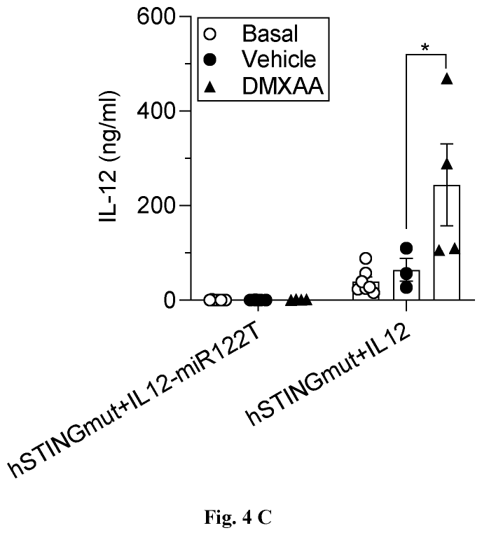

As shown in FIG. 4 A, the IL-12 expression was dose-dependent as higher viral doses caused increased IL-12 production for both AAVs. Notably, the expression was distinctly higher upon intravenous administration of AAV8-ISREx4-IL12 compared to AAV8-pIFN-β-IL12. Hence, although expression levels were higher for AAV8-ISREx4-IL12, both AAVs caused the production of IL-12 in vivo even when no DMXAA was administered. Therefore, measures must be taken to reduce leaky IL-12 production in vivo.

### EXPERIMENT 2: RESTRAINT OF IL-12 LEAKAGE BY MICRO RNA BINDING SITES IN VIVO

As shown in the experiment above, upon intravenous administration of AAV8-ISREx4-IL12, IL-12 is produced irrespective of DMXAA-presence which may cause adverse side effects. Many AAV serotypes, including AAV8, target mainly the liver when present in the blood circulation. Even in the event of intratumor injections, some AAV might enter the blood circulation as tumors are typically highly vascularized, thereby transducing hepatocytes causing them to express IL-12 under the control of an ISRE-promoter. The liver is exposed to multiple pathogen-derived molecules of intestinal origin, which are capable of inducing IFN type I production, thereby causing the production of IL-12. Since IL-12 is a potent immune-stimulatory but cytotoxic cytokine it is critical to limit IL-12 expression to the targeted tissue and to preclude IL-12 production in the liver. One way to limit expression of a certain protein is to intervene on a post-transcriptional level, for example by miRNA binding and subsequent mRNA degradation. Thus, to control IL-12 expression a STING-responsive promoter with little leakage was selected and in an attempt to restrain hepatic IL-12 production the IL-12 sequence was operably linked to binding sites for the micro RNA 122 (miR122) which is highly abundant in liver cells.

### Materials and Methods

A nucleotide sequence was constructed where 5 repetitions of the target sequence of miR122 was placed at the 3′end of the IL-12 sequence. An AAV vector comprising this construct under the control of the ISREx4-promoter was constructed and the respective viral particles produced (AAV8-ISREx4-IL12miR122T). Two groups of C57 wild type mice, aged 6 to 10 weeks (n=4 each), were injected intravenously with either AAV8-ISREx4-IL12 or AAV8-ISREx4-IL12miR122T (each 0.5×10¹¹ viral genomes) and IL-12 serum levels were measured at day 7 after vector injection as described above.

### Results and Discussion

As shown in FIG. 4 B, the animals treated with the vector containing the miR122 binding sites (AAV8-ISREx4-IL12miR122T) had undetectable serum levels of IL-12, while values of more than 10 ng/ml were found in those mice which received AAV8-ISREx4-IL12. Thus, undesired basal liver production of IL-12 can be avoided by controlling its expression via miR122 binding sites.

### EXPERIMENT 3: RESTRAINT OF IL-12 LEAKAGE BY MICRO RNA BINDING SITES IN THE PRESENCE OF DMXAA IN VIVO

Since undesired basal liver production of IL-12 can be avoided by controlling its expression via miR122 binding sites, the objective of the following experiment was to elucidate whether undesired liver production of IL-12 can be achieved even in the presence of the STING agonist DMXAA.

### Materials and Methods

To this end, C57 wild type mice, aged 6 to 10 weeks, were injected intravenously with an AAV8 vector encoding hSTINGmut under the control of a constitutive CAG promoter (AAV8-CAG-hSTINGmut, 1×10¹¹ viral genomes) plus the aforementioned AAV8 vector containing the ISRE-promoter and the single chain IL-12 with (AAV8-ISREx4-IL12miR122T, 0.5×10¹¹ viral genomes) or without 5 miR122 binding sites at the 3′end (AAV8-ISREx4-IL12, 0.5×10¹¹ viral genomes) (n=4 animals each group). One week later these animals were challenged intravenously with DMXAA (20 mg DMXAA/kg bodyweight), a vehicle or not at all (basal). 4 h thereafter 150 μL blood was collected, and IL-12 serum levels quantified by ELISA as described above.

### Results and Discussion

As shown in FIG. 4 C, while mice injected with the IL-12 vector lacking miR122 binding sites (AAV8-ISREx4-IL12) showed increased basal serum levels of IL-12 which further increased upon DMXAA administration, those animals injected with the IL-12 vector containing miR122 binding sites (AAV8-ISREx4-IL12miR122T) exhibited almost undetectable IL-12 serum levels in basal situation and extremely low levels after DMXAA challenge. Hence, AAV8-ISREx4-IL12miR122T shows almost no leakage even in the presence of DMXAA.

### EXAMPLE 5: THERAPEUTIC EFFICACY UPON INTRATUMOR CO-INJECTION OF AAV8-HSTINGMUT PLUS AAV8-ISREX4-IL12MIR122T FOLLOWED BY REPEATED DMXAA ADMINISTRATION

The objective of the following experiment was to analyze whether co-injecting AAV8-hSTINGmut plus AAV8-ISREx4-IL12miR122T into the tumor followed by repeated DMXAA administration shows a therapeutic effect as measured by tumor size reduction.

### Materials and Methods

To this end, a murine model of colon cancer generated by subcutaneous injection of MC38 cells in syngeneic STING deficient C57 mice (C57BL/6J-Tmem173gt/J, STING KO) was used. In these animals DMXAA will not activate STING systemically but only in those tissues that have been transduced with the vector encoding hSTINGmut thus mimicking the human situation. In this experiment MC38 subcutaneous tumors were generated in STING KO mice by subcutaneous injection of 500,000 MC38 cells. When the tumor nodules reached a size of about 70 mm³ they were injected with AAV8-hSTINGmut (1×10¹¹ viral genomes) plus AAV8-ISREx4-IL12miR122T (0.5×10¹¹ viral genomes) and 48 h later the animals were treated with DMXAA (20 mg/kg bodyweight) (n=6 animals) or vehicle two times per week (n=5 animals). In another mouse control group, the tumors were co-injected with AAV8-hSTINGmut (1×10¹¹ viral genomes) plus AAV8-GFP (0.5×10¹¹ viral genomes) and then received vehicle two times per week (n=3 animals).

### Results and Discussion

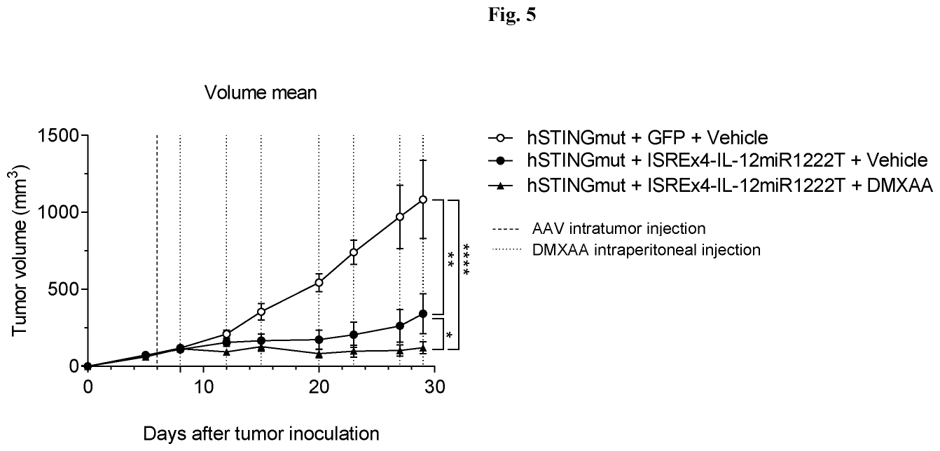

As shown in FIG. 5, a clear antitumor effect was observed in the two groups treated with AAV8-hSTINGmut plus AAV8-ISREx4-IL12miR122T, whereas the antitumor effect was significantly higher in mice treated additionally with DMXAA. Notably, complete tumor elimination was observed in 1 out 6 mice treated with DMXAA but in none given vehicle. In contrast, the tumor volume of mice that received AAV8-GFP plus vehicle treatment continued to grow throughout the time course of the experiment. The antitumor effect in mice treated with AAV8-hSTINGmut plus AAV8-ISREx4-IL12miR122T given vehicle is attributable to low-level intra-tumor production of IL-12 resulting from low-grade basal activation of the ISREx4-promoter in MC38 cells, as mentioned above. Importantly, therapy was well tolerated without symptoms of toxicity in any animal. The strategy here described was designed to exert therapeutic effects without toxicity. In order to avoid toxicity, the AAV vector expressing IL-12 was constructed in such a way that IL-12 will be synthesized within the tumor mass but not in the liver, thus eliminating the risk of sustained basal hepatic production of IL-12 by those AAV particles that gain access to blood and liver after its intra-tumor injection. On the other hand, an agonist is employed, that interacts with the transgenic STING and not with the endogenous human STING. In this way, DMXAA given by intravenous or oral route will activate STING in the tumor but not systemically making the therapy tolerable. The marginal activation of transgenic STING expressed by the small fraction of AAV vector which could reach the liver after intra-tumor injection causes only a slight elevation of type I IFN and IL-12 under 0.2 μg/mL in serum (data not shown) which is very well tolerated by the experimental animals.

### EXAMPLE 6: INTRA-TUMOR TRANSDUCTION EFFICIENCY UPON RADIATION THERAPY

It is known that different DNA damaging agents including topoisomerase inhibitors and radiation have been shown to enhance AAV transduction likely due to the induction of DNA repair synthesis (Russell D W, et al. PNAS 1995; 92, 5719-5723; Alexander I E et al J Virol 1994; 68, 8282-8287). AAVs are single-stranded DNA vectors which need to be converted into double-stranded DNA to form transcription templates. The DNA damage response elicited by radiotherapy helps to convert single stranded DNA into double stranded DNA (Peng D. et al. J Hepatol. 2000 June; 32(6):975-85). The use of radiotherapy to enhance tumor transduction by AAV vectors is an attractive option as radiation is frequently given to treat solid tumors. Published data relating AAV transduction and radiation refer to in vitro studies with different radiation doses and to in vivo studies with high radiation doses. However high radiation doses cause vessel damage and marked alteration of tissue architecture DNA. The objective of the following experiment was to examine whether also low dose radiation enables enhances intra-tumor transduction efficiency. A lower radiation dose preserves tumor vessels, thus, enabling lymphocyte trafficking to the tumor.

### Materials and Methods

MC38 subcutaneous tumors were generated in C57 mice, aged 6 to 10 weeks by subcutaneous injection of 500,000 MC38 cells. When the tumor nodules reached a size of about 70 mm³ the tumor was injected with an AAV8 encoding GFP under a constitutively active CAG promoter (AAV8-GFP, 1×10¹¹ viral genomes). In a group of mice (N=3) the tumors received focal moderate/low-dose radiation (8 Gy) using a SARRP (Small Animal Radiation Research Platform, Xstrahl Inc. Suwanee, GA, USA) immediately before intratumoral AAV-injection whereas in the control group (N=6) the tumors were not irradiated. Two days after injection, immunohistochemical sections of the tumor were prepared. Sections were incubated overnight at 4° C. with anti-GFP (1:2000, Abcam, ab6556). After rinsing in TBS-T, the sections were incubated with goat anti-rabbit labeled polymer EnVision™+ System (Dako, K400311-2; Glostrup, Denmark) for 30 min at room temperature and peroxidase activity was revealed using DAB+ (Dako, K346811-2). Finally, sections were lightly counterstained with Harris hematoxylin, dehydrated, and coverslipped with Eukitt (Labolan, 28500). Sections were examined by a pathologist to obtain a subjective appraisal of tissue transduction and also they were scanned using a CS2 Aperio scanner and the percentage of pixels with intense GFP staining was quantified with Aperio software.

### Results and Discussion

As shown in FIG. 6 A tumors that did not receive any radiation prior to intra-tumoral AAV injection showed transduction efficiencies of about 10-15%. However, tumors that received 8 Gy radiotherapy directly before AAV injection showed transduction efficiencies of about 25-40% of the tumor area. As shown in FIG. 6 B, when tumor sections were analyzed using the Aperio software, a statistically significant increase in the amount of intense GFP-signals in relation to the total area in tumors that received 8 Gy was observed as compared with non-irradiated tumors.

These data show that tumor pre-treatment with a low dose of radiation resulted in marked enhancement of tumor transduction by AAV vectors.

### EXAMPLE 7: RADIOTHERAPY INDUCED STING ACTIVATION IN VITRO

It has been shown that radiation increases IFN-β production by tumor cells and that this effect is mediated by STING. The objective of this experiment was to investigate whether IFN-β production is furthermore increased by exogenic STING.

### Materials and Methods

To this end, lentiviral vectors encoding hSTINGmut were used to generate MC38 cells stably expressing hSTINGmut as described above (example 1) while in a control group the cells had not been transduced. Cells were trypsinized, counted and diluted to 500.000 cells/mL in complete growth media. Cell suspension was divided in equal volumes that were non-irradiated or irradiated in a Gammacell Elan 3000 (Nordion, Canada) for 90 s (8 Gy) immediately before seeding at a density of 750 cells/mm². At indicated time points after radiation, the supernatant was collected, and IFN-β protein levels quantified by ELISA as described above.

### Results and Discussion

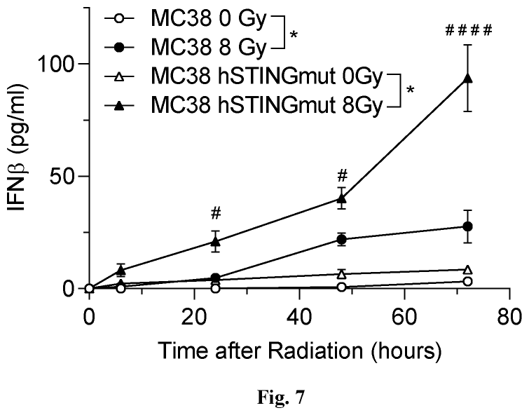

As shown in FIG. 7 IFN-β production remained at low levels throughout the time course of the experiment for cells that neither received radiation nor expressed exogenic hSTINGmut. The same was true for cells that expressed exogenic hSTINGmut but did not receive radiation. However, radiation alone caused an upregulation in IFN-β expression beginning at 24 h after radiation which was furthermore elevated when hSTINGmut was exogenically expressed. Therefore, the effect of radiation on IFN-β production by tumor cells is enhanced by increasing STING cell abundance.

### EXAMPLE 8: LOCAL IRRADIATION FOLLOWED BY INTRATUMOR INJECTION OF VECTORS ENCODING THE EXPRESSION SYSTEM INDUCES TUMOR REGRESSION AND ABSCOPAL EFFECTS

It has been shown that radiotherapy induces STING-mediated IFN-β production in tumor cells (Deng L. et al Immunity, 2014; 41, 843-852). The previous example shows that enrichment in STING increases the production of IFN-β by irradiated cells. By connecting IFN-β expression with IL-12 production, it was analyzed in the following experiments whether a robust anti-tumor effect could be achieved by radiating and transducing the tumor with the expression system.

### EXPERIMENT 1: ANTI-TUMOR ACTIVITY OF COMBINED TREATMENT WITHOUT DMXAA

The objective of the first experiment was to investigate whether the synergistic effects observed in vitro are also applicable to the in vivo situation and whether the combined treatment of radiotherapy plus immuno-gene-therapy exerts vigorous anti-tumor effects. Therefore, MC38 tumors were implanted subcutaneously in wild type C57 mice which were then treated with radiotherapy alone, a combination of immune-gene therapy and radiotherapy or no treatment at all.

### Material and Methods

Group 1 (n=4) had MC38 tumors that were neither irradiated nor transduced with any vector; group 2 (n=5) were mice in which the tumor was irradiated with 8 Gy as described above and that immediately afterwards received an intra-tumor injection with AAV8-hSTINGmut (1.0×10¹¹ viral genomes) plus AAV8-ISREx4-IL12miR122T (0.5×10¹¹ viral genomes), and group 3 (n=4) corresponded to mice with MC38 tumors that were irradiated with 8 Gy as described before and then immediately afterwards received an intra-tumor injection with AAV8-GFP (1.5×10¹¹ viral genomes). Tumor sizes were measured twice weekly using an electronic caliper and the volume was calculated ((length×width²)/2). In addition, in a second part of the experiment blood was collected 17 d after transduction and IL-12 serum levels were quantified by ELISA as described above (example 4). Mice were euthanized when tumors reached 17 mm on the longest axis.

### Results and Discussion

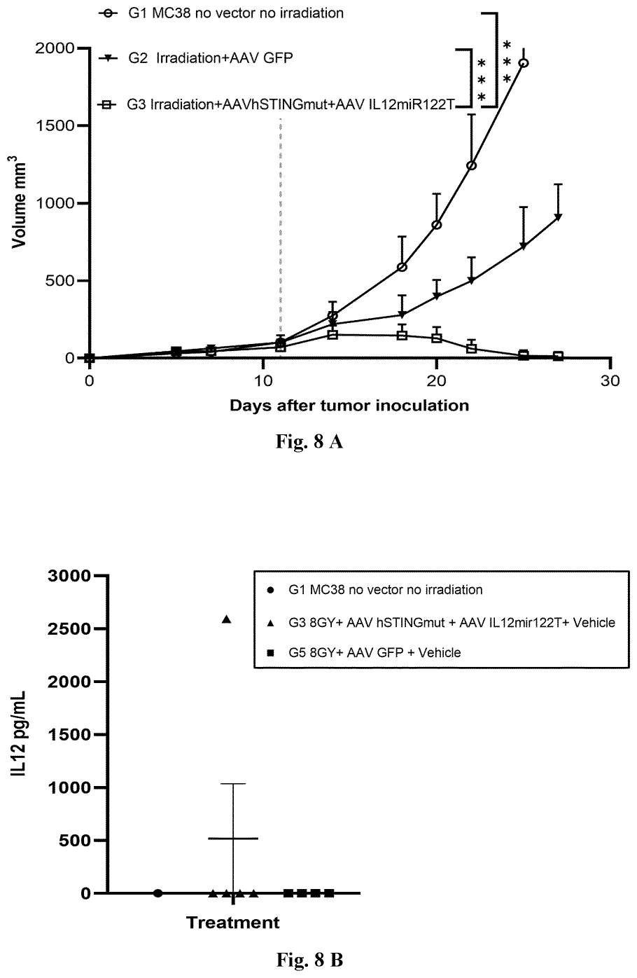

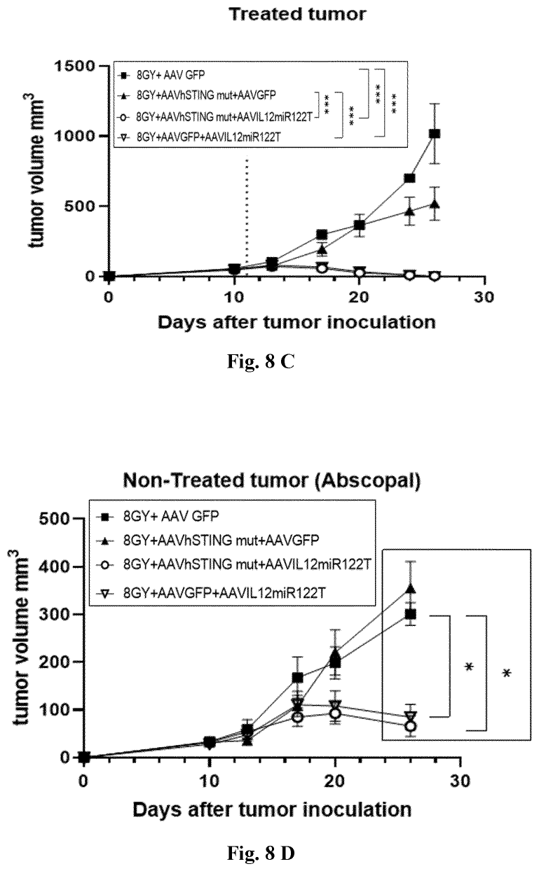

As shown in FIG. 8 A, in the control groups (group 1 and group 3) the tumor continued to grow throughout the time course of the experiment. However, due to the radiation treatment tumor growth in group 3 progressed in a slower rhythm than in group 1. In contrast, animals from group 2 eliminated the tumor very efficiently. As shown in FIG. 8 B, serum IL-12 remained undetectable at the time of tumor elimination in 4 of the 5 mice of group 2. Of note, the IL-12 serum level in this one mouse was distinctly lower than in the previous experiment where no miRNA122 target site was used (example 4, experiment 1). Serum levels in group 1 and 3 were undetectable in all mice. These findings point to the concept that radiotherapy-induced DNA damage activates STING which in turn activates the promoter driving IL-12 expression resulting in increased intra-tumor levels of IFN type I and IL-12 leading to tumor rejection. Furthermore, IL-12 was contained in the tumor thereby preventing unwanted toxicity. Taken together, a synergistic effect is also observed in vivo by combining radiotherapy with the immunogene therapy according to the invention.

### EXPERIMENT 2: ABSCOPAL EFFECT OF COMBINED TREATMENT WITHOUT DMXAA

As described above, combined therapy comprising radiotherapy and immune-gene-therapy caused tumor- elimination in all mice. Not wishing to be bound by therapy, it is thought that immunomodulatory molecules such as IL-12 stimulate the immune system to target the tumor cells which normally manage to evade the immune system (tumor escape). Therefore, this treatment even when it is applied intratumorally could stimulate the immune system to also target tumors outside of the injection site. This is known as the abscopal effect where local treatments also affects untreated tumors (e.g. metastases).

### Materials and Methods

In this study, MC38 tumors were implanted on the right flank by subcutaneous injection of the tumor cells in C57BL/6 wild-type mice, aged 6 to 10 weeks, as described above (example 6). In addition, 2×10⁵ MC38 tumor cells were inoculated in the left flank concurrently. When tumors on the right flank reached a volume of 70-100 mm³, mice were randomized into different treatment groups (n=6 mice per group). All groups received radiation of the right flank with 8 Gy and subsequent intratumor injection of the respective AAVs 11 d after tumor inoculation as described above whereas the tumors in the left flank remained untreated. Group 1 received intratumor injection of AAV8-GFP (1.5×10¹¹ viral genomes), group 2 received intratumor injection of AAV8-hSTINGmut (1.0×10¹¹ viral genomes) plus AAV8-GFP (0.5×10¹¹ viral genomes), group 3 received intratumor injection of AAV8-hSTINGmut (1.0×10¹¹ viral genomes) plus AAV8-ISREx4-IL12miR122T (0.5×10¹¹ viral genomes), and group 4 received intratumor injection of AAV8-GFP (1.0×10¹¹ viral genomes) plus AAV8-ISREx4-IL12miR122T (0.5×10¹¹ viral genomes). Tumor sizes were measured twice weekly by using an electronic caliper and the volume was calculated ((length×width²)/2). Mice were euthanized when tumors reached 17 mm on the longest axis.

### Results and Discussion

As shown in FIG. 8 C, the tumor progression of the treated tumors (right flank) for groups 1 and 3 were similar to that of the previous experiment. Notably, intratumor injection with AAV8-hSTINGmut but without AAV8-ISREx4-IL12miR122T (group 2) did not affect tumor progression significantly compared to the AAV8-GFP control group. However, intratumor injection with AAV8-ISREx4-IL12miR122T but without AAV8-hSTINGmut (group 4) caused the elimination of the tumor in a similar course as the combined treatment (group 3). Of note, the course of tumor progression of the untreated tumors (respective left flank) was similar, although less pronounced, than as for the treated tumors as shown in FIG. 8 D. This study confirms a strong antitumor effect of the therapy on the treated tumor leading to complete tumor rejection in those animals given low-dose radiotherapy together with intratumor injection of AAV8-hSTINGmut plus AAV8-ISREx4-IL12miR122T. And, in addition, it shows that the combined therapy is also accompanied by a significant abscopal effect.

### EXAMPLE 9: SYNERGISM BETWEEN CANONICAL STING PATHWAY ACTIVATED BY DMXAA AND NON-CANONICAL STING PATHWAY ACTIVATED BY RADIOTHERAPY IN VITRO

As indicated before, it is known that radiotherapy-induced DNA damage activates STING. This activation, however, is limited due to low expression of cGAS/STING in many tumors (Dunphy et al., 2018, Molecular Cell 71, 745-760). Recently, it was shown that DNA-damaging agents activate STING by a non-canonical cGAMP-independent route favoring NFκB pathway rather than IFN type I production. Since DMXAA is a canonical activator of STING, the objective of the following experiments was to investigate whether there is a synergism between canonical and non-canonical pathways. It should be mentioned that at the high MOI (10⁵) used in the following experiments there are no significant differences in the cell transduction rate between irradiated and non-irradiated cells and hence strong differences in the effect of DMXAA on irradiated and non-irradiated cells in terms of cytokine induction could not be ascribed to differences in cell transduction rate.

### EXPERIMENT 1: SYNERGISM PROOF OF PRINCIPLE I

The objective of the first experiment was to proof that there is a general synergism between the canonical and non-canonical STING pathways. Therefore, STING was activated in MC38 cells either by radiotherapy (non-canonical) and/or by DMXAA (canonical).

### Materials and Methods

To this end, MC38 cells received 8 Gy or 0 Gy as described above (example 7). Cells were seeded thereafter at 750 cells/mm² density and were then either mock-transduced or transduced with AAV8-hSTINGmut (1×10⁵ viral genomes/cell). For cell transduction 8.5×10⁹ viral genomes of AAV8-hSTINGmut from known concentration stock solutions, were resuspended in 50 μl RPMI 2% FBS. The resuspended viral particles were then added to the wells where the cells were seeded. 48 h later, cells were treated with vehicle or DMXAA (50 μg/mL) and the cells were collected after additional 6 h. The IFN-β mRNA was determined by qPCR as described above (example 1) and expression levels were quantified in reference to non-transduced, non-irradiated cells treated with vehicle.

### Results and Discussion

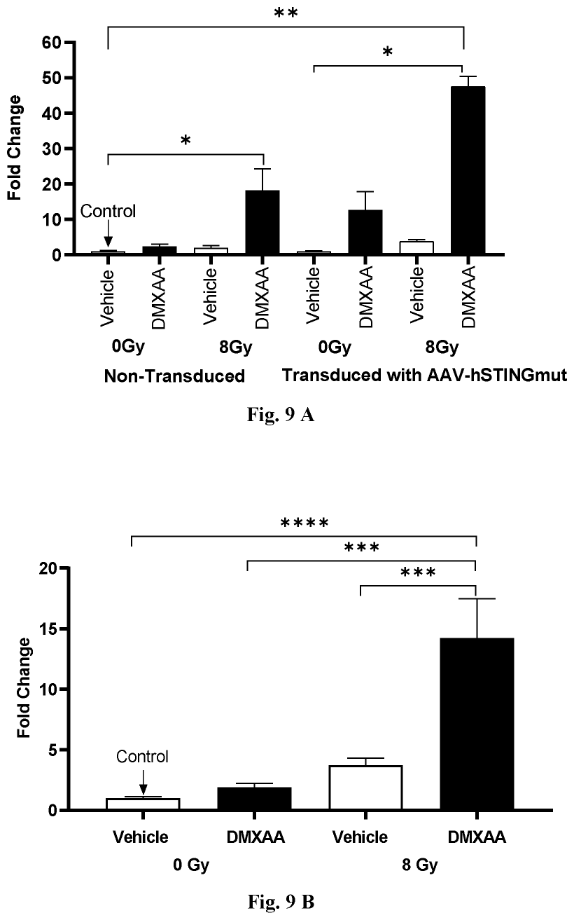

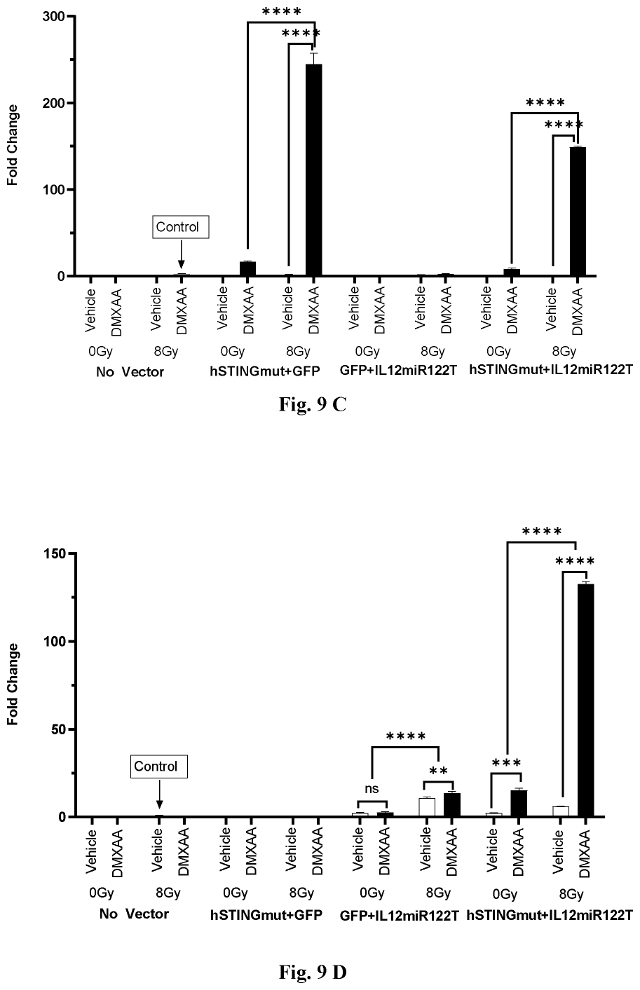

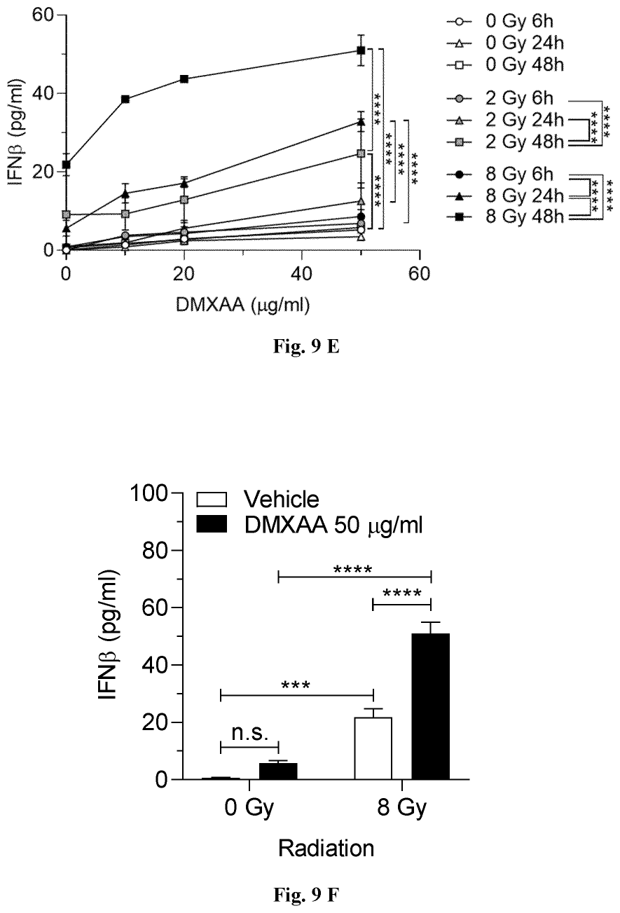

As shown in FIG. 9 A, irradiation combined with DMXAA treatment already caused an increase in IFN-β expression even without exogenic hSTINGmut expression. This is most likely because murine MC38 cells also express DMXAA-sensitive mSTINGwt. However, upon exogenic expression of hSTINGmut IFN-β expression is further increased showing synergistic effects of canonical and non-canonical STING pathways. Notably, the other groups only showed minor IFN-β expression except hSTINGmut-transduced MC38 cells that received DMXAA but no irradiation which showed an intermediate expression.

### EXPERIMENT 2: SYNERGISM PROOF OF PRINCIPLE II

In addition to experiment 1, the objective of the second experiment was to proof that the observed synergism between the canonical and non-canonical STING pathways also causes elevated IL-12 levels when MC38 cells are additionally transduced with AAV8-ISREx4-IL12miR122T. Therefore, MC38 cells were additionally transduced with AAV8-ISREx4-IL12miR122T, and STING was activated in MC38 cells either by radiotherapy (non-canonical) and/or by DMXAA (canonical).

### Materials and Methods

To this end, MC38 cells received 8 Gy or 0 Gy as described above and then were co-transduced with AAV8-hSTINGmut (1×10⁵ viral genomes/cell) and AAV8-ISREx4-IL12miR122T (0.5×10⁵ viral genomes/cell) as described above. For cell transduction, 8.5×10⁹ viral genomes of AAV8-hSTINGmut and 4.25×10⁹ viral genomes of AAV8-scIL12-miR122T, from known concentration stock solutions, were resuspended, each one, in 50 μl RPMI 2% FBS. The resuspended viral particles were then added to the wells where the cells were seeded. 48 h later, cells were treated with vehicle or DMXAA and the cells were collected after additional 6 h. The IL-12 mRNA was determined by qPCR as described above (example 3) and expression levels were quantified in reference to non-transduced, non-irradiated cells treated with vehicle.

### Results and Discussion

As shown in FIG. 9 B, those cells that had been transduced with AAV8-hSTINGmut plus AAV8-ISREx4-IL12miR122T, the administration of DMXAA caused a slight elevation in IL-12 mRNA levels in non-irradiated cells but elicited a strong elevation of this transcript in those cells that had been irradiated with 8 Gy. These findings, in agreement with the previous experiment, confirm that DMXAA and radiotherapy activate STING synergistically.

### EXPERIMENT 3: SYNERGISM PROOF OF PRINCIPLE III

In addition to experiments 1 and 2, the objective of the third experiment was to further investigate the observed synergism between the canonical and non-canonical STING. Therefore, cells of human origin (HEK293) cells were transduced with vectors encoding hSTINGmut, ISREx4-IL12-miR122T and/or GFP as a control, and then treated with radiation (non-canonical) and/or DMXAA (canonical).

### Materials and Methods

To this end, HEK293 cells received 8 Gy or 0 Gy as described above for MC38 cells and then were co-transduced with AAV8-hSTINGmut (1×10⁵ viral genomes), AAV8-ISREx4-IL12miR122T (1×10⁵ viral genomes) and/or AAV-GFP (1×10⁵ viral genomes) as indicated in the figure. 48 h later, cells were treated with vehicle or DMXAA (50 μg/mL), and the cells were collected 6 h thereafter. The IFN-β and the IL-12 mRNA expression levels were determined by qPCR as described above (example 1) and expression levels were quantified in reference to the indicated controls.

### Results and Discussion

As shown in FIG. 9 C, IFN-β expression levels were upregulated for hSTINGmut expressing cells receiving DMXAA agonist treatment. However, expression levels were significantly increased upon additional irradiation of these cells. Cells that did not express hSTINGmut or only received vehicle showed low expression levels. As shown in FIG. 9 D, IL12 expression levels were upregulated for AAV-ISREx4-IL12miR122T transduced cells irrespective of other treatments, whereas non-transduced controls or cells that only received AAV8-hSTINGmut showed nearly no expression of IL-12. However, additional radiation already increased IL-12 expression in cells expressing ISREx4-IL12miR122T. Moreover, further DMXAA treatment caused a significant increase in IL-12 expression levels. Importantly, compared to murine MC38 cells the response of HEK293 cells was much more specific towards irradiation in that the ISREx4-promoter exhibits leakage in MC38 cells but not in 293T cells. The former is a tumor cell line while the latter is a non-malignant cell line of embryonic origin. Tumor cell lines frequently have chromosomal abnormalities and micronuclei in the cytoplasm which may sustain smoldering STING activation leading to low-level IFN-β production and low-grade activation of STING-responsive promoters. In agreement with the previous experiments, these findings confirm that DMXAA and radiation activate STING synergistically.

### EXPERIMENT 4: SYNERGISM PROOF OF PRINCIPLE IV

In addition to the previous experiments, the objective of the fourth experiment was to further investigate the observed synergism between the canonical and non-canonical STING. Therefore, MC38 cells were treated with different doses of radiation (non-canonical) and different doses of DMXAA (canonical).

### Materials and Methods

To this end, native non-transduced MC38 cells received 0 Gy, 2 Gy or 8 Gy as described above. 24 h later, cells were treated with vehicle or different doses of DMXAA (10 μg/mL, 20 μg/mL or 50 μg/mL), and the cells were collected at the indicated time points. The IFN-p protein levels were determined by ELISA as described above. In another part of the experiment MC38 cells received 0 Gy or 8 Gy, received 50 μg/mL DMXAA or vehicle 24 h thereafter and were collected after another 24 h. The supernatants were used to quantify IFN-β levels as described above.

### Results and Discussion

As shown in FIG. 9 E, IFN-β production at 24 h or 48 h after cell treatment with 8 Gy radiation plus DMXAA stimulation (at different doses) induced IFN-β production at levels exceeding the sum of those obtained with radiation alone and DMXAA alone, indicating a synergistic effect. These findings suggest that radiation activates STING and sensitizes this molecule to STING agonists like DMXAA. As shown in FIG. 9 F already a single dose of radiation results in IFN-β production for more than 48 h and this effect is enhanced by DMXAA. In agreement with the previous experiments, these findings confirm that DMXAA and radiation activate STING synergistically.

### EXAMPLE 10: SYNERGISM BETWEEN CANONICAL STING PATHWAY ACTIVATED BY DMXAA AND NON-CANONICAL STING PATHWAY ACTIVATED BY RADIOTHERAPY IN A HUMANIZED MOUSE MODEL IN VIVO

The previous example showed the synergistic effect achieved by radiotherapy in combination with immunogene therapy according to the invention in wild-type mice. The following experiment investigates whether the synergistic effect is also observable in a humanized mouse model where STING is knocked out. This reflects the human situation where DMXAA administration only causes STING activation in the tumor which has been transduced with the expression system whereas systemic STING activation is prevented.

### Materials and Methods

In this study, MC38 tumors were implanted on the right flank by subcutaneous injection of the tumor cells in syngeneic STING deficient C57 mice (C57BL/6J-Tmem173gt/J, STING KO), aged 6 to 10 weeks, as described above (example 6). Mice were then randomly divided into 5 groups and treatment started when the size of the tumor nodules reached 100 mm³. Tumor nodules received local radiation (8 Gy) followed by an intratumor injection of AAV8-hSTINGmut (1.0×10¹¹ viral genomes) plus AAV8-ISREx4-IL12miR122T (0.5×10¹¹ viral genomes) (G2 and G4), or intratumor injection of AAV8-GFP (1.5×10¹¹ viral genomes) (G1), or AAV8-GFP (1.0×10¹¹ viral genomes) plus AAV8-ISREx4-IL12miR122T (0.5×10¹¹ viral genomes) (G3). As a control tumor nodules received mock-radiation (0 Gy) followed by an intratumor injection of AAV8-hSTINGmut (1.0×10¹¹ viral genomes) plus AAV8-ISREx4-IL12miR122T (0.5×10¹¹ viral genomes) (G5). 48 h thereafter, mice were treated i.p. with DMXAA (20 mg/kg) or vehicle and were sacrificed 3 h later (or 30 h in G4). Serum levels of A) IFN-β B) IL-12 and C) IFN-γ were determined by ELISA as described above. D) In a separated group of mice the tumors were collected at 6 h after administration of DMXAA (20 mg/kg) or vehicle and then the tumor tissue was homogenized in RIPA buffer and, after sonication and centrifugation, IL-12 was measured by ELISA in supernatant.

### Results and Discussion

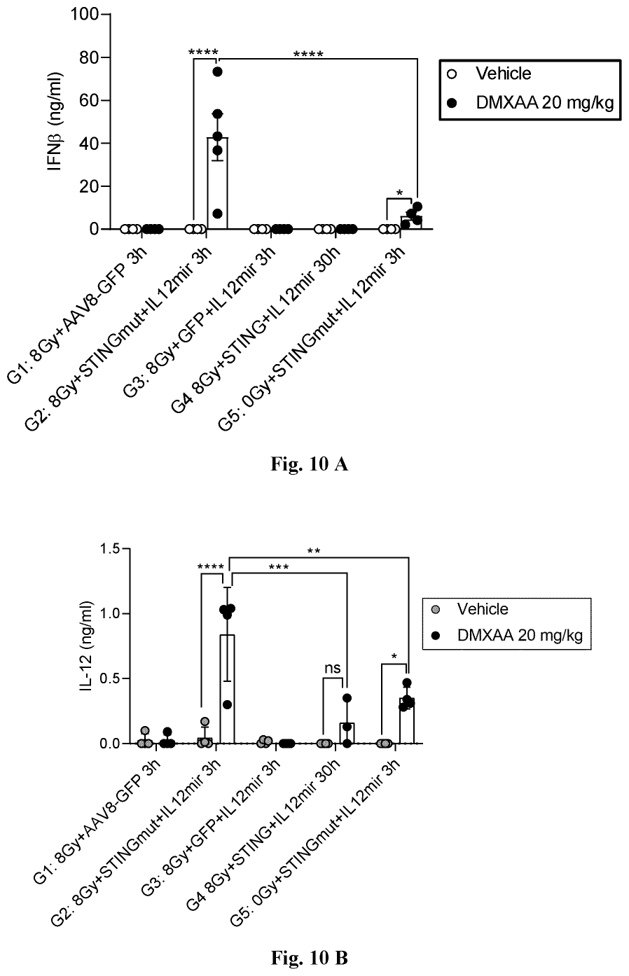

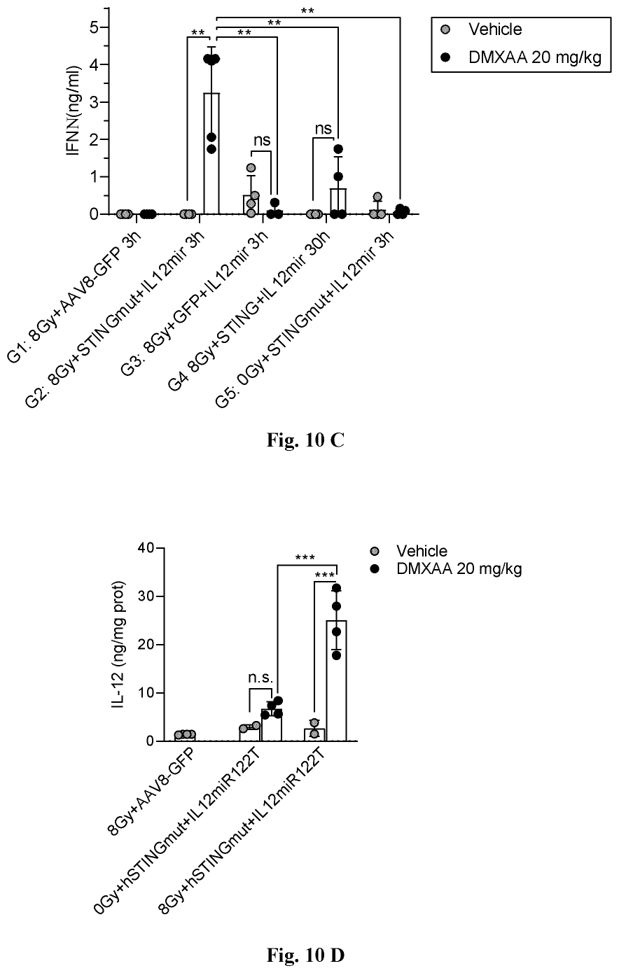

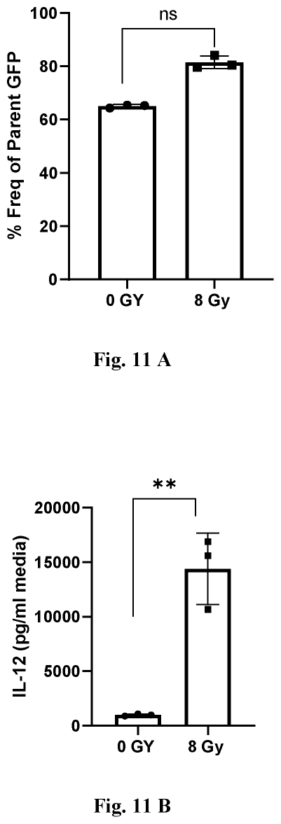

As shown in FIG. 10 A-C, while details differ in between experiments a general trend is observable. The groups G1 and G3 showed no significant induction of either cytokine upon DMXAA-administration. In contrast, throughout the experiments the highest cytokine expression was observed in the groups receiving radiotherapy with intratumor injections of AAV8-hSTINGmut plus AAV8-ISREx4-IL12miR122T (G2 and G4). Notably, the expression was highest at an earlier time point (3 h, G2) compared to a later time point (30 h, G4). However, also the mock-radiated group with intratumor injections of AAV8-hSTINGmut plus AAV8-ISREx4-IL12miR122T (G5) showed response to DMXAA treatment although to a lesser degree than G2 which received both vectors plus radiation. Notably, as shown in FIG. 10 D, in the group of mice in which we have determined the intratumor levels of IL-12 we found that the values were low in those treated with vehicle and increased significantly only in those that were irradiated and treated with DMXAA. Thus, also in a mouse model that resembles the human situation in that STING is specifically activated within the tumor, synergistic effects of radiotherapy combined with the immunogene therapy according to the invention are observable. Taken together, as the experiments showed clear evidence of the feasibility of the therapy according to the invention, further investigations have to examine the effect of this therapy for the treatment of cancer in humans.

### EXAMPLE 11: RADIATION STIMULATES THE INDUCIBLE PROMOTER THAT DRIVES IL-12 EXPRESSION IN AAV8.ISREX4.IL12T-TRANSDUCED CELLS

The following experiments aim to investigate whether radiation prior to transduction affects the transduction efficiency of AAV8 and, thus, the subsequent expression of encoded proteins.

### EXPERIMENT 1: ASSESSMENT OF THE INFLUENCE OF RADIATION ON CELL TRANSDUCTION IN CULTURED TUMOR CELLS WHEN INCUBATED IN VITRO AT HIGH MOI WITH AAV VECTOR ENCODING GFP UNDER THE CONTROL OF A CONSTITUTIVE PROMOTER (CAG)

### Materials and Methods

MC38 (murine colorectal cancer) cells were cultured in DMEM 2% FBS medium and a stock of 5.1×10⁶ cells in 15 ml was divided into 2 tubes which were irradiated with 0 or 8 Gy and incubated with an AAV8 vector encoding GFP under the control of the constitutive CAG promoter (AAV8.CAG.GFP) at MOI 10⁵. Then 250 μl were plated in a 24 well plate and after 48 h GFP expression was analyzed by FACS (flow cytometry).

### Results and Discussion

FIG. 11 A shows that MC38 cells incubated in vitro at high MOI (10⁵) with an AAV vector expressing GFP under the control of a constitutive promoter showed a high transduction efficiency around 70% irrespectively of being irradiated with 8 Gy or no irradiation (0 Gy).

### EXPERIMENT 2: INFLUENCE OF RADIATION ON TRANSGENE EXPRESSION WHEN CULTURED CELLS ARE INCUBATED WITH AN AAV VECTOR ENCODING IL-12 UNDER THE CONTROL OF THE INDUCIBLE PROMOTER ISREX4

### Materials and Methods

MC38 cells were processed as in experiment 1 but AAV8-ISREx4-IL12miR122T was used instead of AAV8-GFP. At 48 h the supernatant was collected, and IL-12 levels were measured by ELISA.

### Results and Discussion

FIG. 11 B shows that IL-12 levels were significantly increased in the supernatant of cultured cells incubated at high MOI with an AAV vector encoding IL-12 under the control of the inducible promoter ISREx4. Since in the previous experiment it was shown that at high MOI transduction efficiency of cultured cells is very high and is not significantly increased by cell irradiation, our findings indicate that the ISREx4 promoter is activated by irradiation of the cells. Thus, while the ISREx4 promoter is suited as a STING-responsive promoter according to the invention, this promoter can be also regarded as a radiation-inducible promoter.

### EXAMPLE 12: SYNERGISTIC EFFECTS OF RADIATION AND IFN SIGNALING ON THE ACTIVATION OF THE ISREX4 PROMOTER

The ISREx4 promoter responds to interferon downstream signaling. The following experiment investigates whether exogenously added IFN-α, IFN-β or IFN-γ activates the promoter and whether this addition has a synergistic effect with radiation of cells treated with AAV8-ISREx4-LucmiR122T.

### Materials and Methods

To this end, B16 cells received 8 Gy or 0 Gy as described above and then were transduced with AAV8-ISREx4-LucmiR122T (1×10⁵ viral genomes/cell) and 48 h later 1,000 IU of either IFN-α, IFN-β or IFN-γ were added to the culture and luciferase activity was measured 48 h later as described above.

### Results and Discussion

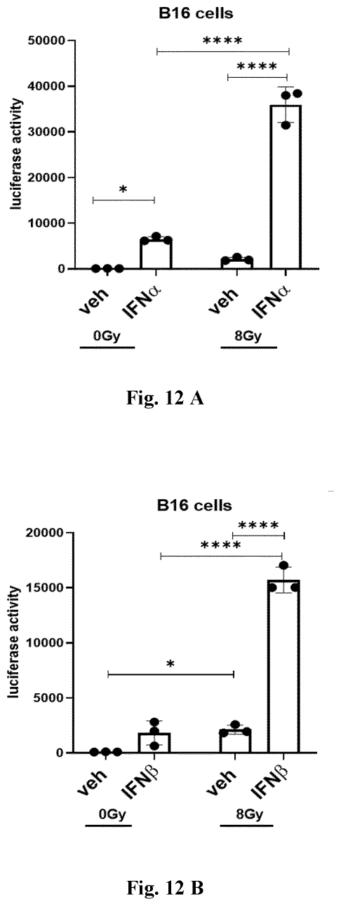

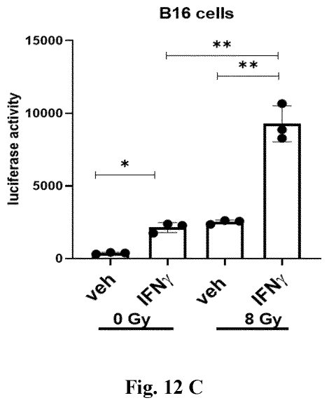

As shown in FIGS. 12 A-C, addition of IFN-α, IFN-β or IFN-γ alone caused a significantly increased luciferase activity, meaning that the interferons alone are able to activate the ISREx4 promoter. Also, radiation alone induced luciferase activity. However, combination of the respective interferon together with radiation induced luciferase activity dramatically. As cell irradiation promotes STING-mediated type I IFN production and IL-12 induces IFN-γ (s. above), the findings point to the concept that the expression of IL-12 initiated by radiotherapy leads to IFN-γ production establishing a feed-back mechanism which maintains IL-12 production in the transduced tumor.

### EXAMPLE 13: AAV8-ISREX4-IL12MIR122T EVEN WITHOUT AAV8-HSTINGMUT DISPLAYS POTENT ANTITUMOR EFFECTS UPON IRRADIATION

In the experiments described above (see FIGS. 8 A and 8 C) it was shown that the combination of tumor irradiation with intratumor injection of AAV8-ISREx4-IL12miR122T plus AAV.hSTINGmut or AAV.GFP induced regression in a murine model of colon cancer even without administering the STING agonist DMXAA. These data indicate that, when used in combination with radiotherapy, AAV8-ISREx4-IL12miR122T is sufficient to induce tumor regression. In the following experiments, this concept was tested in different murine models of solid tumors, including melanoma, hepatocellular carcinoma and glioblastoma. In addition, it was tested whether the combination of radiotherapy and intratumor injection of AAV8-ISREx4-IL12miR122T was able to increase intratumor levels of IL-12 and IFN-γ without causing unduly rise of serum values of these cytokines,

### EXPERIMENT 1: MURINE MODEL OF MELANOMA

### Materials and Methods

3×10⁵ murine melanoma cells B16 were injected subcutaneously into the flank of C57 mice and when tumors reached a size between 100 and 200 mm³ they were irradiated with 8 Gy followed by intratumor injection of AAV8-ISREx4-IL12miR122T or a similar vector encoding Luciferase instead of scIL-12. Tumor size was monitored by measuring tumor diameters with a caliper.

### Results and Discussion

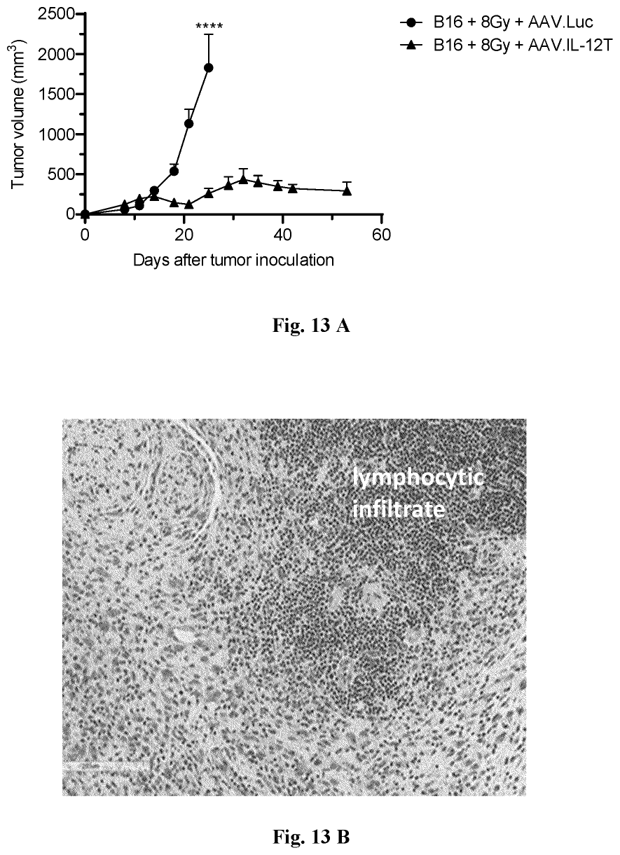

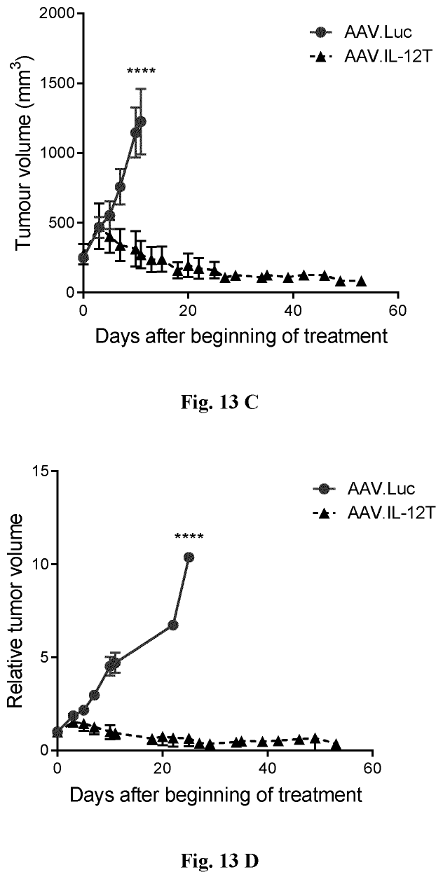

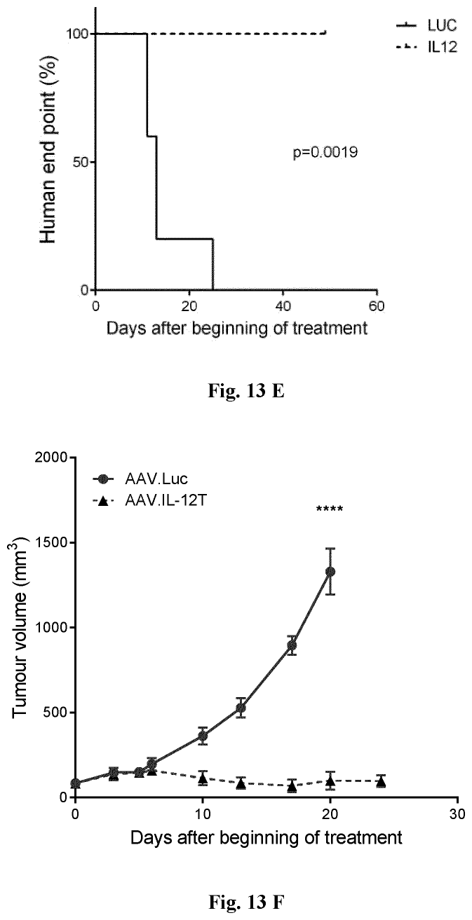

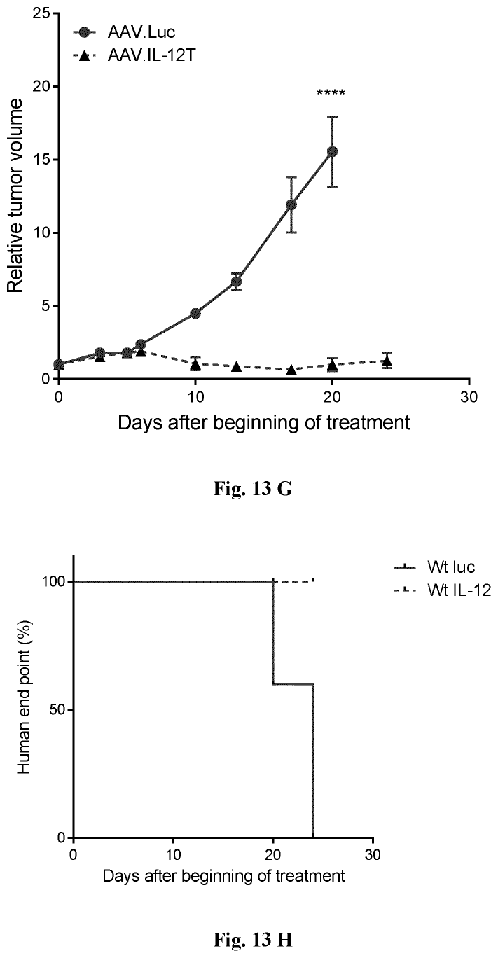

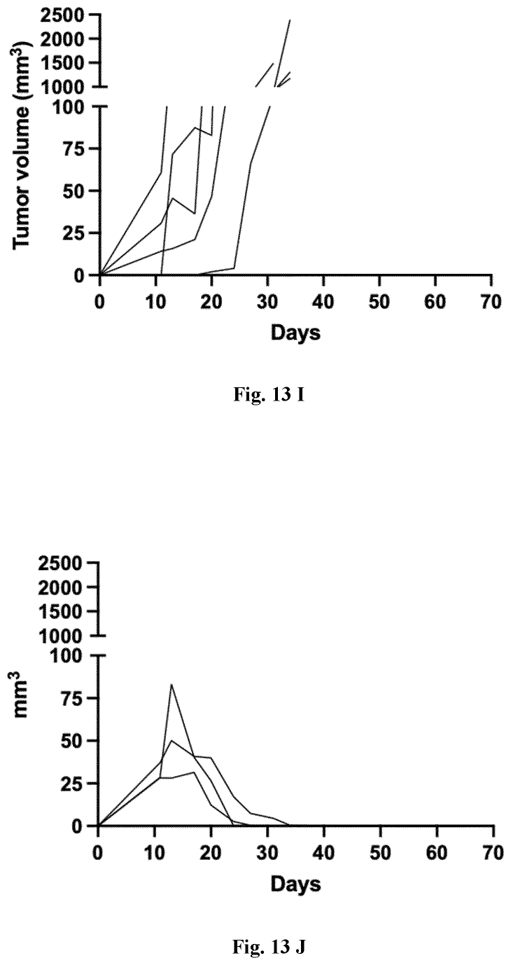

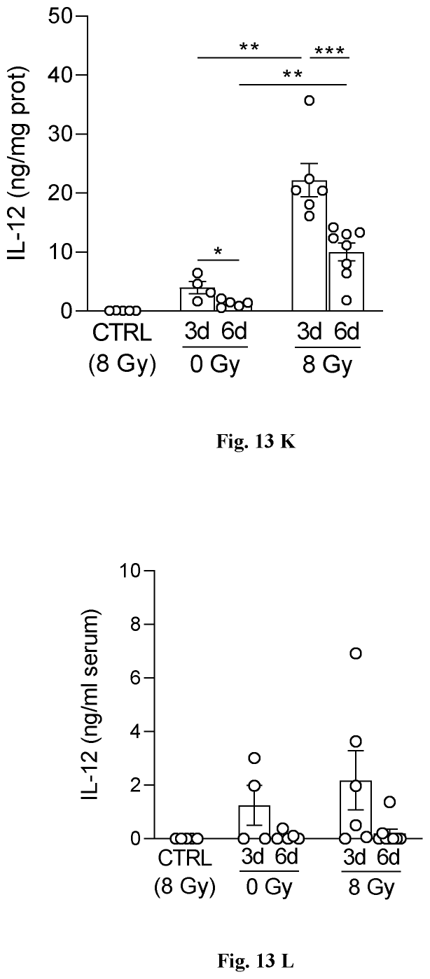

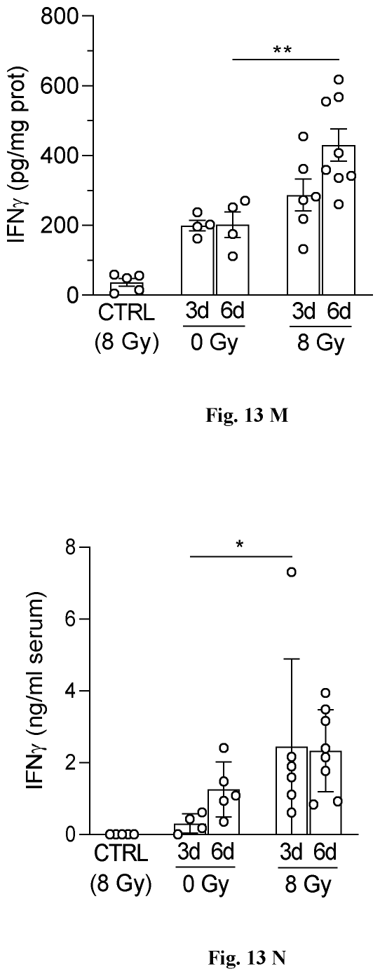

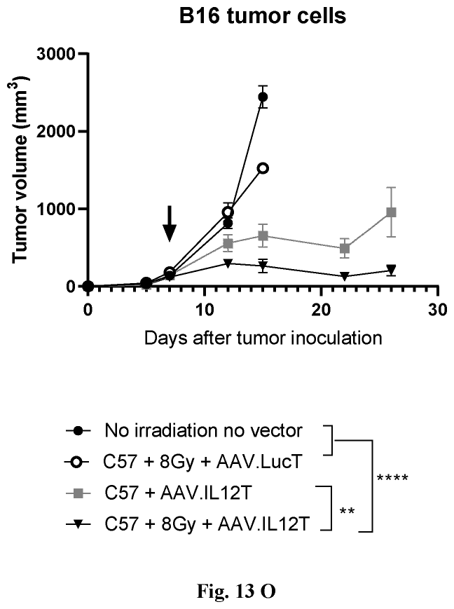

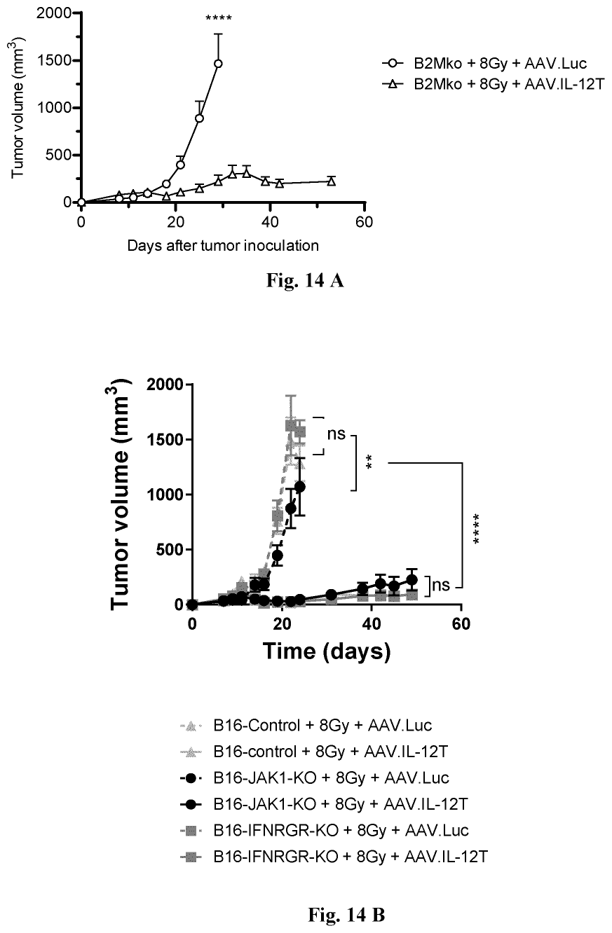

FIG. 13 A shows significant reduction in tumor volume for mice that received AAV8-ISREx4-IL12miR122T combined with radiotherapy (triangles) compared to mice that received AAV8-ISREx4-LucmiR122T combined with radiotherapy (circles) in a murine model of melanoma. Moreover, hematoxylin-eosin staining displayed in FIG. 13 B shows massive infiltration of lymphocytes in the tumor tissue three months after AAV8-ISREx4-IL12miR122T combined with radiotherapy. These data indicate that the expression system of the invention in combination with radiotherapy is able to induce regression of difficult-to-treat tumors (like B16 melanoma) and to promote intense lymphocytic infiltration of the tumor tissue causing tumor elimination or leading to a state of residual tumor where malignant cells are maintained in quiescence during a long period of time. As the response to immune check-point inhibitors is critically dependent of the presence of lymphocytic infiltrate within the tumor, the findings strongly indicate that the expression system of the invention in combination with radiotherapy may synergize with anti-PD1/PDL1 and/or anti-CTLA4 monoclonal antibodies in cancer therapy. These results also point to the concept that the expression system of the invention in combination with radiotherapy may render cold tumors that are unresponsive to immune check-point inhibitors into hot tumors that are sensitive to this therapy.

### EXPERIMENT 2: MURINE MODEL OF HEPATOCELLULAR CARCINOMA (PM299L TUMORS

### Materials and Methods

2×10⁶ murine PM299L hepatocarcinoma cells were injected subcutaneously into the flank of C57 mice and 7 days later (tumor size between 200-500 mm³) they were irradiated with 8 Gy followed by intratumor injection of AAV8-ISREx4-IL12miR122T or a similar vector encoding Luciferase instead of scIL-12 (n=5 per group). Tumor size was monitored by measuring tumor diameters with a caliper. The survival of the animals was checked two times per week.

### Results and Discussion

As shown in FIGS. 13 C and D, while tumors treated with AAV8-ISREx4-LucmiR122T experienced a progressive growth, tumors injected with AAV8-ISREx4-IL12miR122T regressed. These data show that AAV8-ISREx4-IL12miR122T in combination with radiotherapy display potent antitumor effect in a murine model of HCC. Importantly, as shown in FIG. 13 E, all animals that received mock treatment died in the course of the study while all animals treated with AAV8-ISREx4-IL12miR122T survived.

### EXPERIMENT 3: MURINE MODEL OF HEPATOCELLULAR CARCINOMA (NM53 G03 TUMORS)

### Materials and Methods

5×10⁶ murine NM53 G03 hepatocarcinoma cells were injected subcutaneously into the flank of C57 mice and 7 days later (tumor size between 200-500 mm³) they were irradiated with 8 Gy followed by intratumor injection of AAV8-ISREx4-IL12miR122T or a similar vector encoding Luciferase instead of scIL-12 (n=5 per group). Tumor size was monitored by measuring tumor diameters with a caliper. The survival of the animals was checked two times per week.

### Results and Discussion

As shown in FIGS. 13 F and G, while tumors treated with AAV8-ISREx4-LucmiR122T experienced a progressive growth, tumors injected with AAV8-ISREx4-IL12miR122T regressed. These data show that AAV8-ISREx4-IL12miR122T in combination with radiotherapy display potent antitumor effect in a murine model of HCC. Importantly, as shown in FIG. 13 H, all animals that received mock treatment died in the course of the study while all animals treated with AAV8-ISREx4-IL12miR122T survived.

### EXPERIMENT 4: MODEL OF SUBCUTANEOUS MURINE GLIOBLASTOMA

### Materials and Methods

3×10⁵ murine glioblastoma GL261 cells were implanted subcutaneously into the flank of C57 mice and when tumors reached a size between 100 and 200 mm³ they were irradiated with 8 Gy followed by intratumor injection of AAV8-ISREx4-IL12miR122T (n=3) or a similar vector encoding Luciferase instead of scIL-12 (n=5). Tumor size was monitored by measuring tumor diameters with a caliper.

### Results and Discussion

As shown in the FIGS. 13 I and J tumors treated with AAV8-ISREx4-IL12miR122T experienced complete regression while those that received the control vector exhibited progressive growth.

### EXPERIMENT 5: INTRATUMOR INJECTION OF AAV8-ISREX4-IL12MIR122T INTO IRRADIATED TUMORS INCREASES INTRATUMOR LEVELS OF IL-12 AND IFN-γ DURING A PROLONGED PERIOD OF TIME WITH ONLY A MODERATE AND TRANSIENT ELEVATION OF SERUM CONCENTRATION OF THESE CYTOKINES

### Material and Methods

C57 were injected subcutaneously with 5×10⁵ MC38 cells and when tumors reached a volume of approximately 100 mm³ they were irradiated with 0 or 8 Gy and then injected with 0.5×10¹¹ vg of AAV8-ISREx4-IL12miR122T or AAV.GFP (control). In different groups of animals the tumors were excised at 3 days or 6 days after vector injection and serum samples were obtained at the same time points. IL-12 and IFN-γ were determined by ELISA in serum samples and in tumor extracts.

### Results and Discussion

Neither IL-12 nor IFN-γ were detectable in tumors irradiated with 8 Gy injected with control vector AAV.GFP. In contrast both IL-12 and IFN-γ were increased at days 3 and 6 in tumors injected with AAV8-ISREx4-IL12miR122T whereas the intratumor values of both cytokines were significantly higher in those tumors that received 8 Gy as compared to non-irradiated tumors at the two time points. Notably, as shown in FIG. 13 K, although in irradiated tumors treated with the therapeutic vector intratumor IL-12 values were high at both 3 and 6 days, the levels at the latter point were significantly lower than at day 3. In contrast, in the same samples, IFN-γ values were elevated at day 3 and even more at day 6, as shown in FIG. 13 L, likely reflecting the production of endogenous IFN-γ by lymphocytes infiltrating the tumor. Interestingly, as shown in FIG. 13 M, mice receiving intratumor injection of AAV8-ISREx4-IL12miR122T with or without previous irradiation, serum values of IL-12 showed a transient and small peak at day 3 after vector injection, with values generally below 4 ng/ml which reverted to very low or undetectable levels at day 6. In the same samples, serum IFN-γ was slightly increased at day 3 and 6, as shown in FIG. 13 N, the values were higher in those animals in which the tumor was irradiated before vector injection as compared to those in which the tumor was not irradiated. In contrast to IL-12, serum IFN-γ remained elevated to similar levels at day 3 and 6, reflecting the activation of the immune system. When comparing serum and intratumor values it should be considered that tumor values are related to mg protein and serum values to ml and that protein concentration in the tumor is much higher than that in serum, hence the concentration of IL-12 or IFN-γ in the tumor is considerably higher than that in serum. These data indicate that the therapeutic approach allows to obtain high levels of immunostimulatory cytokines inside the tumor with little increase of serum levels thus enabling potent antitumor responses without systemic toxicity.

### EXPERIMENT 5: EFFICACY OF COMBINED RADIOTHERAPY AND IMMUNOGENE THERAPY

The previous experiments of this example showed profound effects when combining radiotherapy with the immunogene therapy of the invention. The following experiment aims to compare the efficacy of the combined approach to the treatments using only one or no component of the combined approach.

### Material and Methods

Mice with subcutaneous B16 tumors were generated by s.c. injection of 500,000 B16 melanoma cells. At day 8 post cell inoculation (when tumors reached a size of about 100-150 mm³) they were divided into 4 groups: no radiation and no vector, no radiation and intratumor injection of AAV8.ISREx4.IL12miR122T, radiation with 8 Gy and intratumor injection of AAV8.ISREx4.LucmiR122T, or radiation with 8 Gy and intratumor injection of AAV8.ISREx4.IL12miR122T. Tumor size was monitored by measuring tumor diameters with a caliper.

### Results and Discussion

This data shows that while AAV8.ISREx4.IL12miR122T administration alone already influences tumor development, the combination of radiotherapy and AAV8.ISREx4.IL12miR122T administration shows the most profound effect on tumor regression. Notably, radiation alone was unable to affect tumor progression, thus, indicating the synergistic effect obtained by combined treatment.

### EXAMPLE 14: AAV8-ISREX4-IL12MIR122T CAUSES TUMOR REGRESSION EVEN IN TUMOR CELLS LACKING MHC CLASS I EXPRESSION OR IFN-γ SIGNALING

Some tumors are able to evade T-cell immunity by different means such as silencing the expression of MHC class I molecules and/or IFN-γ gamma signaling molecules. In the following experiments, it was investigated whether AAV8-ISREx4-IL12miR122T combined with radiotherapy is able to inhibit tumor growth even in the absence of MHC class I molecules or IFN-γ gamma signaling molecules.

### Materials and Methods

2.5×10⁵ wild type B16 cells or B16 B2M.KO cells, that lack MHC class I expression at the cell membrane, were injected subcutaneously into the flank of C57 mice and when the tumors reached a size of 100-200 mm³ they were irradiated with 8 Gy followed by intratumor injection of AAV8-ISREx4-IL12miR122T or similar vector encoding Luciferase. In a second set of experiments, 2.5×10⁵ wild type B16 cells or B16 IFNR.KO cells, that lackIFN-7 gamma receptor expression, or B16 Jak1.KO cells, that lack the essential IFN signaling molecule JAK1, were injected subcutaneously into the flank of C57 mice and when the tumors reached a size of 100-200 mm³ they were irradiated with 8 Gy followed by intratumor injection of AAV8-ISREx4-IL12miR122T or similar vector encoding Luciferase (n=5-6 per group).

### Results and Discussion

FIGS. 14 A to B show that AAV8-ISREx4-IL12miR122T was able to inhibit the growth of tumors formed by wild type B16 cells, B16 B2M.KO cells, B16 IFNR.KO cells or B16 Jak1.KO cells. Although the inhibitory effect was slightly stronger on B16 wild type cells, it was apparent that the therapy was also able to control tumors lacking MHC class I expression and tumors where IFN-γ signaling was blocked. Since, in some cases, tumors silence the expression of MHC class I molecules and/or IFN-γ signaling molecules as a means to evade T-cell immunity, the present data show that the expression system of the invention in combination with radiotherapy is effective even in those cases that might resist other immunotherapies.

### EXAMPLE 15: A BICISTRONIC VECTOR ENCODING TWO IMMUNOMODULATORY TRANSGENES UNDER THE ISREX4 PROMOTER SHOWS POTENT TRANSGENE EXPRESSION UPON RADIATION

To facilitate an adequate immune response in the tumor environment it may be necessary to provide more than one immunomodulatory gene or protein. Hence, the following experiment aims to investigate the feasibility of dual transgene expression.

### EXPERIMENT 1: USING P2A AS A TRANSLATION REGULATORY ELEMENT SEPARATING THE SEQUENCES ENCODING THE IMMUNOMODULATORY PROTEINS

### Materials and Methods

A bicistronic vector, named AAV8-ISREx4-IL12-IFNbmiR122T, was constructed by placing the sequences of scIL-12 and IFN-β separated by a P2A element under control of an ISREx4 promoter. The functionality of this bicistronic vector was tested in different murine tumor cell lines including B16 (melanoma), GL261 (glioblastoma), KPC (pancreatic cancer), MC38 (colorectal cancer) and TSA (breast cancer). Cells (85.000 per well) were irradiated with 0 or 8 Gy and afterwards incubated in the presence (MOI 10⁵) or absence of said vector. The supernatant was collected after 48 h and IL-12 and IFN-β levels were measured by ELISA.

### Results and Discussion

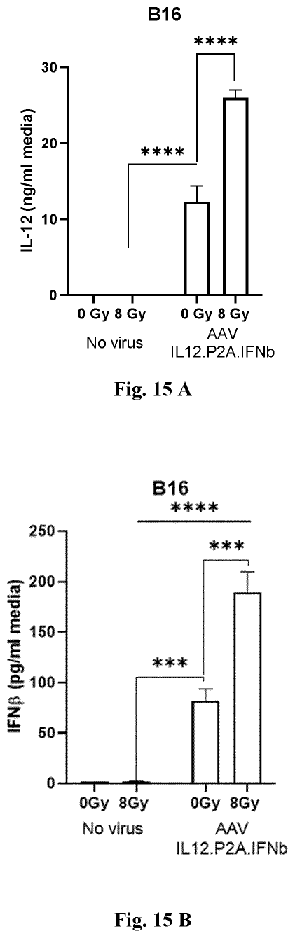

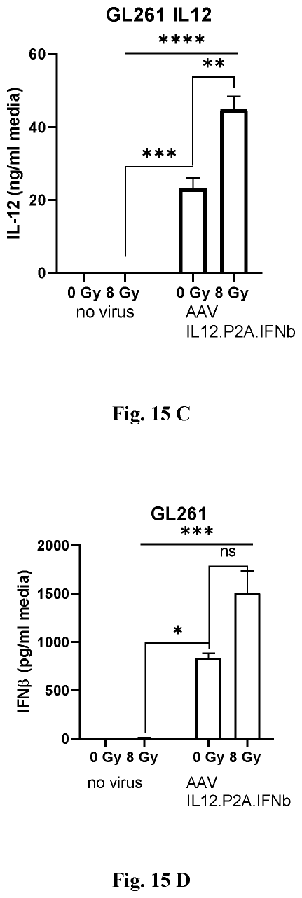

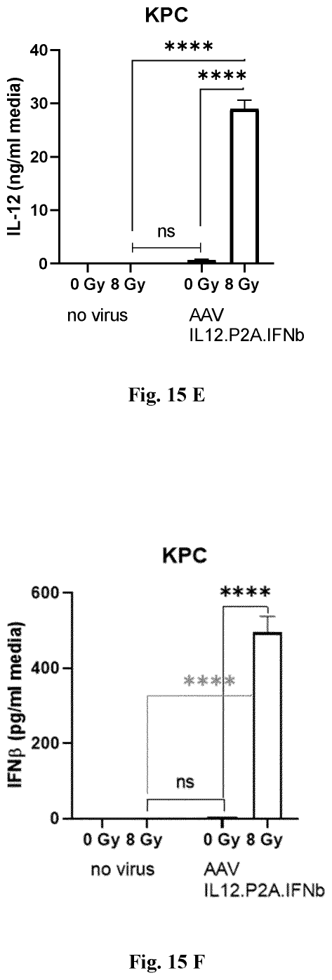

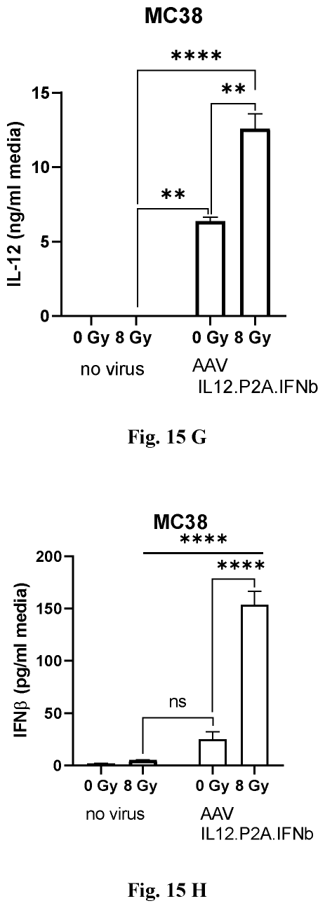

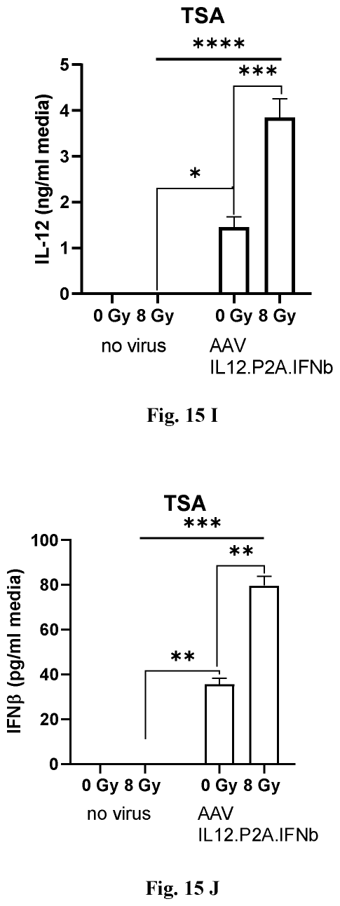

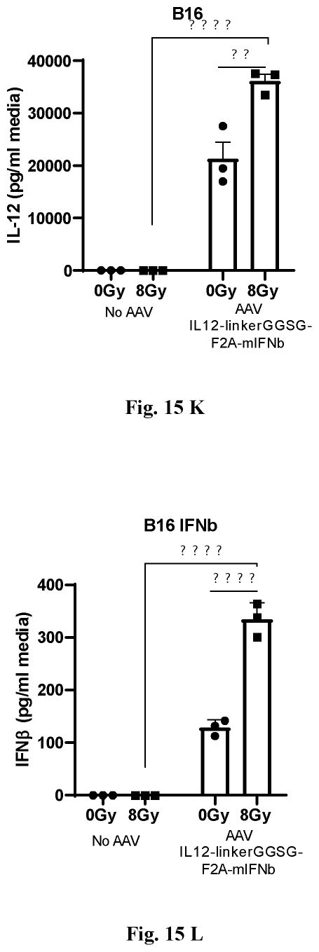

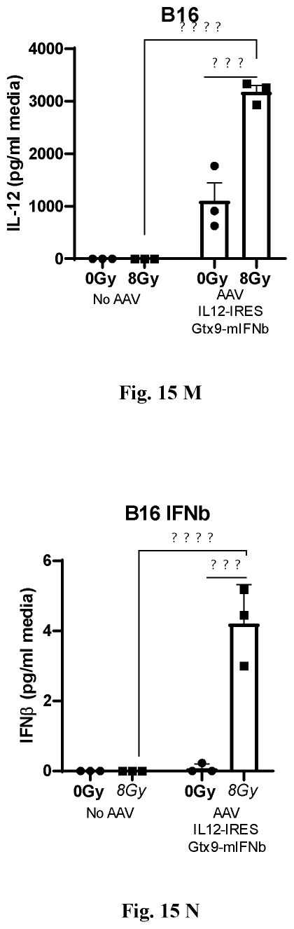

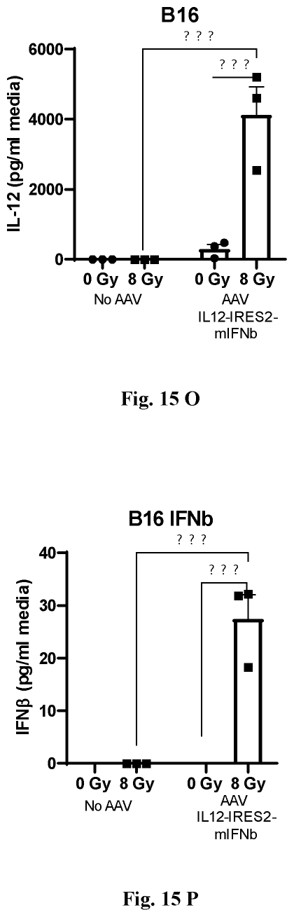

FIGS. 15 A-J show that cells transduced with the bicistronic vector containing the P2A element released IL-12 and IFN-β already in the absence of irradiation, although secretion of both IL-12 and IFN-β were markedly enhanced by prior irradiation.

### EXPERIMENT 2: USING F2A PRECEDED BY A GGSG LINKER AS A TRANSLATION REGULATORY ELEMENT SEPARATING THE SEQUENCES ENCODING THE IMMUNOMODULATORY PROTEINS

This experiment aims to compare the functionality of a bicistronic vector using a different translation regulatory element than experiment 1.

### Materials and Methods

A bicistronic vector, named AAV8-ISREx4-IL12-GGSGF2A-IFNbmiR122T, was constructed by placing the sequences of scIL-12 and IFN-β separated by a F2A element preceded by a linker domain consisting of the amino acids GGSG (SEQ ID NO: 82) under control of an ISREx4 promoter. The functionality of this bicistronic vector was tested in B16 murine tumor cells (melanoma). Cells (85.000 per well) were irradiated with 0 or 8 Gy and afterwards incubated in the presence (MOI 10⁵) or absence of said vector. The supernatant was collected after 48 h and IL-12 and IFN-β levels were measured by ELISA.

### Results and Discussion

FIGS. 15 K-L show that cells transduced with the bicistronic vector containing the GGSGF2A element released IL-12 and IFN-β already in the absence of irradiation, although secretion of both IL-12 and IFN-β were markedly enhanced by prior irradiation. Moreover, the expression levels of either immunomodulatory protein were comparable to that of experiment 1.

### EXPERIMENT 3: USING A SHORT IRES SEQUENCE (IRESGTX9) AS A TRANSLATION REGULATORY ELEMENT SEPARATING THE SEQUENCES ENCODING THE IMMUNOMODULATORY PROTEINS

This experiment aims to compare the functionality of a bicistronic vector using a different translation regulatory element than experiment 1.

### Materials and Methods

A bicistronic vector, named AAV8-ISREx4-IL12-IRESGtx9-IFNbmiR122T, was constructed by placing the sequences of scIL-12 and IFN-β separated by an IRESGtx9 element (SEQ ID NO: 83) under control of an ISREx4 promoter. The functionality of this bicistronic vector was tested in B16 murine tumor cells (melanoma). Cells (85.000 per well) were irradiated with 0 or 8 Gy and afterwards incubated in the presence (MOI 10⁵) or absence of said vector. The supernatant was collected after 48 h and IL-12 and IFN-β levels were measured by ELISA.

### Results and Discussion

FIGS. 15 M-N show that cells transduced with the bicistronic vector containing the IRESGtx9 element released IL-12 and IFN-β already in the absence of irradiation, although secretion of both IL-12 and IFN-β was markedly enhanced by prior irradiation. However, the expression levels of either immunomodulatory protein were reduced compared to these of experiments 1 or 2.

### EXPERIMENT 4: USING AN IRES2 SEQUENCE AS A TRANSLATION REGULATORY ELEMENT SEPARATING THE SEQUENCES ENCODING THE IMMUNOMODULATORY PROTEINS

This experiment aims to compare the functionality of a bicistronic vector using a different translation regulatory element than experiment 1.

### Materials and Methods

A bicistronic vector, named AAV8-ISREx4-IL12-IRES2-IFNbmiR122T, was constructed by placing the sequences of scIL-12 and IFN-β separated by an IRES2 element (SEQ ID NO: 84) under control of an ISREx4 promoter. The functionality of this bicistronic vector was tested in B16 murine tumor cells (melanoma). Cells (85.000 per well) were irradiated with 0 or 8 Gy and afterwards incubated in the presence (MOI 10⁵) or absence of said vector. The supernatant was collected after 48 h and IL-12 and IFN-β levels were measured by ELISA.

### Results and Discussion

FIGS. 15 O-P show that cells transduced with the bicistronic vector containing the IRES2 element released IL-12 and IFN-β already in the absence of irradiation, although secretion of both IL-12 and IFN-β was markedly enhanced by prior irradiation. However, the expression levels of either immunomodulatory protein were reduced compared to these of experiments 1 or 2.

### EXAMPLE 16: INTRAVENOUS ADMINISTRATION OF THE BICISTRONIC AAV8-ISREX4-IL12-IFNBMIR122T DOES NOT CAUSE ANY INCREASE IN SERUM CYTOKINES

A previous set of experiments already showed that the monocistronic AAV8-ISREx4-IL12miR122T does not cause any increase in serum cytokines. The aim of the following experiments was to confirm this for the bicistronic vector described in Example 15.

### Materials and Methods

Different groups of mice (n=5-7 per group) were injected with 5×1⁰ viral genomes of AAV8-ISREx4-IL12 (a vector which expresses scIL12 that does not contain miR122 binding sites at its 3′ end), AAV8-ISREx4-IL12miR122T or AAV8-ISREx4-IL12-IFNbmiR122T and after 2, 6 and 14 days were sampled to determine serum levels of IL-12 and IFN-β.

### Results and Discussion

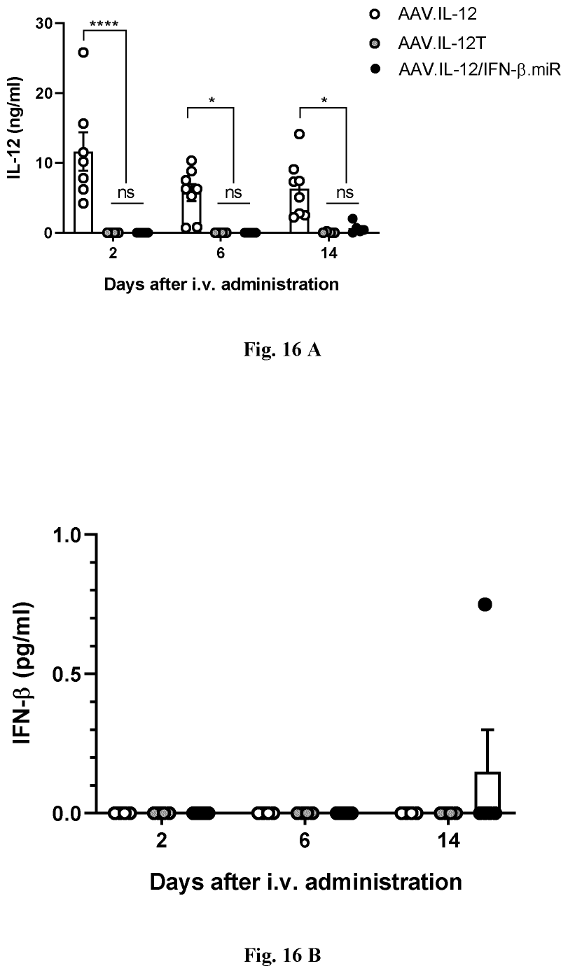

FIGS. 16 A and B show that serum levels of IL-12 were increased at the 3 times points in the animals that received AAV8-ISREx4-IL12 but not in those injected with AAV8-ISREx4-IL12miR122T or AAV8-ISREx4-IL12-IFNbmiR122T. Similarly, IFN-β was undetectable in all animals except at day 14 in only one mouse that had received the bicistronic vector. However, the elevation of serum IFN-β was negligible being below 0.8 μg/ml. These data clearly show that the presence of miR122 binding sites at the 3′ end of the expression cassette efficiently prohibits hepatic expression of the two transgenes contained in the bicistronic vector, arguing in favor of the safety of its administration. These data together with those of the previous section indicate that the two transgenes of the bicistronic vector are expressed in tumor cells (mainly in irradiated tumor cells) but not in liver cells rich in miR122.

### EXAMPLE 17: THE BICISTRONIC VECTOR AAV8-ISREX4-IL12-IFNBMIR122T EXERTS MORE POTENT ANTITUMOR EFFECTS IN DIFFICULT-TO-TREAT CANCERS

The previous examples showed the feasibility of the bicistronic vector in cell culture and its safety in in vivo applications. However, the aim of the following experiments was to examine the antitumor effects in difficult-to-treat cancers.

### Materials and Methods

Subcutaneous melanoma B16 tumors were generated in C57 mice by injecting subcutaneously 2.5×10⁵ B16 cells or the same amount of B16 B2M.KO cells (the latter lacking MHC class I expression and thereby function as an example of especially difficult-to-treat tumor). When tumors reached the size of 100-200 mm³ they were irradiated with 8 Gy and then injected with AAV8-ISREx4-IL12miR122T or AAV8-ISREx4-IL12-IFNbmiR122T and the evolution of tumor size was monitored by measuring tumor diameters with a caliper.

### Results and Discussion

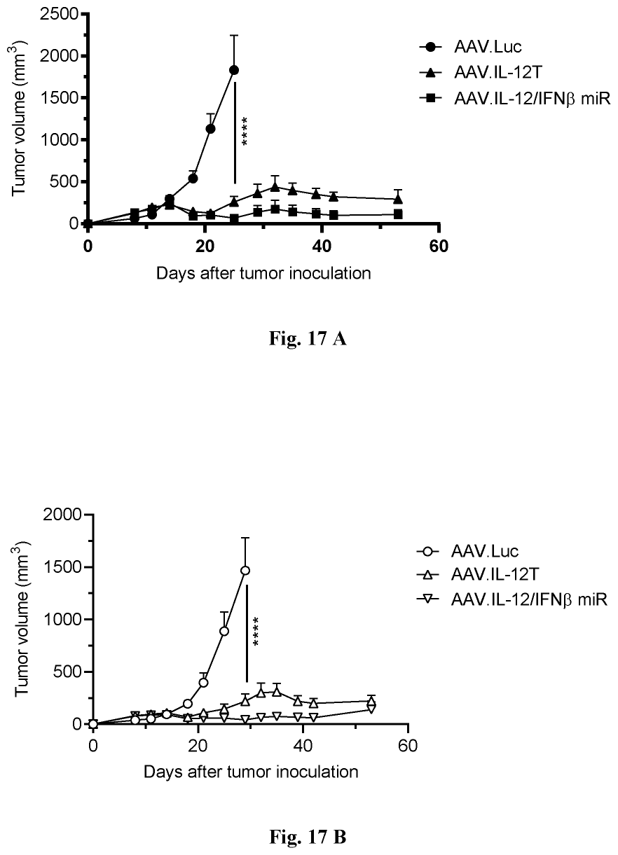

FIGS. 17 A and B show that both vectors exert a similar antitumor effect in B16 tumors compared to mock treated controls (circles). However, AAV8-ISREx4-IL12-IFNbmiR122T (rectangles) outcompeted AAV8-ISREx4-IL12miR122T (triangles) in the control of B16 B2M.KO tumors, although the difference in antitumor activity of the two vectors was slight. These data show that bicistronic vectors constitute a valuable adjunct to radiotherapy in oncology.

### EXAMPLE 18: AAV8-ISREX4-LUCMIR122T OR AAV8-ISREX4-IL12MIR122T ARE ABLE TO TARGET TRANSGENE EXPRESSION TO THE IRRADIATED TUMOR WHEN ADMINISTERED BY INTRAVENOUS ROUTE

The previous experiment confirmed that the mono- and bicistronic vector show potent antitumor effects when injected directly into the tumor. However, the following experiments aim to investigate whether also systemic application of the vectors may be a feasible approach to induce intratumor expression of the transgenes and may be a suitable treatment paradigm.

### EXPERIMENT 1: AAV8-ISREX4-LUCMIR122T IS ABLE TO TARGET TRANSGENE EXPRESSION TO THE IRRADIATED TUMOR WHEN ADMINISTERED BY INTRAVENOUS ROUTE

### Materials and Methods

Mice with two s.c. HCC tumors, one in each flank, (generated by s.c. injection of 5×10⁵ NM53 cells) were used. When the tumors reached the size of 100-200 mm³ the tumor of the right flank, but not the one of the left flank, was irradiated with 20 Gy. Immediately afterwards, the animals received an intravenous injection of 5×10¹⁰ viral genomes of AAV8-ISREx4-LucmiR122T and 5 days later luciferase activity was analyzed in the luminometer.

### Results and Discussion

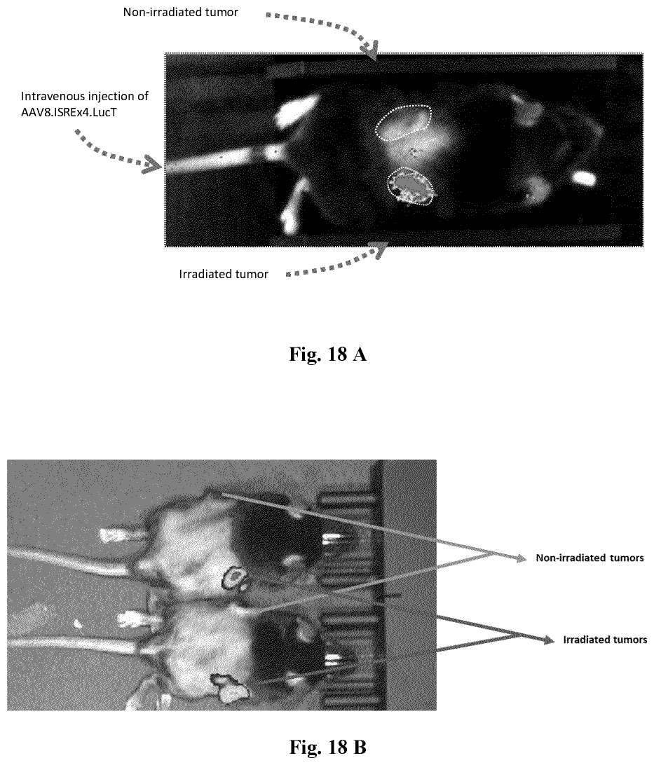

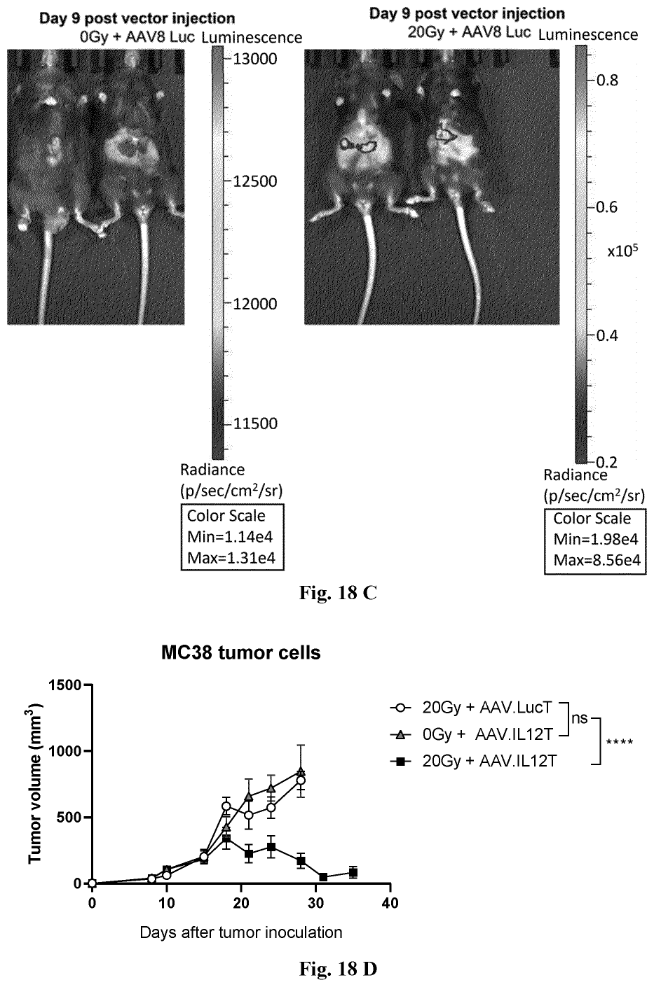

FIGS. 18 A to B show that luminescence signal was only detected in the right flank tumor which was subjected to radiation therapy. No significant signal was observed in any other part of the body, especially not in the contralateral non-irradiated tumor. Figure B is a repetition of the experiment performed in A. These findings indicate that radiotherapy marks the treated tumor for AAV transduction following intravenous vector administration. For this purpose, the vector should possess a radiation-responsive promoter and miR122 binding sites to prohibit transduction of liver cells.

### EXPERIMENT 2: AAV8-ISREX4-LUCMIR122T IS ABLE TO TARGET TRANSGENE EXPRESSION TO THE IRRADIATED TUMOR WHEN ADMINISTERED BY INTRAVENOUS ROUTE

The previous experiment shows that intravenously applied AAV8-ISREx4-LucmiR122T allows tumors to selectively express luciferase but only when said tumors receive irradiation. The following experiment aims to investigate whether also non-subcutaneous tumors may selectively express luciferase upon irradiation.

### Materials and Methods

For this, mice with orthotopic liver cancer were generated by intrahepatic injection of 200,000 NM53 hepatocarcinoma cells. One week later the tumors were irradiated with 0 or 20 Gy followed by intravenous injection of AAV.ISREx4LucmiR122T and the luciferase activity was analyzed in the luminometer.

### Results and Discussion

FIG. 18 C shows that luminescence (transgene expression) was observed in those tumors that were irradiated but not in those that were not irradiated. Importantly, this also shows that cancerous liver cells can be targeted by the expression system according to the invention. This indicates that miR122 levels in these cells are reduced to a degree where the expression of the transgene is not inhibited contrary to inhibition in physiologic hepatic cells.

### EXPERIMENT 3: INTRAVENOUS APPLICATION OF AAV8-ISREX4-IL12MIR122T IN COMBINATION WITH LOCALIZED RADIOTHERAPY CAUSES TUMOR REGRESSION

The experiments above demonstrated that also systemic application of the AAV causes transgene expression upon tumor irradiation. The next experiment aims to analyze whether also the systemic application of AAV8-ISREx4-IL12miR122T, comprising an immunomodulatory protein, may be a suitable paradigm for the treatment of tumors.

### Materials and Methods

Mice with a s.c. tumor in the right flank (generated by s.c. injection of 5×10⁵ MC38 cells) were used. When the tumors reached the size of about 200 mm³ the tumor was either irradiated with 20 Gy or as a control received no radiation (0 Gy). Immediately afterwards, the animals received an intravenous injection of either 1×10¹¹ viral genomes of AAV8-ISREx4-IL12miR122T or 1×10¹¹ viral genomes of AAV8-ISREx4-LucmiR122T. The evolution of tumor size was monitored as indicated.

### Results and Discussion

FIG. 18 D shows that tumors of mice that received intravenous application of AAV8-ISREx4-IL12miR122T continued to grow throughout the course of the experiment when they did not receive additional irradiation (upwards triangles). In contrast, when additional irradiation of 20 Gy was applied, tumor growth was halted (downwards triangles). The tumors of mice that received control virus (AAV8-ISREx4-LucmiR122T) also continued to grow throughout the course of the experiment. These data indicate that also systemic application of AAV8-ISREx4-IL12miR122T causes the expression of the transgene upon irradiation as tumor growth was halted. Thus, even a systemic application of the viral vector may be a suitable paradigm for the treatment of cancer. Moreover, these data corroborate that radiotherapy marks the tumor for transgene expression using the expression system according to the invention.

### EXAMPLE 19: AAV8.ISREX4.LUCMIR122T SHOWS HIGH EXPRESSION IN TUMORS THAT HAD RECEIVED HIGHER DOSES OF RADIATION (60 TO 100 GY)

The previous experiment showed that even relatively small amounts of irradiation allowed for strong and consistent transgene expression. The following experiments aim to analyze the effect of higher radiation doses and treatment paradigms on the transgene expression.

### Materials and Methods

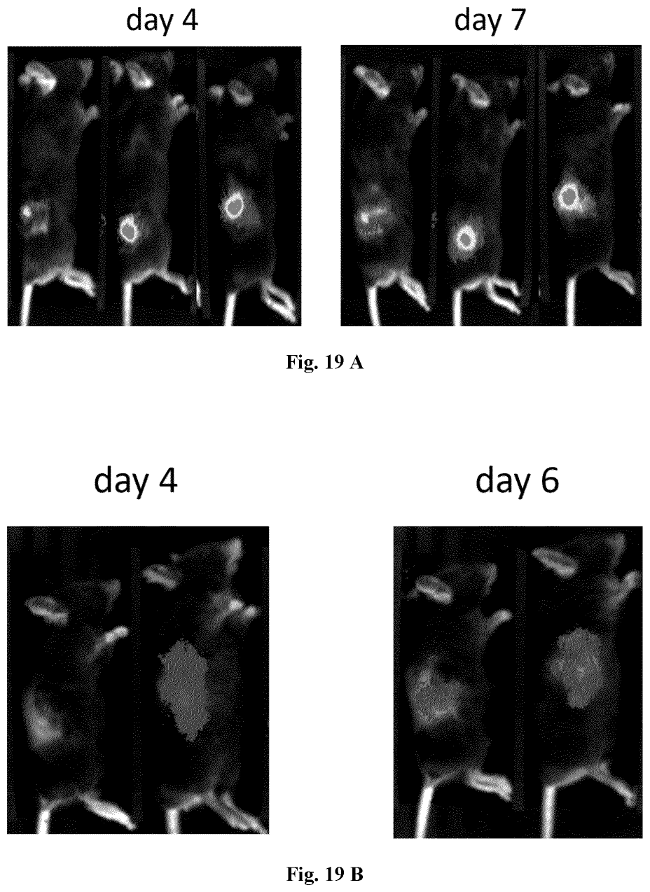

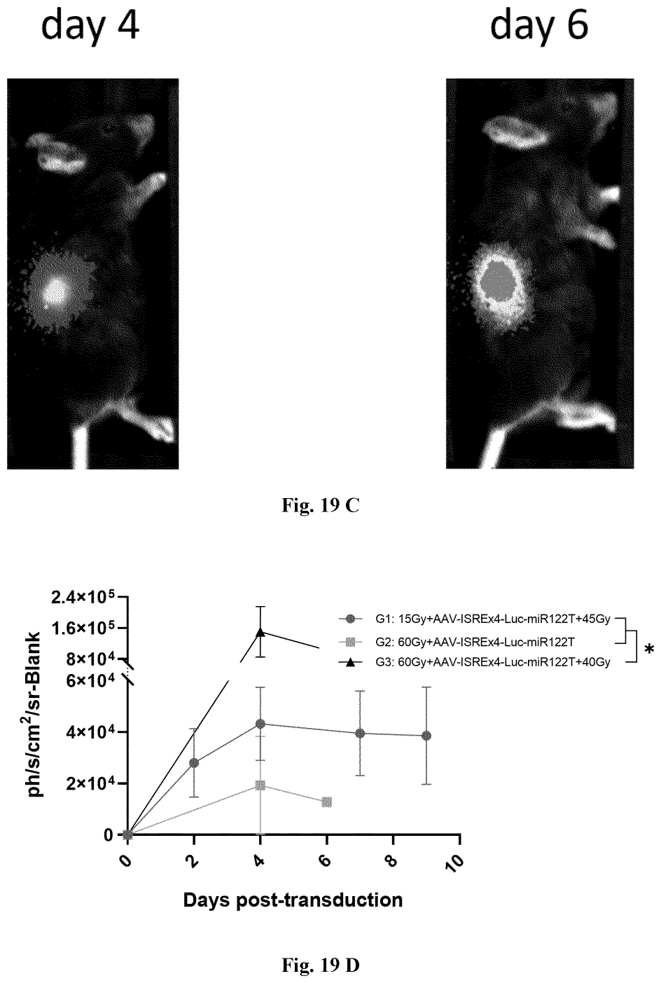

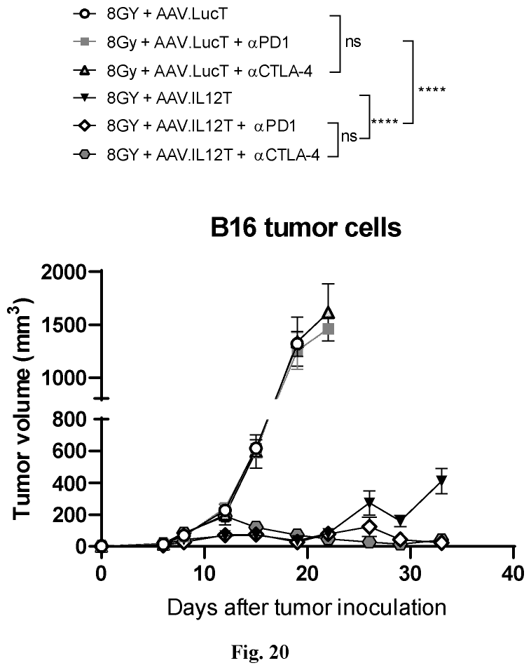

Mice with two s.c. HCC tumors, one in each flank, (generated by s.c. injection of 5×10⁵ MC38 cells) were used. When the tumors reached the size of 250-300 mm³ animals were divided into three groups. In group 1 (FIG. 19 A) tumors were irradiated with 15 Gy and injected immediately afterwards with 1×10¹¹ vg of AAV8.ISREx4.LucmiR122T. Then the tumors were irradiated daily with 15 Gy for 3 more days (total radiation=60 Gy). In group 2 (FIG. 19 B) tumors received 4 doses of 15 Gy (total radiation 60 Gy) followed by intratumor vector injection immediately after the last radiation dose. In group 3 (FIG. 19 C) animals were treated as in group 2 but the animals received two additional daily doses of 20 Gy (total radiation 100 Gy) after vector injection. In all cases luminescence was measured by Photoimager sequentially during the following days after vector injection.

### Results and Discussion

As shown in FIGS. 19 A-C and similarly to observations with lower radiation doses, gene expression from AAV8.ISREx4.LucmiR122T vector was robust and sustained in tumors subjected to higher radiation doses (up to 100 Gy). Detailed analysis of luminescence data (see FIG. 19 D) showed that additional radiation doses boosted transgene expression in irradiated tumors transduced with AAV8.ISREx4.LucmiR122T, indicating that radiation, in addition to facilitating tumor transduction by AAV vectors, stimulates the ISREx4 promoter that controls transgene expression in the AAV8.ISREx4.LucmiR122T.

Taken together, this shows that a wide range of radiation doses (between 2 and 100 Gy) promote AAV8.ISREx4.LucmiR122T transgene expression in tumors and that repeated fractional radiation doses boost the expression of the transgene from the AAV8.ISREx4.LucmiR122T and thus indicates that also transgene expression from other ISREx4-bearing AAV vectors may benefit from repeated fractional radiation doses.

### EXAMPLE 20: FURTHER COMBINATORY APPROACH USING IMMUNE CHECKPOINT INHIBITORS IN ADDITION TO RADIOTHERAPY AND IMMUNOGENE THERAPY

Example 13 already showed that the combination of radiotherapy and intratumoral AAV8.ISREx4-IL12miR122T causes profound infiltration of lymphocytes into the tumor vicinity. The following experiment aims to investigate whether a stimulation of the infiltrating lymphocytes by using immune checkpoint inhibitors causes further effects on tumor development.

### Materials and Methods

Mice with subcutaneous tumors were generated by s.c. injection of 500,000 B16 melanoma cells. Eight days after tumor cell injection, when the tumors reached a size of about 100-150 mm³, they were irradiated with 8 Gy and injected with AAV8.ISREx4.IL12miR122T or control vector AAV8.ISREx4.LucmiR122T (5×10e¹⁰ vg). Mice of each of these groups were subdivided into 3 groups (n=5-6 per group) which were treated respectively with the immune checkpoint inhibitor anti-PD1 or the immune checkpoint inhibitor anti-CTLA4 or nothing. Anti-PD1 or anti-CTLA4 monoclonal antibodies were administered at the dose of 100 micrograms given 3 times every three days, starting on the day of vector injection. The evolution of tumor size was monitored as indicated.

### Results and Discussion

Both anti-PD1 and anti-CTLA4 significantly enhance the antitumor effect of the therapy consisting of radiotherapy and intratumor injection of AAV8.ISREx4.IL12miR122T but not of radiotherapy alone (mice treated with AAV8.ISREx4.LucmiR122T). These data indicate that the therapy consisting of radiotherapy and intratumor injection of AAV8.ISREx4.IL12miR122T but not of radiotherapy alone convert tumors that are resistant to immune-checkpoint inhibitors into tumors that respond to this therapy. Thus cold tumors that are unresponsive to immune check-point inhibitors are converted into hot tumors that are sensitive to this therapy.

TABLE 5
Primers

| | | | | Amplicon |
|---|---|---|---|---|
| Gene | Species | Forward | Reverse | (pb) |
| Actin | Mouse | 5′-CGCGTCCACCCGCGAG-3′ | 5′-CCTGGTGCCTAGGGCG-3′ | 193 |
| | Human | 5′-ATTGCCGACAGGATGCAGAA-3′ | 5′-CGGACTCGTCATACTCCTGC-3′ | 172 |
| IFNb | Mouse | 5′-CCCTATGGAGATGACGGAGA-3′ | 5′-CTGTCTGCTGGTGGAGTTCA-3′ | 161 |
| | Human | 5′-TGCAGCAGTTCCAGAAGGAG-3′ | 5′-AGTCTCATTCCAGCCAGTGC-3′ | 107 |
| INFa | Mouse | 5′-CTTCCAGAACTCCAGGOGGT-3′ | 5′-GGTTTGCTACGACGTGGG-3′ | 227 |
| | Human | 5′-GTGCTTGTTCCTCAGCCTCT-3′ | 5′-ATGGGCTACAGGCTTGTCAC-3′ | 187 |
| STING | Mouse | 5′-CATACTACATTGGGTACTTGCGG-3′ | 5′-TGGAATAAACCCGATTCTTGATGC-3′ | 240 |
| | Human | 5′-GGTGACCGTGCTGGCATCAA-3′ | 5′-AAAGTCTGCAAGGGGGTGGC-3′ | 165 |
| scIL12 | Mouse | 5′-ACTGGACCAAAGGGACTATGAG-3′ | 5′-TGATGAAGAAGCTGGTGCTG-3′ | 149 |
| | Human | 5′-CCAAGTCAAAGAGTTTGGAGAT-3′ | 5′-AGGTGAAACGTCCAGAATAATT-3′ | 194 |
| IL6 | Mouse | 5′-ACAAAGCCAGAGTCCTTCAG-3′ | 5′-TGGATGGTCTTGGTCCTTAG-3′ | 163 |
| | Human | 5′-AGTGAGGAACAAGCCAGAGC-3′ | 5′-GGTCAGGGGTGGTTATTGCA-3′ | 100 |
| CXCL9 | Mouse | 5′-TCGAGGAACCCTAGTGATAAGG-3′ | 5′-TGAGGGATTTGTAGTGGATCG-3′ | 80 |
| | Human | 5′-ACCAACCAAGGGACTATCCAC-3 | 5′-TCAATTTTCTCGCAGGAAGG-3′ | 80 |
| CXCL10 | Mouse | 5′-AAGTGCTGCCGTCATTTTCT-3′ | 5′-GTGGCAATGATCTCAACACG-3 | 186 |
| | Human | 5′-TGCTGCCTTATCTTTCTGACTC-3′ | 5′-TGATGCAGGTACAGCGTACAG-3 | 76 |
| IL15 | Mouse | 5′-CAGAGGCCAACTGGATAGATG-3′ | 5′-GCAATTCCAGGAGAAAGCAG-3′ | 151 |
| | Human | 5′-CAAAGTAACAGCAATGAAGTGC-3′ | 5′-CAAACTGTTGTTTGCTAGGATG-3′ | 118 |
| IL7 | Mouse | 5′-CTGAGTGCCACATTAAAGAçAA-3′ | 5′-GATTTAGAAAAGCAGCTTCCTT-3′ | 171 |
| | Human | 5′-TTCAGAAGGCACAACAATACTG-3′ | 5′-GTTCTTTAGTGCCCATCAAAAT-3 | 209 |
| ITR | AAV2 | 5′-GGAACCCCTAGTGATGGAGTT-3′ | 5′-CGGCCTCAGTGAGCGA-3′ | >1500 |
| Luciferase | | 5′-AACCAGCGCCATTCTGATCA-3′ | 5′-CCAGTGTCTTACCGGTGTCC-3′ | 113 |
| cGFP | | 5′-ATCAAGGTGAACTTCAAGATCC-3′ | 5′-TTACTTGTACAGCTCGTCCATG-3′ | 237 |

1. A recombinant expression system that comprises
c. a first nucleic acid construct comprising a sequence encoding for a target STING, that is responsive to a STING agonist recognizing said target STING but not the human STING and natural variants thereof (human-non-reacting STING agonist), said sequence encoding the target STING is operatively linked to elements allowing expression and/or translation of said target STING; and/or
d. a second nucleic acid construct comprising a sequence encoding at least one immunomodulatory gene or protein, said sequence is operatively linked to a STING-responsive promoter or a radiation inducible promoter.

2. The recombinant expression system of claim 1, wherein the target STING is responsive to a human-non-reacting STING agonist selected from the group consisting of 5,6-dimethylxanthenone-4-acetic acid (DMXAA), 7-bromo-DMXAA, 7-iodo-DMXAA, 7-hydroxy-DMXAA, 7-formyl-DMXAA, 7-hydroxymethyl-DMXAA, 7-(2-hydroxyethyl)-DMXAA, flavone-8-acetic acid (FAA), 2,7-bis(2-dimethylamino ethoxy)fluoren-9-one (Tilorone) and 10-carboxymethyl-9-acridanone (CMA), and wherein preferably the target STING is responsive to DMXAA.

3. The recombinant expression system of claim 1 or 2, wherein the target STING encoded by the sequence of the first nucleic acid construct is
a. a mutated form of a natural variant of human STING that further comprises at least one, at least two, or at least three amino acid substitutions that renders the human STING variant sensitive to the human-non-reacting STING agonist as determined in an in vitro-agonist determination assay; or
b. a non-human STING that is sensitive to the human-non-reacting STING agonist as determined in an in vitro-agonist determination assay, preferably a murine STING or a hybrid STING, even more preferably a murine STING according to SEQ ID NO: 7;
wherein the target STING is preferably a DMXAA-sensitive human STING as determined in an in vitro-DMXAA determination assay.

4. The recombinant expression system of claim 3, wherein the target STING encoded by the sequence of the first nucleic acid construct is a DMXAA-sensitive human STING variant that is selected from the group consisting of:
a. a human STING[R] variant further comprising at least 1, at least 2, or at least 3 amino acid substitutions selected from the group consisting of S162A, G230I, and Q266I;
b. a human STING[AQ] variant further comprising at least 1, at least 2, or at least 3 amino acid substitutions selected from the group consisting of S162A, A230I, Q266I, E260I and E260V;
c. a human STING[H] variant further comprising at least 1, at least 2, or at least 3 amino acid substitutions selected from the group consisting of S162A, G230I, and Q266I; and
d. a truncated human STING[H] variant further comprising at least 1, or at least 2 amino acid substitutions selected from the group consisting of S162A and G2301.

5. The recombinant expression system of claim 4, wherein the DMXAA-sensitive human STING variant encoded by the sequence of the first nucleic acid construct is selected from the group consisting of hSTING[R]-S162A/G230I/Q266I, hSTING[R]-S162A/Q266I, hSTING[R]-G230I, hSTING[R]-S162A, hSTING[R]-Q266I, hSTING[R]-S162A/G230I, hSTING[AQ]-S162A/A230I/Q266I, hSTING[AQ]-S162A, hSTING[AQ]-S162A/R169A, hSTING[AQ]-S162A/T267A, hSTING[AQ]-E260I, hSTING[AQ]-E260V, hSTING[H]-S162A/G230I/Q266I, hSTING[H]-S162A/Q266I, hSTING[H]-G230I, hSTING[H]-S162A, hSTING[H]-S162A/G230I and hSTING[H]-Q2661 and preferably is hSTING[R]-S162A/G230I/Q266I.

6. The recombinant expression system of claim 4, wherein the sequence of the first nucleic acid construct encoding for the DMXAA-sensitive human STING variant is selected from the group consisting of:
a. SEQ ID NO: 5;
b. a polynucleotide sequence having at least 75%, at least 80%, at least 85%, at least 90% or at least 95% sequence identity with respect to SEQ ID NO: 5;
c. a polynucleotide sequence encoding a polypeptide comprising SEQ ID NO: 6;
d. a polynucleotide sequence encoding a polypeptide having at least 75%, at least 80%, at least 85%, at least 90% or at least 95% sequence identity with respect to SEQ ID NO: 6;
e. a polynucleotide sequence encoding a polypeptide to c) or d) with at least one modification of the polypeptide that is an amino acid substitution, amino acid insertion, amino acid deletion, C-terminal truncation or N-terminal truncation;
f. a polynucleotide sequence that hybridizes under at least stringent conditions to the complement of the polynucleotide sequence of any of a) to e);
g. a region of the polynucleotide sequence of a) to f) encoding a fragment of at least 50 amino acid residues of the DMXAA-sensitive human STING variant;
wherein the polypeptide encoded by the polynucleotide of any of a) to g) has the biological activity of being responsive to the agonist DMXAA as determined in an in vitro-DMXAA determination assay.

7. The recombinant expression system of any of the above claims, wherein the promoter of the first nucleic acid construct is a constitutive promoter, preferably selected from the list consisting of SV40, CMV0, UBC, EF1a, PGK, CAG, EFS, CBh, CBA human β actin, Ac5, Polyhedrin, TEF1, GDS, CaMV35S, Ubi, H1 and U6;
or
the promoter of the first nucleic acid construct is an inducible promoter, preferably selected from the list consisting of Telomerase-responsive promoter, TL-6 responsive promoter, TGF-β-inducible promoter, VEGFR-inducible promoter, Epidermal Growth factor receptor-inducible promoter, HER2/Neu-inducible promoter, MUC1 promoter, BRCA1 promoter, BRCA2 promoter, LALBA promoter, PSA promoter, ERBB2 promoter, CEA promoter, uPAR promoter and Thyroid transcription factor1 (TTF1).

8. The recombinant expression system of any of the above claims, wherein the at least one immunomodulatory gene or protein encoded by the sequence of the second nucleic acid construct is selected from the group consisting of interleukin-1, interleukin-2, interleukin-3, interleukin-4, interleukin-5, interleukin-7, interleukin-8, interleukin-9, interleukin-11, single-chain interleukin-12, interleukin-12, interleukin-13, interleukin-14, interleukin-15, interleukin-15-sushi, interleukin-16, interleukin-17, interleukin-18, interleukin-19, interleukin-20, interleukin-21, interleukin-22, interleukin-23, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, granulocyte-macrophage colony-stimulating factor, type I interferons, IFN-γ, TNF-α, FLT3-ligand, a blocking peptide targeting TGF-β, a blocking peptide targeting IL-10, a blocking peptide targeting FoxP3, a monoclonal antibody or single-chain variable fragment (scFv) or nanobody neutralizing PD1, PDL1, CTLA4, CD137, TIM3, LAG3, and a fragment or variant thereof,
or
a shRNA targeting TGF-β, a shRNA targeting IL-10, or a shRNA targeting FoxP3.

9. The recombinant expression system of any of the above claims, wherein the at least one immunomodulatory protein is selected from the group consisting of:
a. the alpha subunit of IL-12;
b. the β-subunit of IL-12;
c. single chain IL-12 comprising the α- and β-subunit of IL-12;
d. a single chain IL-12 comprising or consisting of the amino acid sequence of SEQ ID NO: 9 or 10;
e. a polypeptide having at least 70%, 75%, 80%, 85%, 90%, 95%, or 99% sequence identity with respect to SEQ ID NO: 9 or 10, wherein the polypeptide has the biological activity of interleukin-12;
f. an IFN-β comprising or consisting of the amino acid sequence of SEQ ID NO: 79;
g. a polypeptide having at least 70%, 75%, 80%, 85%, 90%, 95%, or 99% sequence identity with respect to SEQ ID NO: 79, wherein the polypeptide has the biological activity of IFN-β;
h. an IL-15-sushi, comprising or consisting of the amino acid sequence of SEQ ID NO: 80;
i. a polypeptide having at least 70%, 75%, 80%, 85%, 90%, 95%, or 99% sequence identity with respect to SEQ ID NO: 80, wherein the polypeptide has the biological activity of IL-15;
j. a CXCL10, comprising or consisting of the amino acid sequence of SEQ ID NO: 81;
k. a polypeptide having at least 70%, 75%, 80%, 85%, 90%, 95%, or 99% sequence identity with respect to SEQ ID NO: 81, wherein the polypeptide has the biological activity of CXCL10.

10. The recombinant expression system of any of the above claims, wherein the second nucleic acid construct comprises at least two sequences each encoding a different immunomodulatory protein, wherein
the first immunomodulatory protein is a single chain IL-12 comprising or consisting of the amino acid sequence of SEQ ID NO: 9 or 10, or a polypeptide having at least 70%, 75%, 80%, 85%, 90%, 95%, or 99% sequence identity with respect to SEQ ID NO: 9 or 10, wherein the polypeptide has the biological activity of interleukin-12, and the second immunomodulatory protein is an IFN-β comprising or consisting of the amino acid sequence of SEQ ID NO: 79, or a polypeptide having at least 70%, 75%, 80%, 85%, 90%, 95%, or 99% sequence identity with respect to SEQ ID NO: 79, wherein the polypeptide has the biological activity of IFN-β; or
an IL-15-sushi, comprising or consisting of the amino acid sequence of SEQ ID NO: 80, or a polypeptide having at least 70%, 75%, 80%, 85%, 90%, 95%, or 99% sequence identity with respect to SEQ ID NO: 80, wherein the polypeptide has the biological activity of IL-15; or
a CXCL10, comprising or consisting of the amino acid sequence of SEQ ID NO: 81, or a polypeptide having at least 70%, 75%, 80%, 85%, 90%, 95%, or 99% sequence identity with respect to SEQ ID NO: 81, wherein the polypeptide has the biological activity of CXCL10.

11. The recombinant expression system of any of the above claims, wherein the second nucleic acid construct is a bi- or polycistronic expression cassette comprising two or more sequences each encoding a different immunomodulatory protein, or a protein or an RNA with anticancer properties, wherein
a. the sequences are separated by a sequence encoding an IRES; and/or
b. the sequences are separated by a sequence encoding a 2A peptide, preferably a sequence encoding a P2A peptide.

12. The recombinant expression system of any of the above claims, wherein the STING-responsive promoter of the second nucleic acid construct is selected from the group consisting of IFN-β-responsive promoter, IFN-β-minimal promoter, CXCL9-responsive promoter, CXCL10-responsive promoter, STAT-motif promoter, IRSF3-motif promoter, ISRE-motif promoter, NFκB-motif promoter, and STAT6-motif promoter, wherein the STING responsive promoter preferably comprises at least one interferon-stimulated response element (ISRE) and more preferably is selected from the list consisting of:
a. a nucleotide sequence comprising one to ten copies, preferably two to seven copies, more preferably three to five copies and particularly preferably 4 copies of a sequence having at least 75%, at least 80%, at least 85%, at least 90% or at least 95% sequence identity with respect to SEQ ID NO: 11 or is identical to SEQ ID NO: 11;
b. a nucleotide sequence according to a., wherein the copies are interspaced by up to forty nucleotides, preferably four to thirty-five nucleotides, more preferably ten to thirty nucleotides and particularly preferably fifteen to twenty-five nucleotides;
c. a nucleotide sequence according to SEQ ID NO: 12;
d. a nucleotide sequence according to SEQ ID NO: 13.

13. The recombinant expression system of any of the above claims, wherein the second nucleic acid construct further comprises at least one sequence motif that inhibits transgenic expression of the immunomodulatory protein in tissues or cells not intended to express the proteins, the tissue preferably being liver, wherein the sequence motif is preferably a target sequence of a microRNA that is highly abundant in the liver, more preferably the target sequence is selected from the group consisting of miR-122 target sequence, miR-192 target sequence, miR-199a target sequence, miR-101 target sequence, miR-99a target sequence, let7a target sequence, let7b target sequence, let7c target sequence, let7f target sequence, and even more preferably is a miR-122 target sequence selected from the list consisting of:
a. a miR-122 target sequence having at least 90% or at least 95% sequence identity with respect to SEQ ID NO: 14 or is identical to SEQ ID NO: 14;
b. a nucleotide sequence comprising 1 to 12, preferably 3 to 10, more preferably 4 to 8 and particularly preferably 5 copies of the miR-122 target sequence of a.;
c. a nucleotide sequence according to b., wherein the copies are interspaced by one to forty nucleotides, preferably one to thirty nucleotides, more preferably two to twenty nucleotides and particularly preferably two to eight nucleotides;
d. a nucleotide sequence comprising 5 copies of the miR-122 target sequence as depicted in SEQ ID NO: 15.

14. An expression vector encoding the first and/or the second nucleic acid construct according to any of the above claims or a combination of a first and a second expression vector, the first vector encodes the first nucleic acid construct according to any of the above claims and the second vector encodes the second nucleic acid construct according to any of the above claims, wherein the expression vector(s) is (are) preferably a viral vector, more preferably a viral vector selected from the list consisting of adeno-associated virus (AAV) vector, adenoviral vector, lentiviral vector, vaccine virus vector, or herpes simplex virus vector, even more preferably an adeno-associated virus vector having one or more of the following characteristics:
a. the first and/or the second nucleic acid construct sequence further comprise a 5′ITR and a 3′ITR sequence, being preferably AAV ITRs as depicted in SEQ ID NO; 17;
b. the AAV vector has preferably the serotype AAV8, AAV1, AAV3, AAV6, AAV9, AAV2, AAV5, AAVrh.10, or any gain-of-function mutant of AAVrh.10, more preferably the AAV vector has the serotype AAV2.

15. A viral particle comprising the viral vector comprising the first and/or second nucleic acid construct of claim 14 or a combination of a first and a second viral particle, the first viral particle comprises a viral vector encoding the first nucleic acid construct and the second viral particle comprises a viral vector encoding the second nucleic acid construct of claim 14, wherein the viral particle(s) is (are) preferably (an) AAV or (an) adenoviral particle(s); more preferably (an) AAV viral particle(s).

16. A pharmaceutical composition that comprises a pharmaceutically acceptable excipient and:
a. the recombinant expression system of any of claims 1 to 13; or
b. the expression vector(s) of claim 14; or
c. the viral particle(s) of claim 15.

17. The recombinant expression system of any of claims 1 to 13; the expression vector(s) of claim 14; the viral particle(s) of claim 15 or the pharmaceutical composition according to claim 16 for use in a therapy, preferably an immunotherapy, more preferably in the immunotherapy of a proliferative disorder, even more preferably in the immunotherapy of cancer, particularly preferably in the immunotherapy of solid cancer.

18. The recombinant expression system, expression vector(s), the viral particle(s) or pharmaceutical composition for use according to claim 17, wherein the recombinant expression system, the expression vector(s), the viral particle(s) or the pharmaceutical composition is employed in a combination therapy with at least one other therapy selected from the group consisting of
a. a therapy comprising at least one checkpoint inhibitor, preferably selected from the group consisting of an inhibitor of PD-1, PD-L1, PD-L2, CTLA-4, LAG3, BTLA, B7H3, B7H4, TIM3, KIR, or A2aR, more preferably selected from the group consisting of an inhibitor of PD-1, PD-L1, PD-L2, or CTLA-4; and/or
b. a therapy comprising at least one adoptive cell therapy, preferably selected from adoptive NK cell therapy, adoptive dendritic cell therapy, or adoptive T-cell therapy, wherein the adoptive T-cell therapy preferably is a CAR-T-cell therapy or a therapy using TIL; and/or
c. a therapy comprising at least one cancer vaccine; and/or
d. a therapy comprising at least one exogenous cytokine; and/or
e. a radiotherapy.

19. The recombinant expression system, expression vector(s), the viral particle(s) or pharmaceutical composition for use according to claim 17 or 18, wherein the cancer is selected from the list consisting of. bladder cancer, bone cancer, brain cancer, breast cancer, cancer of the thymus, cervical cancer, colon cancer, oesophageal cancer, gastric cancer, head & neck cancers, Hodgkin's lymphoma, primary liver cancer, metastatic liver cancer, gallbladder cancer, lung cancer, melanoma, mesothelioma, multiple myeloma, Merkel cell carcinoma, non-Hodgkin's lymphoma, ovarian cancer, pancreatic cancer, prostate cancer, rectal cancer, renal cancer, sarcoma, skin cancer, testicular cancer, thyroid cancer, uterine cancer, plasma cell tumors, cholangiocarcinoma, and neuroendocrine tumors or carcinoid tumors.

20. The recombinant expression system, expression vector(s), the viral particle(s) or pharmaceutical composition for use according to any of claims 17 to 19, wherein the use in the immunotherapy is a combination of:
a. administration of the recombinant expression system of any of claims 1 to 13; the expression vector(s) of claim 14; the viral particle(s) of claim 15 or the pharmaceutical composition according to claim 16 to a patient suffering from a proliferative disorder, being preferably cancer in order to yield a therapeutically effective transgenic expression of the target STING and/or the immunomodulatory protein, wherein the administration is preferably an intratumoral administration or a systemic administration;
b. optionally treating the patient before and/or concurrently to the administration according to step a) with a radiotherapy, being preferably a radiotherapy directed to the tissue or organ intended to express the transgene(s) from a); and
c. treating the patient after the administration according step a) with a STING agonist recognizing the transgenically expressed target STING but not the human STING and natural variants thereof and/or with a radiotherapy, being preferably a radiotherapy directed to the tissue or organ intended to express the transgene(s) from a).

21. The recombinant expression system, expression vector(s), the viral particle(s) or pharmaceutical composition for use according to claim 20, wherein the patient is treated before, concurrently and/or after the administration according to step a) with at least one other therapy according to claim 18, preferably the patient is treated after the administration according to step a).

22. The recombinant expression system, expression vector(s), the viral particle(s) or pharmaceutical composition for use according to claim 20 or 21, wherein in step c) a STING agonist according to claim 2 is used, being preferably DMXAA.

23. The recombinant expression system, expression vector(s), the viral particle(s) or pharmaceutical composition for use according to any of claims 20 to 22, wherein in step b) a radiotherapy with a dose of less than 100 Gy is used or a low dose radiotherapy with 40 Gy or less, preferably 8 Gy or less is used.

24. The recombinant expression system, expression vector, the viral particle or pharmaceutical composition for use according to any of claims 20 to 23, wherein step c) comprises several separate radiation treatments, wherein the cumulated dose of the separate radiation treatments adds up to less than 100 Gy, preferably to less than 40 Gy and more preferably to less than 25 Gy.

25. A recombinant expression system that consists of a nucleic acid construct comprising a sequence encoding at least two immunomodulatory proteins, said sequence is operatively linked to a STING-responsive promoter or a radiation inducible promoter and at least one sequence motif that inhibits transgenic expression of the immunomodulatory proteins in tissues or cells not intended to express the proteins.

26. The recombinant expression system according to claim 25, wherein the at least two immunomodulatory proteins are selected from the group consisting of interleukin-1, interleukin-2, interleukin-3, interleukin-4, interleukin-5, interleukin-7, interleukin-8, interleukin-9, interleukin-11, single-chain interleukin-12, interleukin-12, interleukin-13, interleukin-14, interleukin-15, interleukin-15-sushi, interleukin-16, interleukin-17, interleukin-18, interleukin-19, interleukin-20, interleukin-21, interleukin-22, interleukin-23, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, granulocyte-macrophage colony-stimulating factor, type I interferons, IFN-γ, TNF-α, FLT3-ligand, a blocking peptide targeting TGF-β, a blocking peptide targeting IL-10, a blocking peptide targeting FoxP3, a monoclonal antibody or single-chain variable fragment (scFv) or nanobody neutralizing PD1, PDL1, CTLA4, CD137, TIM3, LAG3, and a fragment or variant thereof.

27. The recombinant expression system according to claim 25 or 26, wherein the at least two immunomodulatory proteins are selected from the group consisting of
a. the alpha subunit of IL-12;
b. the β-subunit of IL-12;
c. single chain IL-12 comprising the α- and β-subunit of IL-12;
d. a single chain IL-12 comprising or consisting of the amino acid sequence of SEQ ID NO: 9 or 10;
e. a polypeptide having at least 70%, 75%, 80%, 85%, 90%, 95%, or 99% sequence identity with respect to SEQ ID NO: 9 or 10, wherein the polypeptide has the biological activity of interleukin-12;
f. an IFN-β comprising or consisting of the amino acid sequence of SEQ ID NO: 79;
g. a polypeptide having at least 70%, 75%, 80%, 85%, 90%, 95%, or 99% sequence identity with respect to SEQ ID NO: 79, wherein the polypeptide has the biological activity of IFN-β;
h. an IL-15-sushi, comprising or consisting of the amino acid sequence of SEQ ID NO: 80;
i. a polypeptide having at least 70%, 75%, 80%, 85%, 90%, 95%, or 99% sequence identity with respect to SEQ ID NO: 80, wherein the polypeptide has the biological activity of IL-15;
j. a CXCL10, comprising or consisting of the amino acid sequence of SEQ ID NO: 81;
k. a polypeptide having at least 70%, 75%, 80%, 85%, 90%, 95%, or 99% sequence identity with respect to SEQ ID NO: 81, wherein the polypeptide has the biological activity of CXCL10.

28. The recombinant expression system according to any of claims 25 to 27, wherein the first immunomodulatory protein is a single chain IL-12 comprising or consisting of the amino acid sequence of SEQ ID NO: 9 or 10, or a polypeptide having at least 70%, 75%, 80%, 85%, 90%, 95%, or 99% sequence identity with respect to SEQ ID NO: 9 or 10, wherein the polypeptide has the biological activity of interleukin-12, and
the second immunomodulatory protein is an IFN-β comprising or consisting of the amino acid sequence of SEQ ID NO: 79, or a polypeptide having at least 70%, 75%, 80%, 85%, 90%, 95%, or 99% sequence identity with respect to SEQ ID NO: 79, wherein the polypeptide has the biological activity of IFN-β; or
an IL-15-sushi, comprising or consisting of the amino acid sequence of SEQ ID NO: 80, or a polypeptide having at least 70%, 75%, 80%, 85%, 90%, 95%, or 99% sequence identity with respect to SEQ ID NO: 80, wherein the polypeptide has the biological activity of IL-15; or
a CXCL10, comprising or consisting of the amino acid sequence of SEQ ID NO: 81, or a polypeptide having at least 70%, 75%, 80%, 85%, 90%, 95%, or 99% sequence identity with respect to SEQ ID NO: 81, wherein the polypeptide has the biological activity of CXCL10.

29. The recombinant expression system according to any of claims 25 to 28, wherein the nucleic acid construct is a bi- or polycistronic expression cassette comprising two or more sequences each encoding a different immunomodulatory protein, or a protein or an RNA with anticancer properties, wherein
a. the sequences are separated by a sequence encoding an IRES; and/or
b. the sequences are separated by a sequence encoding a 2A peptide, preferably a sequence encoding a P2A peptide.

30. The recombinant expression system according to any of claims 25 to 29, wherein the STING-responsive promoter of the nucleic acid construct is selected from the group consisting of IFN-β-responsive promoter, IFN-β-minimal promoter, CXCL9-responsive promoter, CXCL10-responsive promoter, STAT-motif promoter, IRSF3-motif promoter, ISRE-motif promoter, NFκB-motif promoter, and STAT6-motif promoter, wherein the STING responsive promoter preferably comprises at least one interferon-stimulated response element (ISRE) and more preferably is selected from the list consisting of:
a. a nucleotide sequence comprising one to ten copies, preferably two to seven copies, more preferably three to five copies and particularly preferably 4 copies of a sequence having at least 75%, at least 80%, at least 85%, at least 90% or at least 95% sequence identity with respect to SEQ ID NO: 11 or is identical to SEQ ID NO: 11;
b. a nucleotide sequence according to a., wherein the copies are interspaced by up to forty nucleotides, preferably four to thirty-five nucleotides, more preferably ten to thirty nucleotides and particularly preferably fifteen to twenty-five nucleotides;
c. a nucleotide sequence according to SEQ ID NO: 12;
d. a nucleotide sequence according to SEQ ID NO: 13.

31. The recombinant expression system according to any of claims 25 to 30, wherein the sequence motif that inhibits transgenic expression of the immunomodulatory proteins in tissues or cells not intended to express the proteins, the tissue preferably being liver, wherein the sequence motif is preferably a target sequence of a microRNA that is highly abundant in the liver, more preferably said target sequence is selected from the group consisting of miR-122 target sequence, miR-192 target sequence, miR-199a target sequence, miR-101 target sequence, miR-99a target sequence, let7a target sequence, let7b target sequence, let7c target sequence, let7f target sequence, and even more preferably is a miR-122 target sequence selected from the list consisting of:
a. a miR-122 target sequence having at least 90% or at least 95% sequence identity with respect to SEQ ID NO: 14 or is identical to SEQ ID NO: 14;
b. a nucleotide sequence comprising 1 to 12, preferably 3 to 10, more preferably 4 to 8 and particularly preferably 5 copies of the miR-122 target sequence of a.;
c. a nucleotide sequence according to b., wherein the copies are interspaced by one to forty nucleotides, preferably one to thirty nucleotides, more preferably two to twenty nucleotides and particularly preferably two to eight nucleotides;
d. a nucleotide sequence comprising 5 copies of the miR-122 target sequence as depicted in SEQ ID NO: 15.

32. An expression vector encoding the nucleic acid construct according to any of claims 25 to 31, wherein the expression vector is preferably a viral vector, more preferably a viral vector selected from the list consisting of adeno-associated virus (AAV) vector, adenoviral vector, lentiviral vector, vaccine virus vector, or herpes simplex virus vector, even more preferably an adeno-associated virus vector having one or more of the following characteristics:
a. the nucleic acid construct sequence further comprises a 5′ITR and a 3′ITR sequence, being preferably AAV ITRs as depicted in SEQ ID NO; 17;
b. the AAV vector has preferably the serotype AAV2, AAV1, AAV3, AAV6, AAV9, AAV8, AAV5, AAVrh.10, or any gain-of-function mutant of AAVrh.10, more preferably the AAV vector has the serotype AAV2.

33. A viral particle comprising the viral vector comprising the nucleic acid construct of claim 32, wherein the viral particle is preferably an AAV or an adenoviral particle; more preferably an AAV viral particle.

34. A pharmaceutical composition that comprises a pharmaceutically acceptable excipient and:
a. the recombinant expression system of any of claims 25 to 31; or
b. the expression vector of claim 32; or
c. the viral particle of claim 33.

35. The recombinant expression system of any of claims 25 to 31; the expression vector of claim 32; the viral particle of claim 33 or the pharmaceutical composition according to claim 34 for use in a therapy, preferably an immunotherapy, more preferably in the immunotherapy of a proliferative disorder, even more preferably in the immunotherapy of cancer, particularly preferably in the immunotherapy of solid cancer.

36. The recombinant expression system, expression vector, the viral particle or pharmaceutical composition for use according to claim 35, wherein the recombinant expression system, the expression vector, the viral particle or the pharmaceutical composition is employed in a combination therapy with at least one other therapy selected from the group consisting of
a. a therapy comprising at least one checkpoint inhibitor, preferably selected from the group consisting of an inhibitor of PD-1, PD-L1, PD-L2, CTLA-4, LAG3, BTLA, B7H3, B7H4, TIM3, KIR, or A2aR, more preferably selected from the group consisting of an inhibitor of PD-1, PD-L1, PD-L2, or CTLA-4; and/or
b. a therapy comprising at least one adoptive cell therapy, preferably selected from adoptive NK cell therapy, adoptive dendritic cell therapy, or adoptive T-cell therapy, wherein the adoptive T-cell therapy preferably is a CAR-T-cell therapy or a therapy using TIL; and/or
c. a therapy comprising at least one cancer vaccine; and/or
d. a therapy comprising at least one exogenous cytokine; and/or
e. a therapy comprising a radiotherapy.

37. The recombinant expression system, expression vector, the viral particle or pharmaceutical composition for use according to claim 35 or 36, wherein the cancer is selected from the list consisting of. bladder cancer, bone cancer, brain cancer, breast cancer, cancer of the thymus, cervical cancer, colon cancer, oesophageal cancer, gastric cancer, head & neck cancers, Hodgkin's lymphoma, primary liver cancer, metastatic liver cancer, gallbladder cancer, lung cancer, melanoma, mesothelioma, multiple myeloma, urothelial carcinoma, gallbladder carcinoma, Merkel cell carcinoma, non-Hodgkin's lymphoma, ovarian cancer, pancreatic cancer, prostate cancer, rectal cancer, renal cancer, sarcoma, skin cancer, testicular cancer, thyroid cancer, uterine cancer, plasma cell tumors, cholangiocarcinoma, and, neuroendocrine tumors or carcinoid tumors.

38. The recombinant expression system, expression vector, the viral particle or pharmaceutical composition for use according to any of claims 35 to 37, wherein the use in the immunotherapy is a combination of:
a. administration of the recombinant expression system of any of claims 25 to 31; the expression vector of claim 32; the viral particle of claim 33 or the pharmaceutical composition according to claim 34 to a patient suffering from a proliferative disorder, being preferably cancer in order to yield a therapeutically effective transgenic expression of the immunomodulatory proteins, wherein the administration is preferably an intratumoral administration or a systemic administration;
b. optionally treating the patient before and/or concurrently to the administration according to step a) with a radiotherapy, being preferably a radiotherapy directed to the tissue or organ intended to express the transgene(s) from a); and
c. treating the patient after the administration according step a) with a radiotherapy, being preferably a radiotherapy directed to the tissue or organ intended to express the transgene(s) from a).

39. The recombinant expression system, expression vector(s), the viral particle(s) or pharmaceutical composition for use according to claim 38, wherein the patient is treated before, concurrently and/or after the administration according to step a) with at least one other therapy according to claim 36, preferably the patient is treated after the administration according to step a).

40. The recombinant expression system, expression vector, the viral particle or pharmaceutical composition for use according to claim 38 or 39, wherein in step b) a radiotherapy with a dose of less than 100 Gy is used or a low dose radiotherapy with 40 Gy or less, preferably 8 Gy or less is used.

41. The recombinant expression system, expression vector, the viral particle or pharmaceutical composition for use according to any of claims 38 to 40, wherein step c) comprises several separate radiation treatments, wherein the cumulated dose of the separate radiation treatments adds up to less than 100 Gy, preferably to less than 40 Gy and more preferably to less than 25 Gy.
